(19) United States
(12) Patent Application Publication
(10) Pub. No.: US 2019/0022225 A1
(43) Pub. Date: Jan. 24, 2019

(54) PROTOCOL FOR INCREASING LIFE EXPECTANCY IN A TEST SUBJECT

(71) Applicant: Mayo Foundation for Medical Education and Research, Rochester, MN (US)

(72) Inventors: Darren J. Baker, Rochester, MN (US); Bennett G. Childs, Rochester, MN (US); James L. Kirkland, Rochester, MN (US); Tamar Tchkonia, Rochester, MN (US); Jan M.A. van Deursen, Rochester, MN (US)

(21) Appl. No.: 16/007,825

(22) Filed: Jun. 13, 2018

Related U.S. Application Data
(63) Continuation of application No. 15/742,458, filed on Jan. 5, 2018.
(60) Provisional application No. 62/190,191, filed on Jul. 8, 2015.
(60) Provisional application No. 62/195,209, filed on Jul. 21, 2015.
(60) Provisional application No. 62/289,097, filed on Jan. 29, 2016.

(51) Int. Cl. A61K 45/06; A61K 31/428; A61P 35/00; A61K 31/40; A61K 31/4035; A61K 31/407; C07K 14/47; A61K 31/635; A61K 31/496; A61K 31/4375; A61P 9/10; C12N 15/113

(52) U.S. Cl. CPC A61K 45/06; C12N 2310/531; A61P 35/00; A61K 31/40; A61K 31/4035; A61K 31/407; C07K 14/4747; A61K 31/635; A61K 31/496; A61K 31/4375; A61P 9/10; A01K 2227/105; A01K 2267/0375; C12N 15/113; C12N 2310/14; A61K 31/428

(57) ABSTRACT

Methods are provided herein for selectively killing senescent cells and for treating senescence-associated diseases and disorders by administering a senolytic agent. Senescence-associated diseases and disorders treatable by the methods using the senolytic agents described herein include cardiovascular diseases and disorders associated with or caused by arteriosclerosis, such as atherosclerosis; idiopathic pulmonary fibrosis; chronic obstructive pulmonary disease; osteoarthritis; senescence-associated ophthalmic diseases and disorders; and senescence-associated dermatological diseases and disorders. Also included herein are methods for extending lifespan.

### CROSS-REFERENCE TO RELATED APPLICATIONS

This application is a continuation of U.S. application Ser. No. 15/742,458, filed Jan. 5, 2018, which is a U.S. 371 of International Application PCT/US2016/41646, filed Jul. 8, 2016, which claims the benefit of U.S. provisional patent application Ser. No. 62/190,191, filed Jul. 8, 2015, which is hereby incorporated by reference in its entirety; the present application claims the benefit of U.S. Provisional Application Ser. No. 62/195,209, filed Jul. 21, 2015, which is hereby incorporated by reference in its entirety; and the present application claims the benefit of U.S. Provisional Application Ser. No. 62/289,097, filed Jan. 29, 2016, which is hereby incorporated by reference in its entirety.

### STATEMENT AS TO FEDERALLY SPONSORED RESEARCH

This invention was made with the support of the United States government under Grant No. AG009909, AG017242, HL111121, AG041122 and AG046061 awarded by the National Institutes of Health. The government has certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 44327-735.301-Sequence-Listing.txt. The text file is 5.46 KB, was created on Jun. 13, 2018, and is being submitted electronically via EFS-Web,

### SUMMARY OF THE INVENTION

In some embodiments this disclosure provides a method for extending lifespan of a subject comprising administering to the subject a compound that selectively kills senescent cells over non-senescent cells. In some aspects the extending lifespan of the subject comprises delaying onset of an age-related disease or condition.

In some embodiments this disclosure provides a method for delaying onset or progression of an age-related disease or condition in a subject comprising administering to the subject a compound that selectively kills senescent cells over non-senescent cells. In some aspects the method delays the onset of an age-related disease or condition. In some aspects the method delays the progression of an age-related disease or condition. In some aspects the age-related disease or condition is selected from atherosclerosis, cardiovascular disease, cancer, arthritis, dementia, cataract, osteoporosis, diabetes, hypertension, age-related fat loss, vertebral disc degeneration, age-related muscular atrophy and kidney disease. In some aspects the age related disease or condition is kidney disease. In some aspects the method comprises identifying a patient at risk of developing a kidney disease. In some aspects the method comprises identifying a patient presenting at least one symptom of a kidney disease. In some aspects delaying the onset or progression of kidney disease comprises delaying the onset or progression of at least one symptom of kidney disease. In some aspects a symptom of kidney disease is delayed for at least one month after diagnosis of kidney disease in the subject. In some aspects a symptom of kidney disease is delayed for at least six months after diagnosis of the kidney disease in the subject. In some aspects the symptom is at least one symptom selected from the list consisting of decreased glomerular filtration rate, elevated blood urea nitrogen (BUN) content, increased serum creatinine, proteinuria and formation of sclerotic glomeruli. In some aspects the symptom is decreased glomerular filtration rate. In some aspects delaying the onset or progression of the decreased glomerular filtration rate comprises maintaining a glomerular filtration rate of at least 70. n some aspects delaying the onset or progression of impaired glomerular filtration comprises maintaining a glomerular filtration rate of at least 90. In some aspects the symptom is elevated blood urea nitrogen (BUN) levels. In some aspects delaying the onset or progression of elevated blood urea nitrogen levels comprises maintaining a blood urea nitrogen level of from 5 to 30. In some aspects delaying the onset or progression of elevated blood nitrogen levels comprises maintaining a blood urea level of from 7 to 20. In some aspects delaying the onset or progression of kidney disease comprises ameliorating at least one symptom of kidney disease. In some aspects the symptom is selected from the list of symptoms consisting of decreased glomerular filtration rate, elevated blood urea nitrogen (BUN) content, increased serum creatinine, proteinuria and formation of sclerotic glomeruli. In some aspects the symptom is formation of sclerotic glomeruli. In some aspects administering the compound to the subject reduces the number of sclerotic glomeruli relative to a pre-treatment number of sclerotic glomeruli. In some aspects the number of sclerotic glomeruli are reduced by at least 15% or more relative to a pre-treatment value of sclerotic glomeruli. In some aspects the symptom is decreased glomerular filtration rate. In some aspects the glomerular filtration rate in the subject is increased relative to a pre-treatment value of glomerular filtration rate. In some aspects administering the compound to the subject increases the glomerular filtration rate by at least 20% relative to a pre-treatment value of glomerular filtration rate. In some aspects the symptom is elevated blood urea nitrogen level. In some aspects the blood urea nitrogen level in the subject is reduced relative to a pre-treatment value of blood urea nitrogen level. In some aspects the blood nitrogen level in the subject is reduced by at least 10% relative to a pre-treatment value of blood urea nitrogen level. In some aspects the blood nitrogen level in the subject is reduced by at least 50% relative to a pre-treatment value of blood urea nitrogen level. In some aspects the senescent cells are located in renal proximal tubules of the subject. In some aspects the disease is cardiovascular disease. In some aspects the method comprises identifying a patient at risk of developing a cardiovascular disease. In some aspects the method comprises identifying a patient presenting at least one symptom of a cardiovascular disease. In some aspects the method further comprises administering a cholesterol reducing agent. In some aspects the method further comprises administering a blood-pressure reducing agent. In some aspects delaying the onset or progression of cardiovascular disease comprises delaying onset or progression of at least one symptom of cardiovascular disease. In some aspects a symptom of the cardiovascular disease is delayed for at least one month after diagnosis of cardiovascular disease in the subject. In some aspects a symptom of cardiovascular disease is delayed for at least six months after diagnosis of cardiovascular disease in a subject. In some aspects the symptom is selected from irregularity in heart rhythm, age-related cellular hypertrophy, increase in the cross-sectional area of a cardiomyocyte and decrease in cardiac stress tolerance. In some aspects delaying the onset or progression of cardiovascular disease comprises ameliorating one or more symptoms of cardiovascular disease. In some aspects the symptom is selected from irregularity in heart rhythm, age-related cellular hypertrophy, increase in the cross-sectional area of a cardiomyocyte and decrease in cardiac stress tolerance. In some aspects the symptom is age-related cellular hypertrophy. In some aspects administering the compound to the subject decreases age-related cellular hypertrophy relative to a pre-treatment value of cellular hypertrophy. In some aspects the symptom is an increase in the cross-sectional area of a cardiomyocyte. In some aspects administering the compound to the subject decreases the cross-sectional area of the cardiomyocyte relative to a pre-treatment value of a cross-sectional area of a cardiomyocyte. In some aspects the symptom is a decrease in cardiac stress tolerance. In some aspects administering the compound to the subject increases the cardiac stress tolerance relative to a pre-treatment value of cardiac stress tolerance. In some aspects cardiac stress tolerance is increased by at least 10% relative to the pre-treatment value of cardiac stress tolerance. In some aspects the senescent cells are located on an atrial surface or ventricular surface of the heart. In some aspects the senescent cells are located in a pericardium of the heart. In some aspects the senescent cells comprise epithelial cells. In some aspects the senescent cells comprise fibroblast cells. In some aspects the senescent cells are located on an aortic root wall of the heart. In some aspects the senescent cells are vascular smooth muscle cells. In some aspects the condition is cancer. In some aspects the method comprises identifying a patient at risk of developing cancer. In some aspects the method comprises identifying a patient presenting at least one symptom of cancer. In some aspects the method comprises identifying a patient presenting at least one indicator of cancer. In some aspects the patient has undergone a surgical intervention to address a cancer. In some aspects the method further comprises administering a chemotherapeutic. In some aspects delaying onset or progression of cancer comprises delaying onset or progression of at least one symptom of cancer. In some aspects a symptom of cancer is delayed for at least one month after diagnosis of cancer in the subject. In some aspects a symptom of cancer is delayed for at least six months after diagnosis of cancer in the subject. In some aspects delaying onset or progression of cancer comprises ameliorating at least one symptom of cancer. In some aspects the symptom is tumorigenesis. In some aspects administration of the compound to the subject increases tumor latency. In some aspects the subject has a genetic predisposition to developing cancer. In some aspects the genetic predisposition is selected from BRCA1 mutations, BRCA2 mutations, BARD1 mutations, BRIP1 mutations, Cowden Syndrome, Lynch Syndrome, Garner's Syndrome, Li-Fraumeni Syndrome, Von Hippel-Lindau disease, and multiple endocrine neoplasia. In some aspects wherein the condition is arthritis. In some aspects the method comprises identifying a patient at risk of developing arthritis. In some aspects the method comprises identifying a patient presenting at least one symptom of arthritis. In some aspects the condition is dementia. In some aspects the method comprises identifying a patient at risk of developing dementia. In some aspects the method comprises identifying a patient presenting at least one symptom of dementia. In some aspects the condition is a cataract. In some aspects the method comprises identifying a patient at risk of developing a cataract. In some aspects the method comprises identifying a patient presenting at least one symptom of a cataract. In some aspects the condition is osteoporosis. In some aspects the method comprises identifying a patient at risk of developing osteoporosis. In some aspects the method comprises identifying a patient presenting at least one symptom of osteoporosis. In some aspects the condition is diabetes. In some aspects the method comprises identifying a patient at risk of developing diabetes. In some aspects the method comprises identifying a patient presenting at least one symptom of diabetes. In some aspects the condition is hypertension. In some aspects the method comprises identifying a patient at risk of developing hypertension. In some aspects the method comprises identifying a patient presenting at least one symptom of hypertension. In some aspects the condition is age-related fat loss. In some aspects the method comprises identifying a patient at risk of developing age-related fat loss. In some aspects the method comprises identifying a patient presenting at least one symptom of age-related fat loss. In some embodiments this disclosure provides a method of mimicking a beneficial health effect of calorie restriction in a subject, comprising administering to the subject a compound that selectively kills senescent cells over non-senescent cells. In some aspects caloric intake is not substantially modified. In some aspects the beneficial health effect of calorie restriction is selected from weight loss, improved organ function, and life extension. In some aspects the beneficial health effect of calorie restriction is the prevention of cancer, kidney disease, cardiovascular disease, obesity, type 2 diabetes, neurodegenerative disease, or an autoimmune disease. In some aspects the compound extends the lifespan of a non-human test subject relative to the lifespan of a control subject. In some aspects the compound extends the lifespan of a non-human test subject by at least 10% relative to the lifespan of a control test subject. In some aspects the compound extends the lifespan of a non-human test subject by at least 20% relative to the lifespan of a control test subject. In some aspects the lifespan of a non-human test subject is an average lifespan of multiple test subjects. In some aspects the lifespan of a control subject is the average lifespan of multiple control test subjects. In some aspects wherein practice of the method kills at least about 10% of the senescent cells. In some aspects practice of the method kills at least about 25% of the senescent cells. In some aspects practice of the method kills no more than 10% of non-senescent cells. In some aspects practice of the method kills no more than 5% of non-senescent cells. In some aspects the compound is administered in at least two treatment cycles, wherein each treatment cycle independently comprises a treatment course of from 1 day to 3 months followed by a non-treatment interval of at least 2 weeks; provided that if the compound agent is an MDM2 inhibitor, the MDM2 inhibitor is administered as a monotherapy, and each treatment course is at least 5 days long during which the MDM2 inhibitor is administered on at least 5 days. In some aspects the compound is selected from an MDM2 inhibitor; an inhibitor of one or more BCL-2 anti-apoptotic protein family members wherein the inhibitor inhibits at least BCL-xL; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-1β-converting enzyme-inhibitory protein (c-FLIP); an inhibitor of Serpin; an inhibitor of Ubiquilin-2; an inhibitor of Epiregulin; an inhibitor of Sorting nexin-3 (Snx3); an inhibitor of forkhead box protein O4 (FOXO4); and an inhibitor of Proto-oncogene tyrosine protein kinase Src (Src). In some aspects the compound is an MDM2 inhibitor and is Nutlin-3a or RG-1172. In some aspects the compound is administered as a monotherapy. In some aspects the compound is administered within at least one treatment cycle, which treatment cycle comprises a treatment course followed by a non-treatment interval; and wherein the total dose of the compound administered during the treatment cycle is an amount less than the amount effective for a cancer treatment, wherein the compound is selected from an inhibitor of a Bcl-2 anti-apoptotic protein family member that inhibits at least Bcl-xL; an MDM2 inhibitor; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-1n-converting enzyme-inhibitory protein (c-FLIP); an inhibitor of Serpin; an inhibitor of Ubiquilin-2; an inhibitor of Epiregulin; and an inhibitor of Sorting nexin-3 (Snx3); an inhibitor of forkhead box protein O4 (FOXO4); and an inhibitor of Proto-oncogene tyrosine protein kinase Src (Src). In some aspects the compound is administered during two or more treatment cycles, and wherein the total dose of the compound administered during the two or more treatment cycles is an amount less than the amount effective for a cancer treatment. In some aspects each treatment course is no longer than (a) one month, or (b) no longer than two months, or (c) no longer than 3 months. In some aspects each treatment course is no longer than (a) 5 days, (b) 7 days, (c) 10 days, (d) 14 days, or (e) 21 days. In some aspects each treatment course is selected from 3 days to 12 days. In some aspects the compound is administered every other day of each treatment course. In some aspects the compound is administered daily during each treatment course. In some aspects the non-treatment interval has a duration of at least one month. In some aspects the treatment course is one day and the non-treatment interval is between 0-12 months. In some aspects the compound is administered directly to an organ or tissue that comprises the senescent cells. In some aspects the compound is combined with at least one pharmaceutically acceptable excipient to formulate a pharmaceutically acceptable composition to provide timed-release of the compound. In some aspects the compound is administered as a bolus infusion. In some aspects the compound is administered topically, transdermally, intradermally, intraarticularly, intranasally, intratracheally, intubation, parenterally, or orally. In some aspects the MDM2 inhibitor is a cis-imidazoline compound, a spiro-oxindole compound, or a benzodiazepine compound. In some aspects the cis-imidazoline compound is a nutlin compound. In some aspects the nutlin compound is Nutlin-3a or Nutlin-3b. In some aspects the cis-imidazoline compound is RG-7112, RG7388, RO5503781, or is a dihydroimidazothiazole compound. In some aspects the MDM2 inhibitor is a spiro-oxindole compound selected from MI-63, MI-126, MI-122, MI-142, MI-147, MI-18, MI-219, MI-220, MI-221, MI-773, and 3-(4-chlorophenyl)-3-((1-(hydroxymethyl)cyclopropyl)methoxy)-2-(4-nitrobenzyl)isoindolin-1-one. In some aspects the MDM2 inhibitor is Serdemetan; a piperidinone compound; CGM097; or an MDM2 inhibitor that also inhibits MDMX and which is selected from RO-2443 and RO-5963. In some aspects the piperidinone compound is AM-8553. In some aspects the inhibitor of one or more BCL-2 anti-apoptotic protein family members is a BCL-2/BCL-xL inhibitor; a BCL-2/BCL-xL/BCL-w inhibitor; or a BCL-xL selective inhibitor. In some aspects the BCL-xL selective inhibitor is a benzothiazole-hydrazone compound, an aminopyridine compound, a benzimidazole compound, a tetrahydroquinolin compound, or a phenoxyl compound. In some aspects the benzothiazole-hydrazone compound is WEHI-539. In some aspects the inhibitor of one or more Bcl-2 anti-apoptotic protein family members is A-1155463, A-1331852, ABT-263, ABT-199, or ABT-737. In some aspects the Akt inhibitor is MK-2206. In some aspects the Akt inhibitor is CCT128930. In some aspects the JNK 1, JNK2, JNK, or Kit inhibitor is JNK-IN-8. In some aspects the PP2C or MKP-2 inhibitor is a benzophenanthridine alkaloid. In some aspects the benzophenanthridine alkaloid is sanguinarine chloride. In some aspects the reactive oxygen species (ROS) inducer is methyl 3-(4-nitrophenyl) propiolate (NPP). In some aspects the PKA inhibitor is AT7867. In some aspects the inhibitor of checkpoint kinase 1 or checkpoint kinase 2 is AZD7762. In some aspects the vascular endothelial growth factor receptor (VEGFR)-2 is sunitinib. In some aspects the inhibitor of PI3K is GDC-0980 or BKM120. In some aspects the ASK1 inhibitor is NQDI-1. In some aspects the inhibitor of Syk is R406. In some aspects the inhibitor of EGFR is erlotinib. In some aspects the inhibitor of cathepsin is CYM 7008-00-01. In some aspects the glucosamine analog is GlcNAc. In some aspects the inhibitor of PARP1 or PARP2 is olaparib. In some aspects the compound that selectively kills senescent cells over non-senescent cells is selected from Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. In some aspects the subject suffers from a progeroid syndrome. In some aspects the progeroid syndrome is selected from Werner syndrome, Bloom syndrome, Rothmund-Thomson syndrome, Cockayne syndrome, xeroderma pigmentosum, trichothiodystrophy, combined xeroderma pigmentosum-Cockayne syndrome, restrictive dermopathy, and Hutchinson-Gilford progeria syndrome. In some aspects the progeroid syndrome is selected from Werner syndrome and Hutchinson-Gilford progeria.

In some embodiments the disclosure provides a method of ameliorating the progression of vertebral disc degeneration, comprising identifying an individual susceptible to vertebral disc degeneration, and administering a senolytic agent. In some aspects the senolytic agent is administered at a dose below a dose identified as therapeutically relevant as a cancer treatment for the agent. In some aspects the senolytic agent is selected from an MDM2 inhibitor; an inhibitor of one or more BCL-2 anti-apoptotic protein family members wherein the inhibitor inhibits at least BCL-xL; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-1β-converting enzyme-inhibitory protein (c-FLIP); an inhibitor of Serpin; an inhibitor of Ubiquilin-2; an inhibitor of Epiregulin; an inhibitor of Sorting nexin-3 (Snx3); an inhibitor of forkhead box protein O4 (FOXO4); and an inhibitor of Proto-oncogene tyrosine protein kinase Src (Src) In some aspects the compound is an MDM2 inhibitor. In some aspects the MDM2 inhibitor is administered at a dose below a dose identified as therapeutically relevant as a cancer treatment. In some aspects the individual is not diagnosed as having cancer. In some aspects the MDM2 inhibitor is selected from Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. In some aspects the MDM2 inhibitor is Nutlin 3a. In some aspects the individual presents back pain. In some aspects the individual demonstrates at least one sign of vertebral disc degeneration. In some aspects the individual has undergone at least one back surgery. In some aspects the individual has suffered a back injury. In some aspects the individual has suffered a herniated disc. In some aspects the MDM2 inhibitor is administered within 2 weeks of undergoing back surgery. In some aspects the MDM2 inhibitor is administered in at least three consecutive months. In some aspects the MDM2 inhibitor is administered in at least three consecutive years In some aspects the individual suffers from age-related disc degeneration. In some aspects the method comprises use of a composition.

In some embodiments this disclosure provides a method of delaying muscular atrophy, comprising selecting an individual at risk of muscular atrophy and administering a senolytic agent. In some aspects the senolytic agent is administered at a dose below a dose identified as therapeutically relevant as a cancer treatment for the agent. In some aspects the senolytic agent is selected from an MDM2 inhibitor; an inhibitor of one or more BCL-2 anti-apoptotic protein family members wherein the inhibitor inhibits at least BCL-xL; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-1β-converting enzyme-inhibitory protein (c-FLIP); an inhibitor of Serpin; an inhibitor of Ubiquilin-2; an inhibitor of Epiregulin; an inhibitor of Sorting nexin-3 (Snx3); an inhibitor of forkhead box protein O4 (FOXO4); and an inhibitor of Proto-oncogene tyrosine protein kinase Src (Src) In some aspects the compound is an MDM2 inhibitor. In some aspects the MDM2 inhibitor is administered at a dose below a dose identified as therapeutically relevant as a cancer treatment. In some aspects the individual is not diagnosed as having cancer. In some aspects the MDM2 inhibitor is selected from Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. In some aspects the MDM2 inhibitor is Nutlin 3a. In some aspects the individual suffers from paralysis. In some aspects the paralysis results from a motor nervous system trauma. In some aspects the individual has undergone at least one surgery to address a motor nervous system injury. In some aspects the individual has suffered a back injury. In some aspects the MDM2 inhibitor is administered within 2 weeks of undergoing back surgery. In some aspects the MDM2 inhibitor is administered within 2 weeks of suffering a motor nervous system trauma. In some aspects the MDM2 inhibitor is administered in at least three consecutive months. In some aspects the MDM2 inhibitor is administered in at least three consecutive years. In some aspects the individual suffers from motor neuron degeneration. In some aspects the individual suffers from age-related muscle atrophy. In some aspects the method comprises use of a composition.

In some embodiments this disclosure provides a composition for use in selectively killing senescent cells in a mammal comprising an MDM2 inhibitor and a pharmaceutically acceptable buffer. In some aspects the MDM2 inhibitor binds an MDM2 N-terminus. In some aspects the MDM2 inhibitor blocks E3 ligase activity. In some aspects the MDM2 inhibitor is selected from the list consisting of Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-41-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. In some aspects the MDM2 inhibitor is selected from the list consisting of AMG-232, NVP-CGM097, MI-773, CAY10681, CAY10682, Y239-EE, RG-7112, a Boronate, RO-5963, HLI 373, JNJ 26854165, and MEL23. In some aspects the MDM2 inhibitor comprises MI-773. In some aspects the MDM2 inhibitor comprises RG-7112. In some aspects the MDM2 inhibitor comprises JNJ 26854165. In some aspects the MDM2 inhibitor comprises MEL23. In some aspects the disclosure provides a composition for use in delaying intervertebral disc degeneration. In some aspects the disclosure provides a composition for use in delaying muscular atrophy.

In some embodiments the disclosure provides a method of healthy lifespan extension comprising administering an MDM2 inhibitor in combination with at least one lifespan extending measure to an individual. In some aspects the at least one lifespan extending measure comprises exercise. In some aspects the at least one lifespan extending measure comprises caloric restriction. In some aspects the MDM2 inhibitor is administered at a dose below a dose known to ameliorate symptoms of a cancer. In some aspects the MDM2 inhibitor is at least one compound selected from the list consisting of Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-41-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. In some aspects the MDM2 inhibitor is selected from the list consisting of AMG-232, NVP-CGM097, MI-773, CAY10681, CAY10682, Y239-EE, RG-7112, a Boronate, RO-5963, HLI 373, JNJ 26854165, and MEL23. In some aspects the MDM2 inhibitor comprises MI-773. In some aspects the MDM2 inhibitor comprises RG-7112. In some aspects the MDM2 inhibitor comprises JNJ 26854165. In some aspects the MDM2 inhibitor comprises MEL23. In some aspects the MDM2 inhibitor comprises AD20187. In some aspects the individual does not present a symptom of an age-related disorder.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 provides a schematic of general timelines and procedures for treatment with Nutlin-3a (Nut) of (1) cells induced to senesce by irradiation (Sen(IR)); (2) cells induced to senesce by treatment with doxorubicin (Sen(Doxo)); and (3) non-senescent cells (Non Sen).

FIGS. 2A-D show the effect of Nutlin-3a on survival of fibroblasts induced to senesce by irradiation. FIG. 2A illustrates effect of Nutlin-3a at 0, 2.5 or 10 μM after 9 days of treatment (D9) on irradiated (IR) senescent foreskin fibroblasts (Sen(IR)HCA2). FIG. 2B shows percent survival of irradiated BJ cells (Sen(IR)BJ) treated with Nutlin 3a at the concentrations shown. FIG. 2C shows percent survival of irradiated lung fibroblasts (Sen(IR)IMR90)), and FIG. 2D shows percent survival of irradiated mouse embryonic fibroblasts (MEFs) treated with Nutlin-3a.

FIGS. 3A-B illustrate the effect of Nutlin-3a on survival of cells induced to senesce by treatment with doxorubicin. HCA2 cells were treated with Nutlin-3a for 9 days (D9), and aortic endothelial cells were treated with Nutlin-3a for 11 days (D11), and then percent survival was determined. FIG. 3A shows the effect of Nutlin-3a on doxorubicin-treated (Doxo) senescent foreskin fibroblasts (HCA2). FIG. 3B illustrates the effect of Nutlin-3a on doxorubicin-treated (Doxo) senescent aortic endothelial cells (Endo Aort) (FIG. 3B).

FIGS. 4A-C show percent growth of non-senescent fibroblasts treated with Nutlin-3a. Cells were treated with Nutlin-3a for 9 days and percent growth determined (D9). Nutlin-3a was non-toxic to non-senescent foreskin fibroblasts (Non Sen HCA2) as shown in FIG. 4A, non-toxic to non-senescent lung fibroblasts (Non Sen IMR90) as shown in FIG. 4B, and non-toxic to non-senescent lung mouse embryonic fibroblasts (Non Sen MEFs) as shown in FIG. 4C.

FIGS. 5A-B illustrate percent growth of non-senescent aortic endothelial cells and non-senescent pre-adipocytes treated with Nutlin-3a. Cells were treated with Nutlin-3a for 11 days and percent growth determined (D11). FIG. 5A and FIG. 5B show that Nutlin-3a is non-toxic to non-senescent aortic endothelial (Non Sen Endo Aort) cells and to non-senescent pre-adipocytes (Non Sen Pread), respectively.

FIG. 6 presents a schematic of a timeline for treatment and imaging analysis of p16-3MR mice with Nutlin-3a. On day 35, the mice were sacrificed and fat and skin were collected for RNA, and lungs were collected and flash frozen for immunomicroscopy. RNA was analyzed for expression of SASP factors (mmp3, IL-6) and senescence markers (p21, p16, and p53). Frozen lung tissue was analyzed for DNA damage marker (yH2AX).

FIG. 7 shows a schematic of p16-3MR transgene insertion. 3MR (tri-modality reporter) is a fusion protein containing functional domains of a synthetic Renilla luciferase (LUC), monomeric red fluorescence protein (mRFP), and truncated herpes simplex virus (HSV)-1 thymidine kinase (tTK), which allows killing by ganciclovir (GCV). The 3MR cDNA was inserted in frame with p16 in exon 2, creating a fusion protein containing the first 62 amino acids of p16, but does not include the full-length wild-type p16 protein. Insertion of the 3MR cDNA also introduced a stop codon in the p19^{ARF} reading frame in exon 2.

FIG. 8 illustrates the reduction of luminescence intensity of doxorubicin-induced senescence in mice. Female C57/B16 p16-3MR mice were treated with doxorubicin (DOXO). Luminescence was measured 10 days later and used as baseline for each mouse (100% intensity). Nutlin-3a (NUT) was administered intraperitoneally daily from day 10 to day 24 post-doxorubicin treatment (N=9). Luminescence was then measured at day 7, 14, 21, 28, 35 post-Nutlin-3a treatments, and final values calculated as % of the baseline values. Control animals (DOXO) were injected with equal volume of PBS (N=3).

FIGS. 9A-E illustrate the level of mRNA of endogenous mmp-3, IL-6, p21, p16, and p53 in the skin and fat from animals after treatment with doxorubicin alone (DOXO) or doxorubicin plus Nutlin-3a (DOXO+NUT). The values represent the fold induction of the particular mRNA compared with untreated control animals. FIG. 9A: p21; FIG. 9B: p16^{INK4a}(p16); FIG. 9C: p53; FIG. 9D: mmp-3; and FIG. 9E: IL-6. Data were obtained from doxorubicin-treated mice (Doxo N=3), and doxorubicin+Nutlin-3a-treated mice (Doxo+Nutlin N=6).

FIGS. 10A-B present data showing that Nutlin-3a reduced the number of cells with doxorubicin-induced DNA damage. FIG. 10A presents immunofluorescence microscopy of lung sections from doxorubicin treated animals (DOXO) (left panel) and doxorubicin and Nutlin-3a-treated mice (DOXO+NUTLIN) detected by binding to a primary rabbit polyclonal antibody specific for γH2AX followed by incubation with a secondary goat anti-rabbit antibody, and then counterstained with DAPI. FIG. 10B shows the percent positive cells from immunofluorescence microscopy calculated and represented as percentage of the total number of cells. Data were obtained from doxorubicin-treated mice (Doxo N=3), and doxorubicin+Nutlin-3a-treated mice (Doxo-Nutlin N=3).

FIG. 11 shows that Nutlin-3a treatment reduced senescence-associated (SA) β-galactosidase (β-gal) intensity of fat biopsies from animals first treated with doxorubicin. Female C57/BL6 p16-3MR mice were treated with doxorubicin. A portion of the doxorubicin treated animals received Nutlin-3a (NUT) or PBS (DOXO) daily from day 10 to day 24 post-doxorubicin treatment. Three weeks after the Nutlin-3a treatment, mice were sacrificed and fat biopsies immediately fixed and stained with a solution containing X-Gal. Untreated animals were used as negative control (CTRL).

FIGS. 12A-12C show detection of IL-6 production in nuclei of non-senescent (NS) cells and irradiated (IR) senescent cells treated with Nutlin-3a. Primary human fibroblast (IMR90) cells were irradiated at Day −6 and treated with 10 μM Nutlin-3a or DMSO (vehicle control) in media from Day 0 to Day 9. Cells were cultured for an additional 6 days in media without Nutlin-3a or DMSO (Day 12 and Day 15). IL-6 was detected with an anti-IL-6 antibody in nuclei of cells at Day 9 and at Day 12. The percent IL-6 positive nuclei in each of irradiated Nutlin-3a treated cells and DMSO treated cells is illustrated in FIG. 12A. Immunofluorescence of cells expressing IL-6 detected with an anti-IL-6 antibody is illustrated in FIG. 12B. FIG. 12C illustrates the relative level of IL-6 secretion in senescent cells treated with Nutlin-3a (Sen (IR) Nut3a 10 μM) or vehicle (Sen (IR) DMSO) at Days 9, 12 and 15 (D9, D12, D15, respectively). The fold increase compared to non-senescent cells (Fold NS, y-axis) is shown.

FIGS. 13A-13F illustrate the level of senescence associated proteins (p21, p16, and IL-1a) and SASP factors (CXCL-1, IL-6, and IL-8) expressed by non-senescent (NS) cells and irradiated senescent cells treated with Nutlin-3a. IMR90 cells were irradiated at Day −6 and treated with 10 μM Nutlin-3a or DMSO (vehicle control) in media from Day 0 to Day 9. Cells were cultured for an additional 6 days (Day 12 and Day 15) in media without Nutlin-3a or DMSO, changing media at Day 12. Quantitative PCR was performed, and the levels of p21 (FIG. 13A, p21/actin y-axis on log scale); p16 (FIG. 13B); IL-1a (FIG. 13C); CXCL-1 (FIG. 13D); IL-6 (FIG. 13E); and IL-8 (FIG. 13F) expression were detected in non-senescent cells (NS (i.e., Day −7)) and at Day 9 (d9) and Day 12 (d12) in senescent cells treated with Nutlin-3a (Sen (IR) Nut3A) or vehicle (Sen (IR) DMSO). The data are presented relative to expression of actin.

FIG. 14 presents an immunoblot detecting production of proteins in senescent cells treated with Nutlin-3a. IMR90 cells were irradiated at Day −6 and treated with Nutlin-3a or DMSO (vehicle control) in media from Day 0 to Day 9. Cells were cultured for an additional 6 days (Day 12 and Day 15) in media without Nutlin-3a or DMSO, changing media at Day 12. The levels of each protein were detected using commercially available antibodies. The data are shown for non-senescent cells (NS) and for senescent cells at days 9, 12, and 15 (Xd9, Xd12, and Xd15, respectively) cultured in 10 μM Nutlin-3a (+) or vehicle (−).

FIGS. 15A-B depict an exemplary timeline and treatment protocol in senescent (irradiated cells, FIG. 15A) and non-senescent cells (non-radiated cells, FIG. 15B) for a cell counting assay.

FIG. 16 depicts a graph showing the effect of ABT-263 (“Navi”) treatment on non-senescent IMR90 cells (Non Sen IMR90).

FIG. 17 depicts a graph showing the effect of ABT-263 treatment on senescent IMR90 cells (Sen(IR) IMR90).

FIG. 18 depicts an exemplary timeline and treatment protocol in senescent (irradiated cells) and non-senescent cells (non-radiated cells) in a cell viability assay (CellTiter-Glo® (CTG)).

FIG. 19 illustrates a graph showing the effect of ABT-263 treatment on non-senescent and senescent IMR90 cells.

FIG. 20 illustrates a graph showing the effect of ABT-263 treatment in non-senescent and senescent renal epithelial cells.

FIG. 21 illustrates a graph showing the effect of ABT-263 treatment in non-senescent and senescent foreskin fibroblasts (HCA2) cells.

FIG. 22 illustrates a graph showing the effect of ABT-263 treatment in non-senescent and senescent lung fibroblast cells (IMR90).

FIG. 23 illustrates a graph showing the effect of ABT-263 treatment in non-senescent and senescent pre-adipose cells.

FIG. 24 illustrates a graph showing the effect of ABT-263 treatment in non-senescent and senescent mouse embryonic fibroblasts (MEF) cells.

FIG. 25 illustrates a graph showing the effect of ABT-263 treatment in non-senescent and senescent smooth muscle cells (Smth Mscl).

FIG. 26 illustrates a graph showing the effect of ABT-199 treatment in non-senescent and senescent IMR90 cells.

FIG. 27 illustrates a graph showing the effect of ABT-199 treatment in non-senescent and senescent IMR90 cells.

FIG. 28 illustrates a graph showing the effect of Obatoclax treatment in non-senescent and senescent IMR90 cells.

FIG. 29A and FIG. 29B: FIG. 29A presents a graph showing the effect of ABT-263 (Navi) treatment in combination with 10 nM MK-2206 in non-senescent and senescent IMR90 cells. FIG. 29B illustrates percent survival of non-senescent IMR90 cells (IMR90 NS) and senescent IMR90 cells (IMR90 Sen(IR)) when exposed to MK-2206 alone.

FIGS. 30A-B illustrate the effect of WEHI-539 on percent survival of senescent irradiated lung fibroblasts (Sen(IR)IMR90)) (FIG. 30A) and percent survival of irradiated renal cells (Sen(IR)) (FIG. 30B). NS=Non-senescent cells, which were not exposed to radiation.

FIGS. 31A-B illustrates that in the presence of a caspase inhibitor (panCaspase inhibitor, Q-VD-OPh) the senolytic activity of WEHI-539 is inhibited. FIG. 31A illustrates the effect of WEHI-539 on killing senescent cells (IMR90 Sen(IR)). The data points within the boxed area depict killing of senescent cells at the WEHI-539 concentrations of 1.67 μM and 5 μM of to which non-senescent cells (NS) and senescent cells (Sen (IR)) were exposed in the presence or absence of Q-VC-OPh. The percent survival of non-senescent cells and senescent cells in the presence and absence of the pan-Caspase inhibitor (Q-VD in the figure) is illustrated in FIG. 31B.

FIG. 32 shows the effect of specific shRNA molecules on survival of senescent cells. Senescent cells and non-senescent IMR90 cells were transduced with lentiviral vectors comprising shRNA molecules specific for each of BCL-2, BCL-xL, and BCL-w encoding polynucleotides. The ratio of senescent cell survival to non-senescent cell survival for each shRNA is shown. Each bar represents the average of triplicates. The shRNA sequences introduced into the cells are as follows from left to right: BCL-2: SEQ ID NO:1, 3, 3, 5; BCL-XL: SEQ ID NO: 7, 9, 11, 13; BCL-w: SEQ ID NO:15, 17, 19, 21; two non-transduced (NT) samples.

FIG. 33 illustrates the effect of ABT-737 on viability of non-senescent lung fibroblast cells (IMR90) (IMR90 NS) and senescent lung fibroblast cells (IMR90) (IMR90 Sen(IR)).

FIGS. 34A-B illustrates that in the presence of a caspase inhibitor (panCaspase inhibitor, Q-VD-OPh) the senolytic activity of ABT-263 is inhibited. FIG. 34A illustrates the effect of ABT-263 on killing senescent cells (IMR90 Sen(IR)). Non-senescent cells (NS) and senescent cells (Sen (IR)) were exposed to ABT-263 at concentrations of 0.33 μM and 1 μM in the presence or absence of the pan-Caspase inhibitor, Q-VC-OPh. The percent survival of non-senescent cells and senescent cells in the presence and absence of the pan-Caspase inhibitor (Q-VD in the figure) is illustrated in FIG. 34B.

FIG. 35 depicts animal study designs for assessing the efficacy of removal of senescent cells by Nutlin-3A treatment in C57BL6/J mice or by GCV treatment in 3MR mice in inhibiting signs and progression of osteoarthritis. Group 1 animals (16×C57BL6/J mice; 1×3MR mouse) represent the anterior cruciate ligament (ACL) control group that undergo surgery to cut the ACL (ACL surgery or osteoarthritis surgery (OA)) of one hind limb to induce osteoarthritis. Group 1 animals receive intra-articular injections of vehicle (10 μl) qd for 5 days during week 2 post-surgery and an optional second treatment cycle at week 4 post-surgery, parallel to the GCV treatment in the test animals. Group 2 animals (3×3MR mice) represent one treatment group that receives ACL surgery and intra-articular injections of GCV (2.5 μg/joint) qd for 5 days during week 2 post-surgery and an optional second treatment cycle at week 4 post-surgery. Group 3 animals (12×C57BL6/J) represent a second treatment group that received ACL surgery and intra-articular injections of Nutlin-3A (5.8 μg/joint) qod for 2 weeks starting at week 3 post-surgery. Group 4 animals represent a second control group having a sham surgery that does not sever the ACL and receiving intra-articular injections of vehicle (10 μl) qd for 5 days during week 2 post-surgery and an optional second treatment cycle at week 4 post-surgery, parallel to the GCV treated 3MR mice. This study design can be adapted, such as the dosing amount and dosing schedule (e.g., number of days), for other senolytic agents.

FIG. 36 depicts a timeline for the animal study designs described in FIG. 35.

FIGS. 37A-C illustrate the level of senescence associated proteins (p16) and SASP factors (IL-6 and MMP13) expressed by cells from joints of mice that had osteoarthritis surgery (OA surgery), joints of mice that had OA surgery and received Nutlin-3A treatment (Nutlin-3A), joints that received sham surgery, and joints of control mice that did not receive any surgery (control). Quantitative PCR was performed, and the levels of p16 (FIG. 37A); IL-6 (FIG. 37B); and MMP13 (FIG. 37C) expression were detected in cells extracted from the joints of mice with OA surgery, mice with OA surgery and Nutlin-3A treatment, sham surgery, and control (no surgery). The data are presented relative to expression of actin. The data shows that Nutlin-3A treatment clears senescent cells from the joint.

FIG. 38 illustrates the level of type 2 collagen expressed by cells from joints of mice that had osteoarthritis surgery (OA surgery), joints of mice that had OA surgery and received Nutlin-3A treatment (Nutlin-3A), joints that received sham surgery, and joints of control mice that did not receive any surgery. Quantitative PCR was performed, and the levels of type 2 collagen was detected in cells extracted from the joints of mice with OA surgery, mice with OA surgery and Nutlin-3A treatment, sham surgery, and control (no surgery). The data are presented relative to expression of actin. The data shows that Nutlin-3A treatment drives ab initio collagen production in OA joints.

FIGS. 39A-B illustrate incapacitance measurements 4 weeks after osteoarthritis surgery as measured by a weight bearing test to detect which leg mice favored. The mice were placed in a chamber (FIG. 39A), standing with 1 hind paw on each scale (FIG. 39B). The weight that was placed on each hind limb was then measured over a 3-second period. At least 3 separate measurements were made for each animal at each time point, and the result was expressed as the percentage of the weight placed on the operated limb/the contralateral unoperated limb.

FIG. 40 depicts the results of the weight bearing test shown in FIG. 39. Osteoarthritis causes mice to favor the unoperated leg over the operated leg (4). Clearing senescent with Nutlin-3A abrogates this effect (7).

FIG. 41 depicts the results of a hotplate analysis to provide an assessment of sensitivity and reaction to pain stimulus. Paw-lick response time for the operated hind limb (measured in seconds) due to attainment of pain threshold after placement onto a 55° C. platform was measured 4 weeks after osteoarthritis (OA) surgery. The data shows that Nutlin-3A treatment reduces response time in OA surgery mice (▴) as compared to untreated OA surgery mice (▪).

FIGS. 42A-C presents histopathology results from animals not treated by surgery (No Surgery, FIG. 42A (C57B)); animals that received osteoarthritis surgery and received vehicle (OA surgery, FIG. 42B (3MR)); and animals that received OA surgery and were treated with Nulin-3a (OA surgery+Nutlin-3a, FIG. 42C). Arrows point to intact or destroyed proteoglycan layers in the joint.

FIGS. 43A-B illustrate schematics of two atherosclerosis animal model studies in LDLR^{-/-} transgenic mice fed a high fat diet (HFD). The study illustrated in FIG. 43A assesses the extent to which clearance of senescent cells from plaques in LDLR^{-/-} mice with a senolytic agent (e.g., Nutlin-3A) reduces plaque load. The study illustrated in FIG. 43B assesses the extent to which ganciclovir-based clearance of senescent cells from LDLR^{-/-} /3MR double transgenic mice improves pre-existing atherogenic disease.

FIGS. 44A-D depict graphs of the plasma lipid levels in LDLR^{-/-} mice fed a HFD after one treatment cycle of Nutlin-3A or vehicle. FIG. 44A shows total cholesterol levels in vehicle or Nutlin-3A treated LDLR^{-/-} mice compared to LDLR^{-/-} fed a non-HFD. FIG. 44B shows HDL levels in vehicle or Nutlin-3A treated LDLR^{-/-} mice compared to LDLR^{-/-} fed a non-HFD.

FIG. 44C shows triglyceride levels in vehicle or Nutlin-3A treated LDLR^{-/-} mice compared to LDLR^{-/-} fed a non-HFD. FIG. 44D shows vLDL/LDL/IDL levels in vehicle or Nutlin-3A treated LDLR^{-/-} mice compared to LDLR^{-/-} fed a non-HFD.

FIGS. 45A-D illustrate RT-PCR analysis of SASP factors and senescence markers in aortic arches of LDLR^{-/-} mice fed a HFD after one treatment cycle of Nutlin-3A or vehicle. FIG. 45A illustrates the aortic arch (boxed). FIG. 45B and FIG. 45C show expression levels of SASP factors and senescence markers, normalized to GAPDH and expressed as fold change vs. non-HFD, vehicle-treated, age-matched LDLR^{-/-} mice. FIG. 45D shows the data from FIGS. 45B-C in numerical form.

FIGS. 46A-C illustrate RT-PCR analysis of SASP factors and senescence markers in aortic arches of LDLR^{-/-} mice fed a HFD after two treatment cycles of Nutlin-3A or vehicle. FIGS. 46A-B expression levels of SASP factors and senescence markers, normalized to GAPDH and expressed as fold change vs. non-HFD, vehicle-treated, age-matched LDLR^{-/-} mice.

FIG. 46C shows the data from FIGS. 46A-B in numerical form.

FIGS. 47A-C illustrate staining analysis for aortic plaques in LDLR^{-/-} mice fed a HFD after three treatment cycles of Nutlin-3A or vehicle. FIG. 47A illustrates the aorta. FIG. 47B shows the % of the aorta covered in plaques. FIG. 47C shows Sudan IV staining of the aorta to visualize the plaques and the area covered by the lipid plaque was expressed as a percentage of the total surface area of the aorta in each sample.

FIGS. 48A-B depict plots of platelet (FIG. 48A) and lymphocyte counts (FIG. 48B) from LDLR^{-/-} mice fed a HFD after three treatment cycles of Nutlin-3A or vehicle.

FIGS. 49A-B depict plots of weight (FIG. 49A) and body fat/lean tissue composition (%, FIG. 49B), respectively, of LDLR^{-/-} mice fed a HFD after three treatment cycles of Nutlin-3A or vehicle.

FIG. 50 depicts a graph of the effect of clearance of senescent cells with ganciclovir in LDLR^{-/-} and LDLR^{-/-} MR mice fed a HFD, as measured by the % of the aorta covered in plaques.

FIG. 51 depicts a graph of the effect of clearance of senescent cells with ganciclovir in LDLR^{-/-} and LDLR^{-/-} MR mice fed a HFD, as measured by the plaque cross-sectional area of the aorta.

FIG. 52 shows the effect of senescent cell clearance on resistance to cardiac stress with aging. 12 month old INK-ATTAC transgenic mice on FVB×129Sv/E×C57BL/6 mixed of C57BL/6 pure genetic backgrounds were injected 3×/week with AP20187 (0.2 mg/kg for the mixed cohort and 2 mg/kg for the C57BL/6 cohort, respectively). At 18 months, subsets of male and female mice from each cohort were subjected to a cardiac stress test and time to cardiac arrest was recorded. Control cohort received injections of vehicle.

FIG. 53 shows the RT-PCR analysis of Sur2a expression in female INK-ATTAC transgenic mice described in FIG. 52.

FIGS. 54A-C illustrate staining analysis for aortic plaques in LDLR^{-/-} MR double transgenic mice and LDLR^{-/-} control mice fed a HFD after a 100 day treatment period with ganciclovir. FIGS. 54A-B show Sudan IV staining of the aorta to visualize the plaques in LDLR^{-/-} control mice (FIG. 54A) and LDLR^{-/-} MR mice (FIG. 54B), respectively. FIG. 54C shows the % of the aorta covered in plaques as measured by area of Sudan IV staining.

FIGS. 55A-D illustrate plaque morphology analysis in LDLR^{-/-} MR double transgenic mice and LDLR^{-/-} control mice fed a HFD after a 100 day treatment period with ganciclovir. FIG. 55A and FIG. 55C show Sudan IV staining of the aorta to visualize the plaques in LDLR^{-/-} control mice and LDLR^{-/-} MR mice, respectively. Plaques that are circled were harvested and cut into cross-sections and stained with to characterize the general architecture of the atherosclerotic plaques (FIG. 55B and FIG. 55D). “#” marks fat located on the outside of the aorta.

FIGS. 56A-C shows that SA-β-GAL crystals localize to lipid-bearing foam cells from an atherosclerotic artery of a mouse fed a high-fat diet. The macrophage foam cell, FIG. 56A, is shown by a white dotted outline and adjacent to the macrophage foam cell is a smooth muscle foam cell. The left boxed area in the macrophage foam cell is magnified and shown in FIG. 56B to illustrate lysosomes with SA-β-GAL crystals. The boxed area within the smooth muscle foam cell is magnified and shown in FIG. 56C.

FIG. 57 presents a macrophage foam cell from an atherosclerotic artery of a mouse fed a high-fat diet. Lipid-bearing lysosomes containing SA-β-GAL crystals are noted by the arrows.

FIGS. 58A-B show that SA-β-GAL crystals localize in the lysozomes of smooth muscle foam cells in an atherosclerotic artery of a mouse fed a high-fat diet. The boxed area in FIG. 58B is magnified and shown in FIG. 58A.

FIG. 59 shows the effect of senescent cell clearance on peripheral capillary oxygen saturation (SpO₂) in bleomycin exposed mice.

FIGS. 60A-C illustrate the effect of senescent cell clearance with ganciclovir on lung function in 3MR mice exposed to bleomycin. FIG. 60A shows the effect of ganciclovir treatment on lung elastance of 3MR mice exposed to bleomycin. FIG. 60B shows the effect of ganciclovir treatment on dynamic lung compliance of 3MR mice exposed to bleomycin. FIG. 60C shows the effect of ganciclovir treatment on static lung compliance of 3MR mice exposed to bleomycin.

FIG. 61 shows the effect of senescent cell clearance on peripheral capillary oxygen saturation (SpO₂) in mice after 2 months and 4 months of cigarette smoke (CS) exposure. AP=AP20187; GAN=ganciclovir; Navi=Navitoclax (ABT-263); and Nutlin=Nutlin 3A.

FIGS. 62A-C illustrate the effect of RG-7112 (structure shown in FIG. 62A) on percent survival of senescent irradiated lung fibroblasts IMR90 cells ((IMR90)Sen(IR)) and non-senescent IMR90 cells, which were not exposed to radiation (IMR90 NS) after 3 days of treatment (FIG. 62B) and after six days of treatment with RG-7112 (FIG. 62C).

FIGS. 63A-B illustrates that paclitaxel induces senescence in p16-3MR mice. Groups of mice (n=4) were treated three times every two days with 20 mg/kg paclitaxel or vehicle. The level of luminescence in mice treated with paclitaxel is shown in FIG. 63A. The level of mRNA in skin was determined for each of the target genes: p16, 3MR transgene, and IL-6 in animals treated with paclitaxel as shown in FIG. 63B.

FIGS. 64A-B shows the effect of ABT-263 on mice that were initially treated with paclitaxel. The schematic of the experiment performed in 3MR mice is shown in FIG. 64B. Mice were first treated with paclitaxel, followed by treatment with either vehicle, ganciclovir (gcv) or ABT-263. Wheel counts were measured for each group of mice (n=4) treated with paclitaxel+vehicle (pacli+vehicle); paclitaxel+ganciclovir (pacli+gcv); paclitaxel+ABT-263 (pacli+ABT-263); and control animals that did not receive paclitaxel (see FIG. 64A).

FIG. 65 shows the level of senescence induced in groups of p16-3MR animals (n=4) treated with chemotherapeutic drugs: thalidomide (100 mg/kg; 7 daily injections); romidepsin (1 mg/kg; 3 injections); pomalidomide (5 mg/kg; 7 daily injections); lenalidomide (50 mg/kg; 7 daily injections); 5-azacytidine (5 mg/kg; 3 injections) and doxorubicin (10 mg/kg; 2-4 injections during a week). The level of luminescence was measured in animals treated with the drugs.

FIG. 66 shows an immunoblot showing the level of different cellular proteins in senescent and non-senescent human abdominal subcutaneous preadipocytes. Senescence was induced as described in Example 28. Lysates were prepared at several time points after induction of senescence, and the level of each protein in the lysates detected at 24 hours and at days 3, 5, 8, 11, 15, 20, and 25 (D3, D5, D8, D11, D15, D20, and D25).

FIG. 67A shows senescent cells (by luminescence) for a p16-3MR mouse on a regular chow diet. FIG. 67B shows senescent cells (by luminescence) for a p16-3MR mouse on a high fat diet. FIG. 67C shows that groups of p16-3MR mice (n=6) fed a high fat diet (high fat) for four months have increased numbers of senescence cells compared with mice fed a regular chow diet (chow fed) (n=6).

FIG. 68 illustrates decrease of senescent cells in adipose tissue of p16-3MR mice fed a high fat diet for four months and then treated with ganciclovir compared to mice treated with vehicle. The presence of senescent cells in perirenal, epididymal (Epi), or subcutaneous inguinal (Ing) adipose tissue was detected by SA-β-Gaal staining.

FIGS. 69A-C show the effect of ganciclovir treatment on glucose tolerance in p16-3MR mice fed a high fat diet. A bolus of glucose was given at time zero, and blood glucose was monitored for up to 2 hours to determine efficacy of glucose disposal (FIG. 69A). This is quantified as area under the curve (AUC), with a higher AUC indicating glucose intolerance. The glucose tolerance test (GTT) AUC's of mice treated with ganciclovir is shown in FIG. 69B. Hemoglobin A1c is shown in FIG. 69C. n=9; ANOVA.

FIGS. 70A-70B show insulin sensitivity (Insulin Tolerance Testing (ITT)) of p16-3MR mice fed a high fat diet after ganciclovir administration. Blood glucose levels were measured at 0, 14, 30, 60, and 120 minutes after the administration of glucose bolus at time zero (see FIG. 70A). A change in insulin tolerance testing when ganciclovir was administered to wild-type mice was not observed (see FIG. 70B).

FIG. 71 illustrates the effect of A-1155463 on percent survival of senescent irradiated lung fibroblasts (Sen(IR)IMR90)) and percent survival of non-senescent IMR90 cells (Sen(IR)). NS=Non-senescent cells, which were not exposed to radiation.

FIG. 72A-C show the effect of the removal of senescent cells. FIG. 72A shows an experimental outline. FIG. 72B shows survival data for C57BL/6J×FVB mixed background (Mix) ATTAC mice in males and females (♂+♀), males (♂), or females (♀) treated with vehicle (−AP) or AP (+AP) as described in FIG. 72A. FIG. 72C shows survival data for C57BL/6J pure background (BL/6) ATTAC mice in the treatment groups of in males and females (♂+♀), males (♂), or females (♀) treated with vehicle (−AP) or AP (+AP) as described in FIG. 72A.

FIG. 73A-D show the effect of the removal of senescent cells. FIG. 73A shows cancer survival data for ATTAC mice in the treatment groups described in FIG. 72B. FIG. 73B shows cancer survival data for ATTAC mice in the treatment groups described in FIG. 72C. FIG. 73C shows the cancer type spectrum for ATTAC mice in the treatment groups described in FIG. 72B. FIG. 73D shows the cancer type spectrum for ATTAC mice in the treatment groups described in FIG. 72C.

FIGS. 74A-74K show the effect of the removal of senescent cells. FIGS. 74A-H show expression of the ATTAC transgene and a senescence marker panel in gastrocnemius, eye, kidney, heart (atria), spleen, lung, liver, and colon. FIGS. 74I-K show expression of inflammation markers in iWAT, kidney, and skeletal muscle. FIG. 74A shows transcript expression in gastrocnemius. FIG. 74B shows transcript expression in the eye. FIG. 74C shows transcript expression in the kidney. FIG. 74D shows transcript expression in the heart.

FIG. 74E shows transcript expression in the spleen. FIG. 74F shows transcript expression in the lung. FIG. 74G shows transcript expression in the liver. FIG. 74H shows transcript expression in the colon. FIGS. 74I-K show expression of inflammation markers. FIG. 74I shows transcript expression in iWAT. FIG. 74J shows transcript expression in the kidney. FIG. 74K shows transcript expression in gastrocnemius.

FIGS. 75A-75J show the effect of the removal of senescent cells. FIG. 75A shows duration of time spent balanced on a rotarod. FIG. 75B shows the percentage of investigations of a novel object (% novel object/total). FIG. 75C shows the exercise time to exhaustion in seconds (s) for ATTAC male (columns 1-3) and female (columns 4-6) mice at 12 m (columns 1 and 4), 18 m −AP (columns 2 and 5), or 18 m+AP (columns 3 and 6). FIG. 75D shows the exercise distance to exhaustion in m for ATTAC mice in the treatment groups described in FIG. 75C. FIG. 75E shows the work in Joules (J) for ATTAC mice in the treatment groups described in FIG. 75C. FIG. 75F shows the gastrocnemius weight in g for ATTAC mice in the treatment groups described in FIG. 75C. FIG. 75G shows the gastrocnemius fiber diameter in microns (μm) for ATTAC mice in the treatment groups described in FIG. 75C.

FIG. 75H shows the abdominal muscle fiber diameter in μm for ATTAC mice in the treatment groups described in FIG. 75C. FIG. 75I shows the paraspinal muscle fiber diameter in μm for ATTAC mice in the treatment groups described in FIG. 75C. FIG. 75J shows the grip strength in Newtons (N) for ATTAC mice in the treatment groups described in FIG. 75C.

FIGS. 76A-76K show the effect of the removal of senescent cells in adipose tissue. FIG. 76A shows GFP-non-expressing (GFP−) and GFP-expressing (GFP+) cells in inguinal adipose tissue (TAT) of wild type (WT) or ATTAC mice. FIG. 76B shows the expression of transcripts in cells from GFP+ or GFP− IAT. FIG. 76C shows senescence-associated β-galactosidase (SA-(3-Gal) staining in cells from GFP+ or GFP− IAT and a graph of the percentage (%) of SA-β-Gal-positive cells from GFP− (column 1) or GFP+(column 2) IAT.

FIG. 76D shows the percentage of GFP+ cells in white blood cells, endothelial cells, fat progenitor cells, or remaining cells (“Rest”) from the IAT of ATTAC mice at 18 m −AP or +AP.

FIG. 76E shows whole-mount SA-β-Gal staining of IAT and epididymal fat (EPI) from ATTAC mice at 12 m, 18 m −AP, or 18 m+AP. FIG. 76H shows fat mass measurements from ATTAC mice that are 12 m, 18 m −AP or 18 m+AP that are male (♂) or female (♀). FIG. 76F shows perivascular X-Gal-positive cells from an 18-month-old vehicle-treated C57BL6 ATTAC male. FIG. 76G shows quantification of cells containing X-Gal crystals (n=4 mice per treatment). FIG. 76H shows fat mass measurements from ATTAC mice that are 12 m, 18 m −AP or 18 m+AP that are male (a) or female (♀). FIG. 76I shows IAT (subcutaneous) and perigonadal (visceral) adipose depot weight from ATTAC mice that are 12 m, 18 m −AP or 18 m+AP that are male (a) or female (♀). FIG. 76J shows the mean adipocyte diameter in μm in ATTAC male mice at 12 m, 18 m −AP, or 18 m+AP. FIG. 76K shows expression of Pparg and Cebpa transcripts determined by qRT-PCR in ATTAC male mice at 12 m, 18 m −AP, or 18 m+AP.

FIGS. 77A-77B show the effect of the removal of senescent cells in adipose tissue. FIG. 77A shows the expression of transcripts determined by qRT-PCR in female ATTAC mice at 2 months of age (2 m), 12 m, 18 m −AP, or 18 m+AP. FIG. 77B shows perirenal, mesenteric, subscapular adipose tissue (SSAT), and brown adipose tissue weight in ATTAC male mice at 12 m, 18 m −AP, or 18 m+AP.

FIGS. 78A-781 show the effect of the removal of senescent cells in the kidney. FIG. 78A shows hematoxylin and eosin (H-E) staining and Periodic acid-Schiff (PAS) staining of kidney tissue from ATTAC mice at 18 m −AP or 18 m+AP. The scale bar is 50 μm. FIG. 78B shows the percentage (%) of sclerotic glomeruli in ATTAC mice from the treatment groups described in FIG. 72F. FIG. 78C shows the concentration of blood urea nitrogen in milligrams (mg) per deciliter (dl) in ATTAC mice from the treatment groups described in FIG. 72F. FIG. 78D shows whole-mount SA-β-Gal staining of kidney tissue from ATTAC mice at 18 m −AP or 18 m+AP. The scale bar is 250 μm. FIG. 78E is an electron micrograph of kidney tissue from a BL/6 ATTAC male mouse 18 m −AP. Inset images 1-3 show X-Gal crystals in the kidney tissue. The scale bar is 5 μm for the main micrograph and 200 nanometers (nm) for the inset images. FIG. 78F shows the percentage (%) of cells with X-Gal crystals in BL/6 ATTAC males at 18 m −AP (column 1) or 18 m+AP (column 2). FIG. 78G shows expression of Atgr1a transcript determined by qRT-PCR in the kidneys from BL/6 ATTAC male (columns 1-3) and female (columns 4-6) mice at 12 m (columns 1 and 4), 18 m −AP (columns 2 and 5), or 18 m+AP (columns 3 and 6). FIG. 78H shows expression of Atgr1a protein determined by Western blotting in kidneys from ATTAC mice 18 m −AP (lanes 1-3) or 18 m +AP (lanes 4-6), with Ponceau S staining of the membrane as a loading control. FIG. 78I shows immunofluorescent staining for Atgr1a protein from kidney tissue of ATTAC mice 18 m −AP (left images) or 18 m+AP (right images).

FIGS. 79A-791 show the effect of the removal of senescent cells in the heart. FIG. 79A shows whole-mount SA-β-Gal staining of hearts from BL/6 ATTAC mice at 12 m, 18 m −AP, or 18 m+AP, with the asterisk marking the position of the aortic root. Top inset images show aortic roots (ar) from a transverse plane, with the arrow marking the aortic root wall. Bottom inset images show ventricular (v) and arterial (a) boxed areas. The scale bar is 1 millimeter (mm) for all images. FIG. 79B is a set of electron micrographs of SA-β-Gal positive cells in the pericardium. Inset images show X-Gal images from the boxed areas. The asterisk marks cilia. The circular arrow marks collagen fibers. VSMC is a vascular smooth muscle cell. The scale bar is 2 μm in the main images and 200 nm in the inset images. FIG. 79C shows quantification of cells with X-Gal crystals in the visceral pericardium (n=4 mice per treatment). FIG. 79D shows the left ventricle free-wall thickness in μm of BL/6 ATTAC mice in the treatment groups described in FIG. 78G. FIG. 79E shows representative cardiomyocyte cross-sectional images (n=4 mice per group). FIG. 79F shows cardiomyocyte cross-sectional area in square microns (μm²) of BL/6 ATTAC mice in the treatment groups described in FIG. 78G. FIG. 79G shows expression of Sur2a transcript determined by qRT-PCR in the hearts of BL/6 ATTAC mice in the treatment groups described in FIG. 78G. FIG. 79H shows cardiac stress resistance as measured by the time to death in seconds (s) after injection with a lethal dose of isoproterenol in ATTAC mice from the treatment groups described in FIG. 72F. FIG. 79I shows change in left ventricular mass (LV) in response to sublethal doses of isoproterenol (10 mg/kg) after ten doses administered over five days.

FIGS. 80A-80G show the effect of the removal of senescent cells in the heart. FIG. 80A shows electron micrographs of X-Gal crystal containing cells in the aortic root. VSMC, vascular smooth muscle cell. FIG. 80B shows echocardiograph measurements of heart rate in beats per minute (bpm) for ATTAC mice in the treatment groups described in FIG. 75B. FIG. 80C shows echocardiograph measurements of left ventricular (LV) mass in corrected milligrams (mg) for ATTAC mice in the treatment groups described in FIG. 75B. FIG. 80D shows echocardiograph measurements of posterior wall thickness at diastole in millimeters (mm) for ATTAC mice in the treatment groups described in FIG. 75B. FIG. 80E shows echocardiograph measurements of left ventricular inner diameter at diastole in millimeters (mm) for ATTAC mice in the treatment groups described in FIG. 75B. FIG. 80F shows echocardiograph measurements of percentage (%) ejection fraction of the heart for ATTAC mice in the treatment groups described in FIG. 75B. FIG. 80G shows echocardiograph measurements of percentage (%) fractional shortening of the heart for ATTAC mice in the treatment groups described in FIG. 75B.

FIGS. 81A-81B show the effect of the removal of senescent cells in the eye. FIG. 81A shows the percentage (%) cataract incidence for ATTAC mice in the treatment groups described in FIG. 72B. FIG. 81B shows the percentage (%) cataract incidence for ATTAC mice in the treatment groups described in FIG. 72C.

FIGS. 82A-821 show the effect of the removal of senescent cells. FIG. 82A shows SA-β-Gal staining of IAT from ATTAC mice at 2 m treated with vehicle (−AP) or AP (+AP) beginning at weaning age. FIG. 82B shows the expression of p16 and actin protein determined by Western blotting in IAT from ATTAC mice treated as described in FIG. 82A. FIG. 82C shows the expression of transcripts determined by qRT-PCR in IAT from ATTAC mice treated as described in FIG. 82A. FIG. 82D shows the expression of p16 and actin protein determined by Western blotting in three mouse embryonic fibroblast (MEF) lines untreated (−) or treated with AP (+) at cell passage 3 (P3). FIG. 82E shows cell number over five days (day 0 to day 4) in P3 MEF lines untreated (−AP) or treated with AP (+AP). FIG. 82F shows the expression of transcripts determined by qRT-PCR in P3 MEF lines −AP or +AP. FIG. 82G shows the expression of p16 and actin protein determined by Western blotting in passage 4 (P4) primary ATTAC MEFs and SV40 Large-T antigen (LT) immortalized ATTAC MEFs. FIG. 82H shows the expression of p16 and actin protein determined by Western blotting in SV40 LT immortalized ATTAC MEFs treated with vehicle (−AP), 10 nanomolar AP (+AP (10 nM)), or 500 nM AP (+AP (500 nM)). FIG. 82I shows the expression of transcripts determined by qRT-PCR in P4 primary ATTAC MEFs or SV40 LT immortalized ATTAC MEFs treated as in FIG. 82H.

FIGS. 83A-83E show the effect of AP20187 treatment. FIG. 83A shows the fat mass in g in ATTAC male mice 18 m −AP and wild type mice at 18 months of age treated with AP (18 m+AP) as described in FIG. 72A. FIG. 83B shows the adipose depot weight in gin IAT and EPI in ATTAC and wild type male mice as described in FIG. 83A. FIG. 83C shows the % of sclerotic glomeruli in ATTAC and wild type male mice as described in FIG. 83A. FIG. 83D shows the concentration of blood urea in mg/dl in ATTAC and wild type male mice as described in FIG. 83A. FIG. 83E shows the time to death in s after injection with a lethal dose of isoproterenol in ATTAC and wild type male mice as described in FIG. 83A.

FIGS. 84A-84K show the effect of the removal of senescent cells. FIG. 84A shows wound closure of a 3-mm punch biopsy measured as the percentage (%) of starting wound size in ATTAC female mice 18 m −AP or 18 m+AP. Treatment was stopped two days prior to the biopsy and during wound closure. FIG. 84B shows wound closure of 3-mm punch biopsy wounds in 4-month-old female ATTAC mice after treatment with vehicle or AP following wounding. FIG. 84C shows quantification of total GFP⁺ cells isolated from 3-mm punch biopsy wounds of 4-month-old mice two days into the wound healing process treated with vehicle (black) or AP (grey). FIG. 84D-84K shows phosphotungstic acid haematoxylin (PTAH) staining of tissue sections from ATTAC mice at 18 m −AP or +AP. FIG. 84D shows PTAH staining in the kidney. FIG. 84E shows PTAH staining in the liver. FIG. 84F shows PTAH staining in skeletal muscle. FIG. 84G shows PTAH staining in the heart. FIG. 84H shows PTAH staining in the skin. FIG. 84I shows PTAH staining in the intestine. FIG. 84J shows PTAH staining in the eye. FIG. 84K shows PTAH staining in the lung.

FIG. 85 shows FACS-based quantification of iWAT progenitor cell numbers in 18-month-old ATTAC mice treated with vehicle or AP. ASC, adipocyte stem cells; PAC preadipocytes.

FIGS. 86A-86E show expression of senescence markers. FIG. 86A illustrates the endogenous Ink4a locus and the various Ink4a promoter regions driving ATTAC, 3MR and firefly luciferase (FLUC). FIG. 86B shows p16^{Ink4a} protein levels in p16-3MR MEFs. FIG. 86C shows expression of senescence marker mRNA in p16-3MR MEFs. FIG. 86D shows expression of senescence marker mRNA in p16-FLUC MEFs. FIG. 86E shows expression of ATTAC and senescence markers in CD3⁺ T cells from 12- and 18-month old ATTAC mice.

FIGS. 87A-87D show a comparison of lifespans under different diets and housing facilities. FIG. 87A shows a comparison of lifespans under different diets, FIG. 87B shows a comparison of lifespans with different genetic backgrounds and treatments, FIG. 87C and FIG. 87D show published lifespans.

FIG. 88A, FIG. 88B, and FIG. 88C show median lifespan extensions of AP-treated mice dying without tumors.

FIGS. 89A-89Q illustrate the effect of senescent cell clearance on hematological parameters and age-related changes in leukocyte populations. FIGS. 89A-L show hematology results. FIG. 89A illustrates the effect of senescent cell clearance on white blood cells. FIG. 89B illustrates the effect of senescent cell clearance on platelets. FIG. 89C illustrates the effect of senescent cell clearance on red blood cells. FIG. 89D illustrates the effect of senescent cell clearance on hemoglobin concentration. FIG. 89E illustrates the effect of senescent cell clearance on hematocrit. FIG. 89F illustrates the effect of senescent cell clearance on mean corpuscular volume. FIG. 89G illustrates the effect of senescent cell clearance on mean corpuscular hemoglobin. FIG. 89H illustrates the effect of senescent cell clearance on neutrophils. FIG. 89I illustrates the effect of senescent cell clearance on lymphocytes. FIG. 89J illustrates the effect of senescent cell clearance on basophils. FIG. 89K illustrates the effect of senescent cell clearance on monocytes. FIG. 89L illustrates the effect of senescent cell clearance on eosinophils. FIGS. 89M-Q show assessments for leukocyte subpopulations. FIG. 89M illustrates the effect of senescent cell clearance on CD4+ T cells. FIG. 89N illustrates the effect of senescent cell clearance on CD8+ T cells. FIG. 89O illustrates the effect of senescent cell clearance on CD44^{hi} CD4+ T cells. FIG. 89P illustrates the effect of senescent cell clearance on CD44^{hi} CD8+ T cells. FIG. 89Q illustrates the effect of senescent cell clearance on NK1.1+ cells.

FIGS. 90A-90F illustrate the effect of senescent cell removal on somatotrophic axis signaling in vivo. FIG. 90A illustrates the effect of senescent cell removal on glucose tolerance. FIG. 90B illustrates the effect of senescent cell removal on insulin sensitivity.

FIG. 90C illustrates the effect of senescent cell removal on serum Igf1. FIG. 90D illustrates the effect of senescent cell removal on SK6 and AKT phosphorylation in inguinal white adipose tissue, kidney and skeletal muscle. FIG. 90E illustrates quantification of the SK6 phosphorylation in FIG. 90D. FIG. 90F illustrates quantification of the AKT phosphorylation in FIG. 90D.

FIG. 91 illustrates retention of Bone Volume (BV/TV) with age through chronic senolytic treatment. Micro Computerized Tomography was used to visualize fixed bone from mice that had been treated with either AP20187 or vehicle from 12-28 months of age. All mice used in this example were male. After sacrifice the cadavers were fixed in 10% neutral buffered formalin, and stored at 4° C. Mice were cleaned of excess tissue, and placed in a Bruker 1176 “Skyscanner” for micro computerized tomographic (mCT) scanning. A variety of settings were explored to visualize whole skeletons, the following settings were used in this example: 65 Kv, 385 μA, 17.58 μm, 0.5 mm Al filter, rotation step 0.5, frame averaging 6, smoothing 2, smoothing kernel 2, ring artifact correction 4, and beam hardening correction 30%. Typical acquisition consisted of a whole body scan, followed by additional scans if necessary for further data exploration. Subvoluming of anatomical features of interest was then further performed on the whole skeleton, using DataViewer, or CtAn software (Bruker). Once a feature had been digitally isolated (for example mid cortical shaft), it was then imported into CtAn (Bruker), or Mimics v18 (Materialise) for further analysis/image processing. The percent bone volume (BV/TV—Bone Volume over Total Volume) is a common metric used in the assessment of aging bone. This represents the amount of bone (BV) contained within a specific volume (TV), hence higher numbers indicate a greater mass of bone in the volume measured. Chronic treatment with AP resulted in retention of an extra 6.1% of cortical bone volume with age.

FIG. 92 illustrates cortical thickness of femoral bone with age through chronic senolytic treatment. Using the methods of FIG. 93 cortical thickness of femoral bone was seen to be increased in aged mice treated with AP20187 compared to controls.

FIG. 93A-C shows a method by which intervertebral disc space, FIG. 93A, FIG. 93B, and FIG. 93C, (IVS) can be measured by microCT. While the intervertebral discs cannot be directly visualized by microCT an image segmentation protocol was developed to infer disc volume by determining the volume between adjacent lumbar vertebra and estimating IVS.

FIG. 94 shows the effect of senolysis treatment on intervertebral spacing in vertebra of the aging mouse spine. As in the experiments of FIGS. 92 and 93, mice were treated with AP20187 or vehicle from 12-28 months of age before sacrifice for imaging. Estimation of IVS between lumbar vertebra L6 and L5, L5 and L4, and L4 and L3 showed 25-33% improved IVS in AP20187 treated animals compared to vehicle treated animals.

FIG. 95 shows a device for measuring grip strength of a rodent. The rodent is allowed to grip onto the bars and is then pulled away from the device until it loses grip on the bars. The device measures and records the maximal force exerted by the animal before losing grip.

FIG. 96 shows the effect of senolysis treatment on grip strength of aging mice. Mice were treated with either vehicle or AP20187 from 12-28 months of age. Grip strength was measured in untreated 12 month old mice, and in mice treated with either vehicle or AP20187 at 18 and 28 months of age. Treatment with the senolytic agent AP20187 resulted in increased grip strength at 28 months compared to a vehicle treated mouse at 28 months of age.

FIG. 97 shows the effect of senolysis treatment on exercise duration of aging mice. Mice were treated with either vehicle or AP20187 from 12-28 months of age. Untreated 18 month old mice, and 28 month old vehicle or AP20187 treated mice were allowed to run on a treadmill and duration of voluntary exercise was recorded. Treatment with the senolytic agent AP20187 resulted in increased duration of exercise.

FIG. 98 shows the effect of senolysis treatment on exercise distance of aging mice. Treatment with the senolytic agent AP20187 resulted in increased exercise distance.

FIG. 99 shows the Gastrocnemius (Gas) and Tibialis anterior (TA) muscles of a mouse.

FIG. 100 shows the effect of senolysis treatment on Gastrocnemius muscle weight of aging mice. Mice were treated with vehicle or AP20187 were sacrificed and gastrocnemius muscles were dissected and weighed. Mice treated with the senolytic agent showed decreased muscle loss with age.

FIG. 101 shows the effect of senolysis treatment on Tibialis anterior muscle weight of aging mice.

FIG. 102 shows the effect of senolysis treatment on muscle fiber area of aging mice.

FIG. 103 shows the effect of senolysis treatment on latency to fall from the rotarod of aging mice.

FIG. 104 shows the effect of senolysis treatment on on latency to fall from the rotarod of aging mice expressed as a ratio relative to baseline.

FIG. 105 shows that Nutlin-treated mice exhibit a better performance on the rotarod over the training period.

### DETAILED DESCRIPTION OF THE INVENTION

Aging is a risk factor for most chronic diseases, disabilities, and declining health. Senescent cells, which are cells in replicative arrest, accumulate as an individual ages and can contribute partially or significantly to cell and tissue deterioration that underlies aging and age related diseases. Cells can also become senescent after exposure to an environmental, chemical, or biological insult or as a result of a disease. Because senescent cells contribute to a variety of pathologies, there is currently a need for agents which selectively kill senescent cells over non-senescent cells.

Disclosed herein are methods and agents for selectively killing senescent cells, and treatment of senescent cell associated diseases and disorders.

### Senolytic Agents and Targets

A senolytic agent as used herein is an agent that “selectively” (preferentially or to a greater degree) destroys, kills, removes, or facilitates selective destruction of senescent cells. In other words, the senolytic agent destroys or kills a senescent cell in a biologically, clinically, and/or statistically significant manner compared with its capability to destroy or kill a non-senescent cell. A senolytic agent is used in an amount and for a time sufficient that selectively kills established senescent cells but is insufficient to kill (destroy, cause the death of) a non-senescent cell in a clinically significant or biologically significant manner. Alternately or in combination, a senolytic agent is an agent that demonstrates a lower EC50 dose (that is, does at which 50% of cells are killed) on senescent cells relative to nonsenescent cells, such as quiescent cells, for example nonsenescent cells grown to high density or to confluence, when measured in an in vitro assay. Alternately or in combination, a senolytic agent is an agent that selectively or differentially or disproportionately kills senescent cells over nonsenescent cells when administered systemically or locally to a mammal. In some embodiments, a senolytic agent is a compound.

As used herein, to ‘selectively kill’ describes a situation whereby an agent disproportionately kills or in some cases otherwise acts upon one cell type relatively to another at a given dosage. Selectivity is demonstrated, for example, by measuring EC50 concentrations in vitro and determining that one cell type demonstrates a lower EC50 than another, indicating that the cell type having a lower EC50 is selectively killed or otherwise acted upon. Alternate measurements of selectively killing, relying upon in vitro or in vivo assays, are compatible with the usage of the term herein, so long as an agent differentially acts upon one cell type relative to another. In some cases the differential acting is killing or triggering apoptosis or otherwise triggering cell death in a cell or population. An agent that selectively kills senescent cells compared to another cell type, such as quiescent nonscenescent cells, is in some cases referred to as a senolytic agent.

Without wishing to be bound by a particular theory, the mechanism by which some inhibitors and antagonists described herein selectively kill senescent cells is by inducing (activating, stimulating, removing inhibition of) an apoptotic pathway that leads to cell death. Non-senescent cells may be proliferating cells or may be quiescent cells. In certain instances, exposure of non-senescent cells to the senolytic agent as used in the methods described herein may temporarily reduce the capability of non-senescent cell to proliferate; however, an apoptotic pathway is not induced and the non-senescent cell is not destroyed.

Certain senolytic agents that may be used in the methods described herein have been described as useful for treating a cancer; however, in the methods for treating a senescence associated disorder or disease, the senolytic agents are administered in a manner that would be considered different and likely ineffective for treating a cancer. The method used for treating a senescence associated disease or disorder with a senolytic agent described herein may comprise one or more of a decreased daily dose, decreased cumulative dose over a single treatment cycle, or decreased cumulative dose of the agent from multiple treatment cycles than the dose of an agent required for cancer therapy; therefore, the likelihood is decreased that one or more adverse effects (i.e., side effects) will occur, which adverse effects are associated with treating a subject according to a regimen optimized for treating a cancer. In contrast, as a senolytic agent, the compounds described herein may be administered at a lower dose than presently described in the art or in a manner that selectively kill senescent cells (e.g., intermittent dosing). In certain embodiments, the senolytic agents described herein may be administered at a lower cumulative dose per treatment course or treatment cycle that would likely be insufficiently cytotoxic to cancer cells to effectively treat the cancer. In other words, according to the methods described herein, the senolytic agent is not used in a manner that would be understood by a person skilled in the art as a primary therapy for treating a cancer, whether the agent is administered alone or together with one or more additional chemotherapeutic agents or radiotherapy to treat the cancer. Even though an agent as used in the methods described herein is not used in a manner that is sufficient to be considered as a primary cancer therapy, the methods and senolytic combinations described herein may be used in a manner (e.g., a short term course of therapy) that is useful for inhibiting metastases. A “primary therapy for cancer” as used herein means that when an agent, which may be used alone or together with one or more agents, is intended to be or is known to be an efficacious treatment for the cancer as determined by a person skilled in the medical and oncology arts, administration protocols for treatment of the cancer using the agent have been designed to achieve the relevant cancer-related endpoints. To further reduce toxicity, a senolytic agent may be administered at a site proximal to or in contact with senescent cells (not tumor cells). Localized delivery of senolytic agents is described in greater detail herein.

Senolytic agents described herein may target one or more pathways. Senolytic targets, include, but are not limited to murine double minute 2 (MDM2), one or more BCL-2 anti-apoptotic protein family members, an Akt family member, c-Jun N-terminal kinase (JNK)1, JNK2, JNK3, Kit, a protein phosphatase 2C (PP2C), MAP kinase phosphatase-1 (MKP-1), reactive oxygen species (ROS) inducement pathways, S6 kinase, protein kinase A (PKA), checkpoint kinase (Chk)1, checkpoint kinase 2, platelet-derived growth factor receptor beta (PDGFRB), vascular endothelial growth factor receptor (VEGFR)-2, phosphoinositide 3-kinase (PI3K), apoptosis signal-regulating kinase 1 (ASK1), spleen tyrosine kinase (Syk), epidermal growth factor receptor (EGFR), cathepsin, poly ADP ribose polymerase (PARP)1 or PARP2, Cathepsin H, cellular FADD-like IL-1β-converting enzyme-inhibitory protein (c-FLIP), Serpin, Ubiquilin-2, Epiregulin, Sorting nexin-3 (Snx3), forkhead box protein O4 (FOXO4), and Proto-oncogene tyrosine protein kinase Src (Src).

### MDM2 Inhibitors

In some embodiments, a senolytic agent is an MDM2 inhibitor.

In some embodiments, the MDM2 inhibitor is a cis-imidazoline compound. In some embodiments, the cis-imidazoline compound is a nutlin compound. In some embodiments, the nutlin compound is Nutlin-1, Nutlin-2, Nutlin-3a, or Nutlin-3b. In some embodiments, the cis-imidazoline compound is RG-7112 (Roche) (CAS No: 939981-39-2; IUPAC name: ((4S,5R)-2-(4-(tert-butyl)-2-ethoxyphenyl)-4,5-bis(4-chlorophenyl)-4,5-dimethyl-4,5-dihydro-1H-imidazol-1-yl)(4-(3-(methylsulfonyl)propyl)piperazin-1-yl)methanone). In some embodiments, the cis-imidazoline compound is RG7338 (Roche) (IPUAC Name: 4-((2R,3S,4R,5S)-3-(3-chloro-2-fluorophenyl)-4-(4-chloro-2-fluorophenyl)-4-cyano-5-neopentylpyrrolidine-2-carboxamido)-3-methoxybenzoic acid) (CAS 1229705-06-9). In some embodiments, the cis-imidazoline compound is a dihydroimidazothiazole compound (e.g., DS-3032b; Daiichi Sankyo). In some embodiments the compound is RG7388 (Roche), (CAS No.: 1229705-06-9, IUPAC name: 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid, also known as Idasanutlin), as shown below.

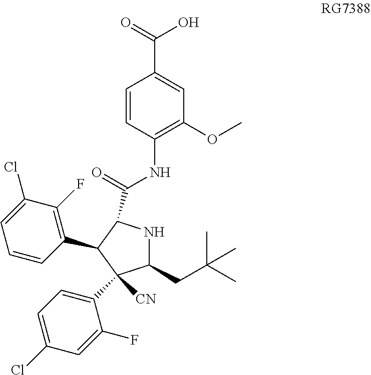 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

In some embodiments, the MDM2 inhibitor is a spiro-oxindole compound. Examples of spiro-oxindole compounds, include, but are not limited to, MI-63, MI-126; MI-122, MI-142, MI-147, MI-18, MI-219, MI-220, MI-221, MI-773, 3-(4-chlorophenyl)-3-41-(hydroxymethyl)cyclopropyl)methoxy)-2-(4-nitrobenzyl)isoindolin-1-one, and MI888.

In some embodiments, the MDM2 inhibitor is a benzodiazepinedione. Benzodiazepinedione compounds that may be used in the methods described herein include 1,4-benzodiazepin-2,5-dione compounds. Examples of benzodiazepinedione compounds include 5-[(3S)-3-(4-chlorophenyl)-4-[(R)-1-(4-chlorophenyl)ethyl]-2,5-dioxo-7-phenyl-1,4-diazepin-1-yl]valeric acid and 5-[(3S)-7-(2-bromophenyl)-3-(4-chlorophenyl)-4-[(R)-1-(4-chlorophenyl)ethyl]-2,5-dioxo-1,4-diazepin-1-yl]valeric acid. Other benzodiazepinedione compounds are called in the art TDP521252 (IUPAC Name: 5-[(3S)-3-(4-chlorophenyl)-4-[(1R)-1-(4-chlorophenyl)ethyl]-7-ethynyl-2,5-dioxo-3H-1,4-benzodiazepin-1-yl]pentanoic acid) and TDP665759 (IUPAC Name: (3S)-4-[(1R)-1-(2-amino-4-chlorophenyl)ethyl]-3-(4-chlorophenyl)-7-iodo-1-[3-(4-methylpiperazin-1-yl)propyl]-3H-1,4-benzodiazepine-2,5-dione).

In some embodiments, the MDM2 inhibitor is a terphenyl compound. In some embodiments, the MDM2 inhibitor is a quilinol. In some embodiments, the MDM2 inhibitor is a chalcone. In some embodiments, the MDM2 inhibitor is a sulfonamide. In some embodiments, the MDM2 inhibitor is a tryptamine such as Serdemetan (JNJ-2684165). In some embodiments, the MDM2 inhibitor is a piperidinone compound. In some embodiments, the piperidinone is AM-8553. In some embodiments, the MDM2 inhibitor is a piperidine. In some embodiments, the MDM2 inhibitor is an imidazole-indole compound. In some embodiments, the MDM2 inhibitor is RO-2443 and RO-5963 ((Z)-2-(4-((6-Chloro-7-methyl-1H-indol-3-yl)methylene)-2,5-dioxoimidazolidin-1-yl)-2-(3,4-difluorophenyl)-N-(1,3-dihydroxypropan-2-yl)acetamide) or CGM097.

In some embodiments the MDM2 inhibitor is AMG232, (CAS No.: 1352066-68-2, IUPAC name: 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid), as shown below.

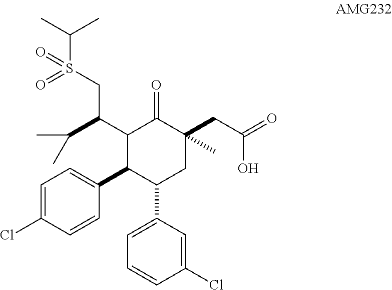

In some embodiments the MDM2 inhibitor is NVP-CGM097, (CAS No.: 1313363-54-0, IUPAC name: (1 S)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one), as shown below.

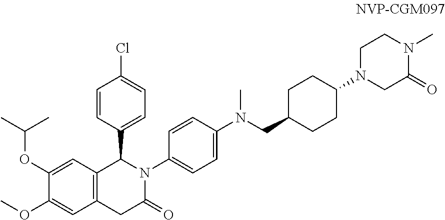 [SMILES: COc1cc2c(cc1OC(C)C)[C@@H](c1ccc(Cl)cc1)N(c1ccc(N(C)C[C@H]3CC[C@H](N4CCN(C)C(=O)C4)CC3)cc1)C(=O)C2]

In some embodiments the MDM2 inhibitor is MI-773, (CAS No.: 1303607-07-9, IUPAC name: (2′R,3S,3′S,5′R)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide), as shown below.

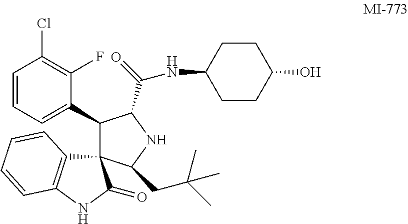 [SMILES: CC(C)(C)C[C@@H]1N[C@@H](C(=O)N[C@H]2CC[C@H](O)CC2)[C@H](c2cccc(Cl)c2F)[C@]12C(=O)Nc1ccccc12]

In some embodiments the MDM2 inhibitor is CAY10681, (CAS No.: 1542066-69-2, IUPAC name: 4-(4-bromophenyl)-4,5-dihydro-5-[3-(1H-imidazol-1-yl)propyl]-3-phenyl-1-(phenylmethyl)-pyrrolo[3,4-c]pyrazol-6(1H)-one), as shown below.

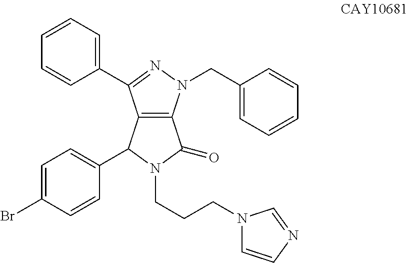 [SMILES: O=C1c2c(c(-c3ccccc3)nn2Cc2ccccc2)C(c2ccc(Br)cc2)N1CCCn1ccnc1]

In some embodiments the MDM2 inhibitor is CAY10682, (CAS No.: 1542066-74-9, IUPAC name: 4-(4-bromophenyl)-1-[(4-fluorophenyl)methyl]-4,5-dihydro-5-[3-(1H-imidazol-1-yl)propyl]-3-phenyl-pyrrolo[3,4-c]pyrazol-6(1H)-one), as shown below.

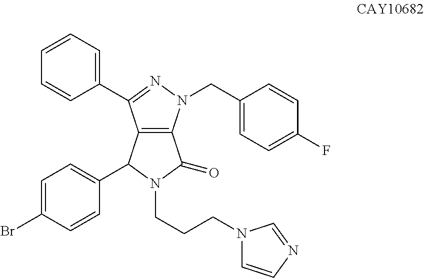 [SMILES: O=C1c2c(c(-c3ccccc3)nn2Cc2ccc(F)cc2)C(c2ccc(Br)cc2)N1CCCn1ccnc1]

In some embodiments the MDM2 inhibitor is YH239-EE, (CAS No.: 1364488-67-4, IUPAC name: Ethyl 3-(2-(tert-butylamino)-1-(N-(4-chlorobenzyl)formamido)-2-oxoethyl)-6-chloro-1H-indole-2-carboxylate), as shown below.

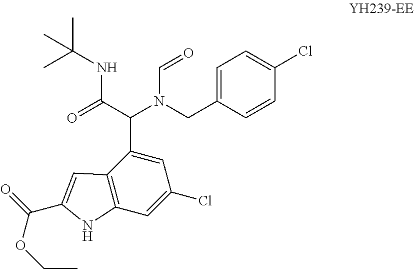 [SMILES: CCOC(=O)c1cc2c(C(C(=O)NC(C)(C)C)N(C=O)Cc3ccc(Cl)cc3)cc(Cl)cc2[nH]1]

In some embodiments the MDM2 inhibitor is RG-7112 (Roche), (CAS No.: 939981-39-2, IUPAC name: ((4S,5R)-2-(4-(tert-butyl)-2-ethoxyphenyl)-4,5-bis(4-chlorophenyl)-4,5-dimethyl-4,5-dihydro-1H-imidazol-1-yl)(4-(3-(methylsulfonyl)propyl)piperazin-1-yl)methanone), as shown below.

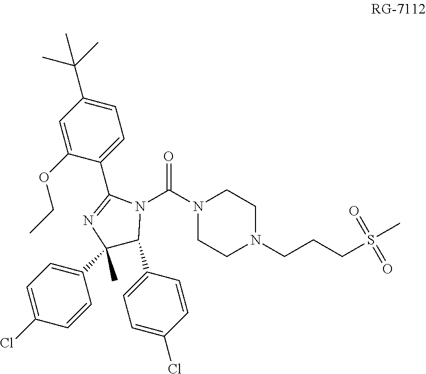 [SMILES: CCOc1cc(C(C)(C)C)ccc1C1=N[C@@](C)(c2ccc(Cl)cc2)[C@@H](c2ccc(Cl)cc2)N1C(=O)N1CCN(CCCS(C)(=O)=O)CC1]

In some embodiments the MDM2 inhibitor is a Boronate. In some embodiments the boronate is trans-4-Iodo, 4′-boranyl-chalcone, (CAS No.: 562823-84-1), as shown below.

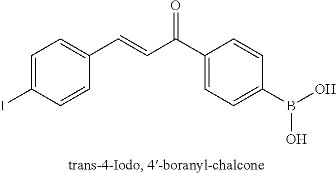 [SMILES: O=C(/C=C/c1ccc(I)cc1)c1ccc(B(O)O)cc1]

In some embodiments the MDM2 inhibitor is RO-5963, (CAS No.: 1416663-77-8, IUPAC name: (Z)-2-(4-((6-Chloro-7-methyl-1H-indol-3-yl)methylene)-2,5-dioxoimidazolidin-1-yl)-2-(3,4-difluorophenyl)-N-(1,3-dihydroxypropan-2-yl)acetamide), as shown below.

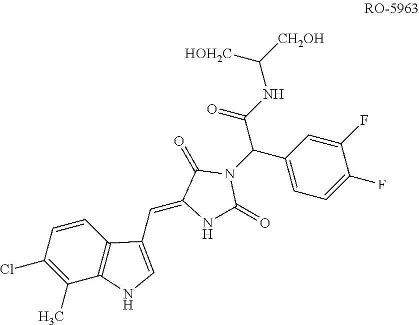 [SMILES: Cc1c(Cl)ccc2c(/C=C3\NC(=O)N(C(C(=O)NC(CO)CO)c4ccc(F)c(F)c4)C3=O)c[nH]c12]

In some embodiments the MDM2 inhibitor is RITA, (CAS No.: 213261-59-7, IUPAC name: 5,5′-(2,5-Furandiyl)bis-2-thiophenemethanol), as shown below.

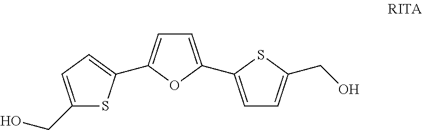 [SMILES: OCc1ccc(-c2ccc(-c3ccc(CO)s3)o2)s1]

In some embodiments the MDM2 inhibitor is HLI 373, (CAS No.: 1782531-99-0, IUPAC name: 5-[[3-(dimethylamino)propyl]amino]-3,10-dimethyl-pyrimido[4,5-b]quinoline-2,4(3H,10H)-dione, dihydrochloride), as shown below.

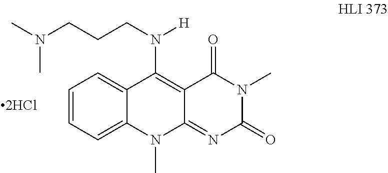 [SMILES: CN(C)CCCNc1c2c(=O)n(C)c(=O)nc-2n(C)c2ccccc12]

In some embodiments the MDM2 inhibitor is JNJ 26854165, (CAS No.: 881202-45-5, IUPAC name: N1-(2-(1H-indol-3-yl)ethyl)-N4-(pyridin-4-yl)benzene-1,4-diamine), as shown below.

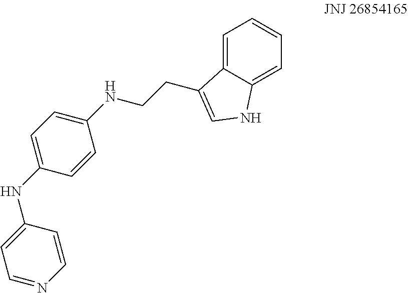 [SMILES: c1ccc2c(CCNc3ccc(Nc4ccncc4)cc3)c[nH]c2c1]

In some embodiments the MDM2 inhibitor is MEL23, (CAS No.: 642072-49-9, IUPAC name: 3-Butyl-6-hydroxy-5-(2,3,4,9-tetrahydro-1H-pyrido[3,4-b]indol-1-yl)pyrimidine-2,4(1H,3H)-dione), as shown below.

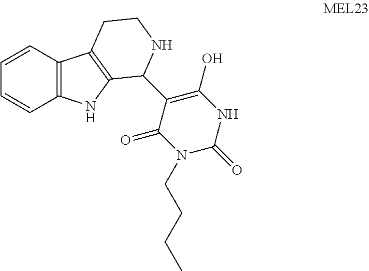 [SMILES: CCCCn1c(=O)[nH]c(O)c(C2NCCc3c2[nH]c2ccccc32)c1=O]

### Bcl-2 Anti-Apoptotic Protein Family Member Inhibitors

In some embodiments, a senolytic agent is a Bcl-2 anti-apoptotic protein family member inhibitor. In some embodiments, a Bcl-2 anti-apoptotic protein family member is Bcl-2, BCL-xL, BCL-w, Al, Mcl-1, BCL-B, or BCL2A1.

In some embodiments, the Bcl-2 anti-apoptotic protein family member inhibitor is a benzothiazole-hydrazone compound, aminopyridine compound, benzimidazole compound, tetrahydroquinoline compound, and a phenoxyl compound and related analogs. Benzothiazole-hydrazone compounds include WEHI-539 (5-[3-[4-(aminomethyl)phenoxy]propyl]-2-[(8E)-8-(1,3-benzothiazol-2-ylhydrazinylidene)-6,7-dihydro-5H-naphthalen-2-yl]-1,3-thiazole-4-carboxylic acid), a BH3 peptide mimetic that selectively targets BCL-xL. An aminopyridine compound that may be used as a selective BCL-xL inhibitor is BXI-61 (3-[(9-amino-7-ethoxyacridin-3-yl)diazenyl]pyridine-2,6-diamine). An example of a benzimidazole compound that may be used as a selective BCL-XL inhibitor is BXI-72 (2′-(4-Hydroxyphenyl)-5-(4-methyl-1-piperazinyl)-2,5′-bi(1H-benzimidazole) trihydrochloride). An example of a phenoxyl compound that may be used as a selective BCL-xL inhibitor is 2[[3-(2,3-dichlorophenoxy) propyl]amino]ethanol (2,3-DCPE).

In some embodiments, the Bcl-2 anti-apoptotic protein family member inhibitor is A-1155463, A-1331852, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-263 (4-[4-[[2-(4-chlorophenyl)-5,5-dimethyl cyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide, ABT-737 (4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]benzamide, or Benzamide, 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]- or 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide).

In some embodiments, the Bcl-2 anti-apoptotic protein family member inhibitor is a quinazoline sulfonamide compound. In some embodiments, the Bcl-2 anti-apoptotic protein family member inhibitor is (R)-4-(4-chlorophenyl)-3-(3-(4-(4-(4-((4-(dimethylamino)-1-(phenylthio)butan-2-yl)amino)-3-nitrophenylsulfonamido)phenyl)piperazin-1-yl)phenyl)-5-ethyl-1-methyl-1H-pyrrole-2-carboxylic acid or (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-ethyl-2-methyl-1H-pyrrole-3-carboxylic acid; Compound 15 (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-isopropyl-2-methyl-1H-pyrrole-3-carboxylic acid). In some embodiments, the BCL-2 anti-apoptotic protein inhibitor is BM-1074, BM-957, BM-1197. In some embodiments, the BCL-2 anti-apoptotic protein inhibitor is an N-acylsufonamide compound, macrocyclic compound, or an isoxazolidine compound.

In certain embodiments, the senolytic agent is a compound that is an inhibitor of Bcl-2, Bcl-w, and Bcl-xL, such as ABT-263 or ABT-737. In certain specific embodiments, the senolytic agent is a compound or a pharmaceutically acceptable salt, stereoisomer, tautomer, or prodrug thereof as illustrated below, which depicts the structure of ABT-263. ABT-263 is also known as Navitoclax in the art.

### Akt Inhibitors

In some embodiments, a senolytic agent is an Akt inhibitor. In some embodiments, the Akt kinase inhibitor inhibits Akt1, Akt2, or Akt3.

In some embodiments, the Akt inhibitor is an ATP competitive inhibitor such as CCT128930.

In some embodiments, the Akt inhibitor is a pan-Akt inhibitor. Examples of pan-Akt inhibitors, include but are not limited to, GDC-0068 (ipatasertib), GSK2110183 (afuresertib), GSK690693, and AT7867.

In some embodiments, the Akt inhibitor is a lipid-based Akt inhibitor that inhibits the generation of PIP3 by PI3K. Examples of a lipid-based Akt inhibitor include, but are not limited to phosphatidylinositol analogs such as Calbiochem Akt Inhibitors I, II and III, or PI3K inhibitors such as PX-866, or Perifosine (KRX-0401).

In some embodiments, the Akt inhibitor is a pseudosubstrate inhibitor. Examples of Akt pseudosubstrate inhibitors include, but are not limited to AKTide-2 T and a FOXO3 hybrid.

In some embodiments, the Akt inhibitor is an allosteric inhibitor. An example of an Akt allosteric inhibitor includes, but is not limited to MK-2206 (8-[4-(1-aminocyclobutyl)phenyl]-9-phenyl-2H-[1,2,4]triazolo[3,4-f][1,6]naphthyridin-3-one; dihydrochloride).

In some embodiments, the Akt inhibitor is an antibody. In some embodiments, the antibody is GST-anti-Akt1-MTS.

In some embodiments, the Akt inhibitor interacts with the PH domain of Akt. In some embodiments, the Akt inhibitor that interacts with the PH domain of Akt is Triciribine or PX-316.

Other compounds described in the art that act as AKT inhibitors include, for example, GSK-2141795 (GlaxoSmithKline), VQD-002, miltefosine, AZD5363, GDC-0068, and API-1. Techniques for determining the activity of AKT inhibitors are routinely practiced by persons skilled in the art.

In other embodiments, the Akt inhibitors inhibit other proteins in addition to Akt kinases. In some embodiments, these other proteins are S6K and PKA. In a particular embodiment, the Akt inhibitor that inhibits at least one Akt kinase, S6K, and PKA is CCT128930 or AT7867.

In a specific embodiment, the senolytic agent is a compound that is an Akt kinase inhibitor, which has the structure as shown below (also called MK-2206 herein and in the art), 8-[4-(1-aminocyclobutyl)phenyl]-9-phenyl-2H-[1,2,4]triazolo[3,4-f][1,6]naphthyridin-3-one; or a pharmaceutically acceptable salt, stereoisomer, tautomer, or prodrug thereof. The dihydrochloride salt is shown.

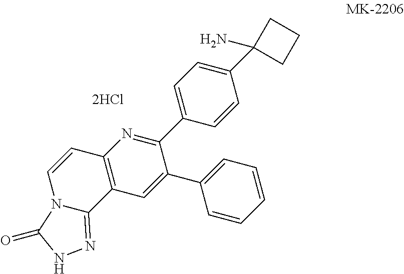 [SMILES: NC1(c2ccc(-c3nc4ccn5c(=O)[nH]nc5c4cc3-c3ccccc3)cc2)CCC1]

### Other Senolytic Agents

In other embodiments, the senolytic agent may be a compound that inhibits JNK1, JNK2, JNK3, or Kit. In a specific embodiment, the senolytic agent that inhibits JNK1, JNK2, JNK3, or Kit is the compound JNK-IN-8 (3-[[4-(dimethylamino)-1-oxo-2-buten-1-yl]amino]-N-[3-methyl-4-[[4-(3-pyridinyl)-2-pyrimidinyl]amino]phenyl]-benzamide).

In other embodiments, the senolytic agent may be a compound that inhibits protein phosphatase 2C (PP2C) or MAP kinase phosphatase-1 (MKP-1). In a specific embodiment, the senolytic agent that inhibits PP2C or MKP-1 is the benzophenanthridine alkaloid sanguinarine chloride (13-Methyl-[1,3]-benzodioxolo[5,6-c]-1,3-dioxolo[4,5-i]phenanthridinium chloride).

In other embodiments, the senolytic agent may be a compound that inhibits apoptosis signal-regulating kinase 1 (ASK1). In a specific embodiment, the senolytic agent that inhibits ASK1 is NQDI-1 (2,7-Dihydro-2,7-dioxo-3H-naphtho[1,2,3-de]quinoline-1-carboxylic acid ethyl ester).

In other embodiments, the senolytic agent may be a compound that inhibits a protein involved in DNA damage repair. In a specific embodiment, the senolytic agent can be a small molecule compound and analogs thereof that inhibits a member in the PARP family of proteins, including PARP1 and PARP2. In a specific embodiment, the senolytic agent that inhibits a PARP family protein is AZD2281, also known as Olaparib, (4-(3-(1-(cyclopropanecarbonyl)piperazine-4-carbonyl)-4-fluorobenzyl)phthalazin-1(2H)-one).

In other embodiments, a senolytic agent may be a small molecule compound and analogs thereof that inhibits a checkpoint kinase, Chk1 or Chk2. In a specific embodiment, the senolytic agent that inhibits Chk1 or Chk2 is the selective ATP-competitive inhibitor AZD7762 (3-[(Aminocarbonyl)amino]-5-(3-fluorophenyl)-N-(3S)-3-piperidinyl-2-thiophenecarboxamide hydrochloride).

In other certain embodiments, the senolytic agent may be a compound that inhibits a protein involved in inflammation. In some embodiments, the senolytic agent is a small molecule compound and analogs thereof that inhibits Syk (spleen tyrosine kinase). In a specific embodiment, the senolytic agent that inhibits Syk is R406 (6-(5-fluoro-2-(3,4,5-trimethoxyphenylamino) pyrimidin-4-ylamino)-2,2-dimethyl-2H-pyrido[3,2-b][1,4]oxazin-3(4H)-one).

In other embodiments, the senolytic agent may be a compound that inhibits a protein involved in proliferation. In some embodiments, the senolytic agent can be a small molecule compound and analogs thereof that inhibits EGFR, which is a receptor tyrosine kinase with downstream signaling pathways involved in proliferation. In a specific embodiment, the senolytic agent that inhibits EGFR is Erlotinib N-(3-ethynylphenyl)-6,7-bis(2-methoxyethoxy)quinazolin-4-amine.

In other embodiments, the senolytic agent may be a small molecule compound and analogs thereof that inhibits multiple receptor tyrosine kinases, such as PDGFRB and VEGFR2. In a specific embodiment, the senolytic agent that inhibitss multiple receptor tyrosine kinases is Sunitinib (N-(2-diethylaminoethyl)-5-[(Z)-(5-fluoro-2-oxo-1H-indol-3-ylidene)methyl]-2,4-dimethyl-1H-pyrrole-3-carboxamide).

In other embodiments, the senolytic agent may be an inhibitor of a protease. In certain embodiments, the protease belongs to the cathepsin family of proteases. In certain embodiments, the compound that inhibits a cathepsin family protease is CYM 7008-00-01.

In other embodiments, the senolytic agent may be a glucosamine or analog thereof. In some embodiments, the senolytic agent is GlcNAc, or N-Acetylglucosamine. In other embodiments, the senolytic agent is 3,4,6-O-Bu3-GlcNAc.

In other embodiments, the senolytic agent may be a small molecule compound that induces apotosis through the production of reactive oxygen species (ROS). In a specific embodiment, the senolytic agent capable of inducing the production of ROS is methyl 3-(4-nitrophenyl) propiolate (NPP).

In certain embodiments, at least one senolytic agent may be administered with at least one other senolytic agent, which two or more senolytic agents act additively or synergistically to selectively kill senescent cells.

Combinations with mTOR, NFκB, and PI3K Pathway Inhibitors

A small molecule compound that may be used together with a senolytic agent described herein in the methods for selectively killing senescent cells and treating a senescence-associated disease or disorder may be a small molecule compound that inhibits one or more of mTOR, NFκB, and PI3-k pathways. As described herein, methods are also provided for selectively killing senescent cells and for treating a senescence-associated disease or disorder, wherein the methods comprise administering to a subject in need thereof at least one senolytic agent, which methods may further comprise administering an inhibitor of one or more of mTOR, NFκB, and PI3-k pathways. Inhibitors of these pathways are known in the art.

Examples of mTOR inhibitors include sirolimus, temsirolimus, everolimus, ridaforolimus, 32-deoxorapamycin, zotarolimus, PP242, INK128, PP30, Torinl, Ku-0063794, WAY-600, WYE-687 and WYE-354. Inhibitors of an NFκB pathway include, for example, NFκB activity abrogation through TPCA-1 (an IKK2 inhibitor); BAY 11-7082 (an IKK inhibitor poorly selective for IKK1 and IKK2); and MLN4924 (an NEDD8 activating enzyme (NAE)-inhibitor); and MG132.

Examples of inhibitors of PI3-k that may also inhibit mTOR or AKT pathways include perifosine (KRX-0401), idelalisib, PX-866, IPI-145, BAY 80-6946, BEZ235, RP6530, TGR 1201, SF1126, INK1117, GDC-0941, BKM120 (NVP-BKM120, Buparlisib), XL147 (SAR245408), XL765 (SAR245409), Palomid 529, GSK1059615, GSK690693, ZSTK474, PWT33597, IC87114, TG100-115, CAL263, RP6503, PI-103, GNE-477, CUDC-907, AEZS-136, BYL719, GDC-0980 (RG7422, Apitolisib), GDC-0032, and MK2206.

### Methods for Treating Senescent Cell Associated Diseases and Disorder

Provided herein are methods for selectively killing senescent cells and thereby treating or preventing (reducing the likelihood of occurrence of), or delaying the onset or progression of a senescent cell-associated disease or disorder and comprises use of a senolytic agent as described herein. As described herein, these senolytic agents are administered in a manner that would be considered ineffective for treating a cancer. Because the method used for treating a senescence associated disease with a senolytic agent described herein comprises one or more of a decreased daily dose, decreased cumulative dose over a single therapeutic cycle, or decreased cumulative dose of the senolytic agent (e.g., an MDM2 inhibitor; an inhibitor of at least one Bcl-2 anti-apoptotic family member that inhibits at least Bcl-xL; an Akt inhibitor) over multiple therapeutic cycles compared with the amount required for cancer therapy, the likelihood is decreased that one or more adverse effects (i.e., side effects) will occur, which adverse effects are associated with treating a subject according to a regimen optimized for treating a cancer.

The treatment regimen of the methods for treating a senescent cell-associated disease or disorder, comprises administering a senolytic agent for a time sufficient and in an amount sufficient that selectively kills senescent cells. In certain embodiments, the senolytic agent is administered within a treatment cycle, which treatment cycle comprises a treatment course followed by a non-treatment interval. A treatment course of administration refers herein to a finite time frame over which one or more doses of the senolytic agent on one or more days are administered. The finite time frame may be also called herein a treatment window.

In one embodiment, a method is provided herein for treating a senescent cell-associated disease or disorder, which is not a cancer, and which method comprises administering to a subject in need thereof a small molecule senolytic agent that selectively kills senescent cells and is administered within a treatment cycle. In a particular embodiment, the methods comprise administering the senolytic agent in at least two treatment cycles. In a specific embodiment, the non-treatment interval may be at least about 2 weeks or between at least about 0.5-12 months, such as at least about one month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months (i.e., 1 year). In other certain particular embodiments, the non-treatment interval is between 1-2 years or between 1-3 years, or longer. In certain embodiments, each treatment course is no longer than about 1 month, no longer than about 2 months, or no longer than about 3 months; or is no longer than 1, 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days.

In certain embodiments, the treatment window (i.e., treatment course) is only one day. In other certain embodiments, a single treatment course occurs over no longer than 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. During such treatment windows, the senolytic agent may be administered at least on two days (i.e., two days or more) with a variable number of days on which the agent is not administered between the at least two days of administration. Stated another way, within a treatment course when the senolytic agent is administered on two or more days, the treatment course may have one or more intervals of one or more days when the senolytic agent, is not administered. By way of non-limiting example, when the senolytic agent is administered on 2 or more days during a treatment course not to exceed 21 days, the agent may be administered on any total number of days between from 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. In certain embodiments, the senolytic agent is administered to a subject during a treatment course of 3 days or more, and the agent may be administered every 2^{nd} day (i.e., every other day). In other certain embodiments when the senolytic agent is administered to a subject for a treatment window of 4 days or more, the senolytic agent may be administered every 3^{rd} day (i.e., every other third day). In one embodiment, the senolytic agent is administered on at least two days (i.e., 2 or more) during a treatment course that is at least 2 days and no more than about 21 days (i.e., from about 2-21 days); at least 2 days and no longer than about 14 days (i.e., from about 2-14 days); at least 2 days and no longer than about 10 days (i.e., from about 2-10 days); or at least 2 days and no longer than about 9 days (i.e., from about 2-9 days); or at least 2 days and no longer than about 8 days (i.e., from about 2-8 days). In other specific embodiments, the senolytic agent is administered on at least two days (i.e., 2 or more) during a treatment window is at least 2 days and no longer than about 7 days (i.e., from about 2-7 days); at least 2 days and no longer than about 6 days (i.e., from about 2-6 days) or at least 2 days and no more than about 5 days (i.e., from about 2-5 days) or at least 2 days and no longer than about 4 days (i.e., from about 2-4 days). In yet another embodiment, the treatment window is at least 2 days and no longer than 3 days (i.e., 2-3 days), or 2 days. In certain particular embodiments, the treatment course is no longer than 3 days. In other embodiments, the treatment course is no longer than 5 days. In still other specific embodiments, the treatment course is no longer than 7 days, 10 days, or 14 days or 21 days. In certain embodiments, the senolytic agent is administered on at least two days (i.e., 2 or more days) during a treatment window that is at least 2 days and no longer than about 11 days (i.e., 2-11 days); or the senolytic agent is administered on at least two days (i.e., 2 or more days) during a treatment window that is at least 2 days and no longer than about 12 days (i.e., 2-12 days); or the senolytic agent is administered on at least two days (i.e., 2 or more days) during a treatment window that is at least 2 days and no more than about 13 days (i.e., 2-13 days); or the senolytic agent is administered on at least two days (i.e., 2 or more days) during a treatment course that is at least 2 days and no more than about 15 days (i.e., 2-15 days); or the senolytic agent is administered on at least two days (i.e., 2 or more days) during a treatment course that is at least 2 days and no longer than about 16 days, 17 days, 18 days, 19 days, or 20 days (i.e., 2-16, 2-17, 2-18, 2-19, 2-20 days, respectively). In other embodiments, the senolytic agent may be administered on at least 3 days over a treatment course of at least 3 days and no longer than any number of days between 3 and 21 days; or is administered on at least 4 days over a treatment course of at least 4 days and no longer than any number of days between 4 and 21 days; or is administered on at least 5 days over a treatment course of at least 5 days and no longer than any number of days between 5 and 21 days; or is administered on at least 6 days over a treatment course of at least 6 days and no longer than any number of days between 6 and 21 days; or is administered at least 7 days over a treatment course of at least 7 days and no longer than any number of days between 7 and 21 days; or is administered at least 8 or 9 days over a treatment course of at least 8 or 9 days, respectively, and no longer than any number of days between 8 or 9 days, respectively, and 21 days; or is administered at least 10 days over a treatment course of at least 10 days and no longer than any number of days between 10 and 21 days; or is administered at least 14 days over a treatment course of at least 14 days and no longer than any number of days between 14 and 21 days; or is administered at least 11 or 12 days over a treatment course of at least 11 or 12 days, respectively, and no longer than any number of days between 11 or 12 days, respectively, and 21 days; or is administered at least 15 or 16 days over a treatment course of at least 15 or 16 days, respectively, and no longer than any number of days between 15 or 16 days, respectively, and 21 days. By way of additional example, when the treatment course is no longer than 14 days, a senolytic agent may be administered on at least 2, 3, 4, 5, 6, 7, 8, 9, 10, 11, 12, 13, and 14 days over a treatment of window of at least 2, 3, 4, 5, 6, 7, 8, 9, 10, 11, 12, 13, and 14 days, respectively, and no longer than 14 days. When the treatment course is no longer than 10 days, a senolytic agent may be administered on at least 2, 3, 4, 5, 6, 7, 8, 9, or 10 days over a treatment of window of at least 2, 3, 4, 5, 6, 7, 8, 9, or 10 days, respectively, and no longer than 10 days. Similarly, when the treatment course is no longer than 7 days, a senolytic agent may be administered on at least 2, 3, 4, 5, 6, or 7 days over a treatment window of at least 2, 3, 4, 5, 6, or 7 days, respectively, and no longer than 7 days. In still another example, when the treatment course is no longer than 5 days, a senolytic agent may be administered on at least 2, 3, 4, or 5 days over a treatment of window of at least 2, 3, 4, or 5 days, respectively, and no longer than 5 days.

With respect to a treatment course of three or more days, doses of the senolytic agent may be administered for a lesser number of days than the total number of days within the particular treatment window. By way of non-limiting example, when a course of treatment has a treatment course of no more than 7, 10, 14, or 21 days, the number of days on which the senolytic agent may be administered is any number of days between 2 days and 7, 10, 14, or 21 days, respectively, and at any interval appropriate for the particular disease being treated, the senolytic agent being administered, the health status of the patient and other relevant factors, which are discussed in greater detail herein. A person skilled in the art will readily appreciate that when the senolytic agent is administered on two or more days over a treatment window, the agent may be delivered on the minimum number days of the window, the maximum number of days of the window, or on any number of days between the minimum and the maximum.

In certain specific embodiments, a treatment course is one day or the treatment course is of a length not to exceed 2, 3, 4, 5, 6, 7, 8, 9, 10, 11, 12, 13, or 14 days, which are examples of a course wherein the senolytic agent is administered on two or more days over a treatment course not to exceed (i.e., no longer than) 2, 3, 4, 5, 6, 7, 8, 9, 10, 11, 12, 13, or 14 days, respectively. In other certain embodiments, the treatment course is about 2 weeks (about 14 days or 0.5 months), about 3 weeks (about 21 days), about 4 weeks (about one month), about 5 weeks, about 6 weeks (about 1.5 months), about 2 months (or about 60 days), or about 3 months (or about 90 days). In a particular embodiment, a treatment course is a single daily dosing of the senolytic agent. In other embodiments, with respect to any treatment course a daily dose of the senolytic agent may be as a single administration or the dose may be divided into 2, 3, 4, or 5 separate administrations to provide the total daily dose of the agent.

As described herein, in certain specific embodiments, within a treatment window when the senolytic agent is administered on two are more days, the treatment course may have one or more intervals of one or more days when the senolytic agent, is not administered. Solely as a non-limiting example, when a treatment window is between two and seven days, a first dose may be administered on the first day of the treatment window and a second dose may be administered on the third day of the course, and a third dose may be administered on the seventh day of the treatment window. A person skilled in the art will appreciate that varying dosing schedules may be used during a particular treatment window. In other specific embodiments, the senolytic agent is administered daily on each consecutive day for the duration of the treatment course. A daily dose may be administered as a single dose or the daily dose may be divided into 2, 3, or 4, or 5 separate administrations to provide the total daily dose of the senolytic agent.

In certain embodiments, the treatment course comprises a length of time during which the senolytic agent is administered daily. In one specific embodiment, the senolytic agent is administered daily for 2 days. In another specific embodiment, the senolytic agent is administered daily for 3 days. In yet another particular embodiment, the senolytic agent is administered daily for 4 days. In one specific embodiment, the senolytic agent is administered daily for 5 days. In yet another particular embodiment, the senolytic agent is administered daily for 6 days. In another specific embodiment, the senolytic agent is administered daily for 7 days. In yet another particular embodiment, the senolytic agent is administered daily for 8 days. In still another specific embodiment, the senolytic agent is administered daily for 9 days. In yet another particular embodiment, the senolytic agent is administered daily for 10 days. In yet another particular embodiment, the senolytic agent is administered daily for 11 days. In yet another particular embodiment, the senolytic agent is administered daily for 12 days. In yet another particular embodiment, the senolytic agent is administered daily for 13 days. In yet another particular embodiment, the senolytic agent is administered daily for 14 days. The treatment window (i.e., course) for each of the above examples is no longer than 2, 3, 4, 5, 6, 7, 8, 9, 10, 11, 12, 13, 14 days, respectively.

In other specific embodiments, the senolytic agent is administered every 2^{nd} day (i.e., every other day) for 3, 4, 5, 6, 7, 8, 9, 10, 11, 12, 13, or 14 days. In still other specific embodiments, the senolytic agent is administered every 2^{nd} day (i.e., one day receiving the agent followed by two days without receiving the agent) for 4, 5, 6, 7, 8, 9, 10, 11, 12, 13, or 14 days. In still other specific embodiments, the senolytic agent may be administered on every 2^{nd}-3^{rd} day during a treatment window of 3, 4, 5, 6, 7, 8, 9, 10, 11, 12, 13, or 14 days. In yet other embodiments, the senolytic agent may be administered every 4^{th} day during a treatment course of 5, 6, 7, 8, 9, 10, 11, 12, 13, or 14 days; or every 5^{th} day during a treatment course of 6, 7, 8, 9, 10, 11, 12, 13, or 14 days. A person skilled in the art can readily appreciate the minimum numbers of days in a treatment window when the senolytic agent is administered every 6^{th}, 7^{th}, etc. day over a treatment window of a finite number of days as described herein.

In certain particular embodiments, a senolytic agent may be administered daily for a longer duration than 14 days and may be administered at least 15, 16, 17, 18, 19, 20, or at least 21 days. In other specific embodiments, the senolytic agent may be administered daily on each of the 15, 16, 17, 18, 19, 20, or 21 days. In another specific embodiment, the senolytic agent may be administered every second day during a treatment window of 15, 16, 17, 18, 19, 20, or 21 days. In another specific embodiment, the senolytic agent may be administered every third day during a treatment window of 15, 16, 17, 18, 19, 20, or 21 days. In still other specific embodiments, the senolytic agent may be administered on every 2^{nd}-3^{rd} day during a treatment window of 15, 16, 17, 18, 19, 20, or 21 days. In yet other embodiments, the senolytic agent may be administered every 4^{th} day during a treatment course of 15, 16, 17, 18, 19, 20, or 21 days; or every 5^{th} day during a treatment course of 15, 16, 17, 18, 19, 20, or 21 days. A person skilled in the art can readily appreciate the minimum numbers of days in a treatment window when the senolytic agent is administered every 6^{th}, 7^{th}, etc. day over a treatment window of a finite number of days as described herein.

In another certain particular embodiment, a senolytic agent may be administered daily for a longer duration than 14 days and may be administered at least 15, 16, 17, 18, 19, 20, or at least 21 days. In other specific embodiments, the senolytic agent may be administered daily on each of the 15, 16, 17, 18, 19, 20, or 21 days. In another specific embodiment, the senolytic agent may be administered every second day during a treatment window of 15, 16, 17, 18, 19, 20, or 21 days. In another specific embodiment, the senolytic agent may be administered every third day during a treatment window of 15, 16, 17, 18, 19, 20, or 21 days. In still other specific embodiments, the senolytic agent may be administered on every 2^{nd}-3^{rd} day during a treatment window of 15, 16, 17, 18, 19, 20, or 21 days. In yet other embodiments, the senolytic agent may be administered every 4^{th} day during a treatment course of 15, 16, 17, 18, 19, 20, or 21 days; or every 5^{th} day during a treatment course of 15, 16, 17, 18, 19, 20, or 21 days. A person skilled in the art can readily appreciate the minimum numbers of days in a treatment window when the senolytic agent is administered every 6^{th}, 7^{th}, etc. day over a treatment window of a finite number of days as described herein.

In another certain particular embodiment, a senolytic agent may be administered in a treatment course daily for a longer duration than 14 days or 21 days and may be administered in a treatment course of about one month, about two months, or about three months. In other specific embodiments, the senolytic agent may be administered daily on each of a one month, two month, or three month treatment course. In another specific embodiment, the senolytic agent may be administered every second day during a treatment course of about one month, about two months, or about three months. In another specific embodiment, the senolytic agent may be administered every third day during a treatment course of about one month, about two months, or about three months. In still other specific embodiments, the senolytic agent may be administered on every 2^{nd}-3^{rd} day during a treatment course of about one month, about two months, or about three months. In yet other embodiments, the senolytic agent may be administered every 4^{th} day during a treatment course of about one month, about two months, or about three months; or every 5^{th} day during a treatment course of about one month, about two months, or about three months s. A person skilled in the art can readily appreciate the minimum numbers of days in a treatment course when the senolytic agent is administered every 6^{th}, 7^{th}, etc. day over a treatment window of a finite number of days as described herein.

By way of non-limiting example, a longer treatment window with a decreased dose per day may be a treatment option for a subject. In other particular embodiments and by way of example, the stage or severity of the senescence associated disease or disorder or other clinical factor may indicate that a longer term course may provide clinical benefit. In certain embodiments, the senolytic agent is administered daily, or optionally, every other day (every 2^{nd} day) or every 3^{rd} day, or greater interval (i.e., every 4^{th} day, 5^{th} day, 6^{th} day) during a treatment course of about 1-2 weeks (e.g., about 5-14 days), about 1-3 weeks (e.g., about 5-21 days), about 1-4 weeks (e.g., about 5-28 days, about 5-36 days, or about 5-42 days, 7-14 days, 7-21 days, 7-28 days, 7-36 days, or 7-42 days; or 9-14 days, 9-21 days, 9-28 days, 9-36 days, or 9-42 days. In other certain embodiments, the treatment course is between about 1-3 months. In a specific embodiment, the senolytic agent is administered daily for at least five days, and in another particular embodiment, the senolytic agent is administered daily for 5-14 days. In other particular embodiments, the senolytic agent is administered for at least seven days, for example, for 7-14, 7-21, 7-28 days, 7-36 days, or 7-42 days. In other particular embodiments, the senolytic agent is administered for at least nine days, for example, for 9-14 days, 9-21 days, 9-28 days, 9-36 days, or 9-42 days.

Even though as discussed herein and above, a treatment course comprising administering a senolytic agent provides clinical benefit, in other certain embodiments, a treatment course is repeated with a time interval between each treatment course when the senolytic agent is not administered (i.e., non-treatment interval, off-drug treatment). A treatment cycle as described herein and in the art comprises a treatment course followed by a non-treatment interval. A treatment cycle may be repeated as often as needed. For example, a treatment cycle may be repeated at least once, at least twice, at least three times, at least four times, at least five times, or more often as needed. In certain specific embodiments, a treatment cycle is repeated once (i.e., administration of the senolytic agent comprises 2 treatment cycles). In other certain embodiments, the treatment cycle is repeated twice or repeated 3 or more times. Accordingly, in certain embodiments, one, two, three, four, five, six, seven, eight, nine, ten, or more treatment cycles of treatment with a senolytic agent are performed. In particular embodiments, a treatment course or a treatment cycle may be repeated, such as when the senescence associated disease or disorder recurs, or when symptoms or sequelae of the disease or disorder that were significantly diminished by one treatment course as described above have increased or are detectable, or when the symptoms or sequelae of the disease or disorder are exacerbated, a treatment course may be repeated. In other embodiments when the senolytic agent is administered to a subject to prevent (i.e., reduce likelihood of occurrence or development) or to delay onset, progression, or severity of senescence associated disease or disorder, a subject may receive the senolytic agent over two or more treatment cycles. Accordingly, in certain embodiments, one cycle of treatment is followed by a subsequent cycle of treatment. Each treatment course of a treatment cycle or each treatment course of two or more treatment cycles are typically the same in duration and dosing of the senolytic agent. In other embodiments, the duration and dosing of the senolytic agent during each treatment course of a treatment cycle may be adjusted as determined by a person skilled in the medical art depending, for example, on the particular disease or disorder being treated, the senolytic agent being administered, the health status of the patient and other relevant factors, which are discussed in greater detail herein. Accordingly, a treatment course of a second or any subsequent treatment cycle may be shortened or lengthened as deemed medically necessary or prudent. In other words, as would be appreciated by a person skilled in the art, each treatment course of two or more treatment cycles are independent and the same or different; and each non-treatment interval of each treatment cycle is independent and the same or different.

As described herein, each course of treatment in a treatment cycle is separated by a time interval of days, weeks, or months without treatment with a senolytic agent (i.e., non-treatment time interval or off-drug interval; called non-treatment interval herein). The non-treatment interval (such as days, weeks, months) between one treatment course and a subsequent treatment course is typically greater than the longest time interval (i.e., number of days) between any two days of administration in the treatment course. By way of example, if a treatment course is no longer than 14 days and the agent is administered every other day during this treatment course, the non-treatment interval between two treatment courses is greater than 2 days, such as 3, 4, 5, 6, 7, 8, 9, 10, 11, 12, 13, or 14 days or about 3 weeks, about 4 weeks, about 6 weeks, or about 2 months or longer as described herein. In particular embodiments, the non-treatment interval between two treatment courses is about 5 days, about 1 week, about 2 weeks, about 3 weeks, about 1 month, about 6 weeks, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In certain specific embodiments, the non-treatment interval is about 2 years or about 3 years. In certain specific embodiments, the non-treatment time interval is at least about 14 days, at least about 21 days, at least about 1 month, at least about 2 months, at least about 3 months, at least about 4 months, at least about 5 months, at least about 6 months, or at least about 1 year. In certain embodiments, a course of treatment (whether daily, every other day, every 3^{rd} day, or other interval between administrations within the treatment course as described above (e.g., 1-14 days, 2-14 days, 2-21 days, or 1-21 days)) is administered about every 14 days (i.e., about every 2 weeks) (i.e., 14 days without senolytic agent treatment), about every 21 days (i.e., about every 3 weeks), about every 28 days (i.e., about every 4 weeks), about every one month, about every 36 days, about every 42 days, about every 54 days, about every 60 days, or about every month (about every 30 days), about every two months (about every 60 days), about every quarter (about every 90 days), or about semi-annually (about every 180 days). In other certain embodiments, a course of treatments (e.g., by way of non-limiting example, administration on at least one day or on at least two days during a course for about 2-21 days, about 2-14, days, about 5-14 days, about 7-14 days, about 9-14 days, about 5-21 days, about 7-21 days, about 9-21 days) is administered every 28 days, every 36 days, every 42 days, every 54 days, every 60 days, or every month (about every 30 days), every two months (about every 60 days), every quarter (about every 90 days), or semi-annually (about every 180 days), or about every year (about 12 months). In other embodiments, a course of treatment (such as by way of non-limiting examples, e.g., for about 5-28 days, about 7-28 days, or about 9-28 days whether daily, every other day, every 3^{rd} day, or other interval between administrations within the treatment course) is administered every 36 days, 42 days, 54 days, 60 days, or every month (about every 30 days), every two months (about every 60 days), every quarter (about every 90 days), or semi-annually (about every 180 days). In other particular embodiments, a course of treatment (e.g., for about 5-36 days, 7-36 days, or 9-36 days whether daily, every other day, every 3^{rd} day, or other interval between administrations within the treatment course) is administered every 42 days, 54 days, 60 days, or every month (about every 30 days), every two months (about every 60 days), every quarter (about every 90 days), or semi-annually (about every 180 days), or about every year (about 12 months).

In a particular embodiment, the treatment course is one day and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In other certain embodiments, the treatment course is at least two days or is at least 3 days and no longer than 10 days, and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In still another embodiment, the treatment course is at least three days and no longer than 10 days, no longer than 14 days, or no longer than 21 days, and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In still another embodiment, a treatment course (e.g., for about 5-42, 7-42, or 9-42 days whether daily, every other day, every 3^{rd} day, or other interval between administrations within the treatment course) is administered every 42 days, 60 days, or every month (about every 30 days), every two months (about every 60 days), every quarter (about every 90 days), or semi-annually (about every 180 days), or about every year (about 12 months). In a particular embodiment, the senolytic agent is administered daily for 5-14 days every 14 days (about every 2 weeks), or every 21-42 days. In another particular embodiment, the senolytic agent is administered daily for 5-14 days quarterly. In another particular embodiment, the senolytic agent is administered daily for 7-14 days every 21-42 days. In another particular embodiment, the senolytic agent is administered daily for 7-14 days quarterly. In still other particular embodiments, the senolytic agent is administered daily for 9-14 days every 21-42 days or every 9-14 days quarterly. In still other embodiments, the non-treatment interval may vary between treatment courses. By way of non-limiting example, the non-treatment interval may be 14 days after the first course of treatment and may be 21 days or longer after the second, third, or fourth (or more) course of treatment. In other particular embodiments, the senolytic agent is administered to the subject in need thereof once every 0.5-12 months. In other certain embodiments, the senolytic agent is administered to the subject in need once every 4-12 months.

Some embodiments relate administering a senolytic agent at a dose that effectively clears senescent cells without harming non senescent cells. In certain embodiments the senolytic agent has an EC50 for killing senescent cells which is significantly lower than the EC50 for killing non senescent cells. Non senescent cells includes stem cells, slow dividing cells, rapidly dividing cells and quiescent cells. Example 42 relates EC50s of various senolytic compounds for senescent cells and high density nonsenescent quiescent cells. Examples of compounds with high specificity for killing senescent cells over quiescent cells include boronate, RG-7112, JNJ 26854165 and Me123.

In certain embodiments, a senolytic agent is administered to a subject to reduce the likelihood or the risk that the subject will develop a particular disorder or to delay onset or progression of one or more symptoms of a senescence-associated disease or disorder. In certain embodiments, the senolytic agent is administered for one or more days (e.g., any number of consecutives days between and including 2-3, -4, -5, -6, -7, -8, -9, -10, -11, -12, -13, -14, -15, -16, -17, -18, -19, -20, and 2-21 days) every 3, 4, 5, 6, 7, 8, 9, 10, 11, or 12 months. In a particular embodiment, the senolytic agent is administered for one or more days (e.g., any number of consecutives days between and including 1-9 days) every 5 or 6 months.

Without wishing to be bound by any particular theory, periodic administration of the senolytic agent kills newly formed senescent cells and thereby reduces (decreases, diminishes) the total number of senescent cells accumulating in the subject. In another embodiment, the total number of senescent cells accumulating in the subject is decreased or inhibited by administering the senolytic agent once or twice weekly or according to any of the other treatment courses described above. The total daily dose of a senolytic agent may be delivered as a single dose or as multiple doses on each day of administration. In other certain particular embodiments, when multiple cycles of the senolytic agent are administered, the dose of a senolytic agent administered on a single day may be less than the daily dose administered if only a single treatment course is intended to be administered.

In certain embodiments, method for treating a senescence-associated disease or disorder comprising administering to a subject in need thereof a small molecule senolytic agent that selectively kills senescent cells; wherein the senescence-associated disease or disorder is not a cancer, and wherein the senolytic agent is administered within one or two treatment cycles, typically two treatment cycles. In certain specific embodiments, the non-treatment interval is at least 2 weeks and each treatment course is no longer than 3 months.

Also provided herein are methods for selectively killing a senescent cell comprising contacting the senescent cell with a senolytic agent described herein (i.e., facilitating interaction or in some manner allowing the senescent cell and senolytic agent to interact) under conditions and for a time sufficient to kill the senescent cell. In such embodiments, the agent selectively kills senescent cells over non-senescent cells (i.e., the agent selectively kills senescent cells compared with killing of non-senescent cells). In certain embodiments, the senescent cell to be killed is present in a subject (e.g., a human or non-human animal). The senolytic agent(s) may be administered to the subject according to the treatment cycles, treatment courses, and non-treatment intervals described above and herein.

In particular embodiments, a single (i.e., only, sole) senolytic agent is administered to the subject for treating a senescence-associated disease or disorder. In certain embodiments, administration of a single senolytic agent may be sufficient and clinically beneficial to treat a senescence-associated disease or disorder. Accordingly, in certain particular embodiments, a senolytic agent is administered as a monotherapy and is the single (i.e., only, sole) active agent administered to the subject for treating the condition or disease. Medications that are not necessarily excluded from administration to the subject when a senolytic agent is administered as a monotherapy include, by way of non-limiting examples, medications for other purposes such as palliative care or comfort (e.g., aspirin, acetominophen, ibuprofen, or prescription pain-killers; anti-itching topical medications) or for treating a different disease or condition, especially if the other medications are not senolytic agents, such as drugs for lowering cholesterol, statins, eye wetting agents, and other such medications familiar to a person skilled in the medical art.

In specific embodiments, if the senolytic agent is an MDM2 inhibitor, the MDM2 inhibitor is administered as a monotherapy (i.e., the only active therapeutic agent), and each treatment course is at least 5 days long during which course the MDM2 inhibitor is administered on at least 5 days. In certain other embodiments, the MDM2 inhibitor is administered on at least 9 days. In still more specific embodiments, the MDM2 inhibitor is Nutlin-3a.

The dosing regimens, treatment courses, and treatment cycles (can be reviewed and modified or adjusted, continued or discontinued, as determined by a person skilled in the art, depending on the responsiveness of the subject to the senolytic agent, the stage of the disease, the general health of the subject, and other factors that are described herein and in the art.

As described herein, certain senolytic agents that may be used in the methods have been described as useful or potentially useful for treating a cancer; however, in embodiments of the methods for treating a senescence associated disorder or disease, the senolytic agents are administered in a manner that would be considered different and likely ineffective for treating a cancer. Accordingly, the methods described herein are useful for treating a senescence-associated disorder or disease but are not described as also useful as a primary therapy (alone or with another chemotherapy agent or radiotherapy) for treating a cancer. In one embodiment, the method used for treating a senescence associated disease or disorder with a senolytic agent may comprise a decreased daily dose compared with the daily dose of the agent as required for cancer therapy. In another embodiment, the method used for treating a senescence associated disease or disorder with a senolytic agent described herein may comprise decreased cumulative dose over a single treatment cycle compared with the cumulative dose of the agent as required for cancer therapy. In still another embodiment, the method used for treating a senescence associated disease or disorder with a senolytic agent described herein may comprise or decreased cumulative dose of the agent administered over multiple treatment cycles compared with the dose of the agent as required for multiple cancer therapy cycles.

By way of example, in certain embodiments, when the senolytic agent is an agent that can be cytotoxic to cancer cells and may be used in the oncology art in a manner for treating a cancer (for example, an MDM2 inhibitor (e.g., Nutlin-3a; RG-7112) or an inhibitor of one or more BCL-2 anti-apoptotic protein family members and which inhibits at least Bcl-xL (e.g., ABT-263, ABT-737, WEHI-539, A-1155463)), the methods for treating a senescence associated disease or disorder comprise administering the senolytic agent in one or two or more treatment cycles, and the total dose of the senolytic agent administered during each treatment course, each treatment cycle, and/or cumulatively over two or more treatment cycles is an amount less than the amount effective for a cancer treatment. The amount of such a senolytic agent administered to a subject over a given time period (such as one week, two weeks, one month, six months, one year) for treating a senescence associated disease or disorder, for example, may be about from a 20-fold decrease to about a 5000-fold decrease in total amount compared with the total amount of the same agent administered to a subject who is receiving the agent for treatment of a cancer. The fold decrease in the amount (i.e., lesser amount) of the senolytic agent administered over a given time period (i.e., number of days, months, years) for treating a senescence associated disease or disorder may be about a 20-fold decrease, about a 25-fold decrease, about a 30-fold decrease, about a 40-fold decrease, about a 50-fold decrease, about a 60-fold decrease, about a 75-fold decrease, about a 100-fold decrease, about a 125-fold decrease, about a 150-fold decrease, about a 175-fold decrease, about a 200-fold decrease, about a 300-fold decrease, about a 400-fold decrease, about a 500-fold decrease, about a 750-fold decrease, about a 1000-fold decrease, about a 1250-fold decrease, about a 1500-fold decrease, about a 1750-fold decrease, about a 2000-fold decrease, about a 2250-fold decrease, about a 2500-fold decrease, about a 2750-fold decrease, about a 3000-fold decrease, about a 3250-fold decrease, about a 3500-fold decrease, about a 3750-fold decrease, about a 3000-fold decrease, about a 3500-fold decrease, about a 4000-fold decrease, about a 4500-fold decrease, or about a 5000-fold decrease compared with the amount of the agent administered to a subject for treating a cancer over the same length of time. A lower dose required for treating a senescence associated disease may also be attributable to the route of administration. For example, when a senolytic agent is used for treating a senescence-associated pulmonary disease or disorder (e.g., COPD, IPF), the senolytic agent may be delivered directly to the lungs (e.g., by inhalation, by intubation, intranasally, or intratracheally), and a lower dose per day and/or per treatment course is required than if the agent were administered orally. Also, by way of another example, when a senolytic agent is used for treating osteoarthritis or a senescence-associated dermatological disease or disorder, the senolytic agent may be delivered directly to the osteoarthritic joint (e.g., intra-articularly, intradermally, topically, transdermally) or to the skin (e.g., topically, subcutaneously, intradermally, transdermally), respectively, at a lower does per day and/or per treatment course than if the senolytic agent were administered orally. When a senolytic agent is delivered orally, for example, the dose of the senolytic agent per day may be the same amount as administered to a patient for treating a cancer; however, the amount of the agent that is delivered over a treatment course or treatment cycle is significantly less than the amount administered to a subject who receives the appropriate amount of the agent for treating a cancer.

In certain embodiments, the methods described herein comprise using the senolytic agent in an amount that is a reduced amount compared with the amount that may be delivered systemically, for example, orally or intravenously to a subject who receives the senolytic agent when the agent is used for treating a cancer. In certain specific embodiments, methods of treating a senescence-associated disease or disorder by selectively killing senescent cells comprises administering the senolytic agent at a dose that is at least 10% (i.e., one-tenth), at least 20% (one-fifth), 25% (one-fourth), 30%-33% (about one-third), 40% (two-fifths), or at least 50% (half) of the dose that is administered to a subject who has cancer for killing cancer cells during a treatment course, a treatment cycle, or two or more treatment cycles that form the cancer therapy protocol (i.e., regimen). In other particular embodiments, the dose of the senolytic agent(s) used in the methods described herein is at least 60%, 70%, 80%, 85%, 90%, or 95% of the dose that is administered to a subject who has cancer. The therapeutic regimen, comprising the dose of senolytic agent and schedule and manner of administration that may be used for treating a senescence-associated disorder or disease is also a regimen insufficient to be significantly cytotoxic to non-senescent cells.

In certain embodiments, a method for treating a senescence-associated disease or disorder that is not a cancer comprises administering to a subject in need thereof a therapeutically effective amount of a small molecule senolytic agent that selectively kills senescent cells (i.e., selectively kills senescent cells over non-senescent cells or compared with non-senescent cells) and which agent is cytotoxic to cancer cells, wherein the senolytic agent is administered within at least one treatment cycle, which treatment cycle comprises a treatment course followed by a non-treatment interval. The total dose of the senolytic agent administered during the treatment course, and/or the total dose of the senolytic agent administered during the treatment cycle, and/or the total dose of the senolytic agent administered during two or more treatment cycles is an amount less than the amount effective for a cancer treatment. In certain embodiments, the senolytic agent is an inhibitor of a Bcl-2 anti-apoptotic protein family member that inhibits at least Bcl-xL; an MDM2 inhibitor; or an Akt specific inhibitor. Examples of these inhibitors are described herein. In other certain embodiments, the senolytic agent is administered as a monotherapy, and is the single active senolytic agent administered to the subject for treating the disease or disorder. The number of days in the treatment course and the treatment interval are described in detail herein.

In one embodiment, a method is provided herein for treating a senescence-associated disease or disorder, wherein the senescence-associated disease is not cancer and the method comprises administering to a subject in need thereof a senolytic agent or small molecule senolytic compound that selectively kills senescent cells, and the administration is for a short duration (e.g., shorter than may be used for a particular agent for treating a cancer), such as a single day, 2 days, 3 days, 4 days, 5 days, 6 days, 7 days, 8 days, 9 days, 10 days, 11 days, 12 days, 13 days, 14 days, or 15 days. In these particular embodiments, this treatment course on any number of days between 1-15 days is a single treatment course and is not repeated. In another particular embodiment, a senolytic agent is administered for 16 days, 17 days, 18 days, 19 days, 20 days, 21 days, 22 days, 23 days, 24 days, 25 days, 26 days, 27 days, 28 days, 29 days, 30 days, or 31 days as a single treatment course that is not repeated.

In certain specific embodiments, the senolytic agent is ABT-263 (navitoclax). In some embodiments, navitoclax is administered in a treatment window comprising 21 days. In some embodiments, navitoclax is administered daily for 14 days followed by 7 days off. In some embodiments, navitoclax is administered daily for 13 days followed by 8 days off. In some embodiments, navitoclax is administered daily for 12 days followed by 9 days off. In some embodiments, navitoclax is administered daily for 11 days followed by 10 days off. In some embodiments, navitoclax is administered daily for 10 days followed by 11 days off. In some embodiments, navitoclax is administered daily for 9 days followed by 12 days off. In some embodiments, navitoclax is administered daily for 8 days followed by 13 days off. In some embodiments, navitoclax is administered daily for 7 days followed by 14 days off. In some embodiments, navitoclax is administered daily for 6 days followed by 15 days off. In some embodiments, navitoclax is administered daily for 5 days followed by 16 days off. In some embodiments, navitoclax is administered daily for 4 days followed by 17 days off. In some embodiments, navitoclax is administered daily for 3 days followed by 18 days off. In some embodiments, navitoclax is administered daily for 2 days followed by 19 days off. In some embodiments, navitoclax is administered for 1 day followed by 20 days off

In some embodiments, navitoclax is administered daily for 21 days in a dose of about 150 mg to 325 mg. In some embodiments, navitoclax is administered daily for 21 days in a dose of about 150 mg to 300 mg. In some embodiments, navitoclax is administered daily for 21 days in a dose of about 150 mg to 275 mg. In some embodiments, navitoclax is administered daily for 21 days in a dose of about 150 mg to 250 mg. In some embodiments, navitoclax is administered daily for 21 days in a dose of about 150 mg to 225 mg. In some embodiments, navitoclax is administered daily for 21 days in a dose of about 150 mg to 200 mg. In some embodiments, navitoclax is administered daily for 21 days in a dose of about 150 mg to 175 mg. In some embodiments, navitoclax is administered daily for 21 days in a dose of about 150 mg. In some embodiments, navitoclax is administered daily for 21 days in a dose of about 125 mg. In some embodiments, navitoclax is administered daily for 21 days in a dose of about 100 mg. In some embodiments, navitoclax is administered daily for 21 days in a dose of about 75 mg. In some embodiments, navitoclax is administered daily for 21 days in a dose of about 50 mg. In some embodiments, navitoclax is administered daily for 21 days in a dose of about 25 mg.

In some embodiments, navitoclax is administered daily for 14 days in a dose of about 150 mg to 325 mg. In some embodiments, navitoclax is administered daily for 14 days in a dose of about 150 mg to 300 mg. In some embodiments, navitoclax is administered daily for 14 days in a dose of about 150 mg to 275 mg. In some embodiments, navitoclax is administered daily for 14 days in a dose of about 150 mg to 250 mg. In some embodiments, navitoclax is administered daily for 14 days in a dose of about 150 mg to 225 mg. In some embodiments, navitoclax is administered daily for 14 days in a dose of about 150 mg to 200 mg. In some embodiments, navitoclax is administered daily for 14 days in a dose of about 150 mg to 175 mg. In some embodiments, navitoclax is administered daily for 14 days in a dose of about 150 mg. In some embodiments, navitoclax is administered daily for 14 days in a dose of about 125 mg. In some embodiments, navitoclax is administered daily for 14 days in a dose of about 100 mg. In some embodiments, navitoclax is administered daily for 14 days in a dose of about 75 mg. In some embodiments, navitoclax is administered daily for 14 days in a dose of about 50 mg. In some embodiments, navitoclax is administered daily for 14 days in a dose of about 25 mg.

In some embodiments, navitoclax is administered daily for 7 days in a dose of about 150 mg to 325 mg. In some embodiments, navitoclax is administered daily for 7 days in a dose of about 150 mg to 300 mg. In some embodiments, navitoclax is administered daily for 7 days in a dose of about 150 mg to 275 mg. In some embodiments, navitoclax is administered daily for 7 days in a dose of about 150 mg to 250 mg. In some embodiments, navitoclax is administered daily for 7 days in a dose of about 150 mg to 225 mg. In some embodiments, navitoclax is administered daily for 7 days in a dose of about 150 mg to 200 mg. In some embodiments, navitoclax is administered daily for 7 days in a dose of about 150 mg to 175 mg. In some embodiments, navitoclax is administered daily for 7 days in a dose of about 150 mg. In some embodiments, navitoclax is administered daily for 7 days in a dose of about 125 mg. In some embodiments, navitoclax is administered daily for 7 days in a dose of about 100 mg. In some embodiments, navitoclax is administered daily for 7 days in a dose of about 75 mg. In some embodiments, navitoclax is administered daily for 7 days in a dose of about 50 mg. In some embodiments, navitoclax is administered daily for 7 days in a dose of about 25 mg. In other particular embodiments, the above doses are administered daily for 1, 2, 3, 4, 5, or 6 days, 8, 9, 10, 11, 12, 13, 15, 16, 17, 18, 19, or 20 days.

In some embodiments, the senolytic agent is nutlin-3a. In some embodiments, nutlin-3a is administered in a treatment window comprising 28 days. In some embodiments, nutlin-3a is administered daily for 10 days, followed by 18 days off. In some embodiments, nutlin-3a is administered daily for 9 days, followed by 19 days off. In some embodiments, nutlin-3a is administered daily for 8 days, followed by 20 days off. In some embodiments, nutlin-3a is administered daily for 7 days, followed by 21 days off. In some embodiments, nutlin-3a is administered daily for 6 days, followed by 22 days off. In some embodiments, nutlin-3a is administered daily for 5 days, followed by 23 days off. In some embodiments, nutlin-3a is administered daily for 4 days, followed by 24 days off. In some embodiments, nutlin-3a is administered daily for 3 days, followed by 25 days off. In some embodiments, nutlin-3a is administered daily for 2 days, followed by 26 days off. In some embodiments, nutlin-3a is administered for 1 day, followed by 27 days off

In some specific embodiments, nutlin-3a is administered daily for 10 days in a dose of about 20 mg/m². In some embodiments, nutlin-3a is administered daily for 10 days in a dose of about 19 mg/m². In some embodiments, nutlin-3a is administered daily for 10 days in a dose of about 18 mg/m². In some embodiments, nutlin-3a is administered daily for 10 days in a dose of about 17 mg/m². In some embodiments, nutlin-3a is administered daily for 10 days in a dose of about 16 mg/m². In some embodiments, nutlin-3a is administered daily for 10 days in a dose of about 15 mg/m². In some embodiments, nutlin-3a is administered daily for 10 days in a dose of about 14 mg/m². In some embodiments, nutlin-3a is administered daily for 10 days in a dose of about 13 mg/m². In some embodiments, nutlin-3a is administered daily for 10 days in a dose of about 12 mg/m². In some embodiments, nutlin-3a is administered daily for 10 days in a dose of about 11 mg/m². In some embodiments, nutlin-3a is administered daily for 10 days in a dose of about 10 mg/m². In some embodiments, nutlin-3a is administered daily for 10 days in a dose of about 9 mg/m². In some embodiments, nutlin-3a is administered daily for 10 days in a dose of about 8 mg/m². In some embodiments, nutlin-3a is administered daily for 10 days in a dose of about 7 mg/m². In some embodiments, nutlin-3a is administered daily for 10 days in a dose of about 6 mg/m². In some embodiments, nutlin-3a is administered daily for 10 days in a dose of about 5 mg/m². In some embodiments, nutlin-3a is administered daily for 10 days in a dose of about 4 mg/m². In some embodiments, nutlin-3a is administered daily for 10 days in a dose of about 3 mg/m². In some embodiments, nutlin-3a is administered daily for 10 days in a dose of about 2 mg/m². In some embodiments, nutlin-3a is administered daily for 10 days in a dose of about 1 mg/m². In some embodiments, nutlin-3a is administered daily for 10 days in a dose of about 0.75 mg/m². In some embodiments, nutlin-3a is administered daily for 10 days in a dose of about 0.5 mg/m². In some embodiments, nutlin-3a is administered daily for 10 days in a dose of about 0.25 mg/m². In some embodiments, nutlin-3a is administered daily for 10 days in a dose of about 0.1 mg/m². In some embodiments, nutlin-3a is administered daily for 10 days in a dose of about 0.01 mg/m². In certain embodiments, nutlin-3a is administered for 5, 6, 7, 8, 9, 11, 12, 13, or for 14 days at the doses described above.

### Senescent Cell Selectivity

In certain embodiments are methods described herein for the selective killing of senescent cells over non-senescent cells. In some embodiments, administration of the compound or the senolytic agent kills at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.5%, at least about 99.6%, at least about 99.7%, at least about 99.8%, at least about 99.9%, at least about 99.95%, at least about 99.99% of the senescent cells or the non-senescent cells.

In some embodiments, administration of the compound or the senolytic agent kills at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, at most about 96%, at most about 97%, at most about 98%, at most about 99%, at most about 99.5%, at most about 99.6%, at most about 99.7%, at most about 99.8%, at most about 99.9%, at most about 99.95%, at most about 99.99% of the senescent cells or the non-senescent cells.

In some embodiments, administration of the compound or the senolytic agent kills about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 96%, about 96% to about 97%, about 97% to about 98%, about 98% to about 99%, about 99% to about 99.5%, about 99.5% to about 99.6%, about 99.6% to about 99.7%, about 99.7% to about 99.8%, about 99.8% to about 99.9%, about 99.9% to about 99.95%, or about 99.95% to about 99.9% of the senescent cells or the non-senescent cells.

In some embodiments methods of extending lifespan described herein comprise treating a subject with a pharmaceutical composition which kills senescent cells while not harming non senescent cells. In one narrowing of this embodiment MDM2 inhibitors are administered at doses which effectively kill senescent cells while sparing quiescent cells. Examples of such senescent cell selective MDM2 inhibitors are provided in Example 42.

### Senescent Cell Associated Diseases and Disorders

As used herein, a “senescent cell associated disease or disorder” is also known as a “senescence-associated disease or disorder,” or any variation therein.

The senescent cell associated disease or disorder treated by a compound described herein includes a cardiovascular disease or disorder, inflammatory disease or disorder, pulmonary disease or disorder, neurological disease or disorder, metabolic disease or disorder, reproductive disease or disorder, dermatological disease or disorder, a metastasis, a chemotherapy or radiotherapy-induced side effect, age-related disease or disorder, a premature aging disease or disorder, Erdheim-Chester Disease, a gastrointestinal disease, and a sleep disorder.

### Cardiovascular Conditions

In some embodiments, the disclosure provides methods for treating a senescent cell-associated condition, the method comprising administering a therapeutically-effective amount of a compound described herein to a subject in need thereof, wherein the senescent cell-associated condition is a cardiovascular condition. Non-limiting examples of cardiovascular conditions, include, but are not limited to angina, arrhythmia, atherosclerosis, cardiomyopathy, congestive heart failure, coronary artery disease (CAD), carotid artery disease, endocarditis, coronary thrombosis, carotid thrombosis, myocardial infarction (MI), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease, peripheral artery disease (PAD), cardiac stress resistance, and stroke.

A cardiovascular condition can be associated with or caused by arteriosclerosis. An atherosclerotic plaque can be stabilized by administering a compound described herein to a subject in need thereof. In some embodiments, the atherosclerotic plaque is stabilized in a blood vessel, for example, an artery, of a subject, thereby reducing the likelihood of occurrence or delaying the occurrence of a thrombotic event, such as a stroke or MI. A compound described herein can reduce the lipid content or fibrous cap thickness of an atherosclerotic plaque in a subject in need thereof. Fibrous cap formation can occur from the migration and proliferation of vascular smooth muscle cells and from matrix depositions. A thin fibrous cap can contribute to plaque instability and to increased risk of rupture. Such methods can reduce the likelihood of occurrence or delay the occurrence of a thrombotic event, such as a stroke or MI.

A compound described herein can inhibit, reduce, or cause a decrease in the formation of an atherosclerotic plaque in a subject in need thereof, or reduce, decrease, or diminish the amount, or level, of a plaque in a subject in need thereof. Reduction in the amount of a plaque in a blood vessel, for example, an artery, can be determined, for example, by a decrease in surface area of the plaque, or by a decrease in the extent, degree, or percent occlusion of a blood vessel, for example an artery, which can be determined by angiography or other visualizing methods.

A compound described herein can increase, improve, promote, or enhance stability of an atherosclerotic plaque in a subject in need thereof.

The effectiveness of a compound described herein in treating a cardiovascular disease or disorder can be assessed by one or any combination of diagnostic methods, including physical examination and assessment, monitoring of clinical symptoms, and performance of analytical tests and methods. Analytical tests and methods include, but are not limited to, angiography, electrocardiography, stress test, or non-stress test.

### Muscle Loss

This disclosure provides methods of preventing or reducing muscle loss or atrophy, or muscle weakness. Muscle loss, atrophy or weakness can be caused by lack of physical activity, temporary immobilization of the muscle, temporary paralysis, aging, or diseases such as cancer. Treatment with senolytic compounds can prevent or reduce muscle loss. The reduction in muscle loss can be measured by functional studies of muscle strength or physical condition, or by muscle size. Examples of a senolytic compound which can reduce age associated muscle loss are AP20187 and nutlin3a. In example 46 treatment with AP20187 reduced age associated muscle weakness, muscle weight and muscle fiber area. In example 47 aged mice treated with nutlin3a showed enhanced performance on a rotarod, a measure of balance and physical condition, compared to vehicle treated controls. In some aspects of this disclosure treatment with senolytic agents may reduce muscle loss or weakness caused by factors such as, but not limited to, lack of physical activity, temporary immobilization of the muscle, temporary paralysis, aging, or diseases such as cancer. Treatment with senolytic agents may also prevent injuries sustained due to age related muscle weakness.

### Inflammatory and Autoimmune Conditions and Bone Loss

In some embodiments, the disclosure provides methods for treating a senescent cell-associated condition, the method comprising administering a therapeutically-effective amount of a compound described herein to a subject in need thereof, wherein the senescent cell-associated condition is an inflammatory condition. Inflammatory conditions include, but are not limited to, osteoarthritis, osteoporosis, lupus, interstitial cystitis, scleroderma, alopecia, oral mucositis, rheumatoid arthritis, inflammatory bowel disease, kyphosis, herniated intervertebral disc, ulcerative colitis, Crohn's disease, ulcerative asthma, renal fibrosis including post-transplant renal fibrosis, liver fibrosis, pancreatic fibrosis, cardiac fibrosis, skin wound healing including diabetes related wound healing, and oral submucosa fibrosis.

In some embodiments, the disclosure provides methods for treating or reducing the likelihood of conditions resulting from a host immune response to an organ transplant in a subject in need thereof. Non-limiting examples of an organ transplant include a kidney organ transplant, a bone marrow transplant, a liver transplant, a lung transplant, and a heart transplant. In some embodiments, the disclosure provides methods for treating graft-vs-host disease in a subject in need thereof.

In some embodiments, the disclosure provides methods for reducing or inhibiting loss or erosion of proteoglycan layers in a joint in a subject in need thereof. The disclosure provides methods for reducing inflammation in an inflamed joint in a subject in need thereof. A compound described herein can stimulate, enhance, or induce production of collagen in a subject in need thereof, for example, type 2 collagen. A compound described herein can reduce an amount, or level, of an inflammatory cytokine in a subject in need thereof, for example, IL-6. A compound described herein can decrease, inhibit, or reduce the production of metalloproteinase 13 (MMP-13) in a subject in need thereof. A compound described herein can reduce the likelihood of, inhibit, or decrease the erosion of bone in a subject in need thereof. A compound described herein can be administered directly to an osteoarthritic joint, for example, intraarticularly, topically, transdermally, intradermally, or subcutaneously. A compound described herein can restore, improve, or inhibit deterioration of strength of a joint in a subject in need thereof. A compound described herein can reduce joint pain in a subject in need thereof. In some embodiments, the joint is an osteoarthritic joint.

This disclosure provides methods for reducing bone loss. Conditions that can cause bone loss include osteopenia, osteoporosis, inflammatory conditions, chronic kidney disease, overactive parathyroid gland, hormone-blocking treatments, steroid medicines, gastric bypass, cystic fibrosis and aging. Administering an agent that selectively targets senescent cells can prevent or reduce bone loss. One example of a senolytic agent that can prevent or reverse bone loss is AP20187. As shown in Example 44 administering AP20187 reduces age related bone loss.

Some embodiments relate to methods for treating or preventing loss of intervertebral discs. Treating a subject with a senolytic agent can prevent reduction of intervertebral size and loss of intervertebral disc function. Senolytic compounds that could be used to prevent loss of intervertebral discs include all senolytic compounds described herein. One particular senolytic compound which reduces intervertebral disc loss is AP20187. MicroCT studies in Example 45 showed that mice treated with a senolytic compound experienced less age related loss of intervertebral disc space—an indicator of disc size and functionality. Senolytic agents could be administered, in combination with surgery, to correct a spinal defect in a subject in need thereof.

The effectiveness of a compound described herein in treating an inflammatory condition can be assessed by one or any combination of diagnostic methods, including physical examination, assessment and monitoring of clinical symptoms, and performance of analytical tests to monitor the health status of a subject. Physical examination can include, for example, determining tenderness, swelling or redness of the affected joint, and assessment and monitoring of clinical symptoms. Performance of analytical tests and methods can include, for example, determining the level of inflammatory cytokines or chemokines, X-ray images to determine loss of cartilage as shown by a narrowing of space between the bones in a joint, magnetic resonance imaging (MRI), and providing detailed images of bone and soft tissues.

### Pulmonary Conditions

In some embodiments, the disclosure provides methods for treating a senescent cell-associated condition, the method comprising administering a therapeutically-effective amount of a compound described herein to a subject in need thereof, wherein the senescent cell-associated condition is a pulmonary condition. Pulmonary conditions include, but are not limited to, idiopathic pulmonary fibrosis (IPF), chronic obstructive pulmonary disease (COPD), asthma, cystic fibrosis, bronchiectasis, emphysema, age-related loss of pulmonary function, and age-associated sleep apnea.

In some embodiments, the subject has been exposed to environmental pollutants, for example, silica. A subject can be exposed to an occupational pollutant, for example, dust, smoke, asbestos, or fumes. In some embodiments, the subject has smoked cigarettes.

In some embodiments, the subject has a connective tissue disease. The connective tissue disease can be, for example, rheumatoid arthritis, systemic lupus erythematosus, scleroderma, sarcoidosis, or Wegener's granulomatosis. In some embodiments, the subject has an infection. In some embodiments, the subject has taken or is taking medication or has received radiation therapy to the chest. The medication can be, for example, amiodarone, bleomycin, busufan, methotrexate, or nitrofurantoin.

The effectiveness of a compound described herein in treating a pulmonary condition can be assessed by one or any combination of diagnostic methods including physical examination, determination of patient's medical history, determination of patient's family's medical history, chest X-ray, lung function test, spirometry test, blood test, arterial blood gas analysis, bronchoal veolar lavage, lung biopsy, CT scan, and exercise testing. Methods and techniques that evaluate mechanical functioning of the lung, for example, techniques that measure lung capacitance, elastance, and airway hypersensitivity can be performed. To determine lung function and to monitor lung function throughout treatment, any one of numerous measurements can be obtained, for example, expiratory reserve volume (ERV), forced vital capacity (FVC), forced expiratory volume (FEV) (e.g., FEV in one second, FEV1), FEV1/FEV ratio, forced expiratory flow 25% to 75%, maximum voluntary ventilation (MVV), peak expiratory flow (PEF), and slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO2) can also be measured. Normal oxygen levels can be between 95% and 100%. An SpO2 level below 90% can suggest that the subject has hypoxemia. Values below 80% can be critical and require intervention to maintain brain and cardiac function and avoid cardiac or respiratory arrest. Neurological Conditions

In some embodiments, the disclosure provides methods for treating a senescent cell-associated condition, the method comprising administering a therapeutically-effective amount of a compound described herein to a subject in need thereof wherein the senescent cell-associated condition is a neurological condition. Neurological conditions include, but are not limited to, Parkinson's disease, Alzheimer's disease, dementia, amyotrophic lateral sclerosis (ALS), bulbar palsy, pseudobulbar palsy, primary lateral sclerosis, motor neuron dysfunction (MND), mild cognitive impairment (MCI), Huntington's disease, ocular diseases, macular degeneration (wet and dry), glaucoma, vision loss, presbyopia, cataracts, progressive muscular atrophy, lower motor neuron disease, spinal muscular atrophy (SMA), Werdnig-Hoffman Disease (SMA1), SMA2, Kugelberg-Welander Disease (SM3), Kennedy's disease, post-polio syndrome, hereditary spastic paraplegia, age-related memory decline, and depression and mood disorders.

Non-limiting examples for monitoring the effect of a therapy on inhibiting progression of glaucoma include automated perimetry, gonioscopy, imaging technology, scanning laser tomography, HRT3, laser polarimetry, GDX, ocular coherence tomography, ophthalmoscopy, and pachymeter measurements that determine central corneal thickness.

A compound described herein can delay or inhibit the onset or progression of cataracts, presybopia, and macular degeneration in a subject in need thereof who is at risk for developing cataracts, presybopia, and macular degeneration. In some embodiments, the subject is a human subject who is at least 40 years of age.

The effectiveness of a compound described herein in treating a neurological condition can be assessed by one or any combination of diagnostic methods including physical examination, assessment and monitoring of clinical symptoms, and performance of analytical tests and methods described herein.

### Metabolic Conditions

In some embodiments, the disclosure provides methods for treating a senescent cell-associated conditions, the method comprising administering a therapeutically-effective amount of a compound described herein to a subject in need thereof wherein the senescent cell-associated condition is a metabolic condition. Metabolic conditions include, but are not limited to, diabetes (Type 1 or Type 2), metabolic syndrome, diabetic ulcers, obesity, renal dysfunction, nephrological pathology, and glomerular disease.

The effectiveness of a compound described herein in treating a metabolic condition can be assessed by one or any combination of diagnostic methods including physical examination assessment and monitoring of clinical symptoms, and performance of analytical tests and methods, such as those described herein. A subject who is receiving a compound described herein for treatment or reduction in the likelihood of developing diabetes can be monitored, for example, by assaying glucose and insulin tolerance, energy expenditure, body composition, fat tissue, skeletal muscle, and liver inflammation, or lipotoxicity.

### Reproductive Conditions

In some embodiments, the disclosure provides methods for treating a senescent cell-associated condition, the method comprising administering a therapeutically-effective amount of a compound described herein to a subject in need thereof wherein the senescent cell-associated condition is a reproductive condition. Reproductive conditions include, but are not limited to, menopause, androgen (testosterone) decline, female infertility, decreased egg viability, male infertility, decreased sperm viability, sexual dysfunction or sexual malfunction, decreased libido, erectile dysfunction, decreased egg supply, and female sexual arousal disorder.

The effectiveness of a compound described herein in treating a reproductive condition can be assessed by one or any combination of diagnostic methods including physical examination assessment and monitoring of clinical symptoms, and performance of analytical tests and methods, such as those described herein. A subject who is receiving a compound described herein for fertility treatment can be monitored, for example, by semen analysis or hormone testing.

### Dermatological Conditions

In some embodiments, the disclosure provides methods for treating a senescent cell-associated condition, the method comprising administering a therapeutically-effective amount of a compound described herein to a subject in need thereof wherein the senescent cell-associated condition is a dermatological condition. Dermatological conditions include, but are not limited to, psoriasis, eczema, rhytides, pruritis, dysesthesia, papulosquamous disorders, erythroderma, lichen planus, lichenoid dermatosis, atopic dermatitis, eczematous eruptions, eosinophilic dermatosis, rashes, photosensitivity and photoaging related diseases and disorders, reactive neutrophilic dermatosis, pemphigus, pemphigoid, immunobullous dermatosis, fibrohistocytic proliferations of skin, skin nevi, urticaria, hyperpigmentation, cutaneous lymphomas, and cutaneous lupus.

The effectiveness of a compound described herein in treating a dermatological condition can be assessed by one or any combination of diagnostic methods including physical examination assessment and monitoring of clinical symptoms, and performance of analytical tests and methods, such as those described herein.

### Metastasis

In some embodiments, the disclosure provides methods for treating a senescent cell-associated condition, the method comprising administering a therapeutically-effective amount of a compound described herein to a subject in need thereof wherein the senescent cell-associated condition comprises metastasis, such as metastasis of a cancer.

A compound described herein can reduce the likelihood of metastasis in a subject in need thereof. The compound described herein can be administered one or more days within a window of treatment. In some embodiments, the treatment window is 1, 2, 3, 4, 5, 6, 7, 8, 9, 10, 11, 12, 13, 14, 15, 16, 17, 18, 19, 20, or 21 days. In some embodiments, a compound described herein is administered on two or more days within a treatment window of no longer than 7 days or 14 days; on 3 or more days within a treatment window of no longer than 7 days or 14 days; on 4 or more days within a treatment window of no longer than 7 days or 14 days; on 5 or more days within a treatment window of no longer than 7 days or 14 days; or on 6, 7, 8, 9, 10, 11, 12, 13, or 14 days within treatment window of no longer than 7 days or 14 days.

Chemotherapy and radiotherapy treatment regimens can comprise a finite number of cycles of on-drug therapy followed by off-drug therapy, or comprise a finite timeframe in which the chemotherapy or radiotherapy is administered. The protocols can be determined by clinical trials, drug labels, and clinical staff in conjunction with the subject to be treated. The number of cycles of a chemotherapy or radiotherapy or the total length of time of a chemotherapy or radiotherapy regimen can vary depending on the subject's response to the cancer therapy. A compound described herein can be administered after the treatment regimen of chemotherapy or radiotherapy has been completed.

In some embodiments, the metastasis is a solid tumor. In some embodiments, the metastasis is a liquid tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

The methods described herein can also be used for inhibiting progression of metastatic cancer tumors. Non-limiting examples of cancers include adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenstrom macroglobulinemia.

### Chemotherapy and Radiotherapy Side Effects

In some embodiments, the disclosure provides methods for treating a senescent cell-associated condition, the method comprising administering a therapeutically-effective amount of a compound described herein to a subject in need thereof wherein the senescent cell-associated condition is a chemotherapy-induced or radiotherapy-induced side effect. Non-limiting examples of chemotherapeutic agents include anthracyclines, doxorubicin, daunorubicin, taxols, paclitaxel, gemcitabine, pomalidomide, and lenalidomide. Chemotherapy-induced side effects or radiotherapy-induced side effects include, but art not limited to, weight loss, endocrine changes, hormone imbalance, changes in hormome signaling, changes is cardiotoxicity, body composition, reduced ability to be physically active, gastrointestinal toxicity, nausea, vomiting, constipation, anorexia, diarrhea, peripheral neuropathy, fatigue, malaise, low physical activity, hematological toxicity, anemia, hepatotoxicity, alopecia, pain, infection, mucositis, fluid retention, dermatological toxicity, rashes, dermatitis, hyperpigmentation, urticaria, photosensitivity, nail changes, mouth, gum or throat problems, and any toxic side effect caused by a chemotherapy or radiotherapy. In some embodiments, the disclosure provides methods for treating or reducing the likelihood of metastasis comprising administering a compound described herein during an off-chemotherapy or off-radiotherapy time interval or after the chemotherapy or radiotherapy treatment regimen has been completed.

In some embodiments, the chemotherapy-induced side effect is cardiotoxicity and is caused by anthracycline.

In some embodiments, the disclosure provides methods for treating chronic or long term chemotherapy-induced or radiotherapy-induced side effects. Certain toxic effects can appear long after treatment and can result from damage to an organ or system by the therapy. Organ dysfunction, for example, neurological, pulmonary, cardiovascular, and endocrine dysfunction, can be observed in subjects who were treated for cancers during childhood. Chronic or late toxic side effects that occur in subjects who received chemotherapy or radiation therapy include, for example, cardiomyopathy, congestive heart disease, inflammation, early menopause, osteoporosis, infertility, impaired cognitive function, peripheral neuropathy, secondary cancers, cataracts and other vision problems, hearing loss, chronic fatigue, reduced lung capacity, and lung disease.

### Age-related Conditions

In some embodiments, the disclosure provides methods for treating a senescent cell-associated condition, the method comprising administering a therapeutically-effective amount of a compound described herein to a subject in need thereof wherein the senescent cell-associated condition is an age-related condition. In some embodiments, the age-related condition is caused by exposure to an agent or factor such as irradiation, chemotherapy, smoking tobacco, high-fat/high sugar diet, or other environmental factor. Age-related diseases and disorders include, but are not limited to, herniated intervertebral disc, frailty, hair loss, hearing loss, vision loss, macular degeneration, muscle fatigue, skin conditions including wound healing, skin nevi, wrinkly skin, hyperpigmentation, scarring, keloid, rosacea, vitiligo, ichthyosis vulgaris, dermatomyositis, actinic keratosis, sarcopenia, benign prostatic hyperplasia (BPH), renal disease and failure, liver fibrosis, pancreatic fibrosis, oral submucosa fibrosis, and age-related sleep disorders. In some embodiments, the age-related condition is caused by a genetic disorder, such as Hutchinson-Gilford progeria syndrome (HPGS), wherein symptoms of aging, including hair loss, wrinkly skin, atherosclerosis, renal failure, ocular degeneration, and cardiovascular complications, appear at an early age.

In some embodiments, the disclosure provides methods for extending the lifespan of a mammal comprising administering to a subject a compound described herein.

The effectiveness of a compound described herein in treating an age-related condition can be assessed by one or any combination of diagnostic methods including physical examination, patient self-assessment, assessment and monitoring of clinical symptoms, performance of analytical tests and methods, including clinical laboratory tests, physical tests, and exploratory surgery.

### Erdheim-Chester Disease

In some embodiments, the disclosure provides methods for treating a senescent cell-associated condition, the method comprising administering a compound described herein to a subject in need thereof wherein the senescent cell-associated condition is Erdheim-Chester Disease. Erdheim-Chester disease (ECD) (also known as Erdheim-Chester syndrome or polyostotic sclerosing histiocytosis) is a rare disease characterized by the abnormal multiplication of a specific type of white blood cells called histiocytes, or tissue macrophages. Usually, the onset of ECD is in middle age. ECD involves an infiltration of lipid-laden macrophages, multinucleated giant cells, an inflammatory infiltrate of lymphocytes and histiocytes in the bone marrow, and a generalized sclerosis of the long bones. Radiologic osteosclerosis and histology can be diagnostic features for ECD. Video-assisted thoracoscopic surgery can be used for diagnostic confirmation and also for therapeutic relief of recurrent pericardial fluid drainage.

### Gastrointestinal

In some embodiments, the disclosure provides methods for treating a senescent cell-associated condition, the method comprising administering a compound described herein to a subject in need thereof wherein the senescent cell-associated condition is Barrett's Esophagus. Barrett's Esophagus is a condition that can result from acid reflux disease, wherein chronic regurgitation of acid causes the tissue in esophagus to be replaced by tissue similar to the intestinal lining. This condition is associated with increased risk of esophageal cancer.

The effectiveness of a compound described herein in treating Barrett's Esophagus can be assessed by one or any combination of diagnostic methods including physical examination, patient self-assessment, assessment and monitoring of clinical symptoms, performance of analytical tests and methods, including clinical laboratory tests, physical tests, and exploratory surgery.

### Premature Aging Conditions

In some embodiments, the disclosure provides methods for treating a senescent cell-associated condition, the method comprising administering a therapeutically-effective amount of a compound described herein to a subject in need thereof wherein the senescent cell-associated condition is a premature aging disease or disorder. Premature aging diseases and disorders include, but are not limited to Hutchinson-Gilford progeria or Werner's Syndrome. Sleep Conditions

In some embodiments, the disclosure provides methods for treating a senescent cell-associated condition, the method comprising administering a therapeutically-effective amount of a compound described herein to a subject in need thereof wherein the senescent cell-associated condition is a sleep condition. Sleep conditions include, but are not limited to, sleep apnea, hypersomnia, cataplexy, sleep fragmentation, sleeping sickness, sleepwalking, night terrors, bed wetting, bruxism, delayed sleep phase disorder (DSPD), hypopnea syndrome, idiopathic hypersomnia, insomnia, Kleine-Levin syndrome, narcolepsy, excessive daytime sleepiness, nocturia, parasomnias, periodic limb movement disorder, nocturnal myoclonus, hypnic jerk, rapid eye movement sleep behavior disorder, restless leg syndrome, obstructive sleep apnea, sleep paralysis, sleepwalking, somniphobia, situational circadian rhythm sleep disorder, shift worker sleep disorder, and jet lag.

The effectiveness of a senolytic agent with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, patient self-assessment, assessment and monitoring of clinical symptoms, performance of analytical tests and methods, including clinical laboratory tests, physical tests, and exploratory surgery, for example, may be used for monitoring the health status of the subject and the effectiveness of the senolytic agent. The effects of the methods of treatment described herein can be analyzed using techniques known in the art, such as comparing symptoms of patients suffering from or at risk of a particular disease or disorder that have received the pharmaceutical composition comprising a senolytic agent with those of patients who were not treated with the senolytic agent or who received a placebo treatment.

### Lifespan and Age-Related Diseases or Conditions

In certain embodiments, are methods described herein for extending lifespan of a subject in need thereof comprising administering to the subject a compound to selectively kill senescent cells over non-senescent cells. In some embodiments, the extending lifespan of the subject comprises delaying onset or progression of an age-related disease or condition. In some embodiments, a compound, pharmaceutical composition, or unit dose formulation described herein can be used in the treatment of or delaying the onset or progression of an age-related disease or condition. In some embodiments, a compound, pharmaceutical composition, or unit dose formulation described herein can be used for the manufacture of a medicament for the treatment of or delaying the onset or progression of an age-related disease or condition. In some embodiments, the age-related disease or condition is selected from atherosclerosis, cardiovascular disease, cancer, arthritis, dementia, cataract, osteoporosis, diabetes, hypertension, age-related fat loss, lipodystrophy, and kidney disease.

In some embodiments, the subject is a young human, an adult human, an elderly human, or an aged human. In some embodiments, the subject is about 10 years old, about 11 years old, about 12 years old, about 13 years old, about 14 years old, about 15 years old, about 16 years old, about 17 years old, about 18 years old, about 19 years old, about 20 years old, about 21 years old, about 25 years old, about 30 years old, about 35 years old, about 40 years old, about 41 years old, about 42 years old, about 43 years old, about 44 years old, about 45 years old, about 46 years old, about 47 years old, about 48 years old, about 49 years old, about 50 years old, about 51 years old, about 52 years old, about 53 years old, about 54 years old, about 55 years old, about 56 years old, about 57 years old, about 58 years old, about 59 years old, about 60 years old, about 61 years old, about 62 years old, about 63 years old, about 64 years old, about 65 years old, about 66 years old, about 67 years old, about 68 years old, about 69 years old, about 70 years old, about 71 years old, about 72 years old, about 73 years old, about 74 years old, about 75 years old, about 76 years old, about 77 years old, about 78 years old, about 79 years old, about 80 years old, about 81 years old, about 82 years old, about 83 years old, about 84 years old, about 85 years old, about 86 years old, about 87 years old, about 88 years old, about 89 years old, about 90 years old, about 91 years old, about 92 years old, about 93 years old, about 94 years old, about 95 years old, about 96 years old, about 97 years old, about 98 years old, about 99 years old, or about 100 years old.

In some embodiments, onset or progression of an age-related disease or condition can be delayed by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 2 years, at least about 30 months, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, at least about 20 years, least about 25 years, at least about 30 years, at least about 35 years, or at least about 40 years.

In some embodiments, onset or progression of an age-related disease or condition can be delayed by at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 1 year, at most about 13 months, at most about 14 months, at most about 15 months, at most about 16 months, at most about 17 months, at most about 18 months, at most about 19 months, at most about 20 months, at most about 21 months, at most about 22 months, at most about 23 months, at most about 2 years, at most about 30 months, at most about 3 years, at most about 4 years, at most about 5 years, at most about 6 years, at most about 7 years, at most about 8 years, at most about 9 years, at most about 10 years, at most about 11 years, at most about 12 years, at most about 13 years, at most about 14 years, at most about 15 years, at most about 16 years, at most about 17 years, at most about 18 years, at most about 19 years, at most about 20 years, at most about 25 years, most about 30 years, at most about 35 years, or at most about 40 years.

In some embodiments, onset or progression of an age-related disease or condition can be delayed by about 1 month to about 2 months, about 2 months to about 3 months, about 3 months to about 4 months, about 4 months to about 5 months, about 5 months to about 6 months, about 6 months to about 7 months, about 7 months to about 8 months, about 8 months to about 9 months, about 9 months to about 10 months, about 10 months to about 11 months, about 11 months to about 1 year, about 1 year to about 13 months, about 13 months to about 14 months, about 14 months to about 15 months, about 15 months to about 16 months, about 16 months to about 17 months, about 17 months to about 18 months, about 18 months to about 19 months, about 19 months to about 20 months, about 20 months to about 21 months, about 21 months to about 22 months, about 22 months to about 23 months, about 23 months to about 2 years, about 2 years to about 30 months, about 30 months to about 3 years, about 3 years to about 4 years, about 4 years to about 5 years, about 5 years to about 6 years, about 6 years to about 7 years, about 7 years to about 8 years, about 8 years to about 9 years, about 9 years to about 10 years, about 10 years to about 11 years, about 11 years to about 12 years, about 12 years to about 13 years, about 13 years to about 14 years, about 14 years to about 15 years, about 15 years to about 16 years, about 16 years to about 17 years, about 17 years to about 18 years, about 18 years to about 19 years, about 19 years to about 20 years, about 20 years to about 25 years, about 25 years to about 30 years, about 30 years to about 35 years, or about 35 years to about 40 years.

In some embodiments, delaying onset or progression of an age-related disease or condition comprises delaying onset or progression of one or more symptoms of the age-related disease or condition after diagnosis of the age-related disease or condition in the subject. In some embodiments, one or more symptoms of the age-related disease or condition after diagnosis of the age-related disease or condition in the subject is delayed by at least about 1 month. In some embodiments, one or more symptoms of the age-related disease or condition after diagnosis of the age-related disease or condition in the subject is delayed by at least about 6 months. In some embodiments, delaying onset or progression of an age-related disease or condition comprises lessening or ameliorating one or more symptoms of the age-related disease or condition. In some embodiments, the symptom of the age-related disease or condition is chosen from: a decrease of glomerular filtration rate, an increase of blood urea nitrogen (BUN), an increase of serum creatinine, a proteinuria, a formation of sclerotic glomeruli, an irregularity in heart rhythm, an age-related cellular hypertrophy, an increase in the cross-sectional area of a cardiomyocyte, a decrease in cardiac stress tolerance, tumorigenesis, metastasis, and cachexia. In some embodiments, the symptom is relative to another subject without the age-related disease or condition. In some embodiments, administering the compound to the subject lessens or ameliorates the symptom relative to a pre-treatment value.

In some embodiments, delaying the onset or progression of one or more symptoms of the age-related disease or condition comprises maintenance of a measured value, wherein maintenance is determined from an average of the measured value collected over a period of time. Non-limiting examples of the measured value are glomerular filtration rate, blood urea nitrogen level, serum creatinine level, number of sclerotic glomeruli, urinary albumin level, cross-sectional area of a cardiomyocyte, metabolic equivalent, adipocyte size, and weight loss. Non-limiting examples of the period of time are about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 25 years, about 30 years, about 35 years, or about 40 years.

In some embodiments, onset or progression of one or more symptoms of the age-related disease or condition after diagnosis of the disease or condition in the subject can be delayed by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 2 years, at least about 30 months, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, at least about 20 years, least about 25 years, at least about 30 years, at least about 35 years, or at least about 40 years.

In some embodiments, onset or progression of one or more symptoms of the age-related disease or condition after diagnosis of the disease or condition in the subject can be delayed by at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 1 year, at most about 13 months, at most about 14 months, at most about 15 months, at most about 16 months, at most about 17 months, at most about 18 months, at most about 19 months, at most about 20 months, at most about 21 months, at most about 22 months, at most about 23 months, at most about 2 years, at most about 30 months, at most about 3 years, at most about 4 years, at most about 5 years, at most about 6 years, at most about 7 years, at most about 8 years, at most about 9 years, at most about 10 years, at most about 11 years, at most about 12 years, at most about 13 years, at most about 14 years, at most about 15 years, at most about 16 years, at most about 17 years, at most about 18 years, at most about 19 years, at most about 20 years, at most about 25 years, most about 30 years, at most about 35 years, or at most about 40 years.

In some embodiments, onset or progression of one or more symptoms of the age-related disease or condition after diagnosis of the disease or condition in the subject can be delayed by about 1 month to about 2 months, about 2 months to about 3 months, about 3 months to about 4 months, about 4 months to about 5 months, about 5 months to about 6 months, about 6 months to about 7 months, about 7 months to about 8 months, about 8 months to about 9 months, about 9 months to about 10 months, about 10 months to about 11 months, about 11 months to about 1 year, about 1 year to about 13 months, about 13 months to about 14 months, about 14 months to about 15 months, about 15 months to about 16 months, about 16 months to about 17 months, about 17 months to about 18 months, about 18 months to about 19 months, about 19 months to about 20 months, about 20 months to about 21 months, about 21 months to about 22 months, about 22 months to about 23 months, about 23 months to about 2 years, about 2 years to about 30 months, about 30 months to about 3 years, about 3 years to about 4 years, about 4 years to about 5 years, about 5 years to about 6 years, about 6 years to about 7 years, about 7 years to about 8 years, about 8 years to about 9 years, about 9 years to about 10 years, about 10 years to about 11 years, about 11 years to about 12 years, about 12 years to about 13 years, about 13 years to about 14 years, about 14 years to about 15 years, about 15 years to about 16 years, about 16 years to about 17 years, about 17 years to about 18 years, about 18 years to about 19 years, about 19 years to about 20 years, about 20 years to about 25 years, about 25 years to about 30 years, about 30 years to about 35 years, or about 35 years to about 40 years.

In some embodiments, one or more symptoms of the age-related disease or condition can be lessened or ameliorated by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.95%, or at least about 99.99%.

In some embodiments, one or more symptoms of the age-related disease or condition can be lessened or ameliorated by at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.1%, at most about 99.2%, at most about 99.3%, at most about 99.4%, at most about 99.5%, at most about 99.6%, at most about 99.7%, at most about 99.8%, at most about 99.9%, at most about 99.95%, or at most about 99.99%.

In some embodiments, one or more symptoms of the age-related disease or condition can be lessened or ameliorated by about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 91%, about 91% to about 92%, about 92% to about 93%, about 93% to about 94%, about 94% to about 95%, about 95% to about 96%, about 96% to about 97%, about 97% to about 98%, about 98% to about 99%, about 99% to about 99.1%, about 99.1% to about 99.2%, about 99.2% to about 99.3%, about 99.3% to about 99.4%, about 99.4% to about 99.5%, about 99.5% to about 99.6%, about 99.6% to about 99.7%, about 99.7% to about 99.8%, about 99.8% to about 99.9%, about 99.9% to about 99.95%, or about 99.95% to about 99.99%.

In some embodiments, the age-related disease or condition is geriatric anxiety disorder. In some embodiments, the age-related disease or condition is age-related inactivity. In some embodiments, the age-related disease or condition is reduction of spontaneous activity. In some embodiments, the age-related disease or condition is reduction of exploratory behavior. Age-Related Kidney Dysfunction

In some embodiments, the age-related disease or condition is a kidney disease. In some embodiments, one or more symptoms of the kidney disease comprises a number of sclerotic glomeruli, a decrease in a glomerular filtration rate, or an increase in a blood urea nitrogen (BUN).

The number of sclerotic glomeruli in the subject can be measured by pathological assessment of a kidney biopsy from the subject. In some embodiments, a reduction of the number of sclerotic glomeruli in the subject can be relative to a pre-treatment value of the number of sclerotic glomeruli in the subject. In some embodiments, the number of sclerotic glomeruli can be maintained at a measured value in the subject.

In some embodiments, the number of sclerotic glomeruli in the subject can be reduced by, relative to a pre-treatment value of the number of sclerotic glomeruli in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, the number of sclerotic glomeruli in the subject can be reduced by, relative to a pre-treatment value of the number of sclerotic glomeruli in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, or at most about 99%.

In some embodiments, the number of sclerotic glomeruli in the subject can be reduced by, relative to a pre-treatment value of the number of sclerotic glomeruli in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 91%, about 91% to about 92%, about 92% to about 93%, about 93% to about 94%, about 94% to about 95%, about 95% to about 96%, about 96% to about 97%, about 97% to about 98%, or about 98% to about 99%.

In some embodiments, administration of a compound described herein can increase a glomerular filtration rate in the subject. The glomerular filtration rate in the subject can be measured from the concentration of creatinine in blood, serum, or plasma, or from the clearance of creatinine from urine. In some embodiments, an increase of the glomerular filtration rate in the subject can be relative to a pre-treatment value of the glomerular filtration rate in the subject. In some embodiments, the glomerular filtration rate can be maintained at a measured value in the subject.

In some embodiments, the glomerular filtration rate in the subject can be increased by, relative to a pre-treatment value of the glomerular filtration rate in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 250%, at least about 300%, at least about 350%, at least about 400%, at least about 450%, at least about 500%, at least about 600%, at least about 700%, at least about 800%, at least about 900%, or at least about 1000%.

In some embodiments, the glomerular filtration rate in the subject can be increased by, relative to a pre-treatment value of the glomerular filtration rate in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, at most about 100%, at most about 110%, at most about 120%, at most about 130%, at most about 140%, at most about 150%, at most about 160%, at most about 170%, at most about 180%, at most about 190%, at most about 200%, at most about 250%, at most about 300%, at most about 350%, at most about 400%, at most about 450%, at most about 500%, at most about 600%, at most about 700%, at most about 800%, at most about 900%, or at most about 1000%.

In some embodiments, the glomerular filtration rate in the subject can be increased by, relative to a pre-treatment value of the glomerular filtration rate in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, about 200% to about 250%, about 250% to about 300%, about 300% to about 350%, about 350% to about 400%, about 400% to about 450%, about 450% to about 500%, about 500% to about 600%, about 600% to about 700%, about 700% to about 800%, about 800% to about 900%, or about 900% to about 1000%.

In some embodiments, the pre-treatment value of the glomerular filtration rate in the subject can be at most about 5 milliliters per minute per 1.73 square meters (mL/min/1.73 m²), at most about 10 mL/min/1.73 m², at most about 15 mL/min/1.73 m², at most about 20 mL/min/1.73 m², at most about 30 mL/min/1.73 m², at most about 40 mL/min/1.73 m², at most about 50 mL/min/1.73 m², at most about 60 mL/min/1.73 m², at most about 70 mL/min/1.73 m², at most about 80 mL/min/1.73 m², or at most about 90 mL/min/1.73 m².

In some embodiments, the pre-treatment value of the glomerular filtration rate in the subject can be about 5 mL/min/1.73 m² to about 10 mL/min/1.73 m², about 10 mL/min/1.73 m² to about 15 mL/min/1.73 m², about 15 mL/min/1.73 m² to about 20 mL/min/1.73 m², about 20 mL/min/1.73 m² to about 30 mL/min/1.73 m², about 30 mL/min/1.73 m² to about 40 mL/min/1.73 m², about 40 mL/min/1.73 m² to about 50 mL/min/1.73 m², about 50 mL/min/1.73 m² to about 60 mL/min/1.73 m², about 60 mL/min/1.73 m² to about 70 mL/min/1.73 m², about 70 mL/min/1.73 m² to about 80 mL/min/1.73 m², or about 80 mL/min/1.73 m² to about 90 mL/min/1.73 m².

In some embodiments, the glomerular filtration rate in the subject after administration of a compound described herein can be at least about 20 mL/min/1.73 m², at least about 30 mL/min/1.73 m², at least about 40 mL/min/1.73 m², at least about 50 mL/min/1.73 m², at least about 60 mL/min/1.73 m², at least about 70 mL/min/1.73 m², at least about 80 mL/min/1.73 m², at least about 90 mL/min/1.73 m², at least about 100 mL/min/1.73 m², at least about 110 mL/min/1.73 m², at least about 120 mL/min/1.73 m², at least about 130 mL/min/1.73 m², at least about 140 mL/min/1.73 m², at least about 150 mL/min/1.73 m², at least about 160 mL/min/1.73 m², at least about 170 mL/min/1.73 m², at least about 180 mL/min/1.73 m², at least about 190 mL/min/1.73 m², or at least about 200 mL/min/1.73 m². In some embodiments, the glomerular filtration rate in the subject can be maintained at any of the foregoing measured values.

In some embodiments, the glomerular filtration rate in the subject after administration of a compound described herein can be about 20 mL/min/1.73 m² to about 30 mL/min/1.73 m², about 30 mL/min/1.73 m² to about 40 mL/min/1.73 m², about 40 mL/min/1.73 m² to about 50 mL/min/1.73 m², about 50 mL/min/1.73 m² to about 60 mL/min/1.73 m², about 60 mL/min/1.73 m² to about 70 mL/min/1.73 m², about 70 mL/min/1.73 m² to about 80 mL/min/1.73 m², about 80 mL/min/1.73 m² to about 90 mL/min/1.73 m², about 90 mL/min/1.73 m² to about 100 mL/min/1.73 m², about 100 mL/min/1.73 m² to about 110 mL/min/1.73 m², about 110 mL/min/1.73 m² to about 120 mL/min/1.73 m², about 120 mL/min/1.73 m² to about 130 mL/min/1.73 m², about 130 mL/min/1.73 m² to about 140 mL/min/1.73 m², about 140 mL/min/1.73 m² to about 150 mL/min/1.73 m², about 150 mL/min/1.73 m² to about 160 mL/min/1.73 m², about 160 mL/min/1.73 m² to about 170 mL/min/1.73 m², about 170 mL/min/1.73 m² to about 180 mL/min/1.73 m², about 180 mL/min/1.73 m² to about 190 mL/min/1.73 m², or about 190 mL/min/1.73 m² to about 200 mL/min/1.73 m². In some embodiments, the glomerular filtration rate in the subject can be maintained at any of the foregoing measured values.

In some embodiments, a decreased glomerular filtration rate in the subject can lead to a failure to clear waste, such as urea or creatinine, from the blood. In some embodiments, the failure to clear waste from the blood in the subject causes an increase in blood urea nitrogen (BUN) level or serum creatinine level in the subject relative to another subject without kidney disease. The BUN level in the subject can be measured from the concentration of urea in blood, serum or plasma. The serum creatinine level in the subject can be measured from the concentration of creatinine in serum. In some embodiments, a decreased glomerular filtration rate in the subject can lead to a proteinuria, or a release of a blood protein, such as albumin, into urine. In some embodiments, the proteinuria is a microalbuminuria. In some embodiments, the proteinuria in the subject causes an increase in urinary albumin level in the subject relative to another subject without kidney disease. The urinary albumin level can be measured from the concentration of albumin in urine. An increase in the BUN level, serum creatinine level, or urinary albumin level in the subject can indicate kidney dysfunction. In some embodiments, administration of a compound described herein can decrease the BUN level, serum creatinine level, or urinary albumin level in the subject. In some embodiments, a decrease of the BUN level, serum creatinine level, or urinary albumin level in the subject can be relative to a pre-treatment value of the BUN level, serum creatinine level, or urinary albumin level in the subject. In some embodiments, the BUN level, serum creatinine level, or urinary albumin level can be maintained at a measured value in the subject.

In some embodiments, the BUN level, serum creatinine level, or urinary albumin level in the subject can be decreased by, relative to a pre-treatment value in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, the BUN level, serum creatinine level, or urinary albumin level in the subject can be decreased by, relative to a pre-treatment value in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, or at most about 99%.

In some embodiments, the BUN level, serum creatinine level, or urinary albumin level in the subject can be decreased by, relative to a pre-treatment value in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 91%, about 91% to about 92%, about 92% to about 93%, about 93% to about 94%, about 94% to about 95%, about 95% to about 96%, about 96% to about 97%, about 97% to about 98%, or about 98% to about 99%.

In some embodiments, the pre-treatment value of the BUN level in the subject can be at least about 60 milligrams per deciliter (mg/dL), at least about 55 mg/dL, at least about 50 mg/dL, at least about 45 mg/dL, at least about 40 mg/dL, at least about 35 mg/dL, at least about 30 mg/dL, at least about 25 mg/dL, or at least about 20 mg/dL. In some embodiments, the pre-treatment value of the BUN level in the subject can be about 20 mg/dL to about 25 mg/dL, about 25 mg/dL to about 30 mg/dL, about 30 mg/dL to about 35 mg/dL, about 35 mg/dL to about 40 mg/dL, about 40 mg/dL to about 45 mg/dL, about 45 mg/dL to about 50 mg/dL, about 50 mg/dL to about 55 mg/dL, or about 55 mg/dL to about 60 mg/dL.

In some embodiments, the BUN level in the subject after administration of a compound described herein can be at most about 6 mg/dL, at most about 7 mg/dL, at most about 8 mg/dL, at most about 9 mg/dL, at most about 10 mg/dL, at most about 11 mg/dL, at most about 12 mg/dL, at most about 13 mg/dL, at most about 14 mg/dL, at most about 15 mg/dL, at most about 16 mg/dL, at most about 17 mg/dL, at most about 18 mg/dL, at most about 19 mg/dL, at most about 20 mg/dL, at most about 25 mg/dL, at most about 30 mg/dL, at most about 35 mg/dL, or at most about 40 mg/dL. In some embodiments, the BUN level in the subject can be maintained at any of the foregoing measured values.

In some embodiments, the BUN level in the subject can be after administration of a compound described herein can be about 6 mg/dL to about 7 mg/dL, about 7 mg/dL to about 8 mg/dL, about 8 mg/dL to about 9 mg/dL, about 9 mg/dL to about 10 mg/dL, about 10 mg/dL to about 11 mg/dL, about 11 mg/dL to about 12 mg/dL, about 12 mg/dL to about 13 mg/dL, about 13 mg/dL to about 14 mg/dL, about 14 mg/dL to about 15 mg/dL, about 15 mg/dL to about 16 mg/dL, about 16 mg/dL to about 17 mg/dL, about 17 mg/dL to about 18 mg/dL, about 18 mg/dL to about 19 mg/dL, about 19 mg/dL to about 20 mg/dL, about 20 mg/dL to about 25 mg/dL, about 25 mg/dL to about 30 mg/dL, about 30 mg/dL to about 35 mg/dL, or about 35 mg/dL to about 40 mg/dL. In some embodiments, the BUN level in the subject can be maintained over any of the foregoing measured value ranges.

In some embodiments, the pre-treatment value of the serum creatinine level in the subject can be at least about 1.2 mg/dL, at least about 1.3 mg/dL, at least about 1.4 mg/dL, at least about 1.5 mg/dL, at least about 1.6 mg/dL, at least about 1.7 mg/dL, at least about 1.8 mg/dL, at least about 1.9 mg/dL, at least about 2 mg/dL, at least about 2.5 mg/dL, at least about 3 mg/dL, at least about 4 mg/dL, or at least about 5 mg/dL. In some embodiments, the pre-treatment value of the BUN level in the subject can be about 1 mg/dL to about 1.1 mg/dL, about 1.1 mg/dL to about 1.2 mg/dL, about 1.2 mg/dL to about 1.3 mg/dL, about 1.3 mg/dL to about 1.4 mg/dL, about 1.4 mg/dL to about 1.5 mg/dL, about 1.5 mg/dL to about 1.6 mg/dL, about 1.6 mg/dL to about 1.7 mg/dL, about 1.7 mg/dL to about 1.8 mg/dL, about 1.8 mg/dL to about 1.9 mg/dL, about 1.9 mg/dL to about 2 mg/dL, about 2 mg/dL to about 2.5 mg/dL, about 2.5 mg/dL to about 3 mg/dL, about 3 mg/dL to about 4 mg/dL, or about 4 mg/dL to about 5 mg/dL.

In some embodiments, the serum creatinine level in the subject after administration of a compound described herein can be at most about 0.3 mg/dL, at most about 0.4 mg/dL, at most about 0.5 mg/dL, at most about 0.6 mg/dL, at most about 0.7 mg/dL, at most about 0.8 mg/dL, at most about 0.9 mg/dL, at most about 1 mg/dL, at most about 1.1 mg/dL, at most about 1.2 mg/dL, at most about 1.3 mg/dL, at most about 1.4 mg/dL, at most about 1.5 mg/dL, at most about 1.6 mg/dL, at most about 1.7 mg/dL, at most about 1.8 mg/dL, at most about 1.9 mg/dL, or at most about 2 mg/dL. In some embodiments, the serum creatinine level in the subject can be maintained at any of the foregoing measured values.

In some embodiments, the serum creatinine level in the subject can be after administration of a compound described herein can be about 0.3 mg/dL to about 0.4 mg/dL, about 0.4 mg/dL to about 0.5 mg/dL, about 0.5 mg/dL to about 0.6 mg/dL, about 0.6 mg/dL to about 0.7 mg/dL, about 0.8 mg/dL to about 0.9 mg/dL, about 0.9 mg/dL to about 1 mg/dL, about 1 mg/dL to about 1.1 mg/dL, about 1.1 mg/dL to about 1.2 mg/dL, about 1.2 mg/dL to about 1.3 mg/dL, about 1.3 mg/dL to about 1.4 mg/dL, about 1.4 mg/dL to about 1.5 mg/dL, about 1.5 mg/dL to about 1.6 mg/dL, about 1.6 mg/dL to about 1.7 mg/dL, about 1.7 mg/dL to about 1.8 mg/dL, about 1.8 mg/dL to about 1.9 mg/dL, or about 1.9 mg/dL to about 2 mg/dL. In some embodiments, the serum creatinine level in the subject can be maintained over any of the foregoing measured value ranges.

In some embodiments, the pre-treatment value of the urinary albumin level in the subject can be at least about 250 milligrams per liter (mg/L), at least about 300 mg/L, at least about 350 mg/L, at least about 400 mg/L, at least about 450 mg/L, at least about 500 mg/L, at least about 600 mg/L, at least about 700 mg/L, at least about 800 mg/L, at least about 900 mg/L, at least about 1000 mg/L, at least about 1500 mg/L, or at least about 2000 mg/L. In some embodiments, the pre-treatment value of the urinary albumin level in the subject can be about 250 mg/L to about 300 mg/L, about 300 mg/L to about 350 mg/L, about 350 mg/L to about 400 mg/L, about 400 mg/L to about 450 mg/L, about 450 mg/L to about 500 mg/L, about 500 mg/L to about 600 mg/L, about 600 mg/L to about 700 mg/L, about 700 mg/L to about 800 mg/L, about 800 mg/L to about 900 mg/L, about 900 mg/L to about 1000 mg/L, about 1000 mg/L to about 1500 mg/L, or about 1500 mg/L to about 2000 mg/L.

In some embodiments, the urinary albumin level in the subject after administration of a compound described herein can be at most about 10 mg/L, at most about 20 mg/L, at most about 30 mg/L, at most about 40 mg/L, at most about 50 mg/L, at most about 60 mg/L, at most about 70 mg/L, at most about 80 mg/L, at most about 90 mg/L, at most about 100 mg/L, at most about 150 mg/L, at most about 200 mg/L, at most about 250 mg/L, at most about 300 mg/L, at most about 350 mg/L, at most about 400 mg/L, at most about 450 mg/L, or at most about 500 mg/L. In some embodiments, the urinary albumin level in the subject can be maintained at any of the foregoing measured values.

In some embodiments, the urinary albumin level in the subject, after administration of a compound described herein, can be about 10 mg/L to about 20 mg/L, about 20 mg/L to about 30 mg/L, about 30 mg/L to about 40 mg/L, about 40 mg/L to about 50 mg/L, about 50 mg/L to about 60 mg/L, about 60 mg/L to about 70 mg/L, about 70 mg/L to about 80 mg/L, about 80 mg/L to about 90 mg/L, about 90 mg/L to about 100 mg/L, about 100 mg/L to about 150 mg/L, about 150 mg/L to about 200 mg/L, about 200 mg/L to about 250 mg/L, about 250 mg/L to about 300 mg/L, about 300 mg/L to about 350 mg/L, about 350 mg/L to about 400 mg/L, or about 400 mg/L to about 450 mg/L, or about 450 mg/L to about 500 mg/L. In some embodiments, the urinary albumin level in the subject can be maintained over any of the foregoing measured value ranges.

In some embodiments, when the subject is administered a compound described herein, the senescent cells that are selectively killed are located in the renal proximal tubules of the subject.

In some embodiments, administration of a compound described herein can attenuate age-related renin-angiotensin-aldosterone system (RAAS) hyperactivity. In some embodiments, administration of a compound described herein can reduce or prevent angiotensin receptor 1 (AGTR1) hyperexpression.

### Age-Related Cardiovascular Disease

In some embodiments, the age-related disease or condition is a cardiovascular disease. Non-limiting examples of symptoms of the cardiovascular disease are irregularity in hearth rhythm, a decrease in Sur2a protein expression, age-related cellular hypertrophy, an increase in a cross-sectional area of a cardiomyocyte, or a decrease in cardiac stress tolerance.

The cross-sectional area of the cardiomyocyte in the subject can be measured by pathological assessment of a cardiac biopsy from the subject. In some embodiments, administration of a compound described herein can decrease the age-related cellular hypertrophy or cross-sectional area of the cardiomyocyte in the subject. In some embodiments, the decrease of the age-related cellular hypertrophy or cross-sectional area of the cardiomyocyte in the subject can be relative to a pre-treatment value of the age-related cellular hypertrophy or cross-sectional area of the cardiomyocyte in the subject. In some embodiments, the cross-sectional area of the cardiomyocyte can be maintained at a measured value in the subject.

In some embodiments, the age-related cellular hypertrophy or cross-sectional area of the cardiomyocyte in the subject can be decreased by, relative to a pre-treatment value in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, the age-related cellular hypertrophy or cross-sectional area of the cardiomyocyte in the subject can be decreased by, relative to a pre-treatment value in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, or at most about 99%.

In some embodiments, the age-related cellular hypertrophy or cross-sectional area of the cardiomyocyte in the subject can be decreased by, relative to a pre-treatment value in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 91%, about 91% to about 92%, about 92% to about 93%, about 93% to about 94%, about 94% to about 95%, about 95% to about 96%, about 96% to about 97%, about 97% to about 98%, or about 98% to about 99%.

In some embodiments, administration of a compound described herein can increase cardiac stress tolerance in the subject. Cardiac stress tolerance in the subject can be measured by an exercise tolerance test. Cardiac stress tolerance in the subject is measured in the exercise tolerance test in units of metabolic equivalents (METs), wherein 1 MET is 3.5 milliliter of oxygen per kilogram per minute (mL 02/kg/min). In some embodiments, an increase of cardiac stress tolerance in the subject can be relative to a pre-treatment value of cardiac stress tolerance in the subject. In some embodiments, the cardiac stress tolerance can be maintained at a measured value in the subject.

In some embodiments, cardiac stress tolerance in the subject can be increased by, relative to a pre-treatment value of cardiac stress tolerance in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 250%, at least about 300%, at least about 350%, at least about 400%, at least about 450%, at least about 500%, at least about 600%, at least about 700%, at least about 800%, at least about 900%, or at least about 1000%.

In some embodiments, cardiac stress tolerance in the subject can be increased by, relative to a pre-treatment value of cardiac stress tolerance in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, at most about 100%, at most about 110%, at most about 120%, at most about 130%, at most about 140%, at most about 150%, at most about 160%, at most about 170%, at most about 180%, at most about 190%, at most about 200%, at most about 250%, at most about 300%, at most about 350%, at most about 400%, at most about 450%, at most about 500%, at most about 600%, at most about 700%, at most about 800%, at most about 900%, or at most about 1000%.

In some embodiments, cardiac stress tolerance in the subject can be increased by, relative to a pre-treatment value of cardiac stress tolerance in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, about 200% to about 250%, about 250% to about 300%, about 300% to about 350%, about 350% to about 400%, about 400% to about 450%, about 450% to about 500%, about 500% to about 600%, about 600% to about 700%, about 700% to about 800%, about 800% to about 900%, or about 900% to about 1000%.

In some embodiments, the pre-treatment value of cardiac stress tolerance in the subject can be at most about 0.5 MET, at most about 1 MET, at most about 1.5 MET, at most about 2 MET, at most about 2.5 MET, at most about 3 MET, at most about 3.5 MET, at most about 4 MET, at most about 4.5 MET, or at most about 5 MET.

In some embodiments, the pre-treatment value of cardiac stress tolerance in the subject can be about 0.5 MET to about 1 MET, about 1 MET to about 1.5 MET, about 1.5 MET to about 2 MET, about 2 MET to about 2.5 MET, about 2.5 MET to about 3 MET, about 3 MET to about 3.5 MET, about 3.5 MET to about 4 MET, about 4 MET to about 4.5 MET, or about 4.5 MET to about 5 MET.

In some embodiments, cardiac stress tolerance in the subject after administration of a compound described herein can be at least about 5 MET, at least about 5.5 MET, at least about 6 MET, at least about 6.5 MET, at least about 7 MET, at least about 7.5 MET, at least about 8 MET, at least about 8.5 MET, at least about 9 MET, at least about 9.5 MET, at least about 10 MET, at least about 10.5 MET, at least about 11 MET, at least about 11.5 MET, or at least about 12 MET. In some embodiments, cardiac stress tolerance can be maintained at a measured value in the subject. In some embodiments, the cardiac stress tolerance in the subject can be maintained at any of the foregoing measured values.

In some embodiments, cardiac stress tolerance in the subject after administration of a compound described herein can be about 5 MET to about 5.5 MET, about 5.5 MET to about 6 MET, about 6 MET to about 6.5 MET, about 6.5 MET to about 7 MET, about 7 MET to about 7.5 MET, about 7.5 MET to about 8 MET, about 8 MET to about 8.5 MET, about 8.5 MET to about 9 MET, about 9 MET to about 9.5 MET, about 9.5 MET to about 10 MET, about 10 MET to about 10.5 MET, about 10.5 MET to about 11 MET, about 11 MET to about 11.5 MET, or about 11.5 MET to about 12 MET. In some embodiments, the cardiac stress tolerance in the subject can be maintained over any of the foregoing measured value ranges.

In some embodiments, when the subject is administered a compound described herein, the senescent cells that are selectively killed comprise epithelial cells, fibroblast cells, or vascular smooth muscle cells of the subject. In some embodiments, when the subject is administered a compound described herein, the senescent cells that are selectively killed are located on an atrial surface, on a ventricular surface, in a pericardium, or on an aortic root wall of the heart of the subject.

In some embodiments, administration of a compound described herein can increase cardiac maintenance, cardiac repair, and/or cardiac regeneration in a subject. In some embodiments, administration of a compound described herein can prevent the depletion of myogenic progenitor cells. In some embodiments, administration of a compound described herein can prevent the depletion of pericardium cells. In some embodiments, the pericardium cells are myogenic stem and/or myogenic progenitor cells. In some embodiments, administration of a compound described herein can decrease or prevent disruption of pericardial signaling. In some embodiments, administration of a compound described herein can decrease or prevent disruption of pericardial signaling through the senescence-associated secretome. In some embodiments, administration of a compound described herein can prevent or reduce cardiomyocyte loss. In some embodiments, administration of a compound described herein can increase or maintain cardiac stress resilience.

### Age-Related Fat Loss

In some embodiments, the age-related disease or condition is age-related fat loss or lipodystrophy. In some embodiments, one or more symptoms of age-related fat loss or lipodystrophy comprises a decrease in adipogenesis, an increase in adipocyte atrophy, or a decrease in adipocyte size. The decrease in adipogenesis or adipocyte size in the subject can be measured by pathological assessment of an adipose tissue biopsy from the subject. In some embodiments, administration of a compound described herein can increase adipogenesis or adipocyte size in the subject. In some embodiments, the increase in adipogenesis or adipocyte size in the subject can be relative to a pre-treatment value of adipogenesis or adipocyte size in the subject. In some embodiments, adipogenesis or adipocyte size can be maintained at a measured value in the subject.

In some embodiments, adipogenesis or adipocyte size in the subject can be increased by, relative to a pre-treatment value of adipogenesis or adipocyte size in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 250%, at least about 300%, at least about 350%, at least about 400%, at least about 450%, at least about 500%, at least about 600%, at least about 700%, at least about 800%, at least about 900%, or at least about 1000%.

In some embodiments, adipogenesis or adipocyte size in the subject can be increased by, relative to a pre-treatment value of adipogenesis or adipocyte size in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, at most about 100%, at most about 110%, at most about 120%, at most about 130%, at most about 140%, at most about 150%, at most about 160%, at most about 170%, at most about 180%, at most about 190%, at most about 200%, at most about 250%, at most about 300%, at most about 350%, at most about 400%, at most about 450%, at most about 500%, at most about 600%, at most about 700%, at most about 800%, at most about 900%, or at most about 1000%.

In some embodiments, adipogenesis or adipocyte size in the subject can be increased by, relative to a pre-treatment value of adipogenesis or adipocyte size in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, about 200% to about 250%, about 250% to about 300%, about 300% to about 350%, about 350% to about 400%, about 400% to about 450%, about 450% to about 500%, about 500% to about 600%, about 600% to about 700%, about 700% to about 800%, about 800% to about 900%, or about 900% to about 1000%.

In some embodiments, the pre-treatment value of adipocyte size in the subject can be at most about 10 microns (μm), at most about 20 μm, at most about 30 μm, at most about 40 μm, at most about 50 μm, at most about 60 μm, at most about 70 μm, or at most about 80 μm. In some embodiments, the pre-treatment value of adipocyte size in the subject can be about 10 μm to about 20 μm, about 20 μm to about 30 μm, about 30 μm to about 40 μm, about 40 μm to about 50 μm, about 50 μm to about 60 μm, about 60 μm to about 70 μm, or about 70 μm to about 80 μm.

In some embodiments, adipocyte size in the subject after administration of a compound described herein can be at least about 80 μm, at least about 90 μm, at least about 100 μm, at least about 110 μm, at least about 120 μm, at least about 130 μm, at least about 140 μm, at least about 150 μm, at least about 160 μm, at least about 170 μm, at least about 180 μm, at least about 190 μm, at least about 200 μm, at least about 210 μm, at least about 220 μm, at least about 230 μm, or at least about 240 μm. In some embodiments, the adipocyte size in the subject can be maintained at any of the foregoing measured values.

In some embodiments, adipocyte size in the subject after administration of a compound described herein can be about 80 μm to about 90 μm, about 90 μm to about 100 μm, about 100 μm to about 110 μm, about 110 μm to about 120 μm, about 120 μm to about 130 μm, about 130 μm to about 140 μm, about 140 μm to about 150 μm, about 150 μm to about 160 μm, about 160 μm to about 170 μm, about 170 μm to about 180 μm, about 180 μm to about 190 μm, about 190 μm to about 200 μm, about 200 μm to about 210 μm, about 210 μm to about 220 μm, about 220 μm to about 230 μm, or about 230 μm to about 240 μm. In some embodiments, the adipocyte size in the subject can be maintained over any of the foregoing measured value ranges.

In some embodiments, administration of a compound described herein can decrease adipocyte atrophy in the subject. The decrease in adipocyte atrophy in the subject can be measured by pathological assessment of an adipose tissue biopsy from the subject. In some embodiments, a decrease of adipocyte atrophy in the subject can be relative to a pre-treatment value of adipocyte atrophy in the subject. In some embodiments, adipocyte atrophy can be maintained at a measured value in the subject.

In some embodiments, adipocyte atrophy in the subject can be decreased by, relative to a pre-treatment value of adipocyte atrophy in the subject, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, adipocyte atrophy in the subject can be decreased by, relative to a pre-treatment value of adipocyte atrophy in the subject, at most about 1%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, or at most about 99%.

In some embodiments, adipocyte atrophy in the subject can be decreased by, relative to a pre-treatment value of adipocyte atrophy in the subject, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 91%, about 91% to about 92%, about 92% to about 93%, about 93% to about 94%, about 94% to about 95%, about 95% to about 96%, about 96% to about 97%, about 97% to about 98%, or about 98% to about 99%.

In some embodiments, when the subject is administered a compound described herein, the senescent cells that are selectively killed are fat progenitor cells of the subject. In some embodiments, when the subject is administered a compound described herein, the senescent cells that are selectively killed are located in subcutaneous fat depots or in visceral fat depots of the subject.

### Age-Related Cataracts

In some embodiments, the age-related disease or condition is a cataract. In some embodiments, onset or progression of the cataract can be delayed by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 2 years, at least about 30 months, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, at least about 20 years, least about 25 years, at least about 30 years, at least about 35 years, or at least about 40 years.

In some embodiments, onset or progression of the cataract can be delayed by at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 1 year, at most about 13 months, at most about 14 months, at most about 15 months, at most about 16 months, at most about 17 months, at most about 18 months, at most about 19 months, at most about 20 months, at most about 21 months, at most about 22 months, at most about 23 months, at most about 2 years, at most about 30 months, at most about 3 years, at most about 4 years, at most about 5 years, at most about 6 years, at most about 7 years, at most about 8 years, at most about 9 years, at most about 10 years, at most about 11 years, at most about 12 years, at most about 13 years, at most about 14 years, at most about 15 years, at most about 16 years, at most about 17 years, at most about 18 years, at most about 19 years, at most about 20 years, at most about 25 years, most about 30 years, at most about 35 years, or at most about 40 years.

In some embodiments, onset or progression of the cataract can be delayed by about 1 month to about 2 months, about 2 months to about 3 months, about 3 months to about 4 months, about 4 months to about 5 months, about 5 months to about 6 months, about 6 months to about 7 months, about 7 months to about 8 months, about 8 months to about 9 months, about 9 months to about 10 months, about 10 months to about 11 months, about 11 months to about 1 year, about 1 year to about 13 months, about 13 months to about 14 months, about 14 months to about 15 months, about 15 months to about 16 months, about 16 months to about 17 months, about 17 months to about 18 months, about 18 months to about 19 months, about 19 months to about 20 months, about 20 months to about 21 months, about 21 months to about 22 months, about 22 months to about 23 months, about 23 months to about 2 years, about 2 years to about 30 months, about 30 months to about 3 years, about 3 years to about 4 years, about 4 years to about 5 years, about 5 years to about 6 years, about 6 years to about 7 years, about 7 years to about 8 years, about 8 years to about 9 years, about 9 years to about 10 years, about 10 years to about 11 years, about 11 years to about 12 years, about 12 years to about 13 years, about 13 years to about 14 years, about 14 years to about 15 years, about 15 years to about 16 years, about 16 years to about 17 years, about 17 years to about 18 years, about 18 years to about 19 years, about 19 years to about 20 years, about 20 years to about 25 years, about 25 years to about 30 years, about 30 years to about 35 years, or about 35 years to about 40 years.

### Age-Related Carcinogenesis

In some embodiments, the age-related disease or condition is a cancer. In some embodiments, delaying onset or progression of the cancer comprises increasing tumor latency. In some embodiments, delaying onset or progression of the cancer comprises lessening or ameliorating one or more symptoms of the cancer. Examples of symptoms of cancer include tumorigenesis, metastasis, and cachexia. In some embodiments, onset or progression of the cancer or the symptom of the cancer can be delayed or tumor latency can be increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 2 years, at least about 30 months, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, at least about 20 years, least about 25 years, at least about 30 years, at least about 35 years, or at least about 40 years.

In some embodiments, onset or progression of the cancer or the symptom of the cancer can be delayed or tumor latency can be increased by at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 1 year, at most about 13 months, at most about 14 months, at most about 15 months, at most about 16 months, at most about 17 months, at most about 18 months, at most about 19 months, at most about 20 months, at most about 21 months, at most about 22 months, at most about 23 months, at most about 2 years, at most about 30 months, at most about 3 years, at most about 4 years, at most about 5 years, at most about 6 years, at most about 7 years, at most about 8 years, at most about 9 years, at most about 10 years, at most about 11 years, at most about 12 years, at most about 13 years, at most about 14 years, at most about 15 years, at most about 16 years, at most about 17 years, at most about 18 years, at most about 19 years, at most about 20 years, at most about 25 years, most about 30 years, at most about 35 years, or at most about 40 years.

In some embodiments, onset or progression of the cancer or the symptom of the cancer can be delayed or tumor latency can be increased by about 1 month to about 2 months, about 2 months to about 3 months, about 3 months to about 4 months, about 4 months to about 5 months, about 5 months to about 6 months, about 6 months to about 7 months, about 7 months to about 8 months, about 8 months to about 9 months, about 9 months to about 10 months, about 10 months to about 11 months, about 11 months to about 1 year, about 1 year to about 13 months, about 13 months to about 14 months, about 14 months to about 15 months, about 15 months to about 16 months, about 16 months to about 17 months, about 17 months to about 18 months, about 18 months to about 19 months, about 19 months to about 20 months, about 20 months to about 21 months, about 21 months to about 22 months, about 22 months to about 23 months, about 23 months to about 2 years, about 2 years to about 30 months, about 30 months to about 3 years, about 3 years to about 4 years, about 4 years to about 5 years, about 5 years to about 6 years, about 6 years to about 7 years, about 7 years to about 8 years, about 8 years to about 9 years, about 9 years to about 10 years, about 10 years to about 11 years, about 11 years to about 12 years, about 12 years to about 13 years, about 13 years to about 14 years, about 14 years to about 15 years, about 15 years to about 16 years, about 16 years to about 17 years, about 17 years to about 18 years, about 18 years to about 19 years, about 19 years to about 20 years, about 20 years to about 25 years, about 25 years to about 30 years, about 30 years to about 35 years, or about 35 years to about 40 years.

Non-limiting examples of cancers include adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sézary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenstrom macroglobulinemia.

In some embodiments, the subject has a genetic predisposition to developing cancer. In some embodiments, the genetic predisposition is selected from the group BRCA1 mutations, BRCA2 mutations, BARD1 mutations, BRIP1 mutations, Cowden Syndrome, Familial Adenomatous Polyposis, Lynch Syndrome, Garner's Syndrome, Li-Fraumeni Syndrome, Von Hippel-Lindau disease, multiple endocrine neoplasia, retinoblastoma, tuberous sclerosis, neurofibromatosis type I, and neurofibromatosis type II.

### Progeroid Syndromes

In certain embodiments are methods described herein for treating a progeroid syndrome. Progeroid syndromes are diseases of premature aging that mimic. These diseases mimic physiological aging at an accelerated rate. These diseases are often caused by mutations in enzymes involved in DNA repair, DNA recombination, nuclear structure, and chromosome segregation. Non-limiting examples of progeroid syndromes include Werner Syndrome, Bloom Syndrome, Rothmund-Thomson Syndrome, Cockayne Syndrome, Xeroderma Pigmentosum, trichothiodystrophy, combined Xeroderma Pigmentosum-Cockayne Syndrome, restrictive dermopathy, and Hutchinson-Gilford Progeria Syndrome (HGPS).

In some embodiments, the progeroid syndrome is Werner Syndrome. Werner syndrome is an autosomal recessive disorder, in which affected individuals grow and develop normally until puberty, during which they do not experience the typical adolescent growth spurt. Non-limiting examples of symptoms of Werner Syndrome are cardiovascular disease, cancer, growth retardation, short stature, premature graying of hair, hair loss, wrinkling, prematurely aging of the face, beaked noses, skin atrophy with scleroderma-like lesions, loss of adipose tissues, abnormal fat deposition, severe ulcerations around the Achilles tendon, changes in the voice, atrophy of gonads with reduced fertility, bilateral cataracts, premature arteriosclerosis, calcinosis, atherosclerosis, type 2 diabetes, loss of bone mass, and telangiectasia. In some embodiments, the cancer is a meningioma.

Werner Syndrome is caused by mutations in the WRN gene, which encodes the WRN protein, which encodes a helicase with similarity in structure to RecQ. The WRN protein unwinds DNA during both DNA repair and DNA replication. Mutations in WRN that cause Werner syndrome can decrease the stability of the transcribed messenger RNA, can lead to the truncation (shortening) of the WRN protein, cause a reduction in DNA helicase activity, accelerate WRN protein degradation, and induce down-regulation p53, suppressing p53-dependent apoptosis to maintain the survival of dysfunctional cells.

In some embodiments, the progeroid syndrome is Hutchinson-Gilford Progeria Syndrome (HGPS). HGPS is an autosomal dominant condition, characterized by premature and accelerated aging beginning at childhood. Non-limiting examples of symptoms of HGPS are myocardial infarction, arteriosclerosis, growth retardation, short stature, low body weight, delayed tooth eruption, enlarged eyes, thin noses, beaked noses, thin lips, micrognathia, protruding ears, protruding scalp hair, protruding eyebrows, protruding lashes, hair loss, large head, large fontanelle, osteolysis, osteoporosis, amyotrophy, lipodystrophy, skin atrophy with sclerodermatous focal lesions, severe atherosclerosis and prominent scalp veins.

HGPS is caused by mutations in the LMNA gene, which encodes for the lamin A protein. These LMNA mutations are dominant, de novo, point mutations that introduce a novel splice site into exon 11 that generates a C-terminal truncation of the protein, preventing processing and maturation of lamin A. The accumulation of unprocessed lamin A in the nucleus alters the nuclear structure, leading to lobulation of the nuclear envelope, thickening of nuclear lamina, loss of peripheral heterochromatin, and clustering of nuclear pores, causing the nucleus to lose its shape and integrity. This loss of nuclear integrity is believed to cause the premature aging phenotypes observed in HGPS.

### Muscle Atrophy

In certain embodiments are methods described herein for preventing or treating muscle atrophy. In some embodiments muscle atrophy is caused by age. In one embodiment

In some embodiments muscle atrophy is caused by temporary immobilization of the muscle. In some embodiments a senolytic agent, as described herein, is administered to a subject suffering from a temporary limb stabilization to prevent muscle atrophy. In some embodiments a senolytic agent, as described herein, is administered to a subject suffering from a temporary paralysis to prevent muscle atrophy. In one narrowing of the preceding embodiment the senolytic agent reduces muscle atrophy. In some embodiments a senolytic agent, as described herein, is administered to a subject suffering from a motor neuron trauma to prevent muscle atrophy.

In some embodiments, onset or progression of muscle atrophy can be delayed by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 2 years, or at least about 30 months.

### Caloric Restriction

In certain embodiments are methods described herein for mimicking a beneficial health effect of caloric restriction in a subject in need thereof without reducing caloric intake, comprising administering to the subject a compound to selectively kill senescent cells over non-senescent cells. In some embodiments, the beneficial health effect of caloric restriction is selected from weight loss, improved organ function, and lifespan extension. In some embodiments, the beneficial health effect can be maintained at a measured value in the subject.

In some embodiments, the weight loss is at least about 0.5 kg, at least about 1 kg, at least about 2 kg, at least about 3 kg, at least about 4 kg, at least about 5 kg, at least about 6 kg, at least about 7 kg, at least about 8 kg, at least about 9 kg, at least about 10 kg, at least about 15 kg, at least about 20 kg, at least about 25 kg, at least about 30 kg, at least about 35 kg, at least about 40 kg, at least about 45 kg, at least about 50 kg, at least about 60 kg, at least about 70 kg, at least about 80 kg, at least about 90 kg, or at least about 100 kg. In some embodiments, the weight loss can be maintained in the subject at any of the foregoing values.

In some embodiments, the weight loss is at most about 0.5 kg, at most about 1 kg, at most about 2 kg, at most about 3 kg, at most about 4 kg, at most about 5 kg, at most about 6 kg, at most about 7 kg, at most about 8 kg, at most about 9 kg, at most about 10 kg, at most about 15 kg, at most about 20 kg, at most about 25 kg, at most about 30 kg, at most about 35 kg, at most about 40 kg, at most about 45 kg, at most about 50 kg, at most about 60 kg, at most about 70 kg, at most about 80 kg, at most about 90 kg, or at most about 100 kg. In some embodiments, the weight loss can be maintained in the subject at any of the foregoing values.

In some embodiments, the weight loss is about 0.5 kg to about 1 kg, about 1 kg to about 2 kg, about 2 kg to about 3 kg, about 3 kg to about 4 kg, about 4 kg to about 5 kg, about 5 kg to about 6 kg, about 6 kg to about 7 kg, about 7 kg to about 8 kg, about 8 kg to about 9 kg, about 9 kg to about 10 kg, about 10 kg to about 15 kg, about 15 kg to about 20 kg, about 20 kg to about 25 kg, about 25 kg to about 30 kg, about 30 kg to about 35 kg, about 35 kg to about 40 kg, about 40 kg to about 45 kg, about 45 kg to about 50 kg, about 50 kg to about 60 kg, about 60 kg to about 70 kg, about 70 kg to about 80 kg, about 80 kg to about 90 kg, or about 90 kg to about 100 kg. In some embodiments, the weight loss can be maintained in the subject over any of the foregoing value ranges.

In some embodiments, the organ with improved function is chosen from adrenal gland, anus, appendix, bladder, bone, brain, bronchus, ear, esophagus, eye, gall bladder, genital, heart, hypothalamus, kidney, large intestine, larynx, liver, lung, lymph node, mouth, nose, pancreas, parathyroid gland, pituitary gland, prostate, rectum, salivary gland, skeletal muscle, skin, small intestine, spinal cord, spleen, stomach, thymus gland, trachea, thyroid, ureter, and urethra.

In some embodiments, the beneficial health effect of caloric restriction is a reduction of a likelihood of a cancer, a reduction of a likelihood of a kidney disease, a reduction of a likelihood of a nephropathy, a reduction of a likelihood of a cardiovascular disease, a reduction of a likelihood of a cardiomyopathy, a reduction of a likelihood of obesity, a reduction of a likelihood of type 2 diabetes, a reduction of a likelihood of a neurodegenerative disease, or a reduction of a likelihood of an autoimmune disease.

### Lifespan Extension of an Animal Model

In some embodiments, the compound or senolytic agent disclosed herein extends a lifespan of a non-human test subject relative to a lifespan of another non-human control subject. In some embodiments, the lifespan of the non-human test subject is an average lifespan of multiple non-human test subjects. In some embodiments, the lifespan of the non-human control subject is the average lifespan of multiple non-human control subjects.

In some embodiments, the lifespan of the non-human test subject is extended by, relative to the lifespan of the non-human control subject, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 35%, at least about 40%, or at least about 45%.

In some embodiments, the lifespan of the non-human test subject is extended by, relative to the lifespan of the non-human control subject, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 35%, at most about 40%, or at most about 45%.

In some embodiments, the lifespan of the non-human test subject is extended by, relative to the lifespan of the non-human control subject, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 25% to about 26%, about 26% to about 27%, about 27% to about 28%, about 28% to about 29%, about 29% to about 30%, about 30% to about 35%, about 35% to about 40%, or about 40% to about 45%.

The effectiveness of a senolytic agent with respect to extending lifespan or treating an age-related disease or condition described herein can readily be determined by a person skilled in the medical and clinical arts. A diagnostic method appropriate for the age-related disease or condition, known to a person skilled in the art, such as physical examination, patient self-assessment, assessment and monitoring of clinical symptoms, performance of analytical tests and methods, including clinical laboratory tests, physical tests, and exploratory surgery, can be used for monitoring the health status of the subject and the effectiveness of the senolytic agent. The effects of the methods of treatment described herein can be analyzed using techniques known in the art, such as comparing symptoms of patients suffering from or at risk of a particular disease or disorder that have received the pharmaceutical composition comprising a senolytic agent with those of patients who were not treated with the senolytic agent or who received a placebo treatment.

Pharmaceutical Compositions

Also provided herein are pharmaceutical compositions that comprise a senolytic agent (e.g., a MDM2 inhibitor; an inhibitor of one or more Bcl-2 anti-apoptotic protein family members wherein the inhibitor inhibits at least Bcl-xL (e.g., a Bcl-xL selective inhibitor, Bch 2/Bcl-xL/Bcl-w inhibitor, a Bcl-2/Bcl-xL or a Bcl-xL/Bcl-w inhibitor); or an Akt specific inhibitor), as described herein and at least one pharmaceutically acceptable excipient, which may also be called a pharmaceutically suitable excipient or carrier (i.e., a non-toxic material that does not interfere with the activity of the active ingredient). A pharmaceutical composition may be a sterile aqueous or non-aqueous solution, suspension or emulsion (e.g., a microemulsion). The excipients described herein are examples and are in no way limiting. An effective amount or therapeutically effective amount refers to an amount of the one or more senolytic agents administered to a subject, either as a single dose or as part of a series of doses, which is effective to produce a desired therapeutic effect.

When two or more senolytic agents are administered to a subject for treatment of a disease or disorder described herein, each of the senolytic agents may be formulated into separate pharmaceutical compositions. A pharmaceutical preparation may be prepared that comprises each of the separate pharmaceutical compositions (which may be referred to for convenience, for example, as a first pharmaceutical composition and a second pharmaceutical composition comprising each of the first and second senolytic agents, respectively). Each of the pharmaceutical compositions in the preparation may be administered at the same time (i.e., concurrently) and via the same route of administration or may be administered at different times by the same or different administration routes. Alternatively, two or more senolytic agents may be formulated together in a single pharmaceutical composition.

In other embodiments, a combination of at least one senolytic agent and at least one inhibitor of an mTOR, NFκB, or PI3-k pathway may be administered to a subject in need thereof. When at least one senolytic agent and an inhibitor of one or more of mTOR, NFκB, or PI3-k pathways are both used together in the methods described herein for selectively killing senescent cells, each of the agents may be formulated into the same pharmaceutical composition or formulated in separate pharmaceutical compositions. A pharmaceutical preparation may be prepared that comprises each of the separate pharmaceutical compositions, which may be referred to for convenience, for example, as a first pharmaceutical composition and a second pharmaceutical composition comprising each of the senolytic agent and the inhibitor of one or more of mTOR, NFκB, or PI3-k pathways, respectively. Each of the pharmaceutical compositions in the preparation may be administered at the same time and via the same route of administration or may be administered at different times by the same or different administration routes.

In particular embodiments, a single senolytic agent is administered to the subject and is the single (i.e., only, sole) active senolytic agent (i.e., monotherapy) used for treating the condition or disease. When a senolytic agent is the single senolytic agent, use of medications for other purposes such as palliative medications or medications that are used for comfort; or medications for treating a particular disease or condition but that are not senolytic agents, such as drugs for lowering cholesterol or an eye wetting agent, and other such medications familiar to a person skilled in the medical art, are not necessarily excluded. Examples of other agents and medications that can be administered to subjects with pulmonary diseases (e.g., COPD) include, by way of non-limiting example, bronchodilators (e.g., anti-cholinergics; beta-2 agonists); pain relief medication; Agents and medications that can be administered to subjects with osteoarthritis include hyaluronan, pain relievers (including topical medications), and steroids. Other agents and medications that can be administered to subjects with a cardiovascular disease include statins, beta blockers, nitroglyercin, aspirin.

Subjects may generally be monitored for therapeutic effectiveness using assays and methods suitable for the condition being treated, which assays will be familiar to those having ordinary skill in the art and are described herein. Pharmacokinetics of a senolytic agent (or one or more metabolites thereof) that is administered to a subject may be monitored by determining the level of the senolytic agent in a biological fluid, for example, in the blood, blood fraction (e.g., serum), and/or in the urine, and/or other biological sample or biological tissue from the subject. Any method practiced in the art and described herein to detect the agent may be used to measure the level of the senolytic agent during a treatment course.

The dose of a senolytic agent described herein for treating a senescence cell associated disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of the senolytic agent for treating a senescence-associated disease or disorder, suitable duration and frequency of administration of the senolytic agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a senolytic agent (including when administered for prophylactic benefit) described herein are well within the skill of a person skilled in the relevant art. When two or more senolytic agents are administered to treat a senescence-associated disease or disorder, the optimal dose of each senolytic agent may be different, such as less, than when either agent is administered alone as a single agent therapy. In certain particular embodiments, two senolytic agents in combination make act synergistically or additively, and either agent may be used in a lesser amount than if administered alone. An amount of a senolytic agent that may be administered per day may be, for example, between about 0.01 mg/kg and 100 mg/kg (e.g., between about 0.1 to 1 mg/kg, between about 1 to 10 mg/kg, between about 10-50 mg/kg, between about 50-100 mg/kg body weight. In other embodiments, the amount of a senolytic agent that may be administered per day is between about 0.01 mg/kg and 1000 mg/kg, between about 100-500 mg/kg, or between about 500-1000 mg/kg body weight. In particular embodiments, the total amount of an MDM2 inhibitor (e.g., Nutlin-3a), the total amount of the senolytic agent administered per course of treatment each treatment cycle does not exceed 2100 mg/kg; in other embodiments, the total amount administered per course of treatment does not exceed 1400 mg/kg. The optimal dose (per day or per course of treatment) may be different for the senescence-associated disease or disorder to be treated and may also vary with the administrative route and therapeutic regimen.

Pharmaceutical compositions comprising a senolytic agent can be formulated in a manner appropriate for the delivery method by using techniques routinely practiced in the art. The composition may be in the form of a solid (e.g., tablet, capsule), semi-solid (e.g., gel), liquid, or gas (aerosol). In other certain specific embodiments, the senolytic agent (or pharmaceutical composition comprising same) is administered as a bolus infusion. In certain embodiments when the senolytic agent is delivered by infusion, the senolytic agent is delivered to an organ or tissue comprising senescent cells to be killed via a blood vessel in accordance with techniques routinely performed by a person skilled in the medical art.

Pharmaceutical acceptable excipients are well known in the pharmaceutical art and described, for example, in Rowe et al., Handbook of Pharmaceutical Excipients: A Comprehensive Guide to Uses, Properties, and Safety, 5th Ed., 2006, and in Remington: The Science and Practice of Pharmacy (Gennaro, 21^{st} Ed. Mack Pub. Co., Easton, Pa. (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the agent(s) of the composition upon administration. In other embodiments, the agent may be encapsulated within liposomes using technology known and practiced in the art. In certain particular embodiments, a senolytic agent (e.g., ABT-263) is not formulated within liposomes for application to a stent that is used for treating highly, though not totally, occluded arteries. Pharmaceutical compositions may be formulated for any appropriate manner of administration described herein and in the art.

A pharmaceutical composition may be delivered to a subject in need thereof by any one of several routes known to a person skilled in the art. By way of non-limiting example, the composition may be delivered orally, intravenously, intraperitoneally, by infusion (e.g., a bolus infusion), subcutaneously, enteral, rectal, intranasal, by inhalation, buccal, sublingual, intramuscular, transdermal, intradermal, topically, intraocular, vaginal, rectal, or by intracranial injection, or any combination thereof. In certain particular embodiments, administration of a dose, as described above, is via intravenous, intraperitoneal, directly into the target tissue or organ, or subcutaneous route. In certain embodiments, a delivery method includes drug-coated or permeated stents for which the drug is the senolytic agent. Formulations suitable for such delivery methods are described in greater detail herein.

In certain particular embodiments, a senolytic agent (which may be combined with at least one pharmaceutically acceptable excipient to form a pharmaceutical composition) is administered directly to the target tissue or organ comprising senescent cells that contribute to manifestation of the disease or disorder. In specific embodiments when treating osteoarthritis, the at least one senolytic agent is administered directly to an osteoarthritic joint (i.e., intra-articularly) of a subject in need thereof. In other specific embodiments, a senolytic agent(s) may be administered to the joint via topical, transdermal, intradermal, or subcutaneous route. In other certain embodiments, methods are provided herein for treating a cardiovascular disease or disorder associated with arteriosclerosis, such as atherosclerosis by administering directly into an artery. In another particular embodiment, a senolytic agent (which may be combined with at least one pharmaceutically acceptable excipient to form a pharmaceutical composition) for treating a senescent-associated pulmonary disease or disorder may be administered by inhalation, intranasally, by intubation, or intracheally, for example, to provide the senolytic agent more directly to the affected pulmonary tissue. By way of another non-limiting example, the senolytic agent (or pharmaceutical composition comprising the senolytic agent) may be delivered directly to the eye either by injection (e.g., intraocular or intravitreal) or by conjunctival application underneath an eyelid of a cream, ointment, gel, or eye drops. In more particular embodiments, the senolytic agent or pharmaceutical composition comprising the senolytic agent may be formulated as a timed release (also called sustained release, controlled release) composition or may be administered as a bolus infusion.

A pharmaceutical composition (e.g., for oral administration or for injection, infusion, subcutaneous delivery, intramuscular delivery, intraperitoneal delivery or other method) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the senolytic agents described herein can be used alone or in combination with appropriate additives to make tablets, powders, granules or capsules, and if desired, with diluents, buffering agents, moistening agents, preservatives, coloring agents, and flavoring agents. The compounds may be formulated with a buffering agent to provide for protection of the compound from low pH of the gastric environment and/or an enteric coating. A senolytic agent included in a pharmaceutical composition may be formulated for oral delivery with a flavoring agent, e.g., in a liquid, solid or semi-solid formulation and/or with an enteric coating.

A pharmaceutical composition comprising any one of the senolytic agents described herein may be formulated for sustained or slow release (also called timed release or controlled release). Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active agent contained within a sustained release formulation depends upon the site of implantation, the rate and expected duration of release, and the nature of the condition, disease or disorder to be treated or prevented.

In certain embodiments, the pharmaceutical compositions comprising a senolytic agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated (intradermally or subcutaneously). The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions comprising a senolytic agent can be formulated as emulsions for topical application. An emulsion contains one liquid distributed the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

In some embodiments, the senolytic agent(s) can be formulated with oleaginous bases or ointments to form a semisolid composition with a desired shape. In addition to the senolytic agent, these semisolid compositions can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

Controlled or sustained release transdermal or topical formulations can be achieved by the addition of time-release additives, such as polymeric structures, matrices, that are available in the art. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

An insert, transdermal patch, bandage or article can comprise a mixture or coating of polymers that provide release of the active agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

Transdermal devices (inserts, patches, bandages) may also comprise a water insoluble polymer. Rate controlling polymers may be useful for administration to sites where pH change can be used to effect release. These rate controlling polymers can be applied using a continuous coating film during the process of spraying and drying with the active compound. In one embodiment, the coating formulation is used to coat pellets comprising the active ingredients that are compressed to form a solid, biodegradable insert.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the senolytic agent. The matrices can be formed by solvent evaporation, spray drying, solvent extraction and other methods known to those skilled in the art.

In certain embodiments of a method described herein for treating a cardiovascular disease associated with or caused by arteriosclerosis, one or more senolytic agents may be delivered directly into a blood vessel (e.g., an artery) via a stent. In a particular embodiment, a stent is used for delivering a senolytic agent to an atherosclerotic blood vessel (an artery). A stent is typically a tubular metallic device, which has thin-metal screen-like scaffold, and which is inserted in a compressed form and then expanded at the target site. Stents are intended to provide long-term support for the expanded vessel. Several methods are described in the art for preparing drug-coated and drug-embedded stents. For example, a senolytic agent may be incorporated into polymeric layers applied to a stent. A single type of polymer may be used, and one or more layers of the senolytic agent permeated polymer may be applied to a bare metal stent to form the senolytic agent-coated stent. The senolytic agent may also be incorporated into pores in the metal stent itself, which may also be referred to herein as a senolytic agent-permeated stent or senolytic agent-embedded stent. In certain particular embodiments, a senolytic agent may be formulated within liposomes and applied to a stent; in other particular embodiments, for example, when the senolytic agent is ABT-263, the ABT-263 is not formulated in liposome. Placement of stents in an atherosclerotic artery is performed by a person skilled in the medical art. A senolytic agent-coated or -embedded stent not only expands the affected blood vessel (e.g., an artery) but also may be effective for one or more of (1) reducing the amount of plaque, (2) inhibiting formation of plaque, and (3) increasing stability of plaque (e.g., by decreasing lipid content of the plaque; and/or causing an increase in fibrous cap thickness), particularly with respect to plaque proximal to the agent coated or agent embedded stent.

In one particular embodiment, the senolytic agent administered to a subject who has an ophthalmic senescence associated or disease or disorder may be delivered intraocularly or intravitreally. In other specific embodiments, a senolytic agent(s) may be administered to the eye by a conjunctival route, applying the senolytic agent to the mucous membrane and tissues of the eye lid, either upper, lower, or both. Any of these administrations may be bolus infusions. In other particular embodiments, a pharmaceutical composition comprising any one of the senolytic agents described herein may be formulated for sustained or slow release (which may also be called timed release or controlled release), which formulations are described in greater detail herein. In certain embodiments, methods are provided herein for treating or preventing (i.e., reducing the likelihood of occurrence of; delaying the onset or development of, or inhibiting, retarding, slowing, or impeding progression or severity of) an ocular disease, disorder, or condition (e.g., presbyopia, cataracts, macular degeneration); for selectively killing senescent cells in an eye of a subject, and/or inducing collagen (such as Type IV collagen) production in the eye of a subject in need thereof by administering at least one senolytic agent (which may be combined with at least one pharmaceutically acceptable excipient to form a pharmaceutical composition) directly to an eye.

In some embodiments, the senolytic agent is formulated within a microparticle matrix. In some embodiments, the microparticle matrix comprises poly(lactic-co-glycolic) acid copolymer (PLGA), PLA, hyaluronic acid, or a hydrogel. In some embodiments, the lactic acid-glycolic acid copolymer has a molar ratio of lactic acid:glycolic acid from the range of about 80:20 to 60:40. In some embodiments, the lactic acid-glycolic acid copolymer has a molar ratio of lactic acid:glycolic acid of 75:25. In some embodiments, the senolytic agent-microparticle matrix formulation provides a controlled or sustained-release of the senolytic agent. In some embodiments, the senolytic agent-microparticle matrix formulation provides an initial rapid release, or “burst” of the senolytic agent. In some embodiments, the length of sustained release is between 21 days and 90 days. In some embodiments, the length of sustained release is between 21 days and 60 days. In some embodiments, the length of sustained release is between 14 days and 30 days. In some embodiments, the length of release of the “burst” component is between 0 and 10 days, for example between the beginning of day 1 through the end of day 10. In some embodiments, the length of release of the initial “burst” component is between 0 and 6 days, for example between the beginning of day 1 through the end of day 6. In some embodiments, the length of initial “burst,” component is between 0 and 2 days, for example between the beginning of day 1 through the end of day 2. In some embodiments, the length of initial “burst” component is between 0 and 1 day, for example between the beginning of day 1 through the end of day 1.

The senolytic agent-microparticle formulations are suitable for administration, for example, local administration by injection into a site at or near the site of a subject's pain and/or inflammation. The senolytic agent-microparticle formulations provided herein are effective in slowing, arresting, reversing or otherwise inhibiting structural damage to tissues associated with progressive disease with minimal long-term side effects. In certain embodiments of the invention, a combination of an immediate release form and a sustained release form of the senolytic agent is administered (e.g., by single injection or as sequential injections) into an intra-articular space for the treatment of pain, for example, due to osteoarthritis, rheumatoid arthritis or other joint disorder(s). In certain embodiments of the invention, a combination of an immediate release form and a sustained release form of the senolytic agent is administered (e.g., by single injection or as sequential injections) into an intra-articular space or into soft tissues to slow, arrest, reverse or otherwise inhibit structural damage to tissues associated with progressive disease such as, for example, the damage to cartilage associated with progression of osteoarthritis. The formulations and methods of embodiments of the invention can achieve immediate relief of the acute symptoms (e.g., pain and inflammation) of these diseases or conditions and additionally provide a sustained or long term therapy (e.g., slowing, arresting, reversing or otherwise inhibiting structural damage to tissues associated with progressive disease), while avoiding long term systemic side effects.

For pharmaceutical compositions comprising a nucleic acid molecule, the nucleic acid molecule may be present within any of a variety of delivery systems known to those of ordinary skill in the art, including nucleic acid, and bacterial, viral and mammalian expression systems such as, for example, recombinant expression constructs as provided herein. Techniques for incorporating DNA into such expression systems are well known to those of ordinary skill in the art. The DNA may also be “naked,” as described, for example, in Ulmer et al., Science 259:1745-49, 1993 and reviewed by Cohen, Science 259:1691-92, 1993. The uptake of naked DNA may be increased by coating the DNA onto biodegradable beads, which are efficiently transported into the cells. Nucleic acid molecules may be delivered into a cell according to any one of several methods described in the art (see, e.g., Akhtar et al., Trends Cell Bio. 2:139 (1992); Delivery Strategies for Antisense Oligonucleotide Therapeutics, ed. Akhtar, 1995, Maurer et al., Mol. Membr. Biol. 16:129-40 (1999); Hofland et al., Handb. Exp. Pharmacol. 137:165-92 (1999); Lee et al., ACS Symp. Ser. 752:184-92 (2000); U.S. Pat. No. 6,395,713; Int'l Patent Appl. Publ. No. WO 94/02595); Selbo et al., Int. J. Cancer 87:853-59 (2000); Selbo et al., Tumour Biol. 23:103-12 (2002); U.S. Patent Appl. Publ. Nos. 2001/0007666, and 2003/077829).

Kits with unit doses of one or more of the agents described herein, usually in oral or injectable doses, are provided. Such kits may include a container containing the unit dose, an informational package insert describing the use and attendant benefits of the drugs in treating the senescent cell associated disease, and optionally an appliance or device for delivery of the composition.

While preferred embodiments of the pharmaceutical composition have been shown and described herein, it will be obvious to those skilled in the art that such embodiments are provided by way of example only. Numerous variations, changes and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art to which this invention belongs.

### NUMBERED EMBODIMENTS

The disclosure is further understood through review of the numbered embodiments recited herein. 1. A method for extending lifespan of a subject comprising administering to the subject a compound that selectively kills senescent cells over non-senescent cells. 2. The method of embodiment 1, wherein the extending lifespan of the subject comprises delaying onset of an age-related disease or condition.

3. A method for delaying onset or progression of an age-related disease or condition in a subject comprising administering to the subject a compound that selectively kills senescent cells over non-senescent cells. 4. The method of embodiment 3, wherein the method delays the onset of an age-related disease or condition. 5. The method of any one of embodiments 3-4, wherein the method delays the progression of an age-related disease or condition. 6. The method of any one of embodiments 3-5, wherein the age-related disease or condition is selected from atherosclerosis, cardiovascular disease, cancer, arthritis, dementia, cataract, osteoporosis, diabetes, hypertension, age-related fat loss, vertebral disc degeneration, age-related muscular atrophy and kidney disease. 7. The method of any one of embodiments 3-6, wherein the age related disease or condition is kidney disease. 8. The method of any one of embodiments 3-7, wherein the method comprises identifying a patient at risk of developing a kidney disease. 9. The method of any one of embodiments 3-8, wherein the method comprises identifying a patient presenting at least one symptom of a kidney disease. 10. The method of any one of embodiments 3-9, wherein delaying the onset or progression of kidney disease comprises delaying the onset or progression of at least one symptom of kidney disease. 11. The method of any one of embodiments 3-10, wherein a symptom of kidney disease is delayed for at least one month after diagnosis of kidney disease in the subject. 12. The method of any one of embodiments 3-11, wherein a symptom of kidney disease is delayed for at least six months after diagnosis of the kidney disease in the subject. 13. The method of any one of any one of embodiments 3-12, wherein the symptom is least one symptom selected from the list consisting of decreased glomerular filtration rate, elevated blood urea nitrogen (BUN) content, increased serum creatinine, proteinuria and formation of sclerotic glomeruli. 14. The method of any one of embodiments 3-13, wherein the symptom is decreased glomerular filtration rate. 15. The method of any one of embodiments 3-14, wherein delaying the onset or progression of the decreased glomerular filtration rate comprises maintaining a glomerular filtration rate of at least 70. 16. The method of any one of embodiments 3-15, wherein delaying the onset or progression of impaired glomerular filtration comprises maintaining a glomerular filtration rate of at least 90. 17. The method of any one of embodiments 3-16, wherein the symptom is elevated blood urea nitrogen (BUN) levels. 18. The method of any one of embodiments 3-17, wherein delaying the onset or progression of elevated blood urea nitrogen levels comprises maintaining a blood urea nitrogen level of from 5 to 30. 19. The method of any one of embodiments 3-18, wherein delaying the onset or progression of elevated blood nitrogen levels comprises maintaining a blood urea level of from 7 to 20. 20. The method of any one of embodiments 3-19, wherein delaying the onset or progression of kidney disease comprises ameliorating at least one symptom of kidney disease. 21. The method of any one of embodiments 3-20, wherein the symptom is selected from the list of symptoms consisting of decreased glomerular filtration rate, elevated blood urea nitrogen (BUN) content, increased serum creatinine, proteinuria and formation of sclerotic glomeruli. 22. The method of any one of embodiments 3-21, wherein the symptom is formation of sclerotic glomeruli. 23. The method of any one of embodiments 3-22, wherein administering the compound to the subject reduces the number of sclerotic glomeruli relative to a pre-treatment number of sclerotic glomeruli. 24. The method of any one of embodiments 3-23, wherein the number of sclerotic glomeruli are reduced by at least 15% or more relative to a pre-treatment value of sclerotic glomeruli. 25. The method of any one of embodiments 3-24, wherein the symptom is decreased glomerular filtration rate. 26. The method of any one of embodiments 3-25, wherein the glomerular filtration rate in the subject is increased relative to a pre-treatment value of glomerular filtration rate. 27. The method of any one of embodiments 3-26, wherein administering the compound to the subject increases the glomerular filtration rate by at least 20% relative to a pre-treatment value of glomerular filtration rate. 28. The method of any one of embodiments 3-27, wherein the symptom is elevated blood urea nitrogen level. 29. The method of any one of embodiments 3-28, wherein the blood urea nitrogen level in the subject is reduced relative to a pre-treatment value of blood urea nitrogen level. 30. The method of any one of embodiments 3-29, wherein the blood nitrogen level in the subject is reduced by at least 10% relative to a pre-treatment value of blood urea nitrogen level. 31. The method of any one of embodiments 3-30, wherein the blood nitrogen level in the subject is reduced by at least 50% relative to a pre-treatment value of blood urea nitrogen level. 32. The method of any one of embodiments 3-31, wherein the senescent cells are located in renal proximal tubules of the subject. 33. The method of any one of the preceding embodiments, wherein the disease is cardiovascular disease 34. The method of any one of the preceding embodiments, wherein the method comprises identifying a patient at risk of developing a cardiovascular disease. 35. The method of any one of the preceding embodiments, wherein the method comprises identifying a patient presenting at least one symptom of a cardiovascular disease. 36. The method of any one of the preceding embodiments, further comprising administering a cholesterol reducing agent. 37. The method of any one of the preceding embodiments, further comprising administering a blood-pressure reducing agent. 38. The method of any one of the preceding embodiments, wherein delaying the onset or progression of cardiovascular disease comprises delaying onset or progression of at least one symptom of cardiovascular disease. 39. The method of any one of the preceding embodiments, wherein a symptom of the cardiovascular disease is delayed for at least one month after diagnosis of cardiovascular disease in the subject. 40. The method of any one of the preceding embodiments, wherein a symptom of cardiovascular disease is delayed for at least six months after diagnosis of cardiovascular disease in a subject. 41. The method of any one of the preceding embodiments, wherein the symptom is selected from irregularity in heart rhythm, age-related cellular hypertrophy, increase in the cross-sectional area of a cardiomyocyte and decrease in cardiac stress tolerance. 42. The method of any one of the preceding embodiments, wherein delaying the onset or progression of cardiovascular disease comprises ameliorating one or more symptoms of cardiovascular disease. 43. The method of any one of the preceding embodiments, wherein the symptom is selected from irregularity in heart rhythm, age-related cellular hypertrophy, increase in the cross-sectional area of a cardiomyocyte and decrease in cardiac stress tolerance. 44. The method of any one of the preceding embodiments, wherein the symptom is age-related cellular hypertrophy. 45. The method of any one of the preceding embodiments, wherein administering the compound to the subject decreases age-related cellular hypertrophy relative to a pre-treatment value of cellular hypertrophy. 46. The method of any one of the preceding embodiments, wherein the symptom is an increase in the cross-sectional area of a cardiomyocyte. 47. The method of any one of the preceding embodiments, wherein administering the compound to the subject decreases the cross-sectional area of the cardiomyocyte relative to a pre-treatment value of a cross-sectional area of a cardiomyocyte. 48. The method of any one of the preceding embodiments, wherein the symptom is a decrease in cardiac stress tolerance. 49. The method of any one of the preceding embodiments, wherein administering the compound to the subject increases the cardiac stress tolerance relative to a pre-treatment value of cardiac stress tolerance. 50. The method of any one of the preceding embodiments, wherein cardiac stress tolerance is increased by at least 10% relative to the pre-treatment value of cardiac stress tolerance. 51. The method of any one of any one of the preceding embodiments, wherein the senescent cells are located on an atrial surface or ventricular surface of the heart. 52. The method of any one of the preceding embodiments, wherein the senescent cells are located in a pericardium of the heart. 53. The method of any one of the preceding embodiments, wherein the senescent cells comprise epithelial cells. 54. The method of any one of the preceding embodiments, wherein the senescent cells comprise fibroblast cells. 55. The method of any one of the preceding embodiments, wherein the senescent cells are located on an aortic root wall of the heart. 56. The method of any one of the preceding embodiments, wherein the senescent cells are vascular smooth muscle cells. 57. The method of any one of the preceding embodiments, wherein the condition is cancer. 58. The method of any one of the preceding embodiments, wherein the method comprises identifying a patient at risk of developing cancer. 59. The method of any one of the preceding embodiments, wherein the method comprises identifying a patient presenting at least one symptom of cancer. 60. The method of any one of the preceding embodiments, wherein the method comprises identifying a patient presenting at least one indicator of cancer. 61. The method of any one of the preceding embodiments, wherein the patient has undergone a surgical intervention to address a cancer. 62. The method of any one of the preceding embodiments, further comprising administering a chemotherapeutic. 63. The method of any one of the preceding embodiments, wherein delaying onset or progression of cancer comprises delaying onset or progression of at least one symptom of cancer. 64. The method of any one of the preceding embodiments, wherein a symptom of cancer is delayed for at least one month after diagnosis of cancer in the subject. 65. The method of any one of the preceding embodiments, wherein a symptom of cancer is delayed for at least six months after diagnosis of cancer in the subject. 66. The method of any one of the preceding embodiments, wherein delaying onset or progression of cancer comprises ameliorating at least one symptom of cancer. 67. The method of any one of any one of the preceding embodiments, wherein the symptom is tumorigenesis. 68. The method of any one of any one of the preceding embodiments, wherein administration of the compound to the subject increases tumor latency. 69. The method of any one of any one of the preceding embodiments, wherein the subject has a genetic predisposition to developing cancer. 70. The method of any one of the preceding embodiments, wherein the genetic predisposition is selected from BRCA1 mutations, BRCA2 mutations, BARD1 mutations, BRIP1 mutations, Cowden Syndrome, Lynch Syndrome, Garner's Syndrome, Li-Fraumeni Syndrome, Von Hippel-Lindau disease, and multiple endocrine neoplasia. 71. The method of any one of any one of the preceding embodiments, wherein the condition is arthritis. 72. The method of any one of the preceding embodiments, wherein the method comprises identifying a patient at risk of developing arthritis. 73. The method of any one of the preceding embodiments, wherein the method comprises identifying a patient presenting at least one symptom of arthritis. 74. The method of any one of the preceding embodiments, wherein the condition is dementia. 75. The method of any one of the preceding embodiments, wherein the method comprises identifying a patient at risk of developing dementia. 76. The method of any one of the preceding embodiments, wherein the method comprises identifying a patient presenting at least one symptom of dementia. 77. The method of any one of the preceding embodiments, wherein the condition is a cataract. 78. The method of any one of the preceding embodiments, wherein the method comprises identifying a patient at risk of developing a cataract. 79. The method of any one of the preceding embodiments, wherein the method comprises identifying a patient presenting at least one symptom of a cataract. 80. The method of any one of the preceding embodiments, wherein the condition is osteoporosis. 81. The method of any one of the preceding embodiments, wherein the method comprises identifying a patient at risk of developing osteoporosis. 82. The method of any one of the preceding embodiments, wherein the method comprises identifying a patient presenting at least one symptom of osteoporosis. 83. The method of any one of the preceding embodiments, wherein the condition is diabetes. 84. The method of any one of the preceding embodiments, wherein the method comprises identifying a patient at risk of developing diabetes. 85. The method of any one of the preceding embodiments, wherein the method comprises identifying a patient presenting at least one symptom of diabetes. 86. The method of any one of any one of the preceding embodiments, wherein the condition is hypertension. 87. The method of any one of the preceding embodiments, wherein the method comprises identifying a patient at risk of developing hypertension. 88. The method of any one of the preceding embodiments, wherein the method comprises identifying a patient presenting at least one symptom of hypertension. 89. The method of any one of the preceding embodiments, wherein the condition is age-related fat loss. 90. The method of any one of the preceding embodiments, wherein the method comprises identifying a patient at risk of developing age-related fat loss. 91. The method of any one of the preceding embodiments, wherein the method comprises identifying a patient presenting at least one symptom of age-related fat loss.

92. A method of mimicking a beneficial health effect of calorie restriction in a subject, comprising administering to the subject a compound that selectively kills senescent cells over non-senescent cells. 93. The method of embodiment 92, wherein caloric intake is not substantially modified. 94. The method of any one of embodiments 92-93, wherein the beneficial health effect of calorie restriction is selected from weight loss, improved organ function, and life extension. 95. The method of any one of embodiments 92-94, wherein the beneficial health effect of calorie restriction is the prevention of cancer, kidney disease, cardiovascular disease, obesity, type 2 diabetes, neurodegenerative disease, or an autoimmune disease. 96. The method of any one of the preceding embodiments, wherein the compound extends the lifespan of a non-human test subject relative to the lifespan of a control subject. 97. The method of any one of the preceding embodiments, wherein the compound extends the lifespan of a non-human test subject by at least 10% relative to the lifespan of a control test subject. 98. The method of any one of the preceding embodiments, wherein the compound extends the lifespan of a non-human test subject by at least 20% relative to the lifespan of a control test subject. 99. The method of any one of embodiments 96-98, wherein the lifespan of a non-human test subject is an average lifespan of multiple test subjects. 100. The method of any one of embodiments 96 to 99, wherein the lifespan of a control subject is the average lifespan of multiple control test subjects. 101. The method of any one of the preceding embodiments, wherein practice of the method kills at least about 10% of the senescent cells. 102. The method of any one of the preceding embodiments, wherein practice of the method kills at least about 25% of the senescent cells. 103. The method of any one of the preceding embodiments, wherein practice of the method kills no more than 10% of non-senescent cells. 104. The method of any one of the preceding embodiments, wherein practice of the method kills no more than 5% of non-senescent cells. 105. The method of any one of the preceding embodiments, wherein the compound is administered in at least two treatment cycles, wherein each treatment cycle independently comprises a treatment course of from 1 day to 3 months followed by a non-treatment interval of at least 2 weeks; provided that if the compound agent is an MDM2 inhibitor, the MDM2 inhibitor is administered as a monotherapy, and each treatment course is at least 5 days long during which the MDM2 inhibitor is administered on at least 5 days. 106. The method of any one of the preceding embodiments, wherein the subject suffers from a progeroid syndrome. 107. The method of any one of the preceding embodiments, wherein the progeroid syndrome is selected from Werner syndrome, Bloom syndrome, Rothmund-Thomson syndrome, Cockayne syndrome, xeroderma pigmentosum, trichothiodystrophy, combined xeroderma pigmentosum-Cockayne syndrome, restrictive dermopathy, and Hutchinson-Gilford progeria syndrome. 108. The method of any one of the preceding embodiments, wherein the progeroid syndrome is selected from Werner syndrome and Hutchinson-Gilford progeria.

109. A method of ameliorating the progression of vertebral disc degeneration, comprising identifying an individual susceptible to vertebral disc degeneration, and administering a senolytic agent. 110. The method of embodiment 109, wherein the senolytic agent is administered at a dose below a dose identified as therapeutically relevant as a cancer treatment for the agent. 111. The method of any one of embodiments 109-110, wherein the senolytic agent is selected from an MDM2 inhibitor; an inhibitor of one or more BCL-2 anti-apoptotic protein family members wherein the inhibitor inhibits at least BCL-xL; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-in-converting enzyme-inhibitory protein (c-FLIP); an inhibitor of Serpin; an inhibitor of Ubiquilin-2; an inhibitor of Epiregulin; an inhibitor of Sorting nexin-3 (Snx3); an inhibitor of forkhead box protein O4 (FOXO4); and an inhibitor of Proto-oncogene tyrosine protein kinase Src (Src) 112. The method of any one of embodiments 109-111, wherein the compound is an MDM2 inhibitor. 113. The method of any one of embodiments 109-112, wherein the MDM2 inhibitor is administered at a dose below a dose identified as therapeutically relevant as a cancer treatment. 114. The method of any one of embodiments 109-113, wherein the individual is not diagnosed as having cancer. 115. The method of any one of embodiments 109-114, wherein the MDM2 inhibitor is selected from Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. 116. The method of any one of embodiments 109-115, wherein the MDM2 inhibitor is Nutlin 3a. 117. The method of any one of embodiments 109-116, wherein the individual presents back pain. 118. The method of any one of embodiments 109-117, wherein the individual demonstrates at least one sign of vertebral disc degeneration. 119. The method of any one of embodiments 109-118, wherein the individual has undergone at least one back surgery. 120. The method of any one of embodiments 109-119, wherein the individual has suffered a back injury. 121. The method of any one of embodiments 109-120, wherein the individual has suffered a herniated disc. 122. The method of any one of embodiments 109-121, wherein the MDM2 inhibitor is administered within 2 weeks of undergoing back surgery. 123. The method of any one of embodiments 109-122, wherein the MDM2 inhibitor is administered in at least three consecutive months. 124. The method of any one of embodiments 109-123, wherein the MDM2 inhibitor is administered in at least three consecutive years 125. The method of any one of embodiments 109-124, wherein the individual suffers from age-related disc degeneration. 126. A composition for use in the method of any one of embodiments 109-125.

127. A method of delaying muscular atrophy, comprising selecting an individual at risk of muscular atrophy and administering a senolytic agent. 128. The method of embodiment 127, wherein the senolytic agent is administered at a dose below a dose identified as therapeutically relevant as a cancer treatment for the agent. 129. The method of any one of embodiments 127-128, wherein the senolytic agent is selected from an MDM2 inhibitor; an inhibitor of one or more BCL-2 anti-apoptotic protein family members wherein the inhibitor inhibits at least BCL-xL; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-1β-converting enzyme-inhibitory protein (c-FLIP); an inhibitor of Serpin; an inhibitor of Ubiquilin-2; an inhibitor of Epiregulin; an inhibitor of Sorting nexin-3 (Snx3); an inhibitor of forkhead box protein O4 (FOXO4); and an inhibitor of Proto-oncogene tyrosine protein kinase Src (Src) 130. The method of any one of embodiments 127-129, wherein the compound is an MDM2 inhibitor. 131. The method of any one of embodiments 127-130, wherein the MDM2 inhibitor is administered at a dose below a dose identified as therapeutically relevant as a cancer treatment. 132. The method of any one of embodiments 127-131, wherein the individual is not diagnosed as having cancer. 133. The method of any one of embodiments 127-132, wherein the MDM2 inhibitor is selected from Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. 134. The method of any one of embodiments 127-133, wherein the MDM2 inhibitor is Nutlin 3a. 135. The method of any one of embodiments 127-134, wherein the individual suffers from paralysis. 136. The method of any one of embodiments 127-135, wherein the paralysis results from a motor nervous system trauma. 137. The method of any one of embodiments 127-136, wherein the individual has undergone at least one surgery to address a motor nervous system injury. 138. The method of any one of embodiments 127-137, wherein the individual has suffered a back injury. 139. The method of any one of embodiments 127-138, wherein the MDM2 inhibitor is administered within 2 weeks of undergoing back surgery. 140. The method of any one of embodiments 127-139, wherein the MDM2 inhibitor is administered within 2 weeks of suffering a motor nervous system trauma. 141. The method of any one of embodiments 127-140, wherein the MDM2 inhibitor is administered in at least three consecutive months. 142. The method of any one of embodiments 127-141, wherein the MDM2 inhibitor is administered in at least three consecutive years. 143. The method of any one of embodiments 127-142, wherein the individual suffers from motor neuron degeneration. 144. The method of any one of embodiments 127-143, wherein the individual suffers from age-related muscle atrophy. 145. A composition for use in the method of any one of embodiments 127-144.

146. A composition for use in selectively killing senescent cells in a mammal comprising an MDM2 inhibitor and a pharmaceutically acceptable buffer. 147. The composition of embodiment 146, wherein the MDM2 inhibitor binds an MDM2 N-terminus. 148. The composition of any one of embodiments 146-147, wherein the MDM2 inhibitor blocks E3 ligase activity. 149. The composition of any one of embodiments 146-148, wherein the MDM2 inhibitor is selected from the list consisting of Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-41-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. 150. The composition of any one of embodiments 146-149, wherein the MDM2 inhibitor is selected from the list consisting of AMG-232, NVP-CGM097, MI-773, CAY10681, CAY10682, Y239-EE, RG-7112, a Boronate, RO-5963, HLI 373, JNJ 26854165, and MEL23. 151. The composition of any one of embodiments 146-150, wherein the MDM2 inhibitor comprises MI-773. 152. The composition of any one of embodiments 146-151, wherein the MDM2 inhibitor comprises RG-7112. 153. The composition of any one of embodiments 146-152, wherein the MDM2 inhibitor comprises JNJ 26854165. 154. The composition of any one of embodiments 146-153, wherein the MDM2 inhibitor comprises MEL23. 155. The composition of any one of embodiments 146-154, for use in delaying intervertebral disc degeneration. 156. The composition of any one of embodiments 146-155, for use in delaying muscular atrophy.

157. A method of healthy lifespan extension comprising administering an MDM2 inhibitor in combination with at least one lifespan extending measure to an individual. 158. The method of embodiment 157, wherein the at least one lifespan extending measure comprises exercise. 159. The method of any one of embodiments 157-158, wherein the at least one lifespan extending measure comprises caloric restriction. 160. The method of any one of embodiments 157-159, wherein the MDM2 inhibitor is administered at a dose below a dose known to ameliorate symptoms of a cancer. 161. The method of any one of embodiments 157-160, wherein the MDM2 inhibitor is at least one compound selected from the list consisting of Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-41-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib. 162. The method of any one of embodiments 157-161, wherein the MDM2 inhibitor is selected from the list consisting of AMG-232, NVP-CGM097, MI-773, CAY10681, CAY10682, Y239-EE, RG-7112, a Boronate, RO-5963, HLI 373, JNJ 26854165, and MEL23. 163. The method of any one of embodiments 157-162, wherein the MDM2 inhibitor comprises MI-773. 164. The method of any one of embodiments 157-163, wherein the MDM2 inhibitor comprises RG-7112. 165. The method of any one of embodiments 157-164, wherein the MDM2 inhibitor comprises JNJ 26854165. 166. The method of any one of embodiments 157-165, wherein the MDM2 inhibitor comprises MEL23. 167. The method of any one of embodiments 157-166, wherein the individual does not present a symptom of an age-related disorder.

168. The method of any one of the preceding embodiments, wherein the compound is selected from an MDM2 inhibitor; an inhibitor of one or more BCL-2 anti-apoptotic protein family members wherein the inhibitor inhibits at least BCL-xL; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like MAO-converting enzyme-inhibitory protein (c-FLIP); an inhibitor of Serpin; an inhibitor of Ubiquilin-2; an inhibitor of Epiregulin; an inhibitor of Sorting nexin-3 (Snx3); an inhibitor of forkhead box protein O4 (FOXO4); and an inhibitor of Proto-oncogene tyrosine protein kinase Src (Src). 169. The method of any one of the preceding embodiments, wherein the compound is an MDM2 inhibitor and is Nutlin-3a or RG-1172. 170. The method of any one of the preceding embodiments, wherein the compound is administered as a monotherapy. 171. The method of any one of the preceding embodiments, wherein the compound is administered within at least one treatment cycle, which treatment cycle comprises a treatment course followed by a non-treatment interval; and wherein the total dose of the compound administered during the treatment cycle is an amount less than the amount effective for a cancer treatment, wherein the compound is selected from an inhibitor of a Bcl-2 anti-apoptotic protein family member that inhibits at least Bcl-xL; an MDM2 inhibitor; an Akt specific inhibitor; an inhibitor of Akt 1, 2, or 3; a c-Jun N-terminal kinase (JNK)1, JNK2, JNK3, or Kit inhibitor; a protein phosphatase 2C (PP2C) or MAP kinase phosphatase-1 (MKP-1) inhibitor; a reactive oxygen species (ROS) inducer; an S6 kinase inhibitor; a protein kinase A (PKA) inhibitor; an inhibitor of a checkpoint kinase (Chk)1 or checkpoint kinase 2; an inhibitor of platelet-derived growth factor receptor beta (PDGFRB); an inhibitor of vascular endothelial growth factor receptor (VEGFR)-2; an inhibitor of phosphoinositide 3-kinase (PI3K); an inhibitor of apoptosis signal-regulating kinase 1 (ASK1); an inhibitor of spleen tyrosine kinase (Syk); an inhibitor of epidermal growth factor receptor (EGFR); an inhibitor of cathepsin; a glucosamine analog; an inhibitor of poly ADP ribose polymerase (PARP)1 or PARP2; an inhibitor of Cathepsin H; an inhibitor of cellular FADD-like IL-1β-converting enzyme-inhibitory protein (c-FLIP); an inhibitor of Serpin; an inhibitor of Ubiquilin-2; an inhibitor of Epiregulin; and an inhibitor of Sorting nexin-3 (Snx3); an inhibitor of forkhead box protein O4 (FOXO4); and an inhibitor of Proto-oncogene tyrosine protein kinase Src (Src). 172. The method of any one of the preceding embodiments, wherein the compound is administered during two or more treatment cycles, and wherein the total dose of the compound administered during the two or more treatment cycles is an amount less than the amount effective for a cancer treatment. 173. The method of any one of the preceding embodiments, wherein each treatment course is no longer than (a) one month, or (b) no longer than two months, or (c) no longer than 3 months. 174. The method of any one of the preceding embodiments, wherein each treatment course is no longer than (a) 5 days, (b) 7 days, (c) 10 days, (d) 14 days, or (e) 21 days. 175. The method of any one of the preceding embodiments, wherein each treatment course is selected from 3 days to 12 days. 176. The method of any one of the preceding embodiments, wherein the compound is administered every other day of each treatment course. 177. The method of any one of the preceding embodiments, wherein the compound is administered daily during each treatment course. 178. The method of any one of the preceding embodiments, wherein the non-treatment interval has a duration of at least one month. 179. The method of any one of the preceding embodiments, wherein the treatment course is one day and the non-treatment interval is between 0.5-12 months. 180. The method of any one of the preceding embodiments, wherein the compound is administered directly to an organ or tissue that comprises the senescent cells. 181. The method of any one of the preceding embodiments, wherein the compound is combined with at least one pharmaceutically acceptable excipient to formulate a pharmaceutically acceptable composition to provide timed-release of the compound. 182. The method of any one of the preceding embodiments, wherein the compound is administered as a bolus infusion. 183. The method of any one of the preceding embodiments, wherein the compound is administered topically, transdermally, intradermally, intraarticularly, intranasally, intratracheally, intubation, parenterally, or orally. 184. The method of any one of the preceding embodiments, wherein the MDM2 inhibitor is a cis-imidazoline compound, a spiro-oxindole compound, or a benzodiazepine compound. 185. The method of any one of the preceding embodiments, wherein the cis-imidazoline compound is a nutlin compound. 186. The method of any one of the preceding embodiments, wherein the nutlin compound is Nutlin-3a or Nutlin-3b. 187. The method of any one of the preceding embodiments, wherein the cis-imidazoline compound is RG-7112, RG7388, RO5503781, or is a dihydroimidazothiazole compound. 188. The method of any one of the preceding embodiments, wherein the MDM2 inhibitor is a spiro-oxindole compound selected from MI-63, MI-126, MI-122, MI-142, MI-147, MI-18, MI-219, MI-220, MI-221, MI-773, and 3-(4-chlorophenyl)-3-((1-(hydroxymethyl)cyclopropyl)methoxy)-2-(4-nitrobenzyl)isoindolin-1-one. 189. The method of any one of the preceding embodiments, wherein the MDM2 inhibitor is Serdemetan; a piperidinone compound; CGM097; or an MDM2 inhibitor that also inhibits MDMX and which is selected from RO-2443 and RO-5963. 190. The method of any one of the preceding embodiments, wherein the piperidinone compound is AM-8553. 191. The method of any one of the preceding embodiments, wherein the inhibitor of one or more BCL-2 anti-apoptotic protein family members is a BCL-2/BCL-xL inhibitor; a BCL-2/BCL-xL/BCL-w inhibitor; or a BCL-xL selective inhibitor. 192. The method of any one of the preceding embodiments, wherein the BCL-xL selective inhibitor is a benzothiazole-hydrazone compound, an aminopyridine compound, a benzimidazole compound, a tetrahydroquinolin compound, or a phenoxyl compound. 193. The method of any one of the preceding embodiments, wherein the benzothiazole-hydrazone compound is WEHI-539. 194. The method of any one of the preceding embodiments, wherein the inhibitor of one or more Bcl-2 anti-apoptotic protein family members is A-1155463, A-1331852, ABT-263, ABT-199, or ABT-737. 195. The method of any one of the preceding embodiments, wherein the Akt inhibitor is MK-2206. 196. The method of any one of the preceding embodiments, wherein the Akt inhibitor is CCT128930. 197. The method of any one of the preceding embodiments, wherein the JNK 1, JNK2, JNK, or Kit inhibitor is JNK-IN-8. 198. The method of any one of the preceding embodiments, wherein the PP2C or MKP-2 inhibitor is a benzophenanthridine alkaloid. 199. The method of any one of the preceding embodiments, wherein the benzophenanthridine alkaloid is sanguinarine chloride. 200. The method of any one of the preceding embodiments, wherein the reactive oxygen species (ROS) inducer is methyl 3-(4-nitrophenyl) propiolate (NPP). 201. The method of any one of the preceding embodiments, wherein the PKA inhibitor is AT7867. 202. The method of any one of the preceding embodiments, wherein the inhibitor of checkpoint kinase 1 or checkpoint kinase 2 is AZD7762. 203. The method of any one of the preceding embodiments, wherein the vascular endothelial growth factor receptor (VEGFR)-2 is sunitinib. 204. The method of any one of the preceding embodiments, wherein the inhibitor of PI3K is GDC-0980 or BKM120. 205. The method of any one of the preceding embodiments, wherein the ASK1 inhibitor is NQDI-1. 206. The method of any one of the preceding embodiments, wherein the inhibitor of Syk is R406. 207. The method of any one of the preceding embodiments, wherein the inhibitor of EGFR is erlotinib. 208. The method of any one of the preceding embodiments, wherein the inhibitor of cathepsin is CYM 7008-00-01. 209. The method of any one of the preceding embodiments, wherein the glucosamine analog is GlcNAc. 210. The method of any one of the preceding embodiments, wherein the inhibitor of PARP1 or PARP2 is olaparib. 211. The method of any one of the preceding embodiments, wherein the compound that selectively kills senescent cells over non-senescent cells is selected from Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-41-(hydroxymethyl)cyclopropyl)methoxy)-2-(4-nitrobenzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, and olaparib.

### Examples

### Example 1 In Vitro Cell Assays for Determining Senolytic Activity of Nutlin-3a

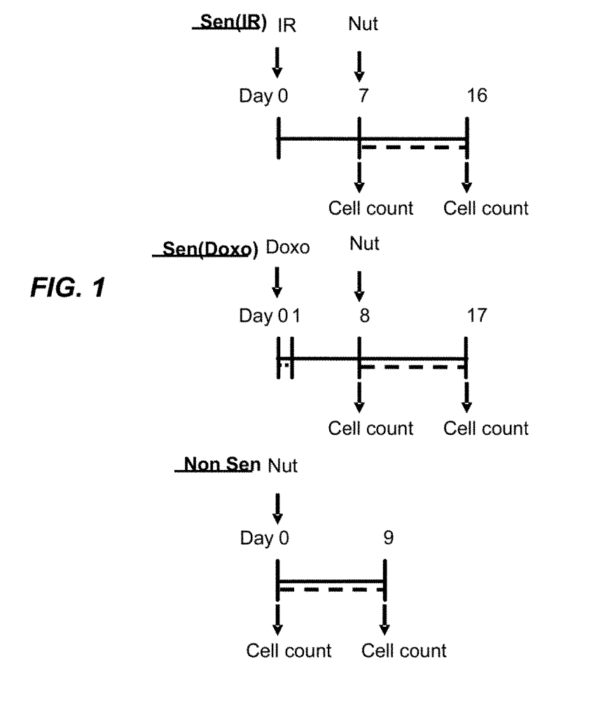

Foreskin fibroblast cell lines HCA2 and BJ, lung fibroblast cell line IMR90, and mouse embryonic fibroblasts were seeded in six-well plates and induced to senesce with 10 Gy of ionizing radiation (IR) or a 24 hr treatment with doxorubicin (Doxo). Senescent phenotype was allowed to develop for at least 7 days, at which point a cell count was made to determine the baseline number of cells. Nutlin-3a treatment was then initiated for a period of at least 9 days. Media alone or media with drug as appropriate was refreshed at least every three days. At the end of the assay time period, cells are counted. Each condition was seeded in three plate wells and counted independently. Initial cell count serves as a control to determine the induction of senescence, as compared to the last day count without nutlin treatment. Initial non-senescent cell count serves as a proxy to determine Nutlin-3a toxicity. FIG. 1 shows a schematic of the experiment design.

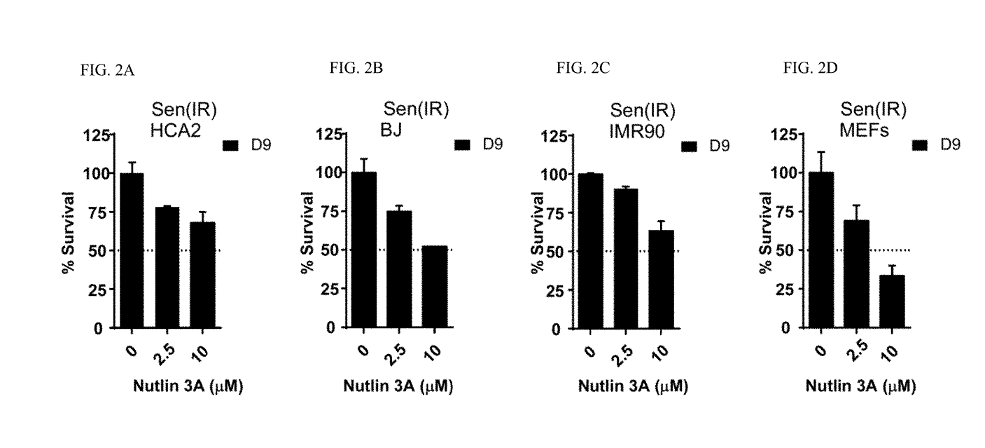

Foreskin fibroblast cell lines HCA2 and BJ, lung fibroblast cell line IMR90, and mouse embryonic fibroblasts were exposed to 10 Gy of ionizing radiation (IR) to induce senescence. Seven days following irradiation, the cell were treated with varying concentrations of Nutlin-3a (0, 2.5 μM, and 10 μM) for a period of 9 days, with the drug refreshed at least every 3 days. Percent survival was calculated as [cell count on day 9 of Nutlin-3a treatment/initial cell count on first day of Nutlin-3a treatment]. The results are shown in FIGS. 2A-D, which show that Nutlin-3a reduced cell survival of senescent foreskin fibroblasts (HCA2 and BJ), lung fibroblasts (IMR90), and mouse embryonic fibroblasts (MEF), indicating Nutlin-3a is a senolytic agent.

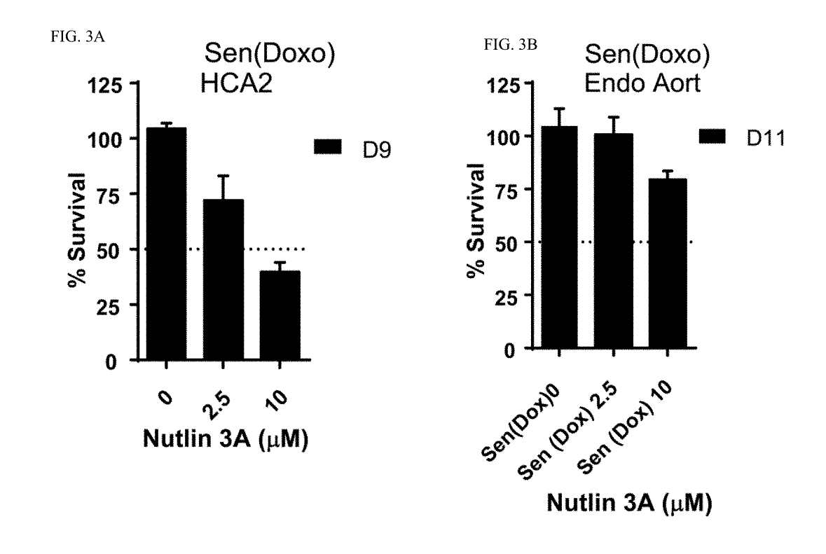

Foreskin fibroblasts (HCA2) and aortic endothelial cells (Endo Aort) were treated with doxorubicin (250 nM) for one day (24 hours) to induce senescence (see FIG. 1). Eight days following doxorubicin treatment, Nutlin-3a treatment was initiated. HCA2 cells were exposed to Nutlin-3a for 9 days, and aortic endothelial cells were exposed to Nutlin-3a for 11 days. Media containing the compound or control media was refreshed at least every 3 days. Percent survival was calculated as [cell count on the last day of Nutlin-3a treatment/initial cell count on first day of Nutlin-3a treatment]. The results are shown in FIGS. 3A-B, which show that doxorubicin-induced senescent cells are sensitive to Nutlin-3a.

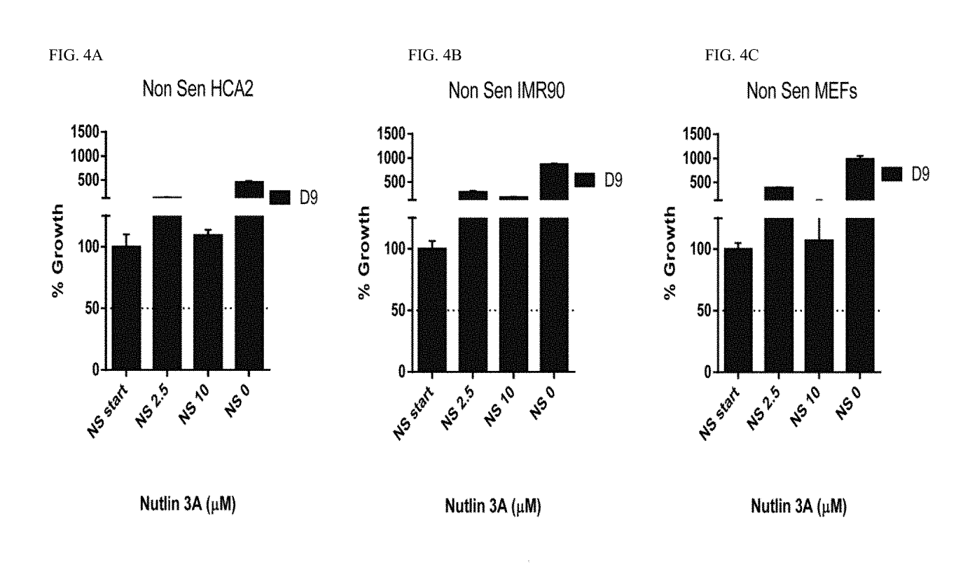

Non-senescent foreskin fibroblasts (HCA2), lung fibroblasts (IMR90), and mouse embryonic fibroblasts (MEF) were treated with varying concentrations (0, 2.5 μM, and 10 μM) of Nutlin-3a for a period of 9 days to assess Nutlin-3a toxicity. Cell counts were taken at the start (NS start) and end of Nutlin-3a treatment. The difference between counts of cells not treated with Nutlin-3a on day 9 (NS 0) and cell counts determined at day zero (NS start) reflects the cell growth over the indicated time period. The results are shown in FIGS. 4A-C, which show that Nutlin-3a treatment reduces proliferation but is non-toxic to non-senescent cells. Nutlin-3a treatment did not decrease the number of cells below the starting level, indicating an absence of toxicity. Decrease in apparent survival between NS 0 and NS 2.5 and between NS 0 and NS 10 reflects a decrease in cell growth. Without wishing to be bound by theory, Nutlin-3a may stabilize p53, leading to cell cycle growth arrest.

Non-senescent aortic endothelial (Endo Aort) cells and pre-adipocytes (Pread) were also treated with varying concentrations (0, 2.5 μM, and 10 μM) of Nutlin-3a for a period of 11 days to assess Nutlin-3a toxicity, as described above. Cell counts were taken at the start at Day 0 (NS start) and at the end of Nutlin-3a treatment (NS 0). The difference between counts of cells not treated with Nutlin-3a on day 11 (NS 0) and cell counts from NS start reflects the cell growth over the indicated time period. The results are shown in FIGS. 5A-B, illustrating that Nutlin-3a treatment reduces proliferation but is non-toxic to non-senescent cells. As observed with fibroblasts, Nutlin-3a treatment does not decrease the number of cells below the starting level, indicating an absence of toxicity. Decrease in apparent survival between NS 0 and NS 2.5 and between NS 0 and NS 10 reflects a decrease in cell growth.

### Example 2 Nutlin-3A Treatment of p16-3MR Transgenic Mice

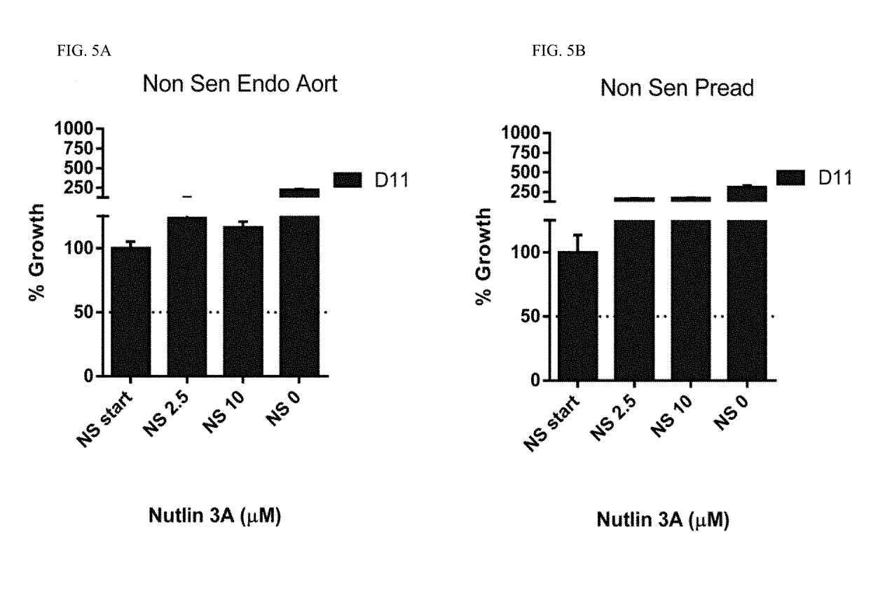

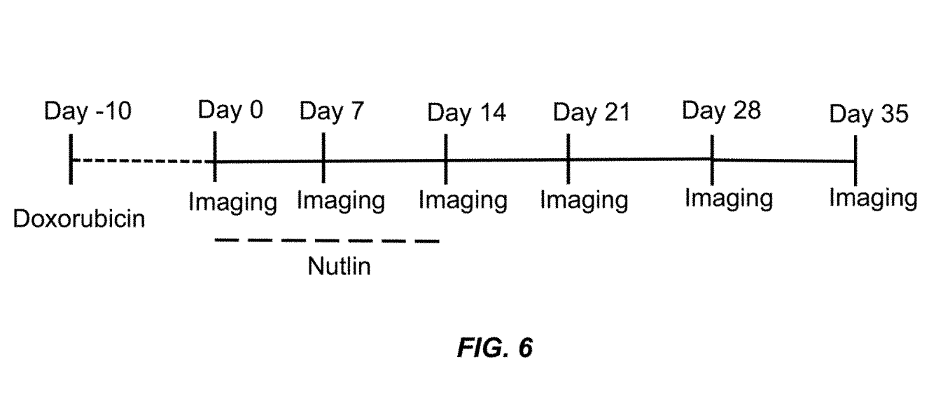

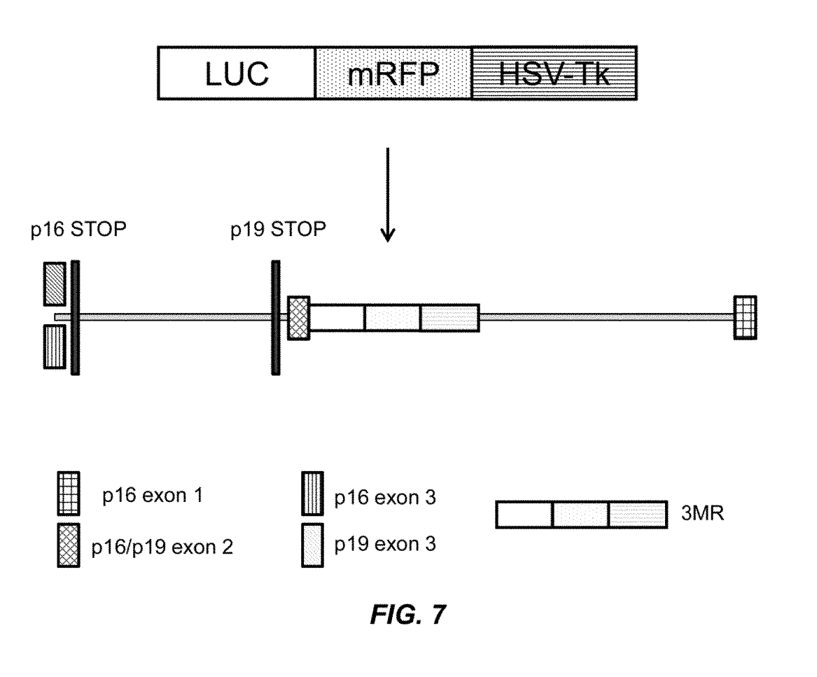

The capability of Nutlin-3a to remove senescent cells in vivo was determined in transgenic p16-3MR mice (see, e.g., International Application Publication No. WO2013/090645). A schematic of the experimental protocol is provided in FIG. 6. The transgenic mouse comprises a p16^{Ink4a} promoter operatively linked to a trimodal fusion protein for detecting senescent cells and for selective clearance of senescent cells in these transgenic mice, which is illustrated in FIG. 7. The promoter, p16^{Ink4a}, which is transcriptionally active in senescent cells but not in non-senescent cells (see, e.g., Wang et al., J. Biol. Chem. 276:48655-61 (2001); Baker et al., Nature 479:232-36 (2011)) was engineered into a nucleic acid construct. 3MR (tri-modality reporter) is a fusion protein containing functional domains of a synthetic Renilla luciferase (LUC), monomeric red fluorescence protein (mRFP), and truncated herpes simplex virus (HSV)-1 thymidine kinase (tTK), which allows killing by ganciclovir (GCV) (see, e.g., Ray et al., Cancer Res. 64:1323-30 (2004))). The 3MR cDNA was inserted in frame with p16 in exon 2, creating a fusion protein containing the first 62 amino acids of p16, but not a full-length wild-type p16 protein. Insertion of the 3MR cDNA also resulted in the occurrence of a stop codon in the p19^{ARF} reading frame in exon 2, thereby preventing full-length p19^{ARF} expression from the BAC as well. The p16^{Ink4a} gene promoter (approximately 100 kilobase pairs) was introduced upstream of a nucleotide sequence encoding a trimodal reporter fusion protein. Alternatively, a truncated p16^{Ink4a} promoter may be used (see, e.g., Baker et al., Nature, supra; International Application Publication No. WO2012/177927; Wang et al., supra).). Thus, the expression of 3MR is driven by the p16^{Ink4a} promoter in senescent cells only. The detectable markers, LUC and mRFP permitted detection of senescent cells by bioluminescence and fluorescence, respectively. The expression of tTK permitted selective killing of senescent cells by exposure to the pro-drug ganciclovir (GCV), which is converted to a cytotoxic moiety by tTK. Transgenic founder animals, which have a C57B16 background, were established and bred using known procedures for introducing transgenes into animals (see, e.g., Baker et al., Nature 479:232-36 (2011)). Female C57/BL6 p16-3MR mice were randomized into doxorubicin+Nutlin-3a treated or doxorubicin only treated groups (see FIG. 6). Senescence was induced by intraperitoneal administration of doxorubicin at 10 mg/kg to the mice ten days prior to administration of Nutlin-3a (Day −10). Nutlin-3a (25 mg/kg) was administered intraperitoneally daily from day 10 to day 24 post-doxorubicin treatment (Group=9 mice). Control mice (doxorubicin treated) were injected with equal volumes of PBS (Group=3 mice). Luminescence imaging (Xenogen Imaging system) was performed at Day 0 (i.e., 10 days post-doxorubicin treatment) as a baseline for each mouse (100% intensity).

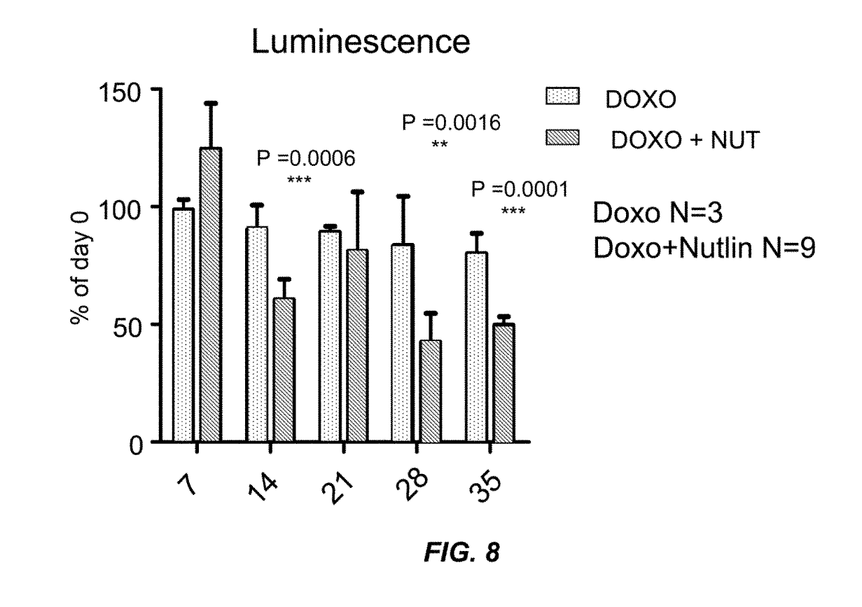

Luminescence imaging of the mice was performed on day 7, 14, 21, 28, and 35 following the initiation of Nutlin-3a treatment. Reduction of luminescence (L) was calculated as: L=(Imaging post-Nutlin-3a treatment)/(Baseline Imaging) %. If L is greater than or equal to 100%, the number of senescent cells was not reduced. If L is less than 100%, then the number of senescent cells was reduced. Every mouse was calculated independently, and background was subtracted from each sample. The results are presented in FIG. 8, which suggest that treatment with Nutlin-3a reduced luminescence associated with doxorubicin-induced senescence. A statistically significant decrease in luminescence was observed at day 14, day 28, and day 35 in Nutlin-3a treated animals.

Experiments were performed to determine the effect of Nutlin-3a treatment on expression of genes associated with senescence. Groups of female C57/BL6 p16-3MR were treated as described above. Three weeks after the end of Nutlin-3a treatment (day 35), the doxorubicin treated mice (control) (N=3) and doxorubicin+Nutlin-3a-treated mice (N=6) were sacrificed. Skin and fat biopsies were collected for RNA extraction; fat biopsies were collected for detection of senescence-associated β-galactosidase; and lungs were flash frozen in cryoprotectant OCT media for cryostat sectioning.

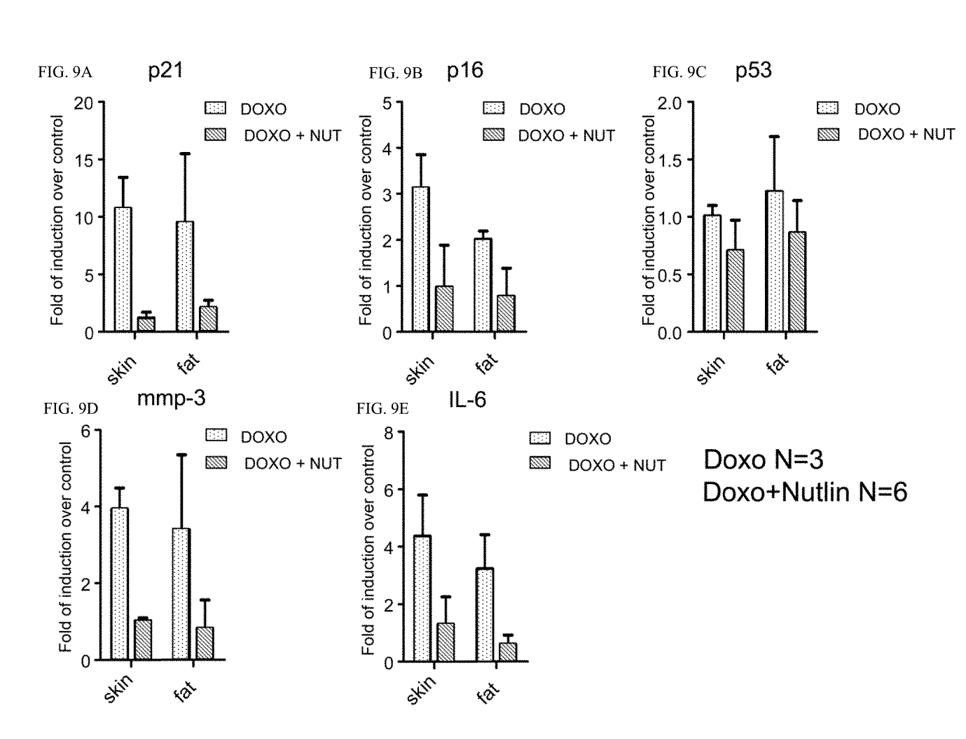

RNA was analyzed for mRNA levels of endogenous senescence markers (p21, p16^{INK4a} (p16), and p53) and SASP factors (mmp-3 and IL-6) relative to actin mRNA (control for cDNA quantity) using the Roche Universal Probe Library for real-time PCR assay. The results are presented in FIGS. 9A-E, which suggest Nutlin-3a treatment reduced expression of SASP factors and senescence markers associated with doxorubicin-induced senescence. Values represent fold of induction of the respective mRNA over untreated control animals.

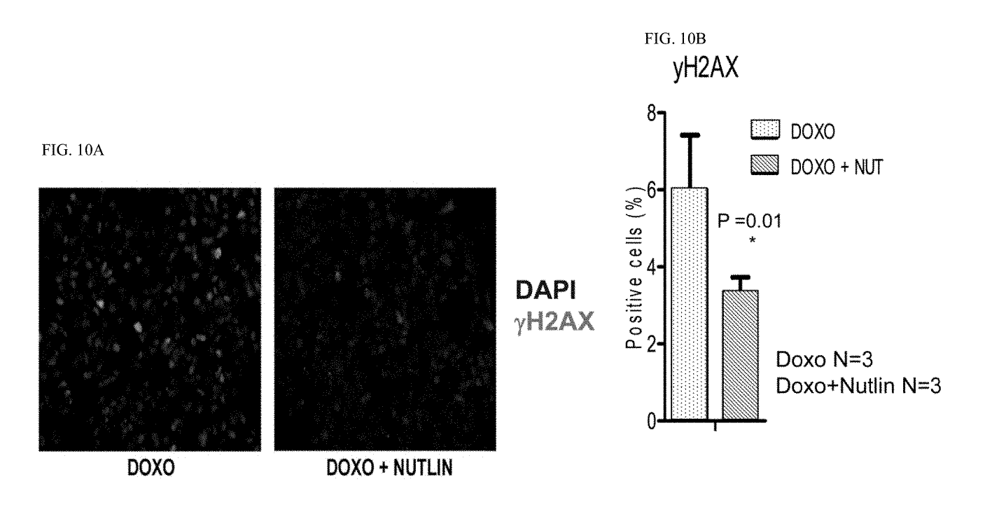

The frozen lung tissue were sectioned to 10 μM thickness and stained with primary rabbit polyclonal antibody against γH2AX (Novus Biologicals, LLC), which is a marker for double-strand breaks in cells (DNA damage). The sections were then stained with ALEXA FLUOR® dye-labeled secondary goat anti-rabbit antibody (Life Technologies) and counterstained with 4′,6-diamidino-2-phenylindole (DAPI) (Life Technologies). The number of positive cells was calculated using ImageJ image processing program (National Institutes of Health, see Internet at imagej.nih.gov/ij/index.html) and represented as a percentage of the total number of cells. The results are presented in FIG. 10A-B, which show that nutlin-3A treatment reduced the number of cells with DNA damage induced by doxorubicin. FIG. 10A shows reduced yH2AX staining in doxorubicin+Nutlin-3a treated cells compared with cells treated with doxorubicin alone. FIG. 10B shows a reduction in the percent γH2AX positive cells in doxorubicin+Nutlin-3a treated cells as compared to cells treated with doxorubicin alone.

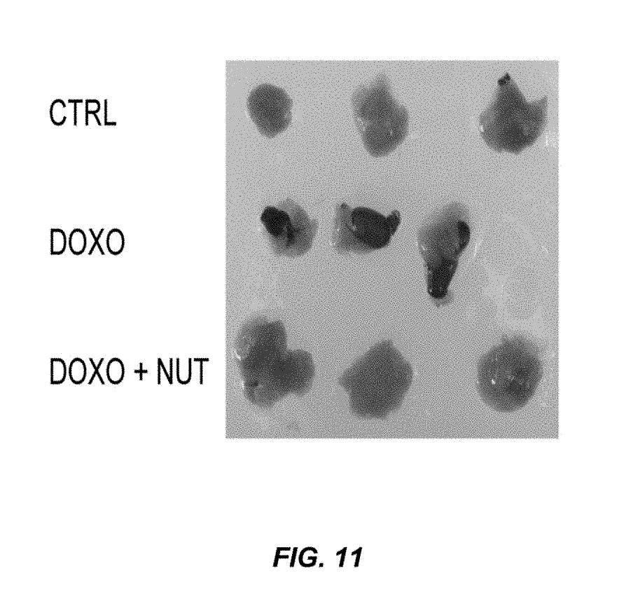

Upon collection, fat biopsies were immediately fixed in 4% formalin and then stained with a solution containing X-gal to detect the presence of senescence-associated β-galactosidase (β-gal). Fat biopsies were incubated overnight at 37° C. in X-gal solution and were photographed the next day. Fat biopsies from untreated animals were used as a negative control (CTRL). The results are presented in FIG. 11, which show that Nutlin-3a treatment reduced senescence-associated β-gal intensity in fat biopsies from animals with doxorubicin-induced senescence similar to untreated negative controls, as compared to mice treated with doxorubicin alone.

### Example 3 MDM2 Inhibitor Removes Senescent Cells with Established SASP

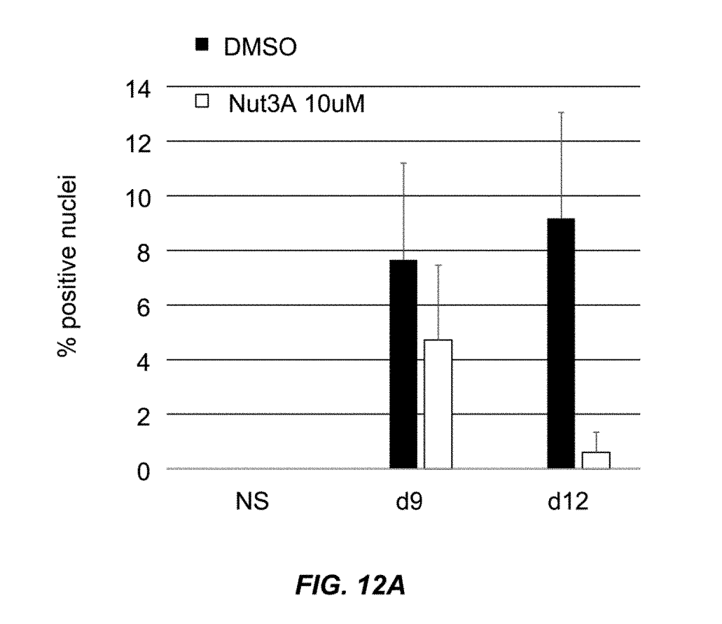

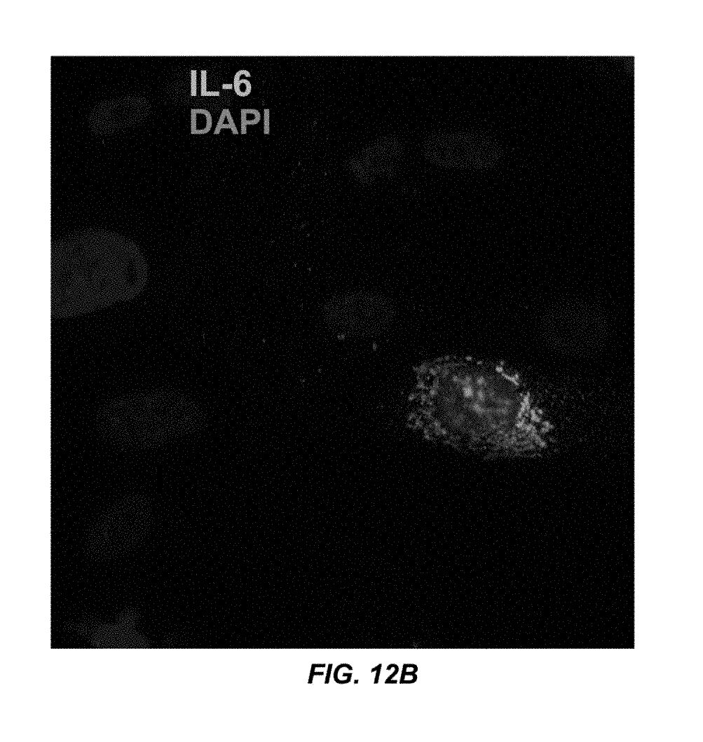

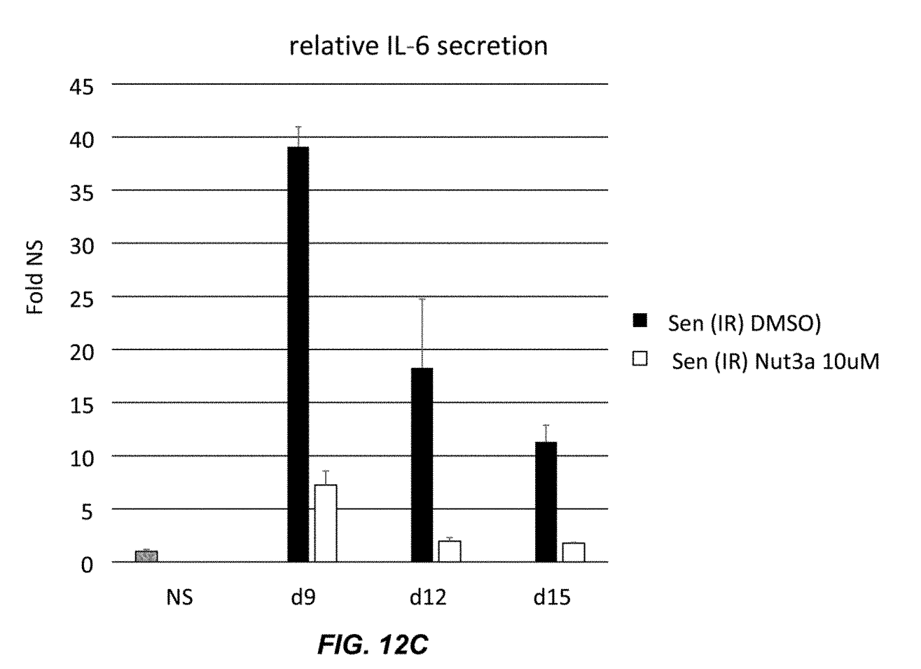

Primary human fibroblast (IMR90) cells were induced to senesce by applying 10 Gy of irradiation. Seven days after irradiation (Day 0), cells were treated with 10 μM Nutlin-3a or vehicle (DMSO) for nine days (Day 9). The drug or vehicle was refreshed every three days. Drug/vehicle was removed at Day 9 and the cells were cultured for an additional three days (Day 12). Cells were then fixed with 4% paraformaldehyde and stained by immunofluorescence with a specific anti-IL-6 antibody (R&D, AF-206-NA). Cells were counterstained with DAPI for nuclear visualization. The percent IL-6 positive cells is illustrated in FIG. 12A. An example of IL-6 positive cell immunofluorescence is shown in FIG. 12B. IL-6 positive cells were determined in an unbiased manner using CellProfiler software. Three different cultures were assessed. Non-senescent cells had no detectable cells IL-6 production while senescent cells were about 8% positive at day 9 after vehicle (DMSO) treatment (16 days after irradiation). Nutlin-3a treatment decreased the percent IL-6 positive cells to a level below 5%. At day 12, 3 days after Nutlin-3a was removed and 19 days after irradiation, IL-6 positive cells in the vehicle control were about 9% and Nutlin3a treated cells were less than 1% positive for IL-6.

In another experiment, IMR90 cells were induced to senesce by irradiation (10 Gy). Seven days after irradiation, cells were treated with 10 μM Nutlin-3a or vehicle (DMSO) for nine days (Day 9). The drug or vehicle was refreshed every three days. Drug/vehicle was removed at Day 9 and the cells were cultured for an additional six days. Conditioned media from the treated cells was collected, and IL-6 measurement by ELISA was performed (Perkin Elmer, AL223F). IL-6 levels in culture media were determined by ELISA using a kit according to manufacturer's instructions (AL223F, Perkin Elmer). Cells were fixed with 4% paraformaldehyde and stained by immunofluorescence with a specific anti-IL-6 antibody (R&D, AF-206-NA). The IL-6 level determined by ELISA was normalized to the number of cells in each well. The data are presented in FIG. 22C as a relative level of IL-6 in the treated cells compared to the level in non-senescent cells (NS). The data are presented as an average of three different cell samples.

The level of IL-6 in senescent cells was between 10-40 fold higher than in non-senescent cells. Nutlin-3a treated senescent cells have a level of IL-6 that is 5-9 fold lower than DMSO treated cells. Cells that survive after Nutlin-3a treatment have a lower IL-6 secretion and by extrapolation, a lower SASP, suggesting that Nutlin-3a preferably kills senescent cells with a well-established SASP.

### Example 4 MDM2 Inhibitor Removes Senescent Cells with Established SASP: SASP Factor Expression

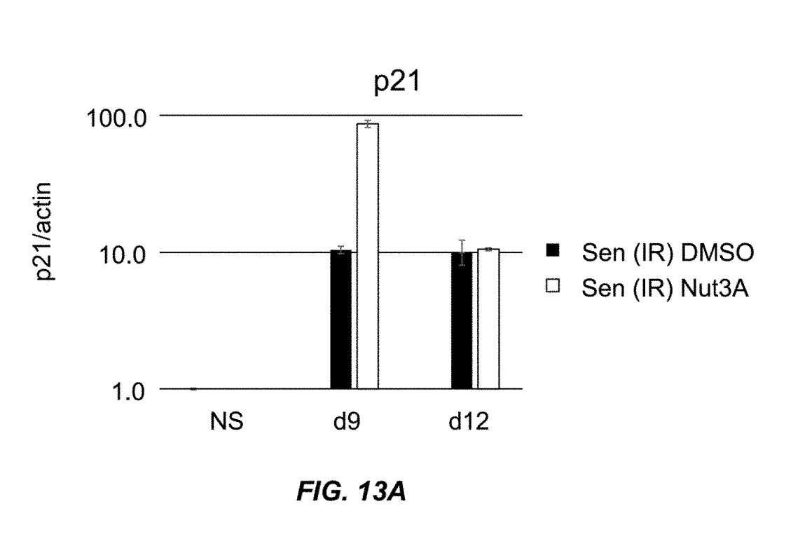

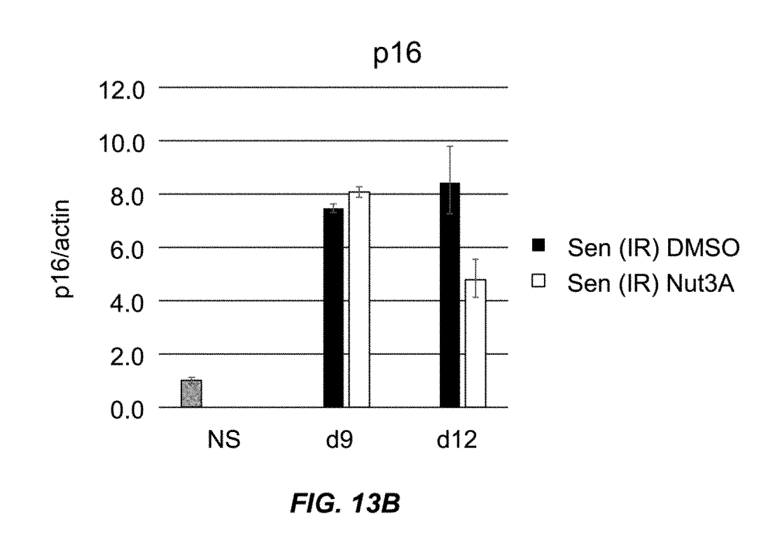

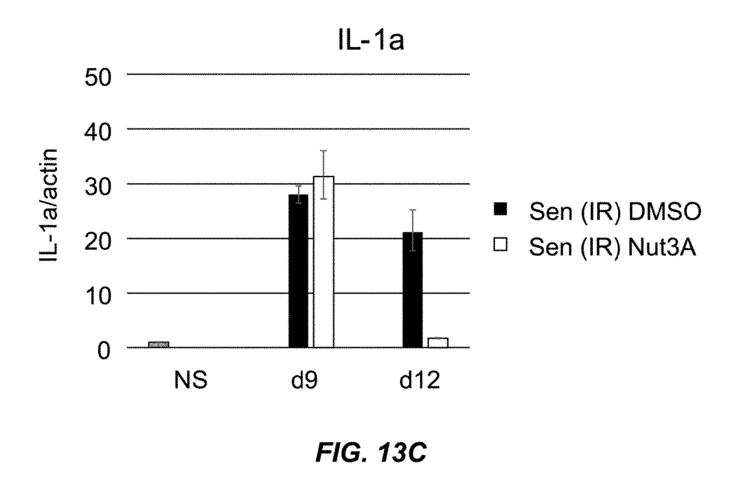

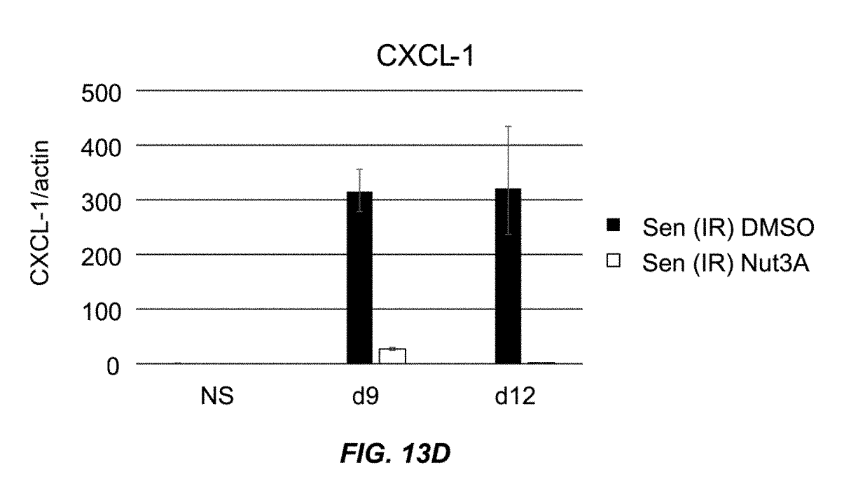

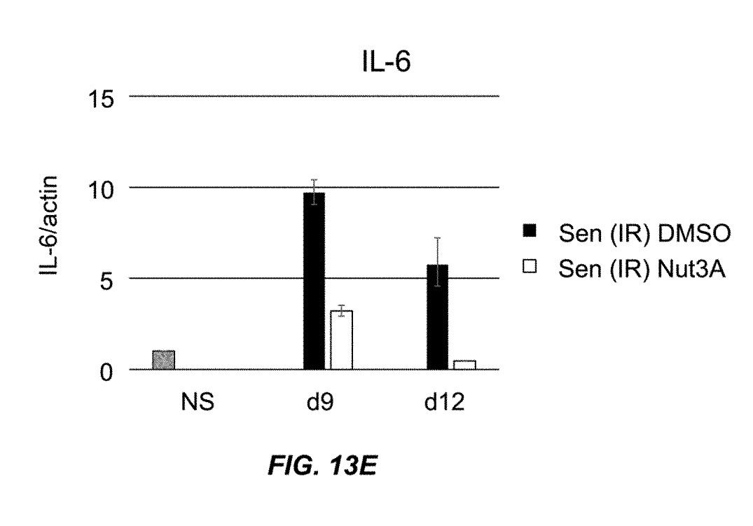

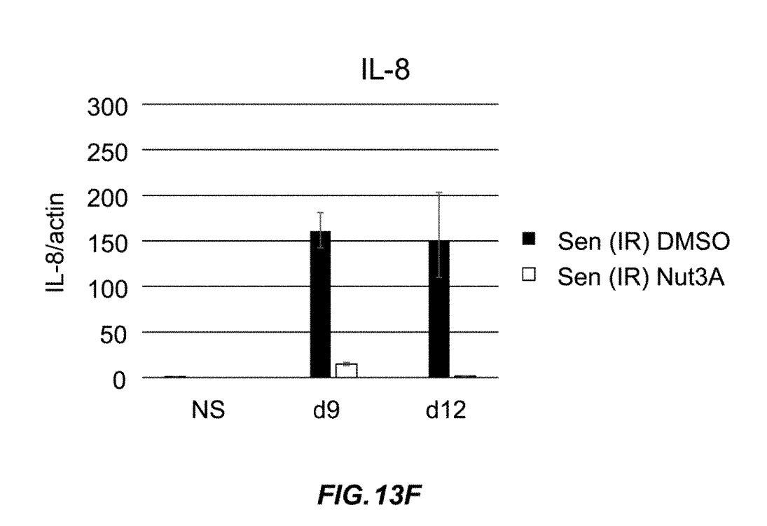

Primary human fibroblast (IMR90) cells were induced to senesce by applying 10 Gy of irradiation. Seven days after irradiation (Day 0), cells were treated with 10 μM Nutlin-3a or vehicle (DMSO) for nine days (Day 9). The drug or vehicle was refreshed every three days. Drug/vehicle was removed at Day 9 and the cells were cultured for an additional three days (Day 12) in media without drug or DMSO. Cells were then collected, mRNA extracted, and cDNA prepared. Quantitative PCR (qPCR) was then performed to detect expression of various genes. Cells were also collected at Day 12 after drug/vehicle had been removed for three days. The data are presented as an average of three samples. Data were normalized to actin and depicted as a ratio to non-senescent cells. The data are presented in FIGS. 13A-13F.

The level of p21 was approximately 10-fold greater in senescent cells, and was higher (approximately 90 fold) when cells were treated with Nutlin-3a. Nutlin-3a stabilizes p53, and p53 is a transcription factor activating the expression of the cyclin dependent kinase inhibitor p21. At day 12, the level of p21 in the DMSO treated cells was comparable to the level at day 9, which was also comparable to the level in the Nutlin-3a treated cells at day 12. These data suggest the acute effect of Nutlin-3a on cells is abrogated after three days after removal of drug exposure. The level of P16, another senescence marker, increased in irradiated cells and did not change in the presence of Nutlin-3a. Three days after the drug has been removed (Day 12), a decrease in p16 level was observed. The level of IL-1a, a regulator of the SASP, decreased only after Nutlin-3a had been removed. CXCL-1, IL-6 and IL-8 are three other SASP factors. The levels of all three were reduced when Nutlin-3a was present and remained lower after drug removal. These data show that cells surviving Nutlin-3a treatment have a lower p16 level, suggesting that Nutlin-3a preferably kills cells that are high p16 expressers. Similarly, SASP factors were reduced in surviving cells, also suggesting that Nutlin-3a preferably kills cells with a higher SASP.

### Example 5 MDM2 Inhibitor Removes Senescent Cells with Elevated DNA Damage Response

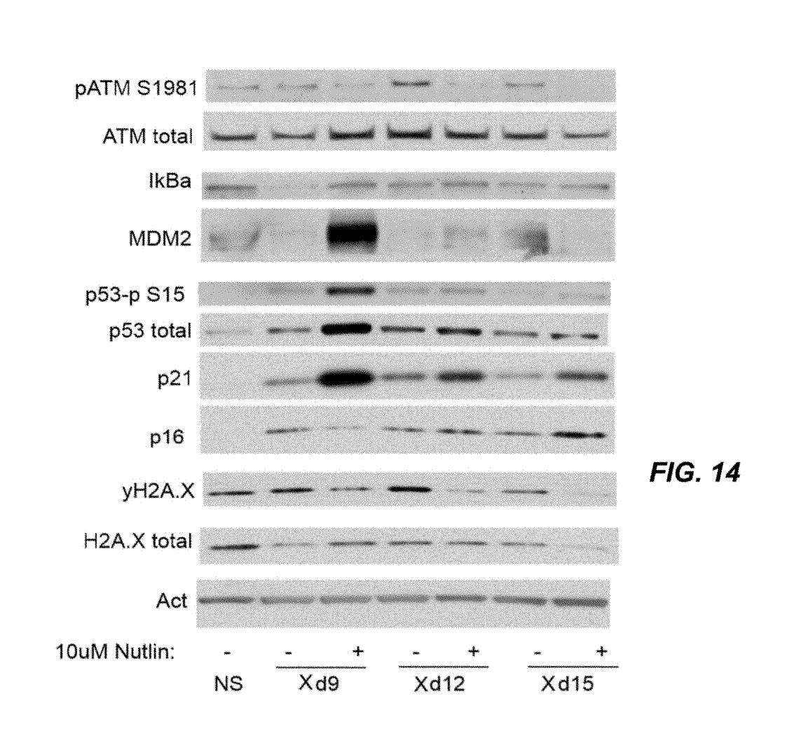

Primary human fibroblast (IMR90) cells were induced to senesce by applying 10 Gy of irradiation. Seven days after irradiation (Day 0), cells were treated with 10 μM Nutlin-3a or vehicle (DMSO) for nine days (Day 9). The drug or vehicle was refreshed every three days. Drug/vehicle was removed at Day 9 and the cells were cultured for an additional six days in media without drug or DMSO, changing media every three days. Cells were collected at Day 0 (non-senescent cells), Day 9, Day 12, and Day 15, and protein extracted and processed for immunoblotting (Western blotting). Two samples were processed at each time point; the results are provided for one sample in FIG. 14.

The data show that phosphorylation of the kinase ATM is lower in cells that have been treated with Nutlin-3a even when the drug has been removed (see pATM S1981). Similarly, the substrate of ATM, H2AX, had declining levels of phosphorylation (see yH2AX) after Nutlin 3A treatment and also after drug removal. In senescent cells, IkBa gets degraded as the NF-kB pathway is activated, which leads to SASP. The data show that after drug is removed, the level of IkBa in Nutlin-3a treated cells approaches the level of IkBa in non-senescent cells. The levels of each of MDM2, p53 and p21 were elevated in the Nutlin-3a treated samples and decreased when the drug was removed.

These data also support that Nutlin-3a preferentially kills cells with a higher SASP. In addition, because a lower level of activated ATM is produced in surviving cells after drug treatment, these data suggest that DNA damage response-activated senescent cells are the cells that are sensitive to Nutlin-3a.

### Example 6 Selective Toxicity of ABT-263 for Senescent Cells Using a Cell Counting Assay

To determine whether ABT-263 is selectively toxic to senescent cells compared to non-senescent cells, a cell counting assay was used to determine cell survival following treatment with ABT-263. The general timelines and procedures for the cell counting assay are shown in FIG. 15. IMR90 cells (human primary lung fibroblasts (IMR90) (IMR-90 (ATCC® CCL-186™, Mannassas, Va.) were seeded in six well plates, and cells were induced to senescence with 10 Gy of ionizing radiation (IR) (Day 0). The media was refreshed every 3 days. The senescent phenotype is allowed to develop for 7 days at which point a cell count was made to determine the baseline number of cells. In the senescent cells (irradiated) and the non-senescent cells (the non-radiated cells), 3 μM ABT-263 was introduced into the media. Some cells were administered a media that did not contain any ABT-263 as a control to account for any ABT-263 toxicity. Each condition was seeded in three wells and counted independently. Cells were counted after a 24 hour exposure to ABT-263 (or control culture).

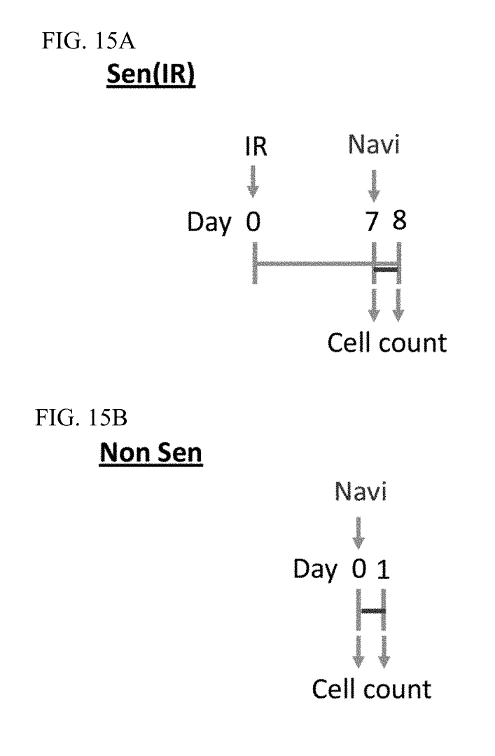

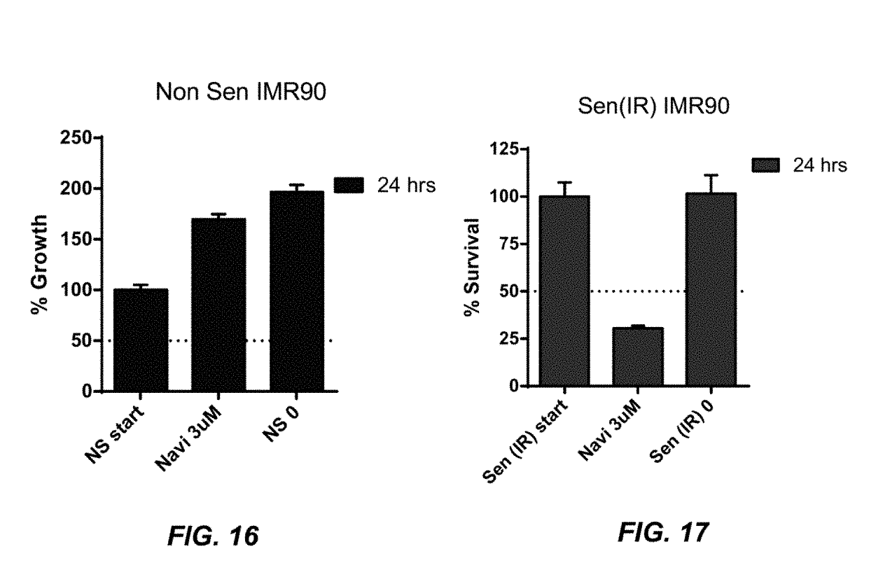

FIG. 16 demonstrates the effect of ABT-263 on non-senescent cells as measured as a percentage of survival of cells after 24 hours. The addition of ABT-263 to non-senescent (middle bar) did not decrease the cell growth below the starting level (left-most bar) indicating an absence of toxicity in non-senescent cells. Non-ABT-263 treated cells are shown as a control at the far-most right.

FIG. 17 demonstrates the effect of ABT-263 on senescent cells as measured as a percentage of survival of cells after 24 hours. The addition of ABT-263 to senescent cells (middle bar) had decreased cell growth below that of the starting level number of cells (left most bar). The ABT-263 treated cells had 28% of the cell counts before ABT-263 treatment. Non-ABT-263 treated cells are shown as a control at the far-most right.

### Example 7 Selective Toxicity of ABT-263 for Senescent Cells Using a CellTiter-Glo® Cell Viability Assay

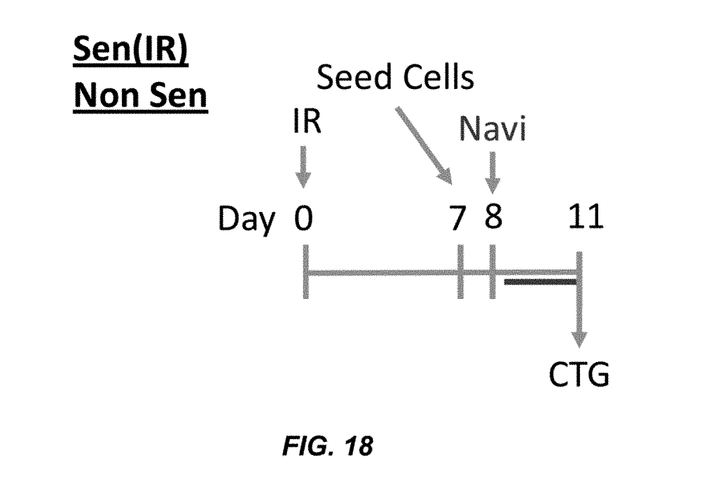

To determine whether ABT-263 is selectively toxic to senescent cells compared to non-senescent cells, a cell viability assay was used to assess cell survival following treatment with ABT-263. The general timelines and procedures for the cell counting assay are shown in FIG. 18. IMR90 cells (human primary lung fibroblasts (IMR90) (IMR-90 (ATCC® CCL-186™, Mannassas, Va.) were seeded in six well plates, and cells were induced to senescence with 10 Gy of ionizing radiation (IR) (Day 0). The media was refreshed every 3 days. The senescent phenotype is allowed to develop for 7 days at which point a cell count was made to determine the baseline number of cells followed by seeding into 96-well plates. On day 8, the senescent cells (irradiated) and the non-senescent cells (the non-radiated cells), were exposed to serial dilutions of ABT-263 for a period of 3 days. ABT-263 concentrations ranged from 0.5 nM to 3 μM. Each condition was seeded in triplicate.

After three days of treatment (Day 11), cells were assayed for cell survival using the commercially available CellTiter-Glo® (CTG) Luminescent Cell Viability Assay (Promega Corporation, Madison, Wis.). The assay determines the number of viable cells in culture based on the quantitation of ATP present which is an indicator of metabolically active cells.

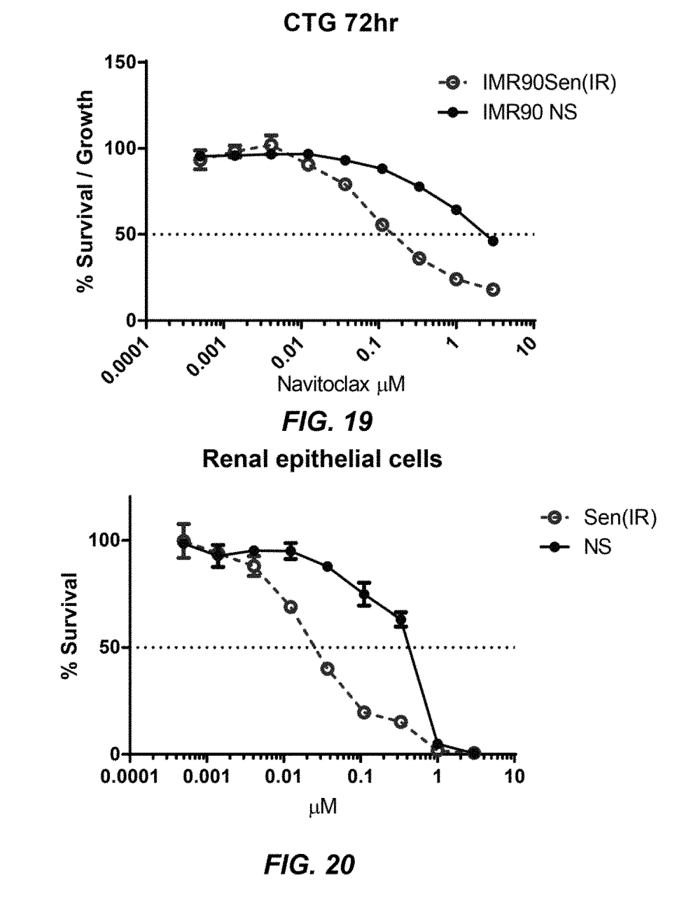

FIG. 19 shows IC50 curves of ABT-263 in senescent cells, and in non-senescent cells. The IC50 curve is a plot of the percentage of cell survival following treatment of ABT-263 as determined by the cell viability assay. The plot shows the effect of the various concentration levels of ABT-263 on cell survival. The IC50 of ABT-263 on non-senescent cells was 2.4 μM compared to an IC50 value of 140 nM on senescent cells, demonstrating the selective toxicity of ABT-263 for senescent cells. An in vitro theoretical therapeutic index of 17 was observed.

### Example 8 Assessment of Selective Toxicity of ABT-263 for Senescent Cells of Various Cell Types

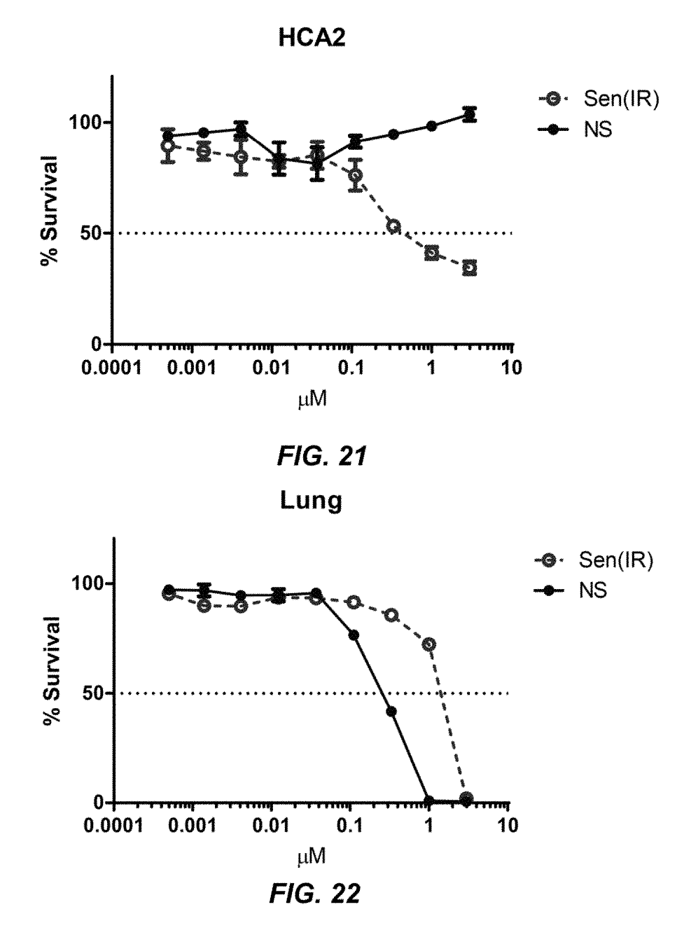

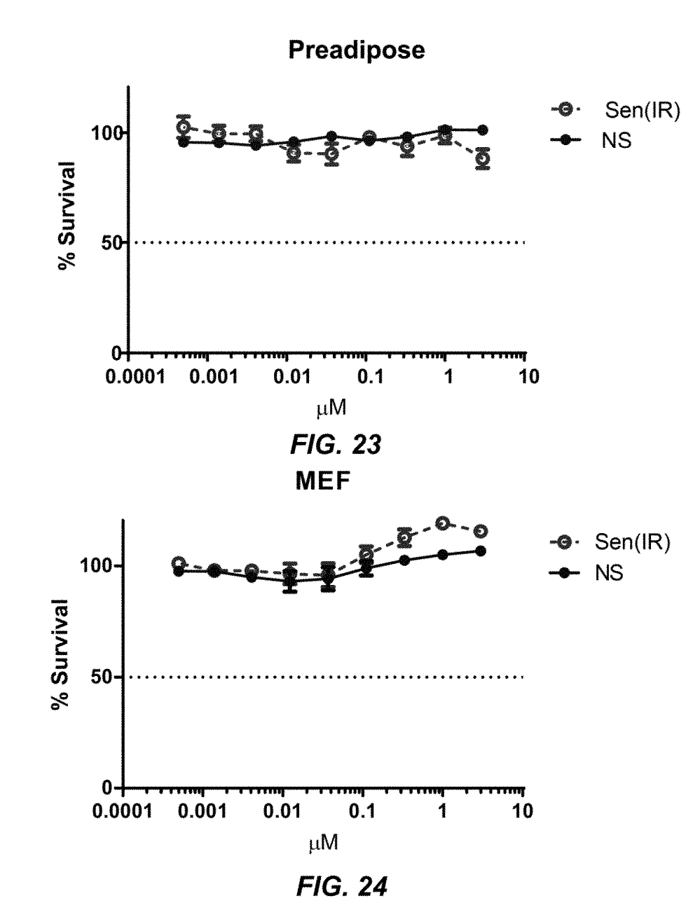

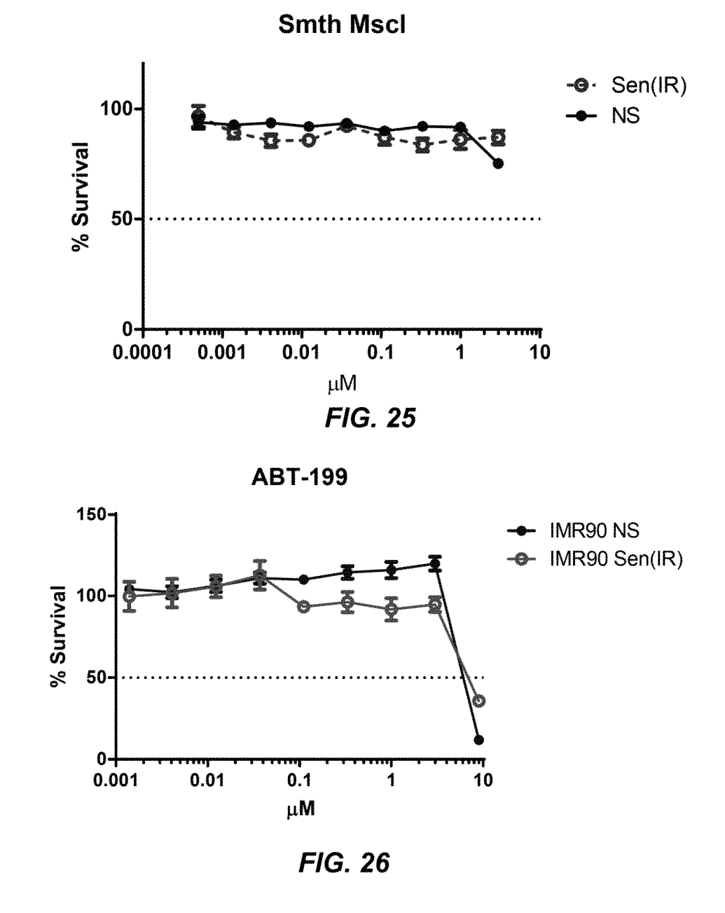

The methods of Example 7 were repeated in other cell strains. Cell strains included Primary Renal Cortical Cells, ATCC Cat# PCS-400-011 (FIG. 20), HCA2 foreskin fibroblast cells (FIG. 21), Primary Small Airway Epithelial Cells, ATCC Cat# PCS-301-010 (lung) (FIG. 22), human pooled Preadipocyte from patients (Pread) (FIG. 23), Mouse embryonic fibroblast extracted from C57B16 mice (MEF) (FIG. 24), Primary Coronary Artery Smooth Muscle, ATCC Cat# PCS-100-021 (Smth Mscl) (FIG. 25).

The experiments performed in these other cell strains were performed essentially as described in Example 7. As shown in FIG. 20, the IC50 of ABT-263 on non-senescent cells was 430 nM compared to an IC50 value of 25 nM on senescent cells, demonstrating the selective toxicity of ABT-263 for senescent cells in renal epithelial cells.

As shown in FIG. 21, the IC50 of ABT-263 on non-senescent cells was not toxic as up to 3 μM compared to an IC50 value of 410 nM on senescent cells, demonstrating the selective toxicity of ABT-263 for senescent cells in HCA2 cells.

### Example 9 Assessment of Selective Toxicity of ABT-263 and Other Bcl-2 Inhibitors for Senescent Human Primary Lung Fibroblasts

To determine whether other Bcl-2 inhibitors demonstrate selective toxicity for senescent cells over non-senescent cells, cells were treated with ABT-199 (Selleckem Cat#58048, Houston, Tex.) or Obatoclax (Selleckem Cat# S1057). ABT-199 and Obatoclax are known Bcl-2 inhibitors.

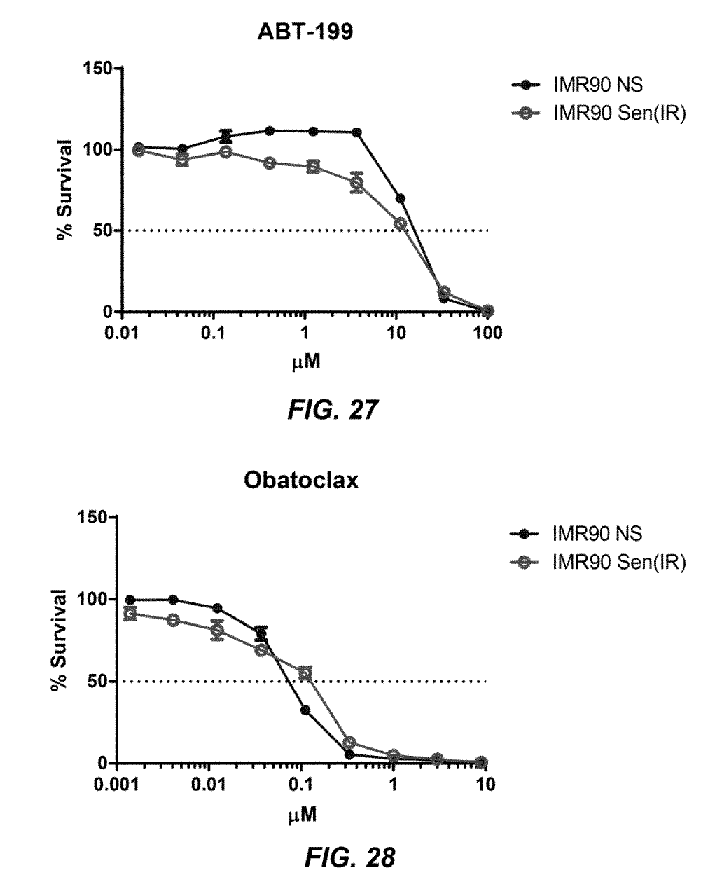

The experiments performed for assessing the effect of these other Bcl-2 inhibitors were performed essentially as described in Example 7. Cells were exposed to ABT-199 at serial dilution concentrations ranging from 15 nM to 100 μM (FIGS. 26 and 27). Cells were exposed to Obatoclax at concentrations ranging from 1.4 nM to 9 μM (FIG. 28).

As shown in FIGS. 26-27, ABT-199 had an IC50 value of 6 μM-15.8 μM in non-senescent cells compared to an IC50 value of 6.9 μM-12.4 μM in senescent cells. As shown in FIG. 28, Obatoclax had an IC50 value of 75 nM in non-senescent cells compared to an IC50 value of 125 nM in senescent cells. FIG. 26-28 demonstrate the inability of ABT-199 and Obatoclax to selectively target senescent cells over non-senescent cells.

A compound specific for Bcl-2A1 also did not selectively kill senescent cells. IMR90 cells were induced to senescence by irradiation as described in Example 7. The irradiated IMR90 cells and non-senescent IMR90 cells were then exposed to a compound called ML214 that is a Bcl-2A1 specific inhibitor. The level of killing of senescent cells was comparable to the level of killing of non-senescent cells.

### Example 10 Selective Toxicity for Senescent Cells of the Akt Inhibitor, MK-2206 Alone and in Combination with ABT-263

The effect of ABT-263 in combination with the Akt inhibitor MK-2206 was tested for selective toxicity of senescent cells compared to non-senescent cells in IMR90 cells. The methods of Example 7 were repeated except that cell cultures were exposed to 10 nM MK-2206 (Selleckem, Cat# S1078) in addition to serial dilutions of ABT-263.

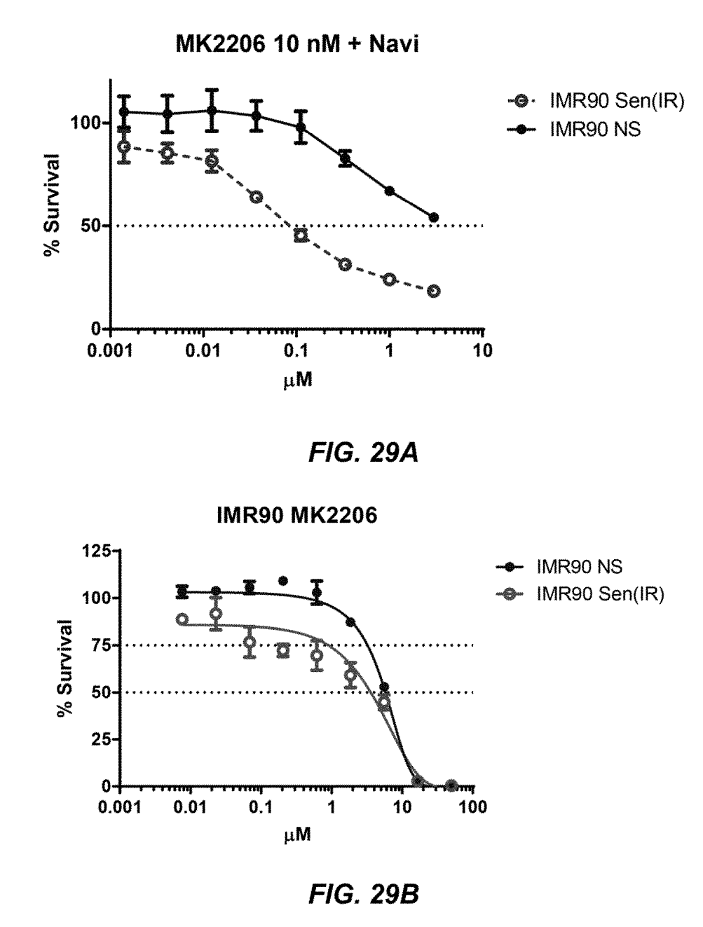

FIG. 29A shows the dose dependence plots of ABT-263 treatment in combination with 10 nM MK-2206 on senescent cells and non-senescent cells. ABT-263+MK-2206-treated senescent cells had an IC50 value of 0.083 μM, whereas ABT-263+MK-2206 cells in non-senescent cells had an IC50 value >3 μM, yielding a selectivity index of >36 for senescent cells.

The senolytic effect of MK-2206 alone was determined by exposing senescent IMR90 cells and non-senescent IMR90 cells (see procedures in Example 7) and to serial dilutions of MK-2206. The percent survival was determined, and the results are present in FIG. 29B.

### Example 11 An Animal Study for Determining the Senolytic Effect of ABT-263 in Mice

The senolytic effect of senolytic agents, e.g., ABT-263, can be assessed in animal models of senescence. An example of such an animal study is described here. Senescence in animals can be induced through the administration of doxorubicin followed by treatment of a senolytic agent. On day 35, mice are sacrificed, and fat and skin are collected for RNA analysis, while lungs are collected and flash frozen for immunomicroscopy analysis. RNA is analyzed for expression of SASP factors (mmp3, IL-6) and senescence markers (p21, p16, and p53). Frozen lung tissue is analyzed for DNA damage marker (yH2AX).

The mice to be tested contain a transgene insertion of p16-3MR. 3MR (tri-modality reporter) is a fusion protein containing functional domains of a synthetic Renilla luciferase (LUC), monomeric red fluorescence protein (mRFP), and truncated herpes simplex virus (HSV)-1 thymidine kinase (tTK), which allows killing by ganciclovir (GCV). The 3MR cDNA is inserted in frame with p16 in exon 2, creating a fusion protein containing the first 62 amino acids of p16, but does not include the full-length wild-type p16 protein. Insertion of the 3MR cDNA also introduces a stop codon in the p19^{ARF} reading frame in exon 2.

The effect of ABT-263 is analyzed by the reduction of luminescence intensity. Female C57/B16 p16-3MR mice are treated with Doxorubicin. Luminescence is measured 10 days later and used as baseline for each mouse (100% intensity). ABT-263 is administered intraperitoneally daily from day 10 to day 24 post-doxorubicin treatment. Luminescence is then measured at day 7, 14, 21, 28, 35 post-ABT-263 treatments, and final values calculated as % of the baseline values. Control animals (DOXO) are injected with equal volume of PBS.

The level of mRNA of endogenous mmp-3, IL-6, p21, p16, and p53 in the skin and fat from animals after treatment with doxorubicin alone (DOXO) or doxorubicin plus ABT-263 is plotted. The values represent the fold induction of the particular mRNA compared with untreated control animals.

Immunofluorescence microscopy of lung sections from doxorubicin treated animals (DOXO) and doxorubicin and ABT-263 can be detected by binding to a primary rabbit polyclonal antibody specific for yH2AX followed by incubation with a secondary goat anti-rabbit antibody, and then counterstained with DAPI. The percent positive cells from immunofluorescence microscopy are calculated and can be represented as percentage of the total number of cells. Data can be obtained from doxorubicin-treated mice (Doxo), and doxorubicin+ABT-263-treated mice).

ABT-263 can be analyzed for reduced senescence-associated (SA) β-galactosidase (f3-gal) intensity of fat biopsies from animals first treated with doxorubicin. Female C57/BL6 p16-3MR mice are treated with doxorubicin. A portion of the doxorubicin treated animals receive ABT-263 or PBS (DOXO) daily from day 10 to day 24 post-doxorubicin treatment. Three weeks after the ABT-263 treatment, mice are sacrificed and fat biopsies immediately fixed and stained with a solution containing X-Gal. Untreated animals are used as negative control (CTRL). Example 12 In Vitro Cell Assays for Determining Senolytic Activity of WEHI-539

Lung fibroblast cell line IMR90 (human primary lung fibroblasts, ATCC® CCL-186™, Manassas, Va.) and a renal cell line (Primary Renal Cortical Cells, ATCC Cat. No. PCS-400-011) were seeded in six-well plates and induced to senesce with 10 Gy of ionizing radiation (IR). Senescent phenotype was allowed to develop for at least 7 days.

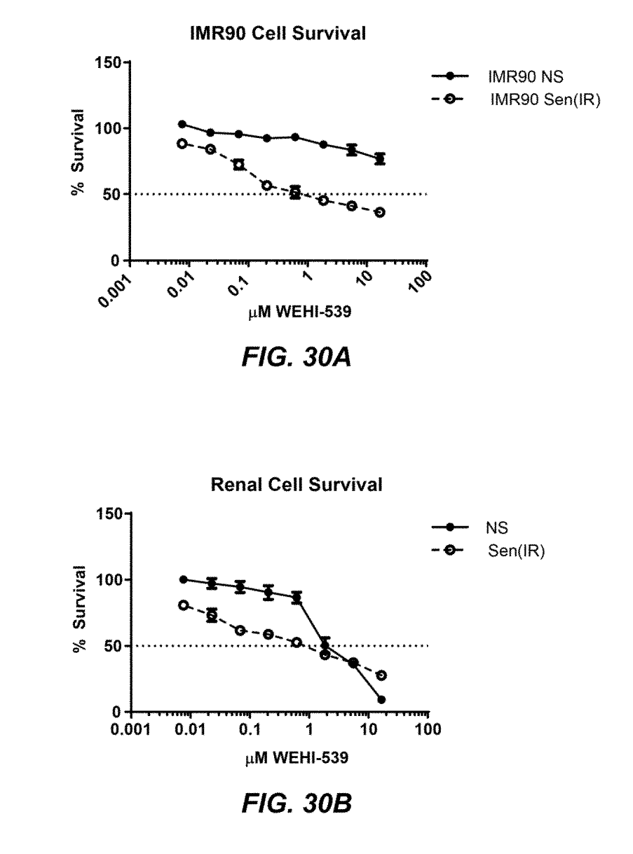

After senescence phenotype had developed, cells were re-seeded into 96 well plates, and senescent cells (irradiated) and non-senescent cells (the non-radiated cells), were exposed to three-fold serial dilutions of WEHI-539 for a period of 3 days. WEHI-539 concentrations ranged from 0.0075 μM to 15 μM. After the three days, cell survival was determined using the commercially available CellTiter-Glo® Luminescent Cell Viability Assay (Promega Corporation, Madison, Wis.). The assay determines the number of viable cells in culture based on the quantitation of ATP present which is an indicator of metabolically active cells. FIG. 30 presents the IMR90 cell survival (see FIG. 30A) and renal cell survival (see FIG. 30B).

### Example 13 WEHI-539 Treatment of p16-3MR Transgenic Mice

This example describes an animal model useful for determining the capability of a senolytic agent to selectively kill senescent cells in vivo. The capability of WEHI-539 or another senolytic agent to remove senescent cells in vivo is determined in transgenic p16-3MR mice (see, e.g., International Application Publication No. WO2013/090645). An experiment is performed in a similar manner to the procedure illustration in the schematic provided in FIG. 6. The transgenic mouse comprises a p16^{Ink4a} promoter operatively linked to a trimodal fusion protein for detecting senescent cells and for selective clearance of senescent cells in these transgenic mice, which is illustrated in FIG. 7. The promoter, p16^{Ink4a}, which is transcriptionally active in senescent cells but not in non-senescent cells (see, e.g., Wang et al., I Biol. Chem. 276:48655-61 (2001); Baker et al., Nature 479:232-36 (2011)), was engineered into a nucleic acid construct. 3MR (tri-modality reporter) is a fusion protein containing functional domains of a synthetic Renilla luciferase (LUC), monomeric red fluorescence protein (mRFP), and truncated herpes simplex virus (HSV)-1 thymidine kinase (tTK), which allows killing by ganciclovir (GCV) (see, e.g., Ray et al., Cancer Res. 64:1323-30 (2004)). The 3MR cDNA was inserted in frame with p16 in exon 2, creating a fusion protein containing the first 62 amino acids of p16, but not a full-length wild-type p16 protein. Insertion of the 3MR cDNA also resulted in the occurrence of a stop codon in the p19^{ARF} reading frame in exon 2, thereby preventing full-length p19^{ARF} expression from the BAC as well. The p16^{Ink4a} gene promoter (approximately 100 kilobase pairs) was introduced upstream of a nucleotide sequence encoding a trimodal reporter fusion protein. Alternatively, a truncated p16^{Ink4a} promoter may be used (see, e.g., Baker et al., Nature, supra; International Application Publication No. WO2012/177927; Wang et al., supra). Thus, the expression of 3MR is driven by the p16^{Ink4a} promoter in senescent cells only. The detectable markers, LUC and mRFP permitted detection of senescent cells by bioluminescence and fluorescence, respectively. The expression of tTK permitted selective killing of senescent cells by exposure to the pro-drug ganciclovir (GCV), which is converted to a cytotoxic moiety by tTK. Transgenic founder animals, which have a C57B16 background, were established and bred using known procedures for introducing transgenes into animals (see, e.g., Baker et al., Nature 479:232-36 (2011)).

To determine the senolytic activity of an agent, such as WEHI-539, female C57/BL6 p16-3MR mice are randomized into doxorubicin+WEHI-539 treated or doxorubicin only treated groups. Senescence is induced by intraperitoneal administration of doxorubicin at 10 mg/kg to the mice ten days prior to administration of WEHI-539 (Day −10). WEHI-539 is administered intraperitoneally daily from day 10 to day 24 post-doxorubicin treatment (Group=9 mice). Control mice (doxorubicin treated) are injected with equal volumes of PBS (Group=3 mice). Luminescence imaging (Xenogen Imaging system) is performed at Day 0 (i.e., 10 days post-doxorubicin treatment) as a baseline for each mouse (100% intensity).

Luminescence imaging of the mice is performed on day 7, 14, 21, 28, and 35 following the initiation of WEHI-539 treatment. Reduction of luminescence (L) is calculated as: L=(Imaging post-WEHI-539 treatment)/(Baseline Imaging) %. If L is greater than or equal to 100%, the number of senescent cells was not reduced. If L is less than 100%, then the number of senescent cells was reduced. Every mouse is calculated independently, and background is subtracted from each sample.

Experiments are performed to determine the effect of WEHI-539 treatment on expression of genes associated with senescence. Groups of female C57/BL6 p16-3MR are treated as described above. Three weeks after the end of WEHI-539 treatment (day 35), the doxorubicin treated mice (control) (N=3) and doxorubicin+WEHI-539-treated mice (N=6) are sacrificed. Skin and fat biopsies are collected for RNA extraction; fat biopsies are collected for detection of senescence-associated β-galactosidase; and lungs are flash frozen in cryoprotectant OCT media for cryostat sectioning.

RNA is analyzed for mRNA levels of endogenous senescence markers (e.g., p21, p16^{INK4a} (p16), and p53) and SASP factors (e.g., mmp-3 and IL-6) relative to actin mRNA (control for cDNA quantity) using the Roche Universal Probe Library for real-time PCR assay.

The frozen lung tissue is sectioned to 10 μM thickness and stained with primary rabbit polyclonal antibody against yH2AX (Novus Biologicals, LLC), which is a marker for double-strand breaks in cells (DNA damage). The sections are then stained with ALEXA FLUOR® dye-labeled secondary goat anti-rabbit antibody (Life Technologies) and counterstained with 4′,6-diamidino-2-phenylindole (DAPI) (Life Technologies). The number of positive cells is calculated using ImageJ image processing program (National Institutes of Health, see Internet at imagej.nih.gov/ij/index.html) and represented as a percentage of the total number of cells.

Upon collection, fat biopsies are immediately fixed in 4% formalin and then stained with a solution containing X-gal to detect the presence of senescence-associated β-galactosidase (α-gal). Fat biopsies are incubated overnight at 37° C. in X-gal solution and are photographed the next day. Fat biopsies from untreated animals are used as a negative control (CTRL).

### Example 14 Capability of BCL-XL Inhibitor to Remove Senescent Cells with Established SASP

This example describes a method for determining the effect of a senolytic agent on killing of senescent cells that have established SASP. Primary human fibroblast (IMR90) cells are induced to senesce by applying 10 Gy of irradiation. Seven days after irradiation (Day 0), cells are treated with 10 μM of a BCL-XL inhibitor (e.g., WEHI-539) or a BCL-2/BCL-XL inhibitor or vehicle (DMSO) for nine days (Day 9). The drug or vehicle is refreshed every three days. Drug/vehicle is removed at Day 9 and the cells are cultured for an additional three days (Day 12). Cells are then fixed with 4% paraformaldehyde and stained by immunofluorescence with a specific anti-IL-6 antibody (R&D, AF-206-NA). Cells are counterstained with DAPI for nuclear visualization. IL-6 positive cells are determined in an unbiased manner using CellProfiler software.

In another experiment, IMR90 cells are induced to senesce by irradiation (10 Gy). Seven days after irradiation, cells are treated with senolytic agent (e.g., a BCL-XL inhibitor (e.g., WEHI-539) or a BCL-2/BCL-XL inhibitor; MDM2 inhibitor; Akt inhibitor) or vehicle (DMSO) for nine days (Day 9). The drug or vehicle is refreshed every three days. Drug/vehicle is removed at Day 9 and the cells are cultured for an additional six days. Conditioned media from the treated cells is collected, and IL-6 measurement by ELISA is performed (Perkin Elmer, AL223F). IL-6 levels in culture media are determined by ELISA using a kit according to manufacturer's instructions (AL223F, Perkin Elmer). Cells are fixed with 4% paraformaldehyde and stained by immunofluorescence with a specific anti-IL-6 antibody (R&D, AF-206-NA). The IL-6 level determined by ELISA is normalized to the number of cells in each well.

### Example 15 Capability of a Senolytic Agent to Remove Senescent Cells with Established SASP: SASP Factor Expression

This example describes a method for determining the effect of a senolytic agent on SASP factor expression. Primary human fibroblast (IMR90) cells are induced to senesce by applying 10 Gy of irradiation. Seven days after irradiation (Day 0), cells are treated with a senolytic agent (e.g., a BCL-XL inhibitor (e.g., WEHI-539) or a BCL-2/BCL-XL inhibitor; MDM2 inhibitor; Akt inhibitor) or vehicle (DMSO) for nine days. The drug or vehicle is refreshed every three days. After drug/vehicle is removed prior to evaluation of SASP expression at Day 9, the cells are cultured for an additional three days in media without drug or DMSO. Cells are then collected, mRNA extracted, and cDNA prepared. Quantitative PCR (qPCR) is then performed to detect expression of various genes. Cells are also collected at Day 12 after drug/vehicle had been removed for three days. Data are normalized to actin and depicted as a ratio to non-senescent cells.

### Example 16 Capability of a Senolytic Agent to Remove Senescent Cells with Elevated DNA Damage Response

This example describes a method for determining the effect of a senolytic agent on selectively killing senescent that that have an elevated DNA damage response. Primary human fibroblast (IMR90) cells are induced to senesce by applying 10 Gy of irradiation. Seven days after irradiation (Day 0), cells are treated with a senolytic agent (for example, a BCL-XL inhibitor (e.g., WEHI-539) or a BCL-2/BCL-XL inhibitor, MDM2 inhibitor; Akt inhibitor) or vehicle (DMSO) for nine days (Day 9). The drug or vehicle is refreshed every three days. Drug/vehicle is removed at Day 9 and the cells are cultured for an additional six days in media without drug or DMSO, changing media every three days. Cells are collected at Day 0 (non-senescent cells), Day 9, Day 12, and Day 15, and protein extracted and processed for immunoblotting (Western blotting). Two samples are processed at each time point.

### Example 17 BCL-XL Selective Inhibitor Kills Senescent Cells Via Apoptosis

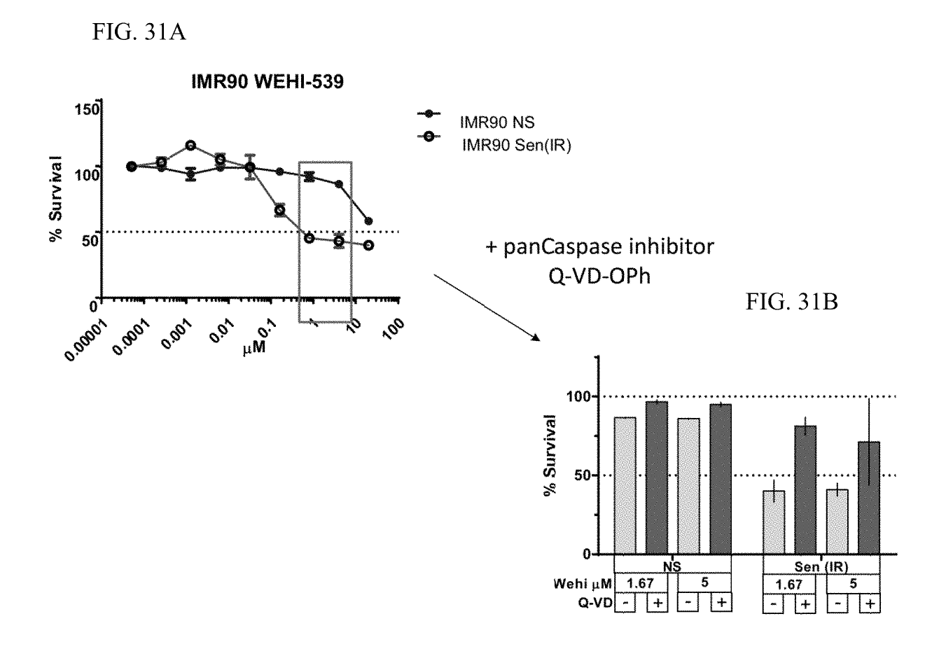

Lung fibroblast cell line IMR90 (human primary lung fibroblasts, ATCC® CCL-186™, Manassas, Va.) were seeded in six-well plates and induced to senesce with 10 Gy of ionizing radiation (IR) as described in Example 12. After senescence was established, cells were re-seeded into 96 well plates. The pan-caspase inhibitor Q-VD-OPh (20 μM) was added to wells of senescent cells (irradiated) (IMR90 Sen(IR)) and to wells containing non-senescent cells (the non-radiated cells) (IMR90 NS). Four hours later, the senescent and non-senescent cells were each exposed for a period of 3 days to 1.67 or 5 μM WEHI-539. At the end of the assay time period, cells were counted. Each condition was seeded in three plate wells and counted independently. Initial cell count served as a control to determine the induction of senescence, as compared to the last day count without WEHI-539 treatment. Initial non-senescent cell count serves as a proxy to determine WEHI-539 toxicity. Cell survival was determined using the commercially available CellTiter-Glo® Luminescent Cell Viability Assay (Promega Corporation, Madison, Wis.). The assay determines the number of viable cells in culture based on the quantitation of ATP present which is an indicator of metabolically active cells. FIG. 31 (left side) is an illustration that WEHI-539 selectively kills senescent cells (see Example 12) and illustrates the WEHI-539 concentrations used in this experiment. In the presence of the pan-caspase inhibitor, the percent of surviving senescent cells increased (FIG. 31, right side).

### Example 18 Effective Killing of Senescent Cells by Inhibiting BCL-XL

This example demonstrates that BCL-XL is the BCL-2 anti-apoptotic family member important for apoptosis of senescent cells. Short hairpin RNAs (shRNA) comprising sequences specific for BCL-2, BCL-XL (also called BCL2L1), and BCL-w (also called BCL2L2) were prepared and introduced into lentiviral vectors. Four different shRNAs for each of BCL-XL and BCL-w and three for BCL-2 were synthesized by the Broad Institute of MIT and Harvard (Cambridge, Mass.). Lentiviral vectors comprising each respective shRNA were purchased from Sigma Aldrich (St. Louis, Mo.). The shRNA sequences and the target sequences are provided in the table below. The nucleotide sequence of each protein can be readily obtained from public databases (see, e.g., Bcl-xL at GenBank NM_001191.2 and NM_138578.1 (BCL2-like 1 (BCL2L1)); Bcl-w at GenBank NM_004050.3 (BCL2-like 2 (BCL2L2)); and Bcl-2 at NM_000633.2, NM_000657 (B-cell CLL/lymphoma 2 (BCL2)).

Triplicate samples of senescent cells and non-senescent cells were transduced with each of the different lentiviral vectors and with two control vectors according to methods practiced in the art. Control samples include senescent and non-senescent cells that were not transduced (NT) with a lentivirus. IMR90 cells were induced to senesce by exposure to 10 Gy of ionizing radiation (IR) as described in Example 12. After senescence phenotype had developed, cells were re-seeded into 96 well plates, and shRNA was added. After 24 hrs, the shRNA was removed and media was refreshed. Media was again refreshed after 3 days. After the last media refresh (6 days after shRNA removal), survival was measured with CellTiter-Glo® Luminescent Cell Viability Assay.

TABLE
shRNA Sequences

| | Protein | | |
|---|---|---|---|
| SYMBOL | Encoded | shRNA Sequence | Target Sequence |
| BCL2 | Bcl-2 | CCGGCCGGGAGATAGTGATGAAGTACTCGAGTACT | CCGGGAGATAGTGAT |
| | | TCATCACTATCTCCCGGTTTTTG | GAAGTA |
| | | (SEQ ID NO: 1) | (SEQ ID NO: 2) |
| BCL2 | Bcl-2 | CCGGGTGATGAAGTACATCCATTATCTCGAGATAA | GTGATGAAGTACATC |
| | | TGGATGTACTTCATCACTTTTTG | CATTAT |
| | | (SEQ ID NO: 3) | (SEQ ID NO: 4) |
| BCL2 | Bcl-2 | CCGGGTGATGAAGTACATCCATTATCTCGAGATAA | GTGATGAAGTACATC |
| | | TGGATGTACTTCATCACTTTTTG | CATTAT |
| | | (SEQ ID NO: 3) | (SEQ ID NO: 4) |
| BCL2 | Bcl-2 | CCGGAGAGTGACAGTGGATTGCATTCTCGAGAATG | AGAGTGACAGTGGAT |
| | | CAATCCACTGTCACTCTTTTTTG | TGCATT |
| | | (SEQ ID NO: 5) | (SEQ ID NO: 6) |
| BCL2L1 | Bcl-xL | CCGGGCTCACTCTTCAGTCGGAAATCTCGAGATTT | GCTCACTCTTCAGTC |
| | | CCGACTGAAGAGTGAGCTTTTTG | GGAA |
| | | (SEQ ID NO: 7) | (SEQ ID NO: 8) |
| BCL2L1 | Bcl-xL | CCGGGTGGAACTCTATGGGAACAATCTCGAGATTG | GTGGAACTCTATGGG |
| | | TTCCCATAGAGTTCCACTTTTTG | AACA |
| | | (SEQ ID NO: 9) | (SEQ ID NO: 10) |
| BCL2L1 | Bcl-xL | CCGGGTTTAGTGATGTGGAAGAGAACTCGAGTTCT | GTTTAGTGATGTGGA |
| | | CTTCCACATCACTAAACTTTTTG | AGAG |
| | | (SEQ ID NO: 11) | (SEQ ID NO: 12) |
| BCL2L1 | Bcl-xL | CCGGGCTCACTCTTCAGTCGGAAATCTCGAGATTT | GCTCACTCTTCAGTC |
| | | CCGACTGAAGAGTGAGCTTTTG | GGAAAT |
| | | (SEQ ID NO: 13) | (SEQ ID NO: 14) |
| BCL2L2 | Bcl-w | CCGGTGGCAGACTTTGTAGGTTATACTCGAGTATA | TGGCAGACTTTGTAG |
| | | ACCTACAAAGTCTGCCATTTTG | GTTA |
| | | (SEQ ID NO: 15) | (SEQ ID NO: 16) |
| BCL2L2 | Bcl-w | CCGGGTCAACAAGGAGATGGAACCACTCGAGTGGT | GTCAACAAGGAGATG |
| | | TCCATCTCCTTGTTGACTTTTTG | GAAC |
| | | (SEQ ID NO: 17) | (SEQ ID NO: 18) |
| BCL2L2 | Bcl-w | CCGGCAGAAGGGTTATGTCTGTGGACTCGAGTCCA | CAGAAGGGTTATGTC |
| | | CAGACATAACCCTTCTGTTTTTG | TGTG |
| | | (SEQ ID NO: 19) | (SEQ ID NO: 20) |
| BCL2L2 | Bcl-w | CCGGCCATTAGATGAGTGGGATTTACTCGAGTAAA | CCATTAGATGAGTGG |
| | | TCCCACTCATCTAATGGTTTTTTG | GATTTA |
| | | (SEQ ID NO: 21) | (SEQ ID NO: 22) |

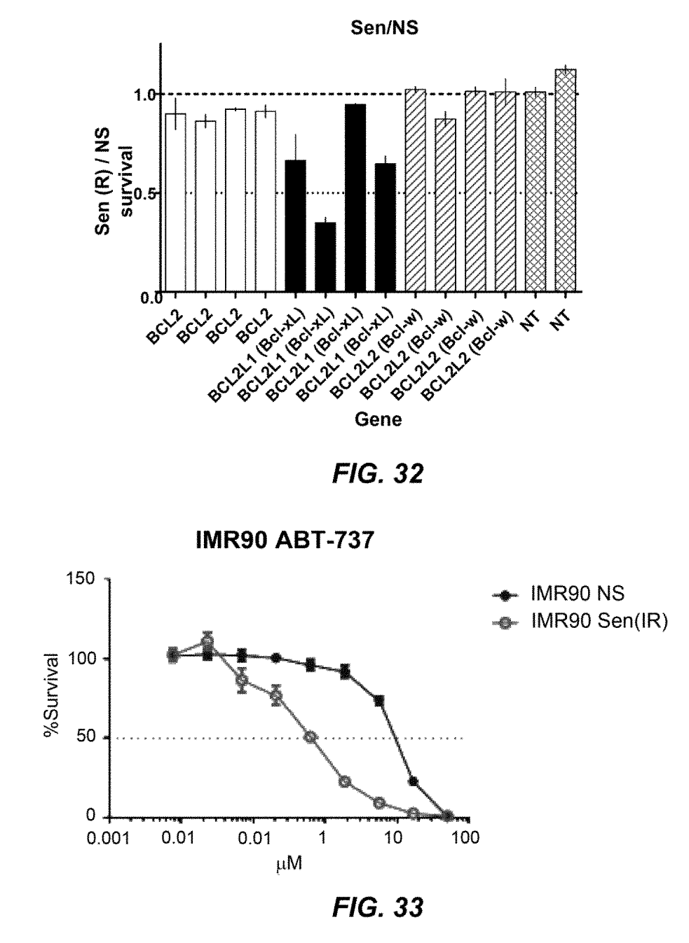

Survival of senescent cells and non-senescent cells was then determined in triplicate for each shRNA tested. The shRNAs as listed in order in the table are represented in the figure from left to right. The second and third shRNA sequences specific for BCL-2 are identical. The ratio of senescent cell survival to non-senescent cell survival is presented for each shRNA in FIG. 32. A ratio of 1.0 indicates no difference in the proportion of survival of senescent cells compared with non-senescent cells. Introduction of three of the four BCL-XL specific shRNA molecules into senescent cells resulted in significant senescent cell death compared with senescent cells into which Bcl-w or BCL-2 specific shRNAs were introduced. The data illustrate that BCL-XL expression is important to survival of senescent cells.

### Example 19 Effective Killing of Senescent Cells by Inhibiting Bcl-2 Anti-Apoptotic Protein Family Members

To determine whether other Bcl-2/Bcl-xL/Bcl-w inhibitors are selectively toxic to senescent cells compared to non-senescent cells, a cell viability assay was used to assess cell survival following treatment with ABT-737. The general timelines and procedures for the cell counting assay are shown in FIG. 18 and described in Example 7. IMR90 cells (human primary lung fibroblasts) were seeded in six well plates, and cells were induced to senescence with 10 Gy of ionizing radiation (IR) (Day 0). The media was refreshed every 3 days. The senescent phenotype is allowed to develop for 7 days at which point a cell count was made to determine the baseline number of cells followed by seeding into 96-well plates. On day 8, the senescent cells (irradiated) and the non-senescent cells (the non-radiated cells), were exposed to serial dilutions of ABT-737 for a period of 3 days. ABT-737 concentrations were serially diluted starting at 50 μM. Each condition was seeded in triplicate.

After three days of treatment (Day 11), cells were assayed for cell survival using CellTiter-Glo® (CTG) Luminescent Cell Viability Assay. The assay determines the number of viable cells in culture based on the quantitation of ATP present, which is an indicator of metabolically active cells.

FIG. 33 shows IC50 curves of ABT-737 in senescent cells and in non-senescent cells. The IC50 curve is a plot of the percentage of cell survival following treatment of ABT-737 as determined by the cell viability assay. The plot shows the effect of the various concentration levels of ABT-737 on cell survival.

### Example 20 BCL-2/BCL-xL/BCL-w Inhibitor Kills Senescent Cells Via Apoptosis

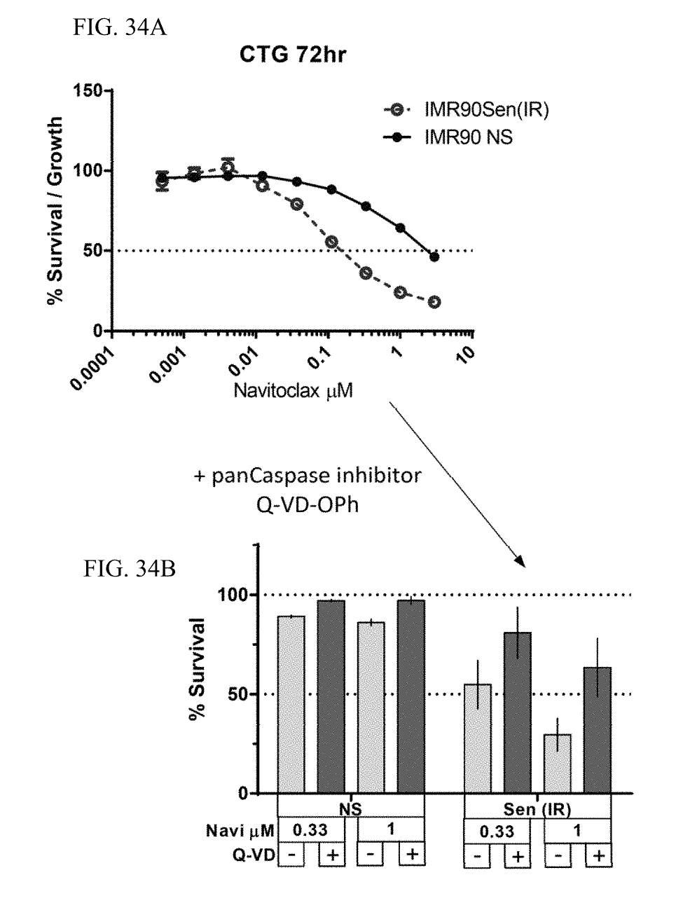

An experiment as described in Example 17 was performed to determine whether other inhibitors of one or more BCL-2 anti-apoptotic family members kill senescent cells by apoptosis. Lung fibroblast cell line IMR90 (human primary lung fibroblasts, ATCC® CCL-186™, Manassas, Va.) were seeded in six-well plates and induced to senesce with 10 Gy of ionizing radiation (IR) as described in Example 12. After senescence was established, cells were re-seeded into 96 well plates. The pan-caspase inhibitor Q-VD-OPh (20 μM) was added to wells of senescent cells (irradiated) (IMR90 Sen(IR)) and to wells containing non-senescent cells (the non-radiated cells) (IMR90 NS). Four hours later, the senescent and non-senescent cells were each exposed for a period of 3 days to 0.33 or 1 μM ABT-263 (Navitoclax). At the end of the assay time period, cells were counted. Each condition was seeded in three plate wells and counted independently. Initial cell count served as a control to determine the induction of senescence, as compared to the last day count without ABT-263 treatment. Initial non-senescent cell count serves as a proxy to determine ABT-263 toxicity. Cell survival was determined using CellTiter-Glo® Luminescent Cell Viability Assay (Promega Corporation, Madison, Wis.). The assay determines the number of viable cells in culture based on the quantitation of ATP present which is an indicator of metabolically active cells. FIG. 34 (top graphic) is an illustration that ABT-263 selectively kills senescent cells and illustrates the ABT-263 concentrations used in this experiment. In the presence of the pan-caspase inhibitor, the percent of surviving senescent cells increased (FIG. 34, lower graphic).

### Example 21 Effect of Removal of Senescent Cells in Animal Model of Osteoarthritis

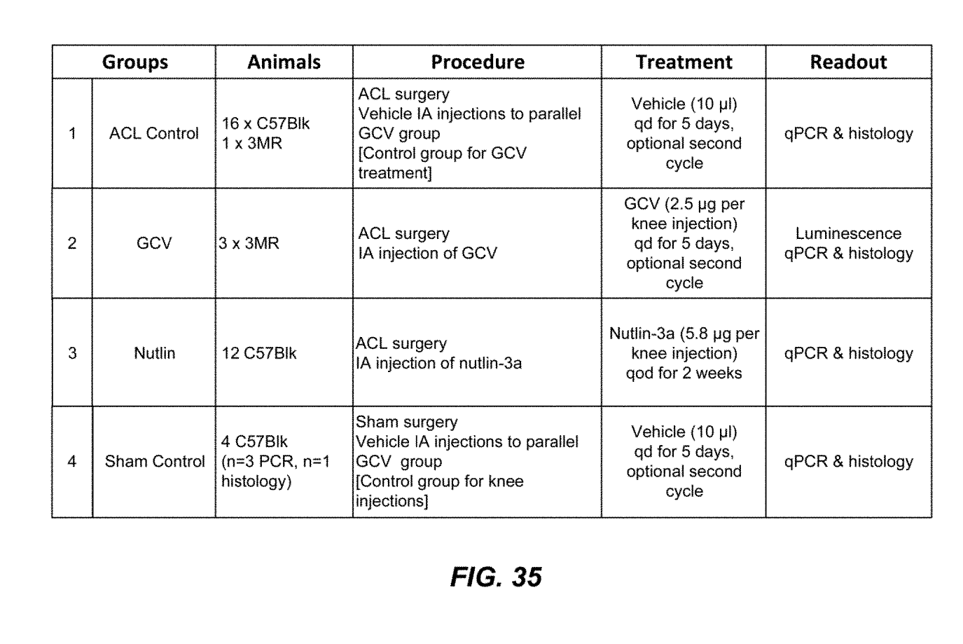

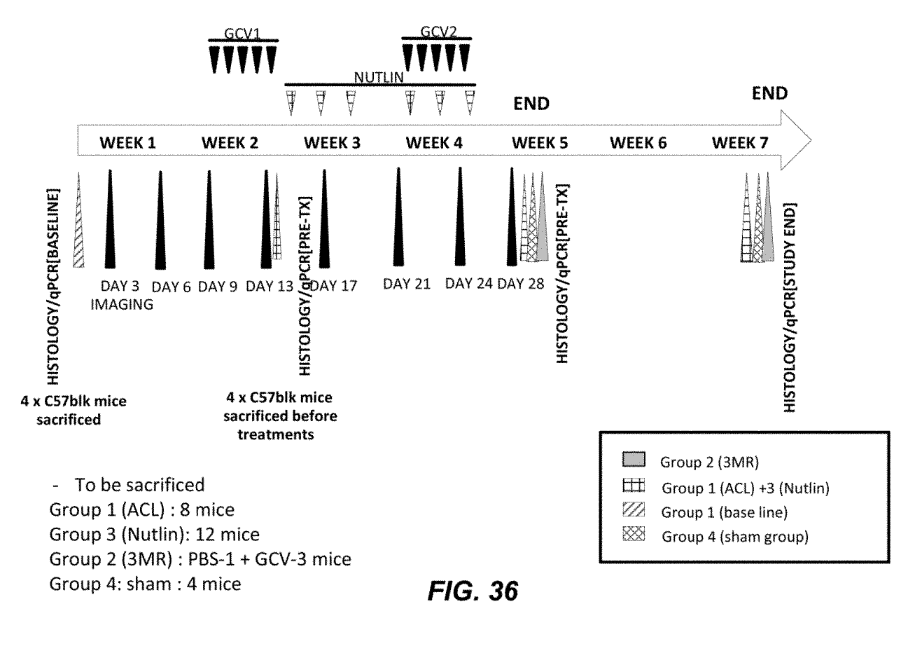

A table and schematic of two osteoarthritis mouse model study designs are presented in FIGS. 35 and 36, respectively. The two treatment studies were designed to determine the effect of removing senescent cells in an animal model of osteoarthritis.

Parallel studies were performed. One study investigated the effect of eliminating senescent cells with ganciclovir (GCV) in 3MR mice. Mice underwent surgery to cut the anterior cruciate ligament of one rear limb to induce osteoarthritis in the joint of that limb. During week 2 post-surgery, 3MR mice received 2.5 μg GCV to the operated knee by intra-articular injection, qd for 5 days, with a 2^{nd} treatment (2.5 μg GCV qd for 5 days) during week 4 post-surgery. At the end of 4 weeks post-surgery, operated joints of the mice were monitored for presence of senescent cells, assessed for function, monitored for markers of inflammation, and underwent histological assessment.

In a parallel study, C57BL/6J mice underwent surgery to cut the anterior cruciate ligament of one rear limb to induce osteoarthritis in the joint of that limb. During week 3 and week 4 post-surgery, the mice were treated with 5.8 μg of Nutlin-3A (n=7) per operated knee by intra-articular injection, qod for 2 weeks. At the end of 4 weeks post-surgery, joints of the mice were monitored for presence of senescent cells, assessed for function, monitored for markers of inflammation, and underwent histological assessment.

Two control groups of mice were included in the studies performed: one group comprising C57BL/6J or 3MR mice that had undergone a sham surgery (n=3) (i.e., surgical procedures followed except for cutting the ACL) and intra-articular injections of vehicle parallel to the GCV-treated group; and one group comprising C57BL/6J or 3MR mice that had undergone an ACL surgery and received intra-articular injections of vehicle (n=5) parallel to the GCV-treated group.

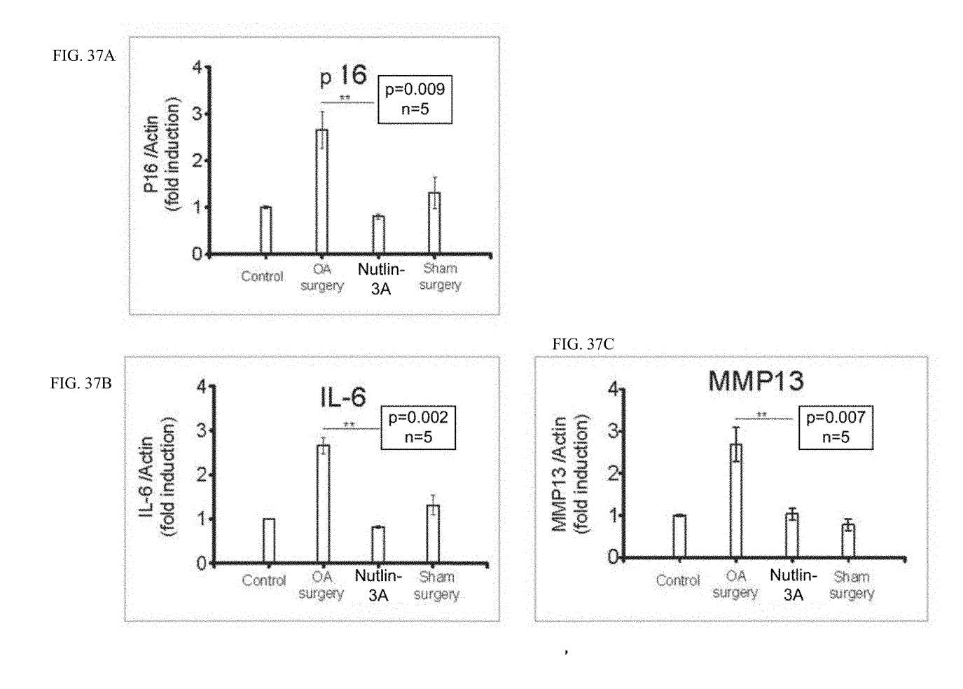

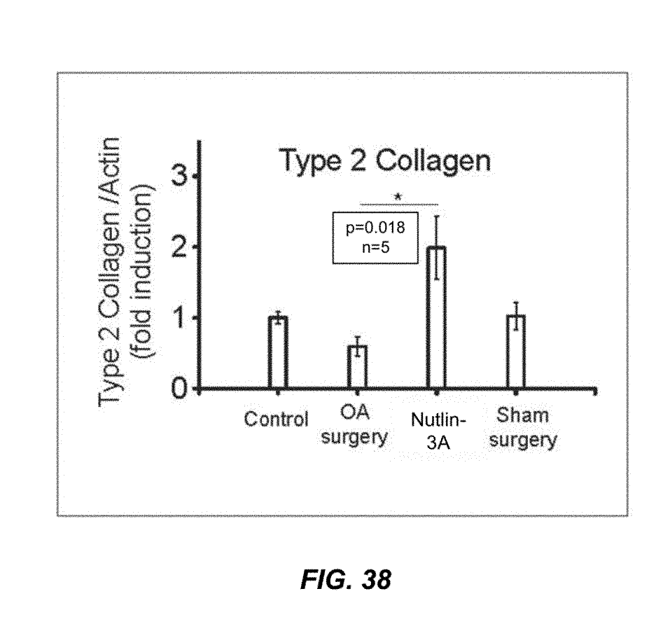

RNA from the operated joints of mice from the Nutlin-3A treated mice was analyzed for expression of SASP factors (mmp3, IL-6) and senescence markers (p16). qRT-PCR was performed to detect mRNA levels. As shown in FIGS. 37A-C, treatment with Nutlin-3A clears senescent cells from the joint. RNA from the operated joints of mice was also analyzed for expression of type 2 collagen and compared with expression of actin as a control. As shown in FIG. 38, treatment with Nutlin-3A in mice that have undergone osteoarthritis surgery drives collagen production as compared to untreated mice.

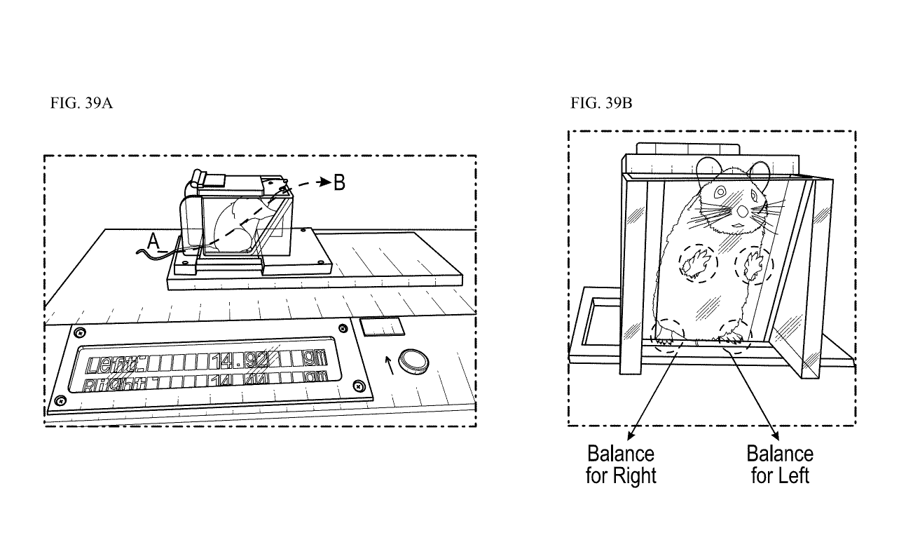

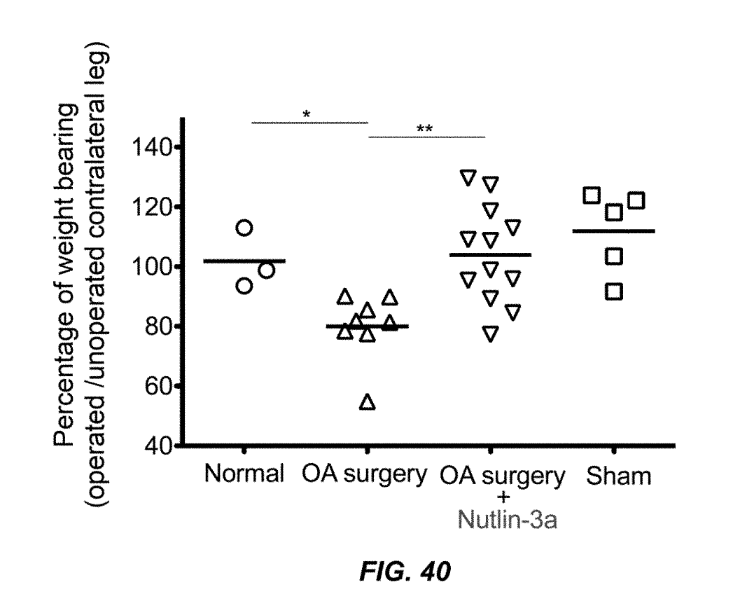

Function of the limbs was assessed 4 weeks post-surgery by a weight bearing test to determine which leg the mice favored (FIG. 39). The mice were allowed to acclimate to the chamber on at least 3 occasions prior to taking measurements. Mice were maneuvered inside the chamber to stand with 1 hind paw on each scale. The weight that was placed on each hind limb was measured over a 3-second period. At least 3 separate measurements were made for each animal at each time point. The results were expressed as the percentage of the weight placed on the operated limb versus the contralateral unoperated limb. As shown in FIG. 40, untreated mice that have undergone osteoarthritis surgery favor the unoperated hind limb over the operated hind limb (▴). However, clearing senescent cells with Nutlin-3A abrogates this effect in mice that have undergone surgery (∇).

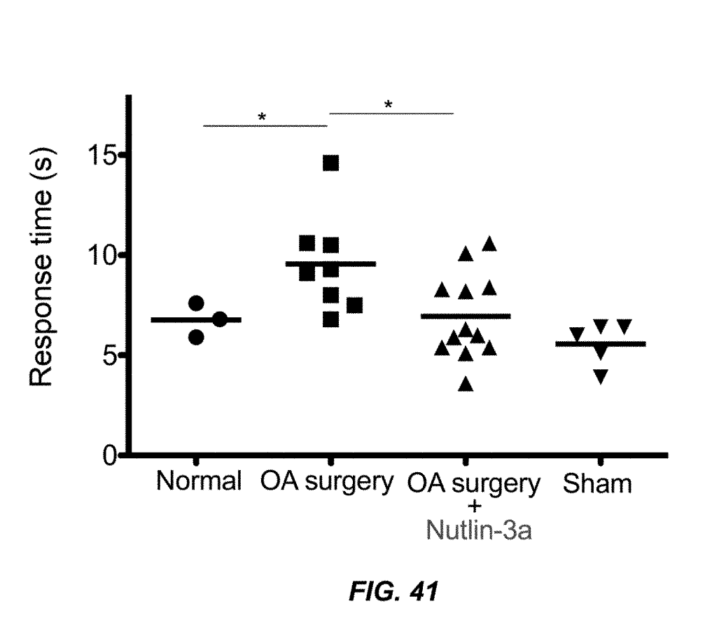

The function of the limbs was also assessed at 4 weeks post-surgery by hotplate analysis to show sensitivity and reaction to pain stimulus. In brief, a mouse was placed on a hotplate at 55° C. When placed on the hot surface of the plate, mice will lift their paws and lick them (paw-lick response) due to attainment of pain threshold. The latency period for the hind limb response (paw-lick response) is recorded as response time. As shown in FIG. 41, untreated mice that have undergone osteoarthritis surgery have an increased response time as compared to normal mice that have not been surgically altered (−). However, treatment of mice that have undergone osteoarthritis surgery with Nutlin-3A decreases the response time in a significant manner (▴).

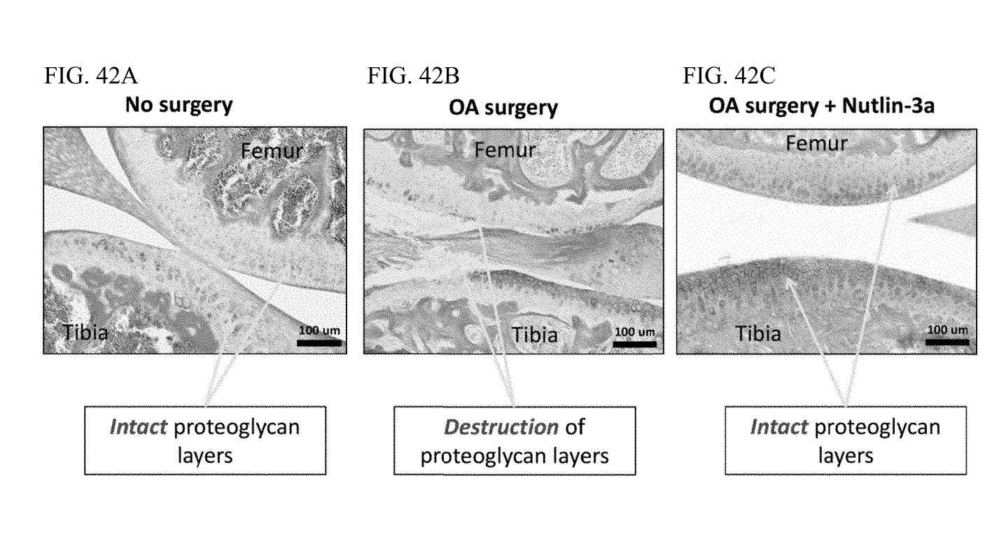

Histopathology of osteoarthritis induced by ACL surgery illustrated that the proteoglycan layer was destroyed. Clearing of senescent cells with Nutlin-3A completely abrogated this effect. Clearing of senescent cells from the 3MR mice treated with GCV, which kills senescent cells, had the same impact on pathophysiology of osteoarthritis as Nutlin-3A. See FIG. 42.

### Example 22 Effect of Removal of Senescent Cells in Animal Models of Atherosclerosis

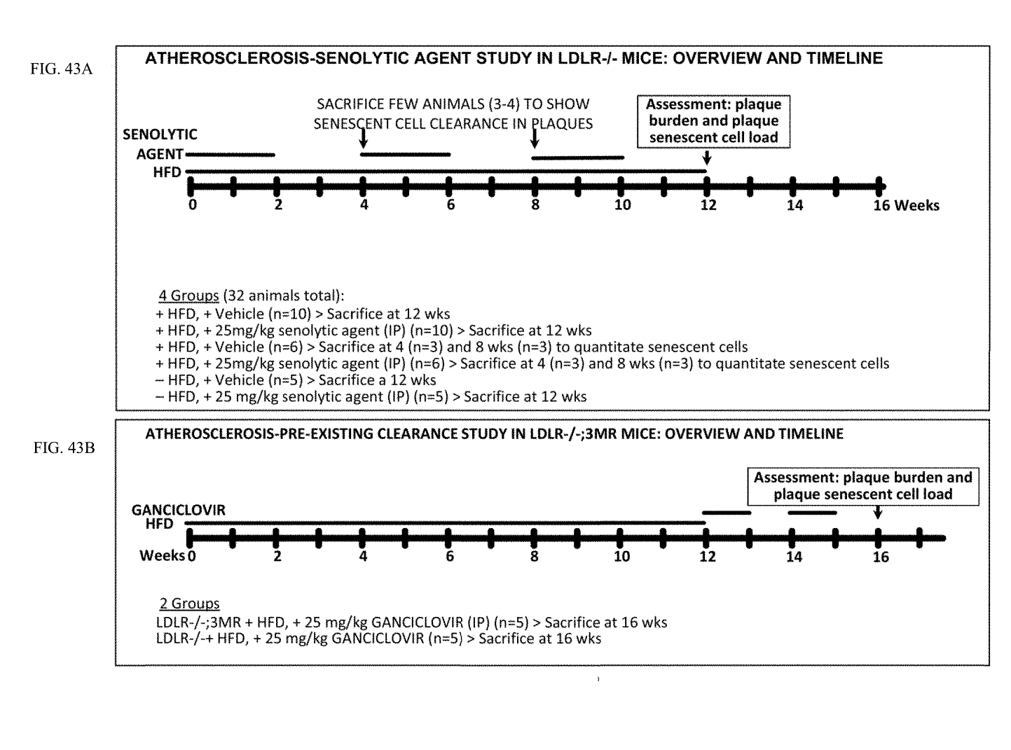

Schematics of two atherosclerosis mouse models are presented in FIGS. 43A-B. The study illustrated in FIG. 43A assessed the extent to which clearance of senescent cells from plaques in LDLR^{-/-} mice with Nutlin-3A reduces plaque load. Two groups of LDLR^{-/-} mice (10 weeks) are fed a high fat diet (HFD) (Harlan Teklad TD.88137) having 42% calories from fat, beginning at Week 0 and throughout the study. Two groups of LDLR^{-/-} mice (10 weeks) are fed normal chow (−HFD). From weeks 0-2, one group of HFD mice and −HFD mice are treated with Nutlin-3A (25 mg/kg, intraperitoneally). One treatment cycle is 14 days treatment, 14 days off. Vehicle is administered to one group of HFD mice and one group of −HFD mice. At week 4 (timepoint 1), one group of mice are sacrificed and to assess presence of senescent cells in the plaques. For the some of the remaining mice, Nutlin-3A and vehicle administration is repeated from weeks 4-6. At week 8 (timepoint 2), the mice are sacrificed and to assess presence of senescent cells in the plaques. The remaining mice are treated with Nutlin-3A or vehicle from weeks 8-10. At week 12 (timepoint 3), the mice are sacrificed and to assess the level of plaque and the number of senescent cells in the plaques.

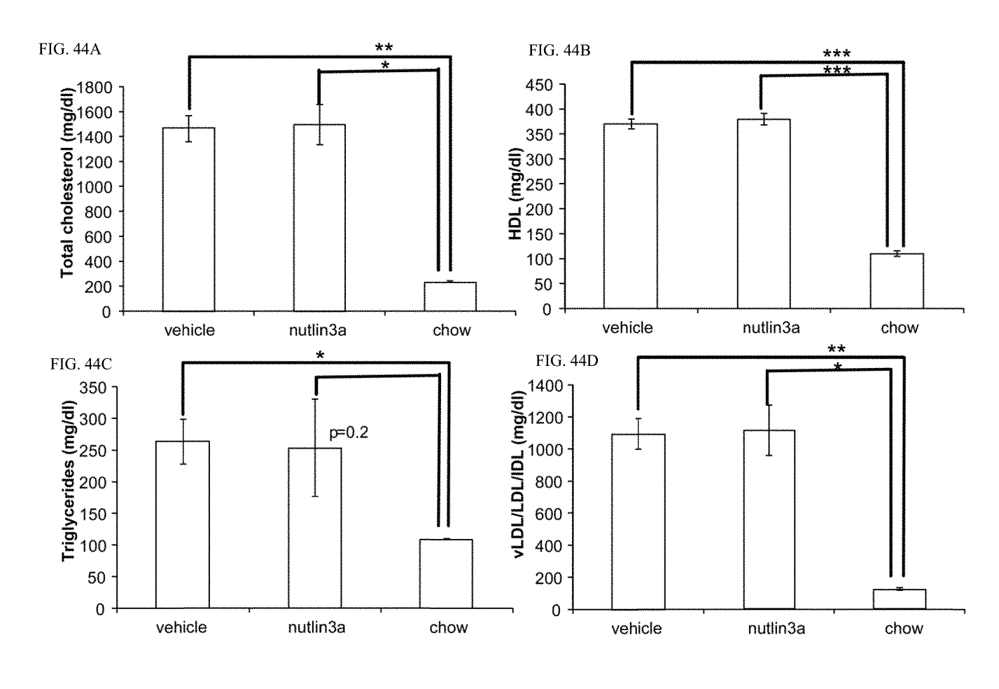

Plasma lipid levels were measured in LDLR^{-/-} mice fed a HFD and treated with Nutlin-3A or vehicle at timepoint 1 as compared with mice fed a −HFD (n=3 per group). Plasma was collected mid-afternoon and analyzed for circulating lipids and lipoproteins. The data are shown in FIG. 44A-D.

At the end of timepoint 1, LDLR^{-/-} mice fed a HFD and treated with Nutlin-3A or vehicle were sacrificed (n=3, all groups), and the aortic arches were dissected for RT-PCR analysis of SASP factors and senescent cell markers. Values were normalized to GAPDH and expressed as fold-change versus age-matched, vehicle-treated LDLR^{-/-} mice on a normal diet. The data show that clearance of senescent cells with Nutlin-3A in LDLR^{-/-} mice fed a HFD reduced expression of several SASP factors and senescent cell markers, MMP3, MMP13, PAIL p21, IGFBP2, IL-1A, and IL-1B after 1 treatment cycle (see FIGS. 45A-D).

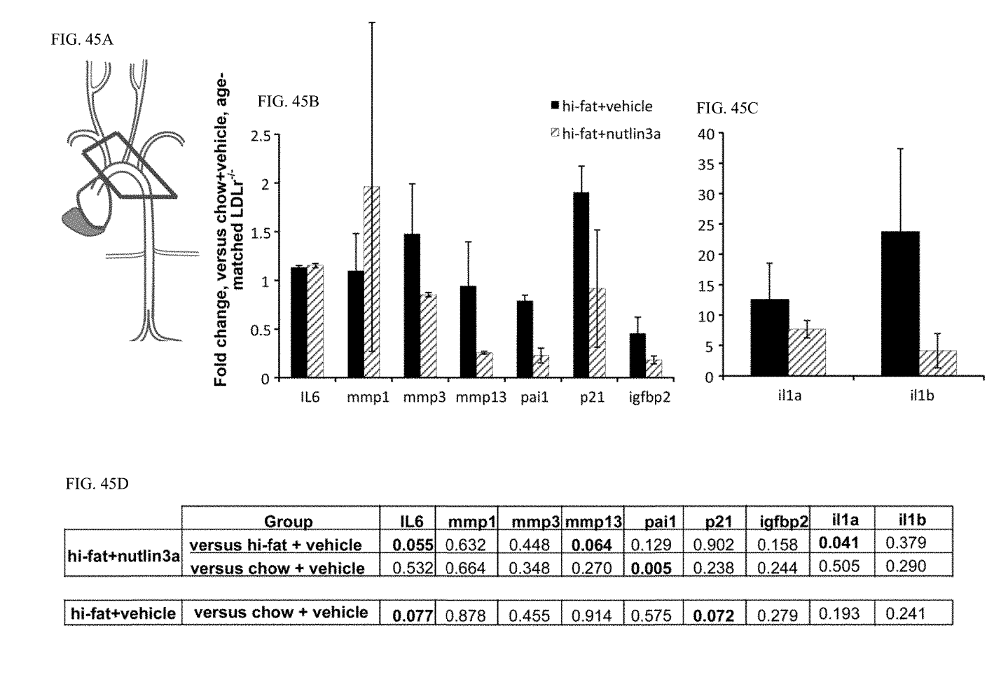

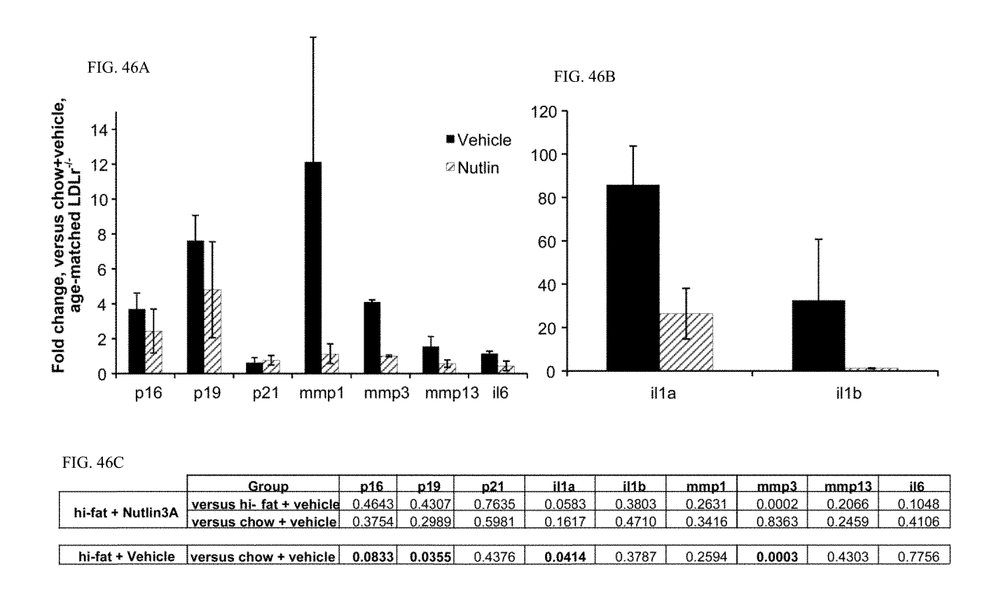

At the end of timepoint 2, LDLR^{-/-} mice fed a HFD and treated with Nutlin-3A or vehicle (n=3 for all groups) were sacrificed, and aortic arches were dissected for RT-PCR analysis of SASP factors and senescent cell markers. Values were normalized to GAPDH and expressed as fold-change versus age-matched, vehicle-treated LDLR^{-/-} mice on a normal diet. The data show expression of some SASP factors and senescent cell markers in the aortic arch within HFD mice (FIGS. 46A-C). Clearance of senescent cells with multiple treatment cycles of Nutlin-3A in LDLR^{-/-} mice fed a HFD reduced expression of most markers (FIGS. 46A-B).

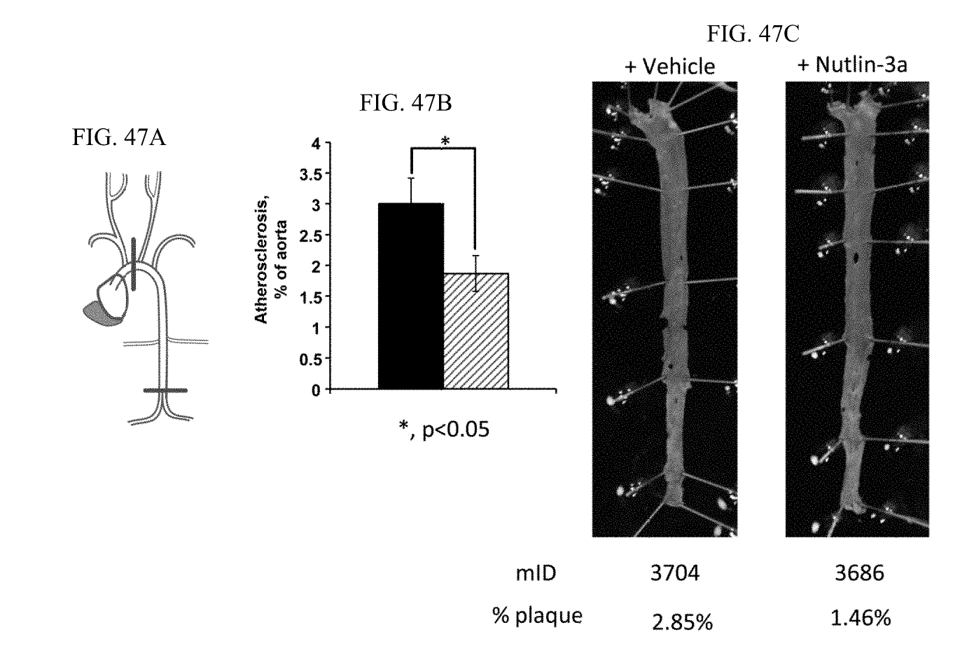

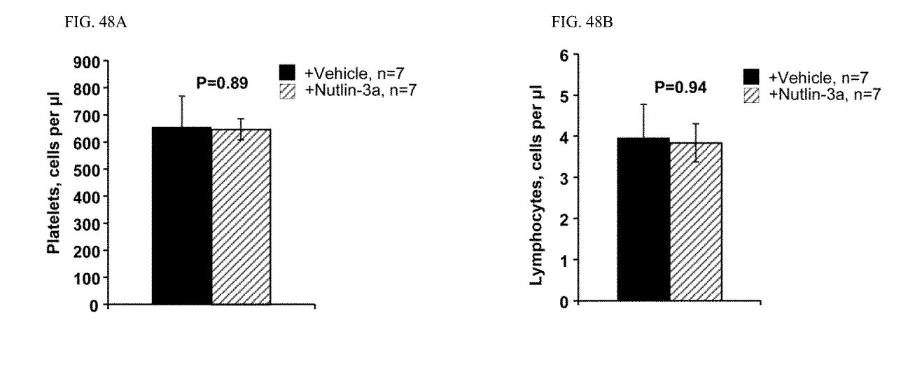

At the end of timepoint 3, LDLR^{-/-} mice fed a HFD and treated with Nutlin-3A or vehicle (n=3 for all groups) were sacrificed, and aortas were dissected and stained with Sudan IV to detect the presence of lipid. Body composition of the mice was analyzed by MRI, and circulating blood cells were counted by Hemavet. The data show that treatment with Nutlin-3A reduces plaques in the descending aorta by ˜45% (FIGS. 47A-C). As shown in FIGS. 48A-B, the platelet and lymphocyte counts were equivalent between the Nutlin-3A and vehicle treated mice. As shown in FIGS. 49A-B, treatment with Nutlin-3A also decreased mass and body fat composition in mice fed a HFD.

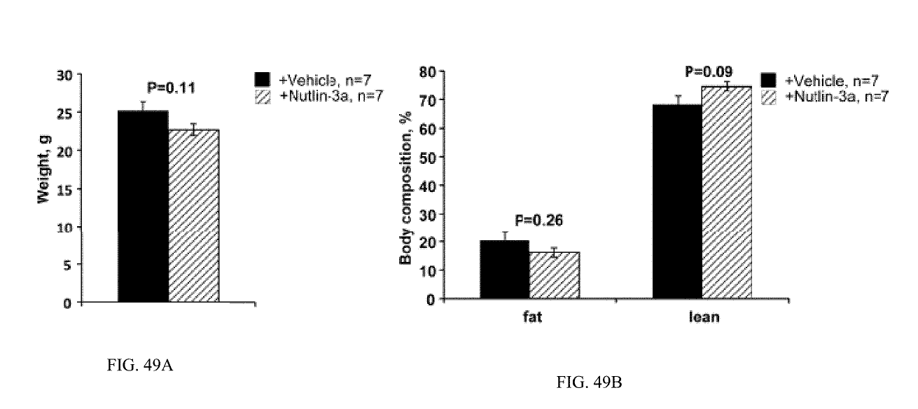

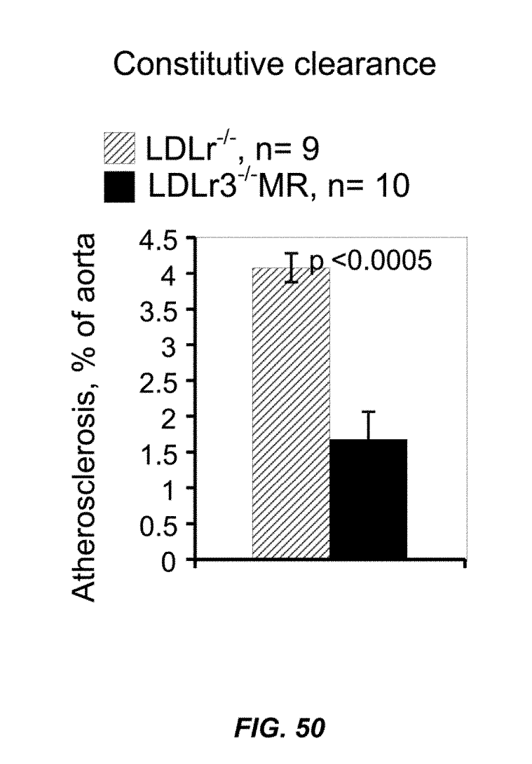

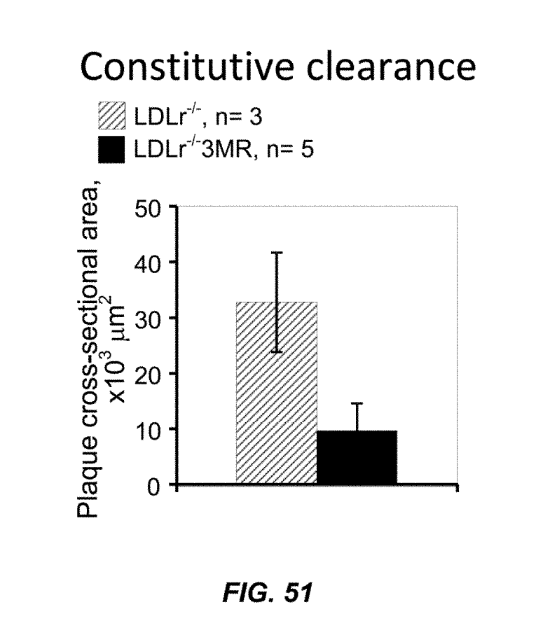

The study illustrated in FIG. 43B assessed the extent to which acyclovir based clearance of senescent cells from LDLR^{-/-} /3MR double transgenic mice improves pre-existing atherogenic disease. LDLR^{-/-} MR double transgenic mice (10 weeks) and LDLR^{-/-} single transgenic mice (10 weeks) are fed a high fat diet beginning at Week 0 until Week 12. Gancyclovir is administered to both groups of mice (25 mg/kg intraperitoneally) from weeks 12-13 and weeks 14-15. At week 16, the level of plaque and the number of senescent cells in the plaques are determined. As shown in FIG. 50, clearance of senescent cells with GCV in LDLR^{-/-} MR double transgenic mice fed a HFD (n=10) reduces the % of the aorta covered with plaque as compared to LDLR^{-/-} mice/HFD controls (n=9). As shown in FIG. 51, clearance of senescent cells with GCV also reduced the plaque cross-sectional area in in LDLR^{-/-} MR double transgenic mice fed a HFD (n=3) as compared to LDLR^{-/-} mice/HFD controls (n=5).

### Example 23 Senescent Cell Clearance Sustains Cardiac Stress Resistance with Aging

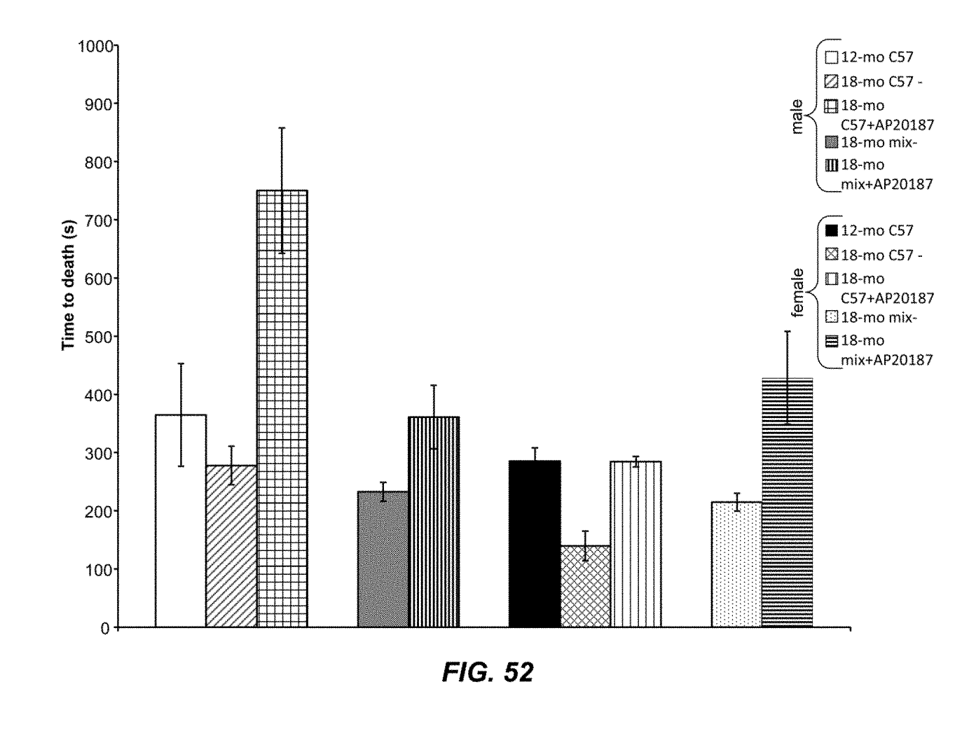

To study the impact of senescent cell clearance on health and lifespan, cohorts of INK-ATTAC transgenic mice on FVB×129Sv/E×C57BL/6 mixed or C57BL/6 pure genetic backgrounds were established. At 12 months age, one half of each cohort was injected three times/week with AP20187 to induce apoptosis of p16-positive senescent cells (0.2 mg/kg and 2 mg/kg AP20187 for the mixed and the pure C57BL/6 cohorts, respectively), while the other half of each cohort received vehicle. At 18 months, subsets of male and female mice from each cohort were subjected to a cardiac stress test, in which mice were injected with a lethal dose of isoproterenol (680 mg/kg) and the time to cardiac arrest was recorded. While 18-month-old untreated (vehicle) mice consistently showed a marked acceleration of cardiac arrest compared to 12-month-old control mice, AP20187-treated mice sustained youthful cardio-protection against isoproterenol, regardless of gender and genetic background (see FIG. 52).

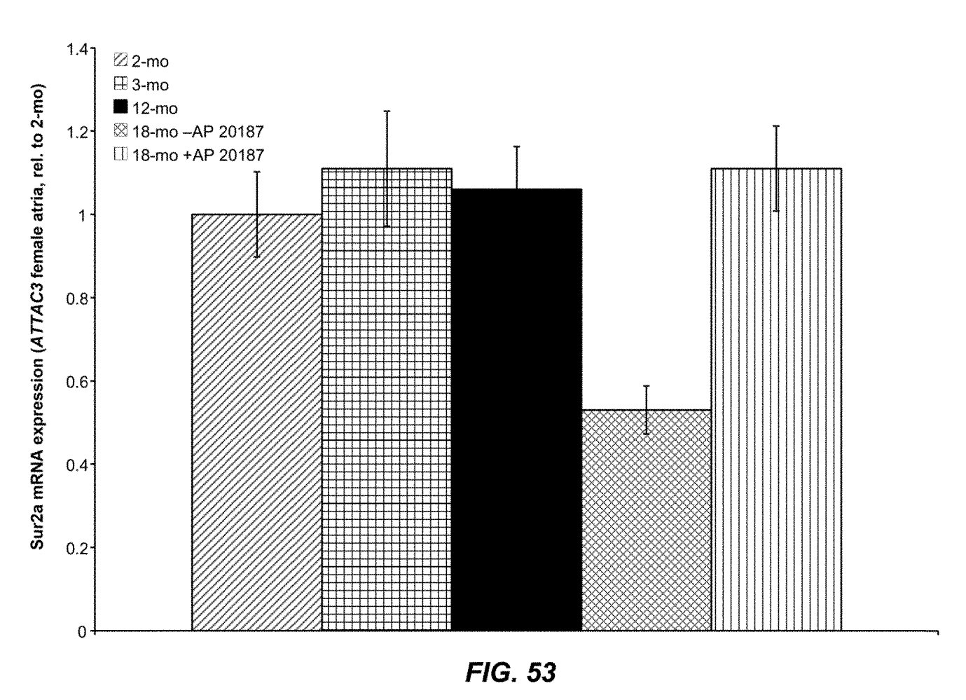

Cardio-protective signaling pathways are known to provide tolerance to metabolic stresses such as ischemia and hypoxia decline (Granfeldt et al., 2009, Cardiovasc. Res. 83:234-246). However, cardio-protective signaling deteriorates with aging, thus decreasing the functional and adaptive reserve capacity of the heart (Ogawa et al., 1992, Circulation 86:494-503; Wiebe et al., 1998, Clin. J. Sport Med. 8:272-279).). ATP-dependent K channels (KATP) play a central role in cardio-protective signaling (Gross and Auchampach, 1992, Cardiovasc. Res. 26:1011-1016).). These KATP channels are composed of the pore-forming subunit Kir6.2/Kir6.1, the regulatory subunit Sur2a, and additional accessory proteins. KATP channels are thought to decline with aging due to decreased expression of Sur2a (Du et al., 2006, FASEB J. 20:1131-1141; Jovanovic and Jovanovic, 2009, Curr. Opin. Pharmacol. 9:189-193; Ranki et al., 2002, Mech. Ageing Dev. 123:695-705). Elevated expression of Sur2a, either through diet alteration (Sukhodub et al., 2011, J. Cell. Mol. Med. 15:1703-1712) or transgenic approaches (Sudhir et al., 2011, Biogerentology 12:147-155), has been shown to sustain cardiac stress resistance in aged mice. Thus, the contribution of senescent cells to the age-related decline in Sur2a expression was examined in 18 month old AP20187-treated and vehicle treated mice from subjected to the cardiac stress test previously described. Indeed, youthful performance in the isoproterenol stress test of 18-month-old female AP20187-treated animals consistently correlated with sustained Sur2a expression (see FIG. 53). Taken together, these experiments indicate that the presence of senescent cells with aging negatively impacts KATP channel function, and senescent cell clearance is an effective therapy to counteract this deterioration. Sustained cardiac performance could contribute to the median lifespan extension observed in AP20187-treated INK-ATTAC mice.

### Example 24 Clearance of Senescent Cells Ameliorates Atherosclerosis in LDLR^{-/-}3/MR Mice

The impact of clearance of senescent cells on the stability and size of mature atherosclerotic plaques was studied in LDLR^{-/-} MR double transgenic mice. From 10 weeks of age, LDLR^{-/-} MR double transgenic mice (10 weeks) and LDLR^{-/-} single transgenic mice (control) were fed a high fat diet (Harlan Teklad TD.88137) having 42% calories from fat beginning at Week 0 until Week 12.5, when the mice were switched to normal chow diet. Both groups of mice were treated with ganciclovir from week 12.5 over the next 100 days, with each treatment cycle comprising 5 days of ganciclovir (25 mg/kg intraperitoneally daily) and 14 days off. At the end of the 100 day treatment period, the mice were sacrificed, plasma and tissues were collected, and atherosclerosis was quantitated.

Descending aortas were dissected and stained with Sudan IV to visualize the plaque lipids. As shown in FIGS. 54A-B, ganciclovir-treated LDLR^{-/-} MR double transgenic mice had fewer atherosclerotic plaques with less intense staining than the LDLR^{-/-} control mice fed a HFD. The % of the aorta covered in plaques as measured by area of Sudan IV staining was also significantly lower in the ganciclovir-treated LDLR^{-/-} MR mice as compared to the LDLR^{-/-} control mice (see FIG. 54C).

Plaques from ganciclovir-treated LDLR^{-/-} control and LDLR^{-/-} MR mice (see dashed circled plaques in FIGS. 55A-B, respectively) were harvested and cut into cross-sections and stained with to characterize the general architecture of the atherosclerotic plaques. “#” indicates fat located on the outside of the aorta (see FIG. 55A). The plaques marked with an “*” and “*” in FIGS. 55A and B, respectively, are shown as stained cross-sections in FIGS. 55B and D, respectively. As illustrated in FIGS. 55B and D, clearance of senescent cells in ganciclovir-treated LDLR^{-/-} MR mice has an effect on plaque morphology as compared to LDLR^{-/-} control mice. The plaque from the control mice has identifiable “lipid pockets” accumulating within. The plaque from the ganciclovir treated LDLR^{-/-} MR mice shows the presence of a thick fibrin cap and the absence of lipid pockets. Disruption or tear in the cap of a lipid-rich plaque is a trigger for coronary events through exposure of plaque thrombogenic components to platelets and clotting components of the blood. Plaques that grow more rapidly as a result of rapid lipid deposition and have thin fibrin caps are prone to rupture. Slowly growing plaques with mature fibrin caps tend to stabilize and are not prone to rupture. Taken together, these experiments indicate that removal of senescent cells may affect atherosclerotic plaque architecture and have a stabilizing effect.

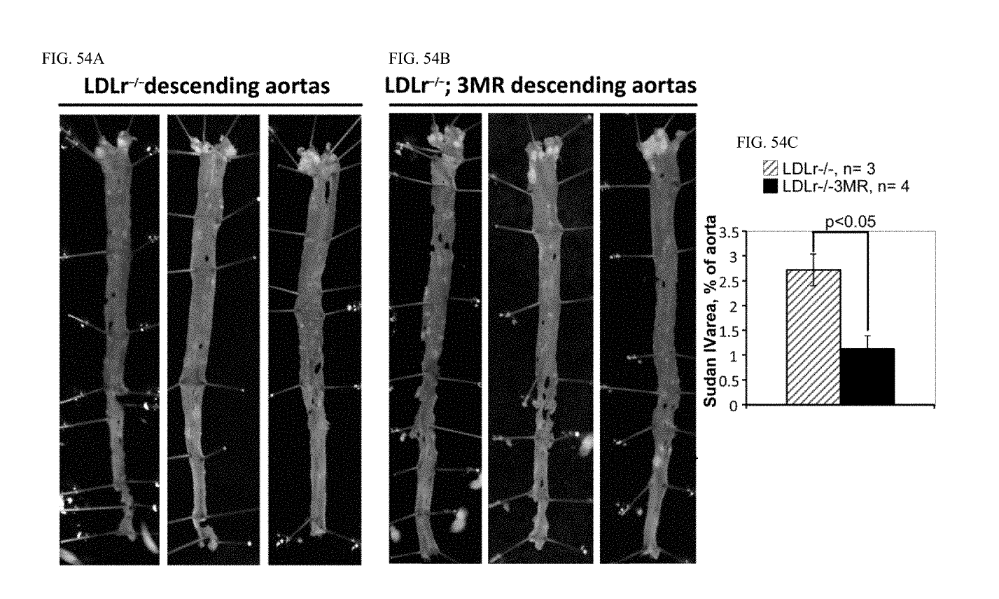

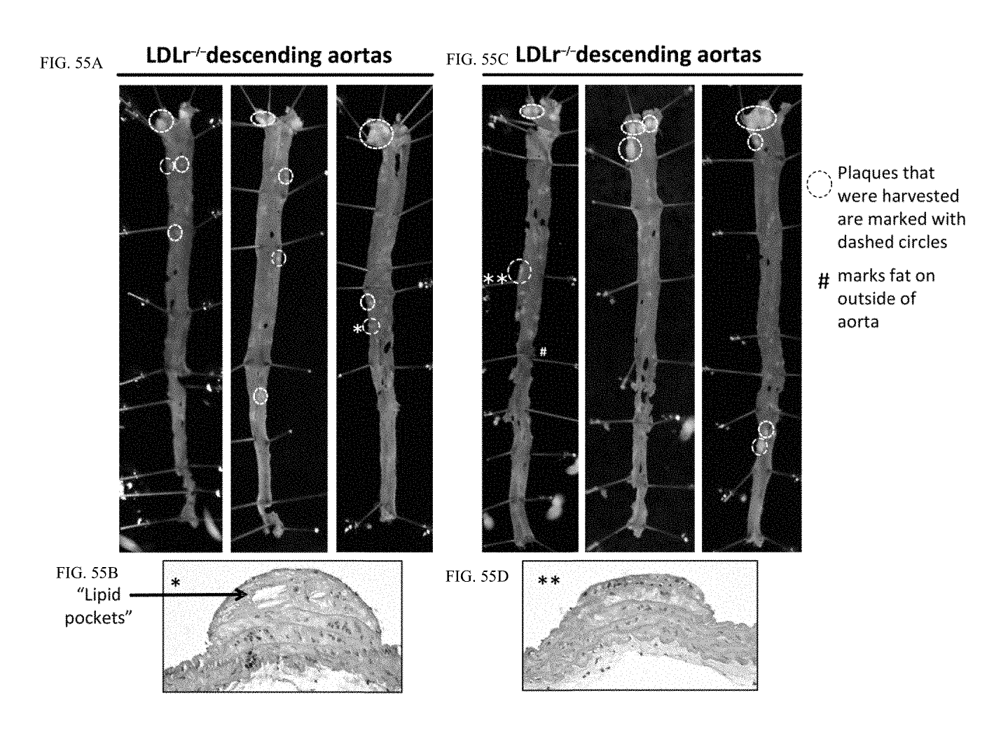

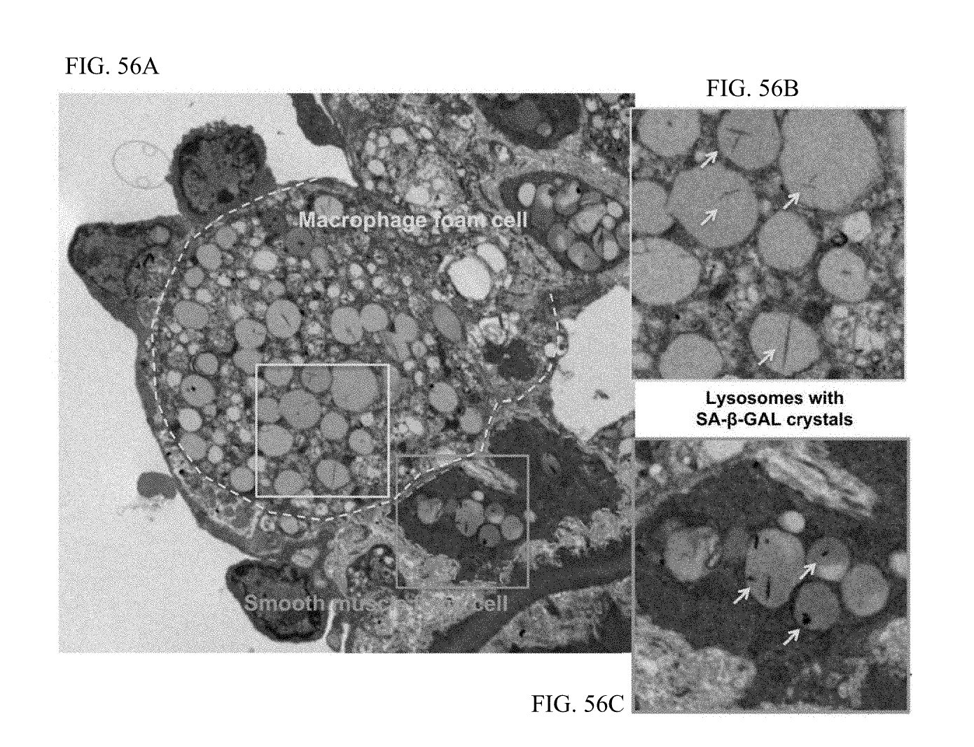

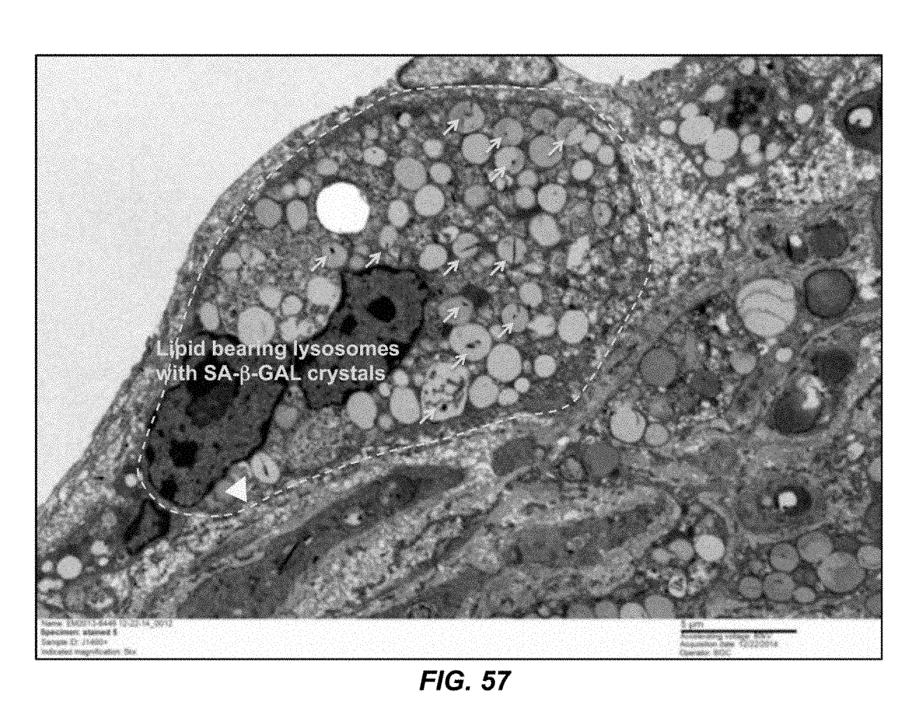

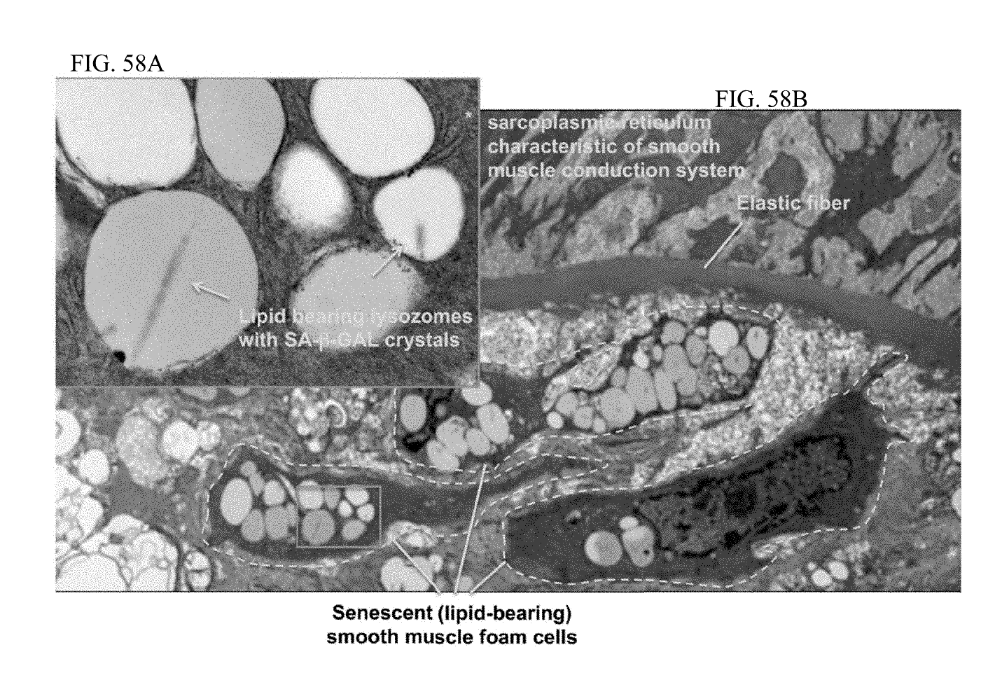

Tissue sections of atherosclerotic aortas were prepared and stained to detect SA-β-GAL. X-GAL crystals were located in the lysosomes of lipid-bearing macrophage foam cells and smooth muscle foam cells (see FIGS. 56-58).

### Example 25 Effect of Clearance of Senescent Cells in Pulmonary Disease Models

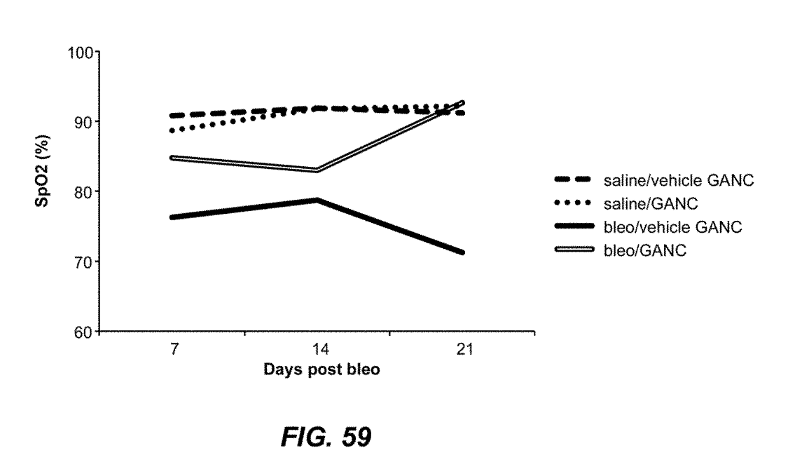

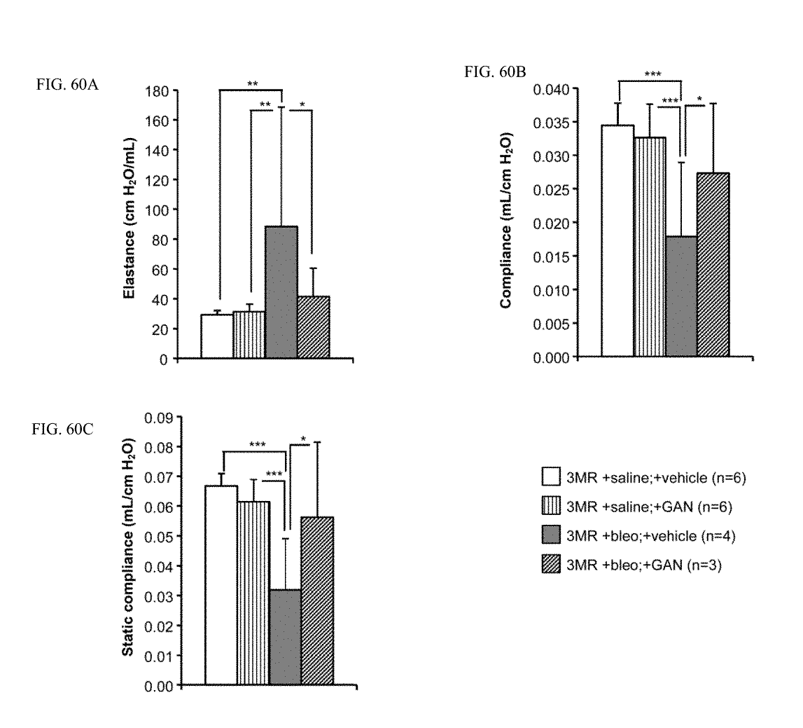

One animal model study assessed the effect of clearance of senescence cells in the transgenic mouse strain 3MR that has bleomycin induced lung injury. In the bleomycin injury model for idiopathic pulmonary fibrosis, mice develop lung fibrosis within 7-14 days after bleomycin treatment (see, e.g., Limjunyawong et al., 2014, Physiological Reports 2:e00249; Daniels et al., 2004, J. Clin. Invest. 114:1308-1316). Bleomycin was administered to anesthetized 6-8 week old 3MR mice by intratracheal aspiration (2.5 U/kg of bleomycin in 50 μl PBS) using a microsprayer syringe (Penn-Century, Inc.) as described in Daniels et al. (2004, J. Clin. Invest. 114:1308-1316). Control mice were administered saline. The day following bleomycin treatment, ganciclovir (GCV) (25 mg/kg in PBS) was administered. 3MR mice were treated via intraperitoneal injection with ganciclovir for 5 consecutive days, followed by 5 days of rest, followed by a second treatment cycle of 5 consecutive days. Untreated mice received an equal volume of vehicle. At 7, 14, and 21 days post-bleomycin treatment, lung function was assessed by monitoring oxygen saturation using the MouseSTAT PhysioSuite pulse oximeter (Kent Scientific). Animals were anesthetized with isoflurane (1.5%) and a toe clip was applied. Mice were monitored for 30 seconds and the average peripheral capillary oxygen saturation (SpO₂) measurement over this duration was calculated. As shown in FIG. 59, bleomycin administration significantly reduced SpO₂ levels in vehicle treated mice, and removal of senescent cells resulted in higher SpO₂ levels, which approached normal levels at 21 days post bleomycin administration. At 21 days post-bleomycin treatment, airway hyper-reactivity (AHR) of mice was examined. AHR of mice was measured by methacholine challenge while other parameters of lung function (airway mechanics, lung volume and lung compliance) were determined using a SCIREQ flexiVent ventilator. While under ketamine/xylazine anesthesia and subjected to cannulation of the trachea via a tracheostomy (19Fr blunt Luer cannula), airway resistance (elastance) and compliance of mice were assessed at baseline and in response to increasing concentrations of methacholine (0 to 50 mg/ml in PBS) delivered via nebulization (AeroNeb) as described in Aravamudan et al. (Am. J. Physiol. Lung Cell. Mol. Physiol. (2012) 303:L669-L6810). Animals were maintained at 37° C., and while under muscle paralysis (pancuronium); airway function was measured by using the FlexiVent ventilator and lung mechanics system (SCIREQ, Montreal, Quebec, Canada), which was housed on Stabile 8. As shown in FIG. 60A, in vehicle treated mice, bleomycin administration increased lung elastance, whereas ganciclovir treatment reduced lung elastance. As shown in FIGS. 60B-C, bleomycin administration reduced static compliance and (dynamic) compliance in vehicle treated mice. Clearance of senescent cells with ganciclovir in bleomycin exposed mice improved compliance values significantly (FIGS. 60B-C). Although not statistically significant because the animal group size was too small, data suggested that clearance of senescent cells with a senolytic agent (Nutlin-3A) also reduced lung elastance and increased lung compliance in a bleomycin exposed mouse. Mice were euthanized by i.p injection of pentobarbital. Bronchoalveolar lavage (BAL) fluids and lungs is obtained and analyzed. Hydroxyproline content of lungs is measured as described in Christensen et al. (1999, Am. J. Pathol. 155:1773-1779), and quantitative histopathology is performed. RNA is extracted from lung tissue to measure senescence cell markers by qRT-PCR in treated and control mice.

The effect of clearance of senescence cells in the bleomycin induced lung injury model of IPF may also be studied in INK-ATTAC transgenic mice in the study design described above. INK-ATTAC (p16^{Ink4a} apoptosis through targeted activation of caspase) transgenic mice have an FK506-binding protein (FKBP)-caspase 8 (Casp8) fusion polypeptide under the control of the p16^{Ink4a} promoter (see, e.g., Baker et al., Nature, supra; Intl Patent Application Publication No. WO/2012/177927). In the presence of AP20187, a synthetic drug that induces dimerization of a membrane bound myristoylated FKBP-Casp8 fusion protein, senescent cells specifically expressing the FKBP-Casp8 fusion protein via the p16^{Ink4a} promoter undergo programmed cell death (apoptosis) (see, e.g., Baker, Nature, supra, FIG. 1 therein).

A second study also assesses the effect of clearance of senescence cells using a senolytic agent in C57BL6/J mice that have bleomycin induced lung injury. Bleomycin is administered to 6 week old C57BL6/J mice as described above. A senolytic agent is administered during the first and third week post-bleomycin treatment. Control mice are treated with vehicle. At 21 days post-bleomycin treatment, clearance of senescent cells and lung function/histopathology is assessed.

In a second animal model for pulmonary diseases (e.g., COPD), mice were exposed to cigarette smoke. The effect of a senolytic agent on the mice exposed to smoke is assessed by senescent cell clearance, lung function, and histopathology.

Six week-old 3MR (n=35) or INK-ATTAC (n=35) mice were chronically exposed to cigarette smoke generated from a Teague TE-10 system, an automatically-controlled cigarette smoking machine that produces a combination of side-stream and mainstream cigarette smoke in a chamber, which is transported to a collecting and mixing chamber where varying amounts of air is mixed with the smoke mixture. The COPD protocol was adapted from the COPD core facility at Johns Hopkins University (at Internet site web.jhu.edu/Biswal/exposure_core/copd.html#Cigarette Smoke) (Rangasamy et al., 2004, 1 Clin. Invest. 114:1248-1259; Yao et al., 2012, J. Clin. Invest. 122:2032-2045). Mice received a total of 6 hours of cigarette smoke exposure per day, 5 days a week for 6 months. Each lighted cigarette (3R4F research cigarettes containing 10.9 mg of total particulate matter (TPM), 9.4 mg of tar, and 0.726 mg of nicotine, and 11.9 mg carbon monoxide per cigarette [University of Kentucky, Lexington, Ky.]) was puffed for 2 seconds and once every minute for a total of 8 puffs, with the flow rate of 1.05 L/min, to provide a standard puff of 35 cm³. The smoke machine was adjusted to produce a mixture of side stream smoke (89%) and mainstream smoke (11%) by smoldering 2 cigarettes at one time. The smoke chamber atmosphere was monitored for total suspended particulates (80-120 mg/m³) and carbon monoxide (350 ppm). Beginning at day 7, (10) INK-ATTAC and (10) 3MR mice were treated with AP20187 (3×per week) or gancyclovir (5 consecutive days of treatment followed by 16 days off drug, repeated until the end of the experiment), respectively. An equal number of mice received the corresponding vehicle. The remaining 30 mice (15 INK-ATTAC and 15 3MR) were evenly split with 5 of each genetically modified strain placed into three different treatment groups. One group (n=10) received Nutlin-3A (25 mg/kg dissolved in 10% DMSO/3% Tween-20 in PBS, treated 14 days consecutively followed by 14 days off drug, repeated until the end of the experiment). One group (n=10) received ABT-263 (Navitoclax) (100 mg/kg dissolved in 15% DMSO/5% Tween-20, treated 7 days consecutively followed by 14 days off drug, repeated until the end of the experiment), and the last group (n=10) received only the vehicle used for ABT-263 (15% DMSO/5% Tween-20), following the same treatment regimen as ABT-263. An additional 70 animals that did not receive exposure to cigarette smoke were used as controls for the experiment.

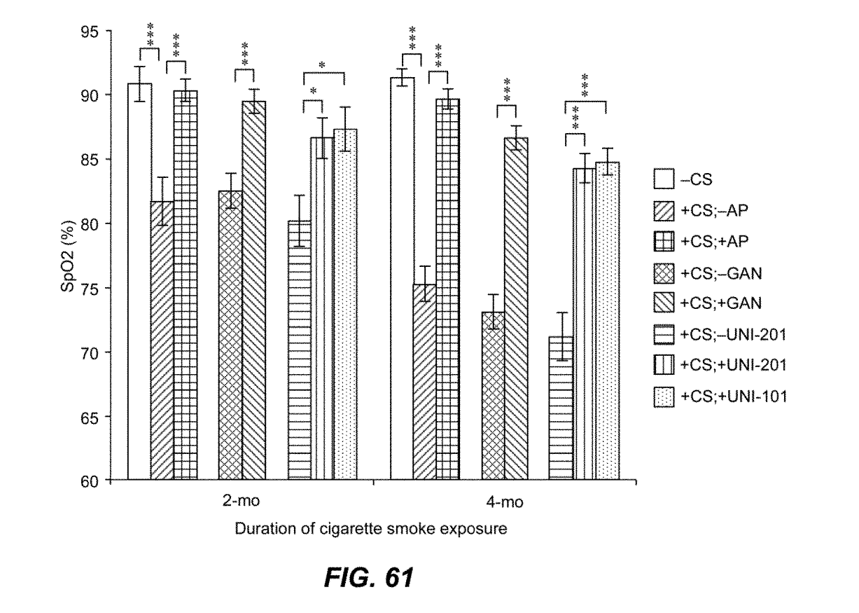

After two months of cigarette smoke exposure, lung function was assessed by monitoring oxygen saturation using the MouseSTAT PhysioSuite pulse oximeter (Kent Scientific). Animals were anesthetized with isoflurane (1.5%) and the toe clip was applied. Mice were monitored for 30 seconds and the average peripheral capillary oxygen saturation (SpO₂) measurement over this duration was calculated. As shown in FIG. 61, clearance of senescent cells via AP20187, ganciclovir, ABT-263 (Navi), or Nutlin-3A resulted in statistically significant increases in SpO₂ levels in mice after 2 months of cigarette smoke exposure compared to untreated controls.

At the end of the experimental period, airway hyper-reactivity (AHR) of mice to methacholine challenge using a SCIREQ flexiVent ventilator and lung mechanics system is examined as described above. After AHR measurement, mice are killed by i.p. injection of pentobarbital for in-depth analysis of lung histopathology as previously described (Rangasamy et al., 2004, J Clin. Invest. 114:1248-1259). Briefly, lungs are inflated with 0.5% low-melting agarose at a constant pressure of 25 cm. Part of the lung tissue is collected for RNA extraction and qRT-PCR analysis of senescent markers. Other parts of lungs are fixed in 10% buffered formalin and embedded in paraffin. Sections (5 μm) are stained with hematoxylin and eosin. Mean alveolar diameter, alveolar length, and mean linear intercepts are determined by computer-assisted morphometry with Image Pro Plus software (Media Cybernetics).

The potential therapeutic effect of clearance of senescent cells after COPD is fully developed may be assessed in 3MR or INK-ATTAC mice. Six week-old 3MR or INK-ATTAC mice are chronically exposed to cigarette smoke for 6 months as described above. At 6 months following the start of smoke exposure, 3MR or INK-ATTAC mice are treated with ganciclovir (5 consecutive days of treatment followed by 16 days off drug) or AP20187 (3×/week), respectively, until 9 months following the start of smoke exposure, when assessment of senescent cell clearance, lung function, and histopathology is performed.

### Example 26 In Vitro Cell Assays for Determining Senolytic Activity of MDM2 Inhibitor RG-7112

Lung fibroblast cell line IMR90 (human primary lung fibroblasts, ATCC® CCL-186™, Manassas, Va.) was seeded in six-well plates and induced to senesce with 10 Gy of ionizing radiation (IR). Senescent phenotype was allowed to develop for at least 7 days.

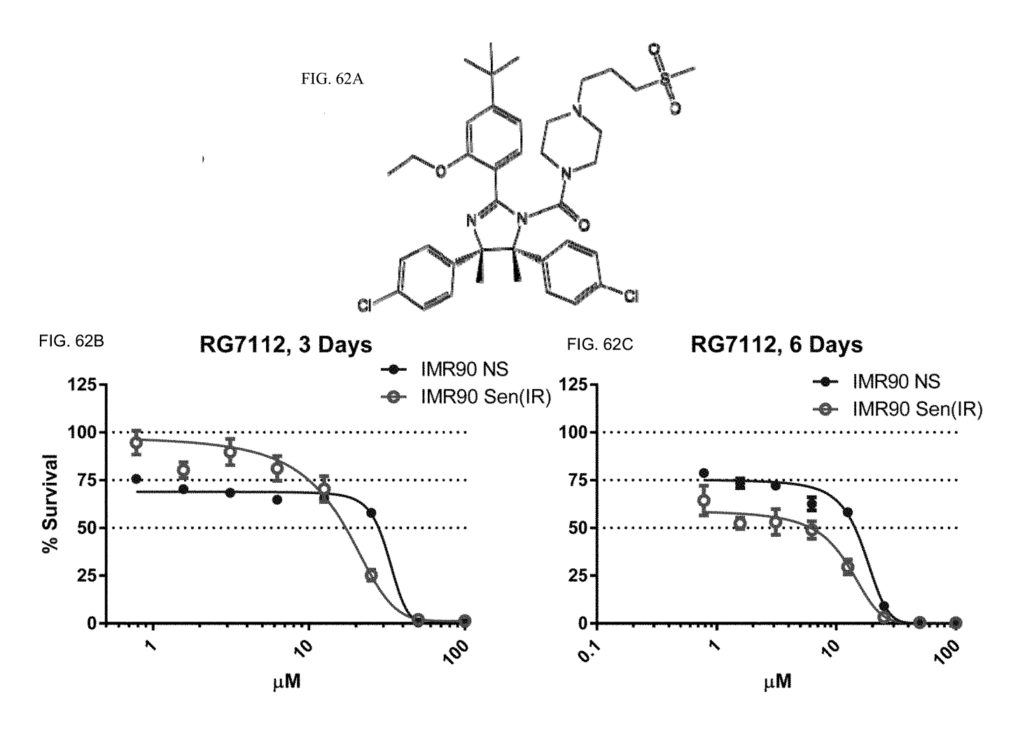

After senescence phenotype had developed, cells were re-seeded into 96 well plates, and senescent cells (irradiated) and non-senescent cells (the non-radiated cells), were exposed to eight two-fold serial dilutions starting at 100 μM of the MDM2 inhibitor RG-7112 (see structure in FIG. 62A) for a period of 3 or 6 days. After the three days, cell survival was determined using the commercially available CellTiter-Glo® Luminescent Cell Viability Assay (Promega Corporation, Madison, Wis.). The assay determines the number of viable cells in culture based on the quantitation of ATP present which is an indicator of metabolically active cells. FIG. 62 presents the IMR90 cell survival after 3 days exposure to RG-7112 (see FIG. 62B) and after six days (see FIG. 62C).

### Example 27 Effect of Clearance of Senescent Cells by ABT-263 to Reduce Chemotherapy Related Side Effects

The capability of a senolytic agent, such as ABT-263, to reduce chemotherapy related side effects, such as fatigue, was examined in p16-3MR transgenic mice. In addition to doxorubicin, paclitaxel also induces cellular senescence when administered to animals. See Example 2 for a description of the p16-3MR transgenic mouse model.

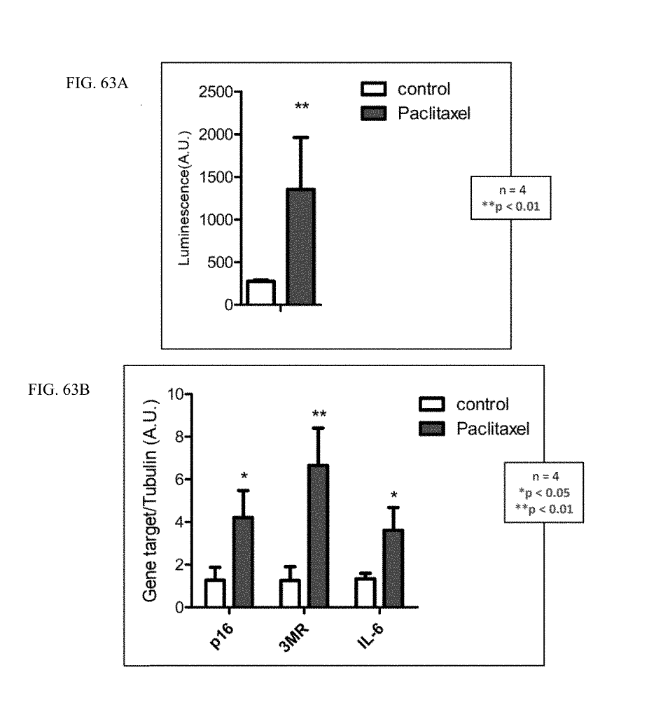

Paclitaxel induces senescence and SASP in p16-3MR transgenic mice. Groups of mice (n=4) were treated three times every two days with 20 mg/kg paclitaxel or vehicle. Senescence was observed as shown by luminescence in mice treated with paclitaxel (see FIG. 63A). The level of mRNA in skin was determined for each of the target genes: p16, 3MR transgene, and IL-6. As shown in FIG. 63B, the levels of mRNA for each of p16, 3MR, and IL-6 increased in paclitaxel treated animals compared with vehicle treated animals.

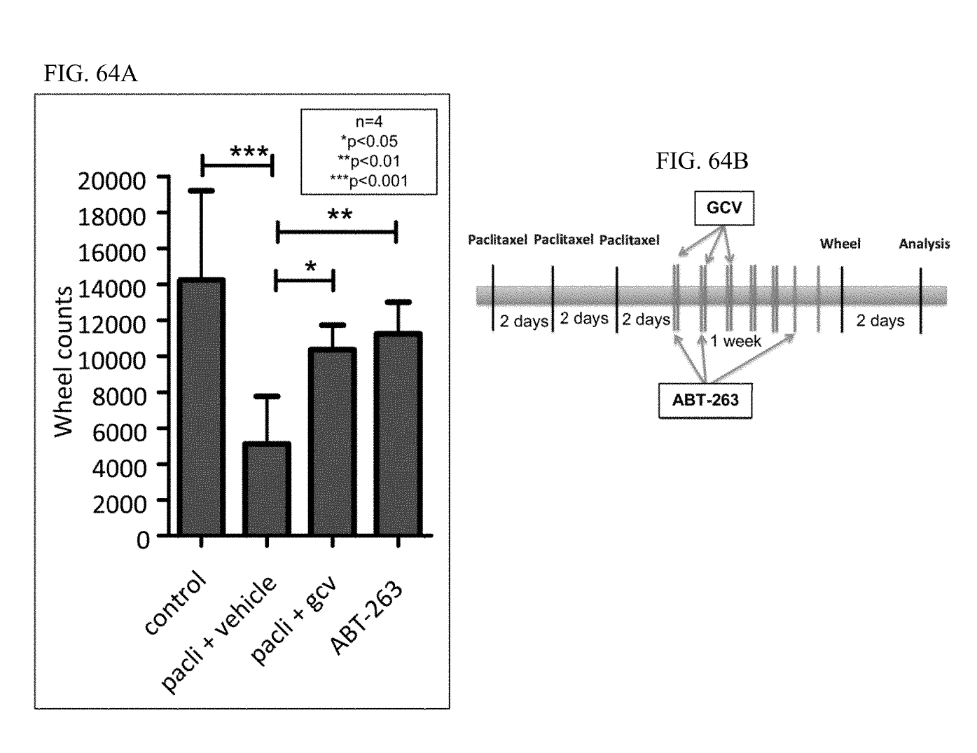

A schematic of the experiment is presented in FIG. 64. In this experiment, paclitaxel was administered to groups of p16-3mr mice (n=4) three times, every two days. Two days after the third dose of paclitaxel, ganciclovir was administered daily for three days (days 1, 2, and 3) intraperitoneally at 25 mg/kg. ABT-263 (100 mg/kg) was administered intraperitoneally daily for seven days after paclitaxel administration. Two days after the last dose of ABT-263, all groups of animals were housed in metabolic cages (promethion, sable systems international, Las Vegas, Nev.) to monitor voluntary exercise as determined by wheel counts. Data were collected and analyzed two days later. The data are shown in FIG. 64 (left hand side). Clearance of senescent cells by ABT-263 and ganciclovir restored approximately 70% of wheel count reduction caused by chemotherapy treatment.

### Example 28 Chemotherapy Drugs That Induce Senescence

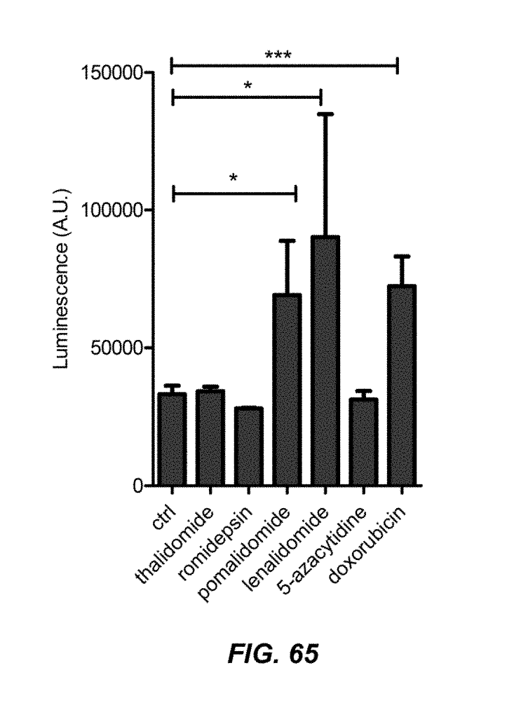

To examine senescence induced by different chemotherapeutic drugs, groups of p16-3MR animals (n=4) were treated with thalidomide (100 mg/kg; 7 daily injections); romidepsin (1 mg/kg; 3 injections); pomalidomide (5 mg/kg; 7 daily injections); lenalidomide (50 mg/kg; 7 daily injections); 5-azacytidine (5 mg/kg; 3 injections) and compared with doxorubicin (10 mg/kg, 2-4 injections over 7 days). The level of luminescence in animals treated with the drugs is shown in FIG. 65. Treatment of animals with omalidomide, lenalidomide, and doxorubicin resulted in significant levels of senescent cells (p<0.05).

### Example 29 Senescence Associated Pathways

Proteomic analyses by nano LC MS/MS were performed on lysates on human abdominal subcutaneous preadipocytes that were senescent or non-senescent. Preadipocytes, one of the most abundant cell types in humans susceptible to senescence, were extracted from fat tissues of nine different healthy kidney transplant donors by collagenase digestion. Prior consent from the donors was obtained. Senescence was induced by 10 Gy radiation or by serial subculturing. Bioinformatics methods were used to identify pathways that were susceptible to existing drugs and that could mediate cell death.

Senescence-associated β-galactosidase (SA-B gal) activity was used to assess the percentage of senescent cells present in the irradiated cell cultures. To be considered a senescent culture in this experiment, 75% or more of the cells needed to demonstrate SA-B gal activity. Both whole cell lysates and cellular supernatants were collected. Proteins were separated on 1D SDS-PAGE. Sections of the gels were destained, reduced, alkylated, and trypsin-digested. Extracted peptides were analyzed by nano-LC-MS/MS on a THERMO SCIENTIFIC™ Q Exactive mass spectrometer. LC Progenesis software (Nonlinear Dynamics, UK) was used to identify and quantify proteins. The data were then submitted to Ingenuity, Metacore, Cytoscape, and other software for pathway and protein network analysis. Among the pathways altered during senescence were those involved in cell survival signaling and inflammatory pathways. These pathways include at least PI3K/AKT, Src kinase signaling, insulin/IGF-1 signaling, p38/MAPK, NF-κB signaling, TGF signaling, and mTOR/protein translation.

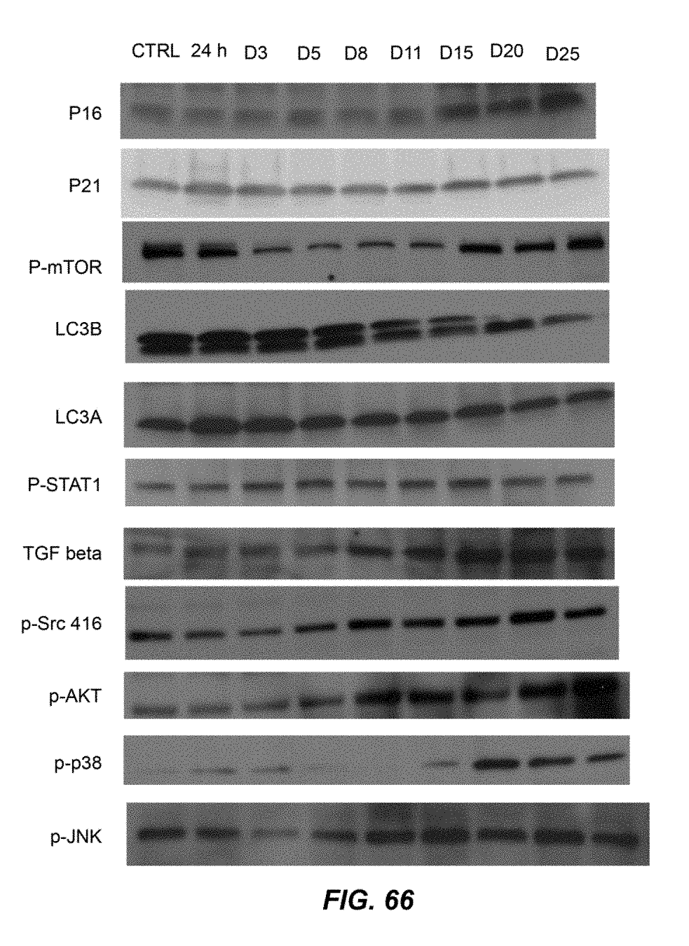

FIG. 66 shows a confirmatory Western immunoblot of proteins involved in these and related pathways at various times (24 hr; 3, 6, 8, 11, 15, 20, and 25 days) after radiation. Phosphorylated polypeptides in the senescent cell samples were detecting using horseradish peroxidase labeled antibodies (Cell Signaling Technology, Danvers, Mass.) specific for the polypeptides indicated in FIG. 66. Senescence is fully established between day 25 to day 30 in these cells.

### Example 30 High Fat Feeding-Induced Senescence Reduced by a Senolytic Agent in P16-3MR Mice

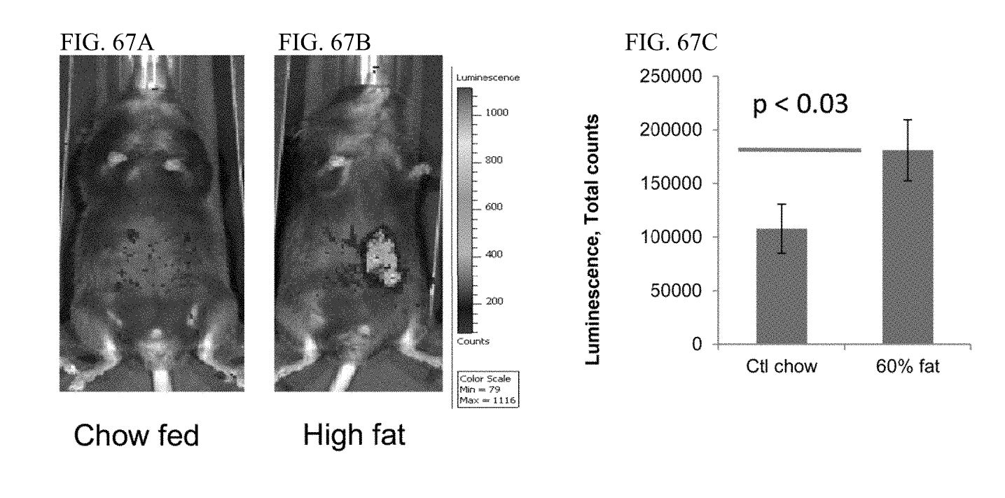

Groups of p16-3MR mice (n=6) were fed a high fat diet (60% fat) for four months mice or a regular chow diet. The presence of senescence cells was determined by measuring luminescence (i.e., p16 positive cells). As shown in FIG. 67, animals fed a high fat diet have increased numbers of senescence cells compared with the regular chow fed animals.

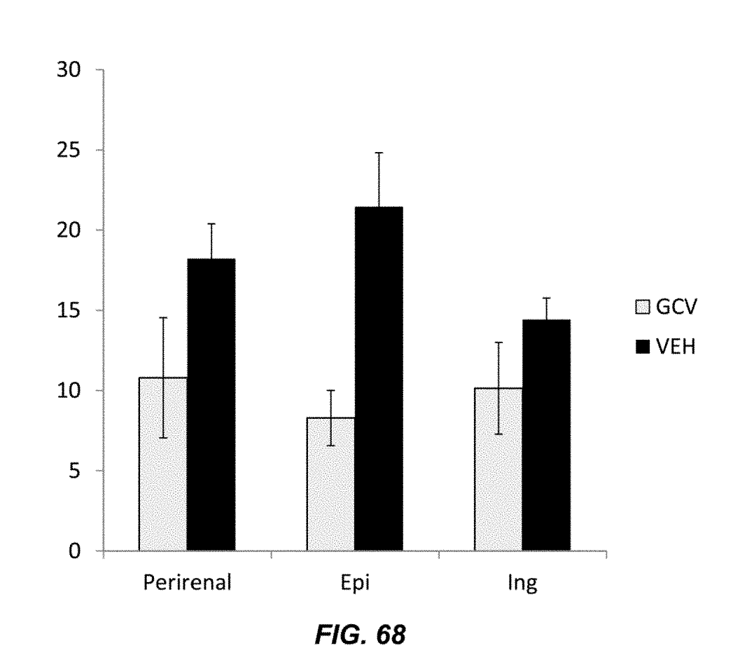

Animals were then treated with ganciclovir or vehicle to determine if removal of senescent cells reduced the presence of senescent cells in adipose tissue. Groups of animals were treated with ganciclovir or vehicle. Ganciclovir (25 mg/kg) was administered daily for five consecutive days. The presence of senescent cells in perirenal, epididymal, or subcutaneous inguinal adipose tissue was detected by SA-β-Gal staining. Data were analyzed by ANOVA. The results are presented in FIG. 68. A significant reduction in presence of senescent cells was observed in epididymal fat. p=<0.004.

### Example 31 Clearance of Senescent Cells Improves Glucose Tolerance and Insulin Sensitivity

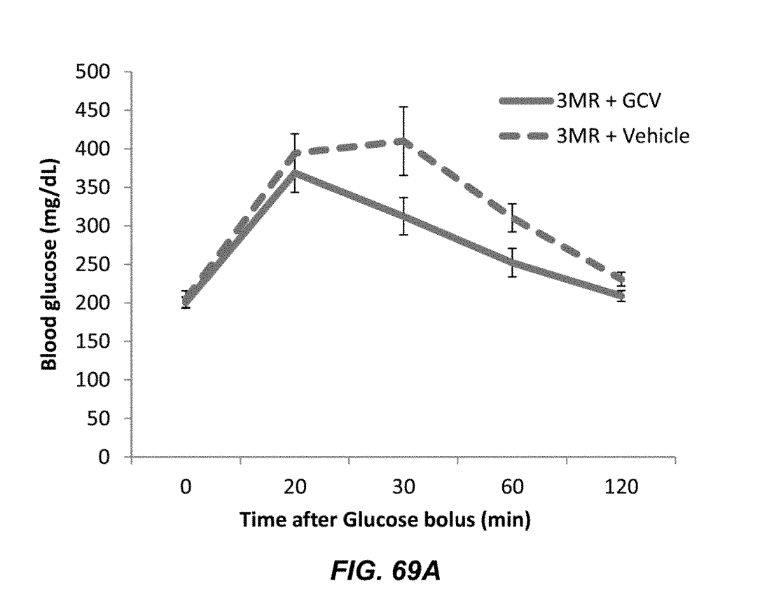

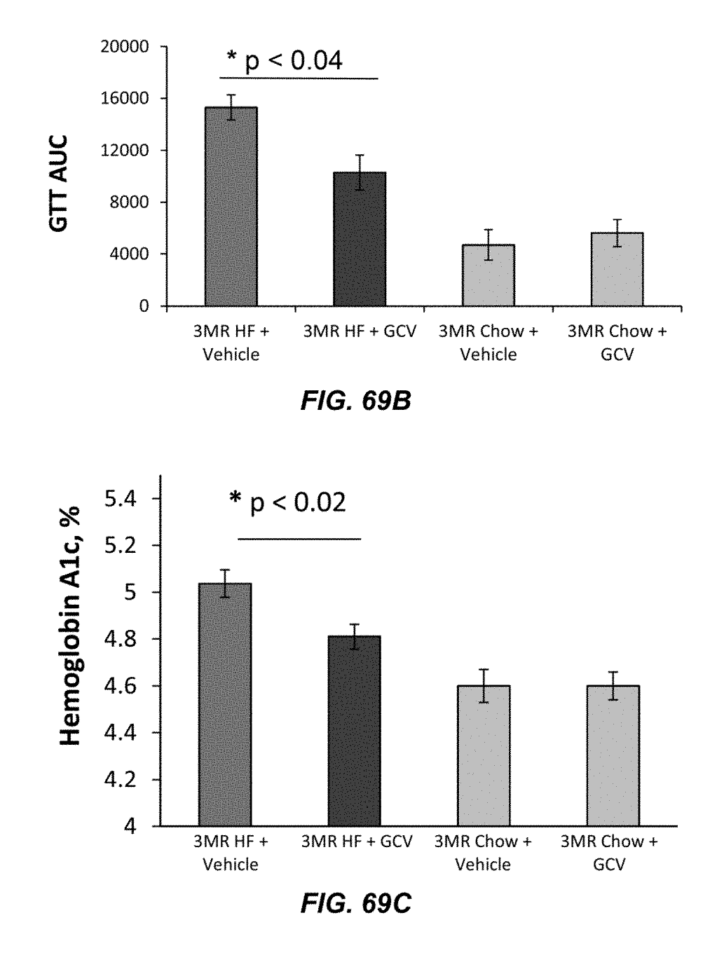

Groups of p16-3MR mice (n=9) were fed a high fat diet for four months mice or a regular chow diet. Animals were then treated with ganciclovir (3 rounds of 25 mg/kg ganciclovir administered daily for five consecutive days) or vehicle. A glucose bolus was given at time zero, and blood glucose was monitored at 20, 30, 60, and 120 minutes after delivering glucose to determine glucose disposal (see FIG. 69A). This was also quantified as “area under the curve” (AUC) (see FIGS. 69B and 69C), with a higher AUC value indicating glucose intolerance. AUCs of mice treated with ganciclovir were significantly lower than their vehicle-treated counterparts although not as low as chow-fed animals. Hemoglobin Al c was lower in ganciclovir-treated mice (see FIG. 69C), suggesting that the animals' longer-term glucose handling was also improved.

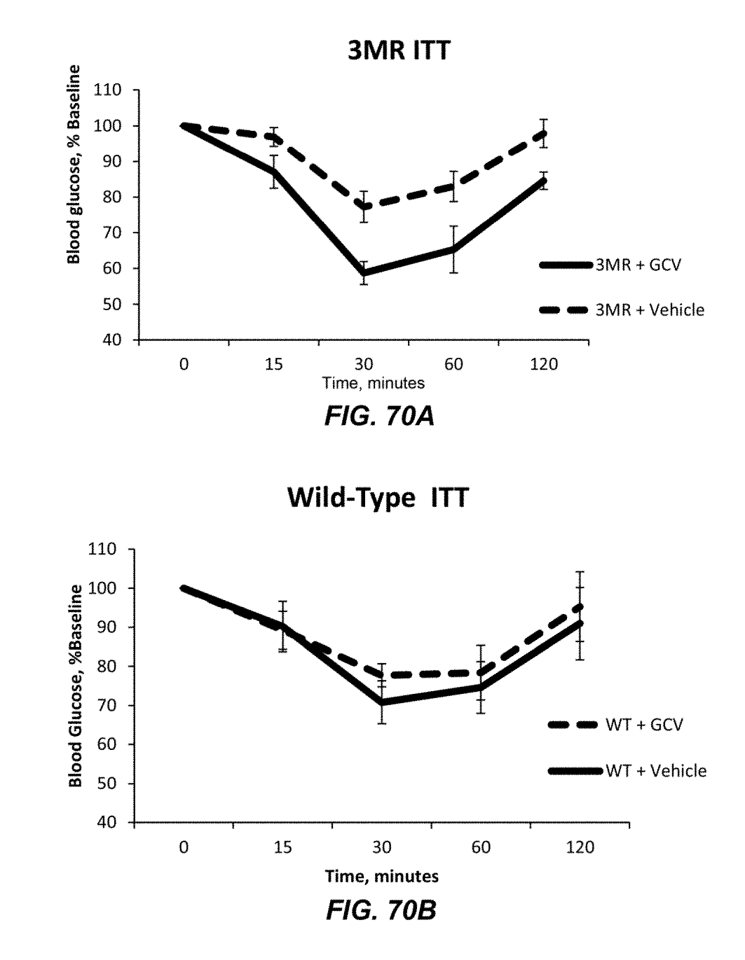

Insulin sensitivity was also determined (Insulin Tolerance Testing (ITT)). The results are presented in FIG. 70. Ganciclovir-treated mice showed a greater decrease in blood glucose at 0, 14, 30, 60, and 120 minutes after the administration of glucose bolus at time zero (see FIG. 70A), suggesting that senescent cell clearance improved insulin sensitivity. A change in insulin tolerance testing when ganciclovir was administered to wild-type mice was not observed (see FIG. 70B).

Changes in weight, body composition, and food intake were also monitored. Treatment by ganciclovir did not alter body weight, body composition monitored by measuring percent of fat, or food intake (measured in grams per week).

### Example 32 Senolytic Activity of a Bcl-2/Bcl-xL Inhibitor

A cell viability assay was used to assess cell survival following treatment with A-1155463. The general timelines and procedures for the cell counting assay are shown in FIG. 18 and described in Example 7. IMR90 cells (human primary lung fibroblasts) were seeded in six well plates, and cells were induced to senescence with 10 Gy of ionizing radiation (IR) (Day 0). The media was refreshed every 3 days. The senescent phenotype is allowed to develop for 7 days at which point a cell count was made to determine the baseline number of cells followed by seeding into 96-well plates. On day 8, the senescent cells (irradiated) and the non-senescent cells (the non-radiated cells), were exposed to serial dilutions of A-1155463 for a period of 24 hours. Each condition was seeded in triplicate. Cells were assayed for cell survival using CellTiter-Glo® (CTG) Luminescent Cell Viability Assay. The assay determines the number of viable cells in culture based on the quantitation of ATP present, which is an indicator of metabolically active cells.

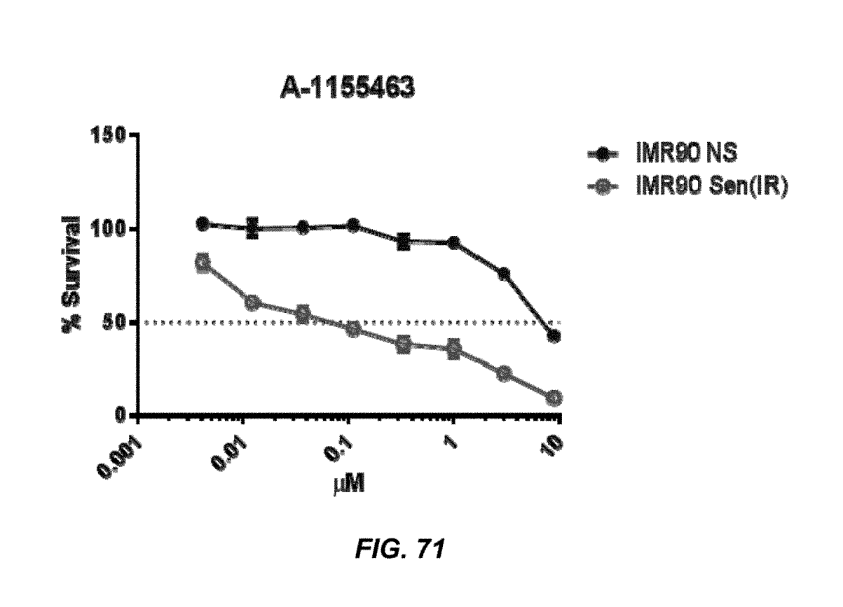

FIG. 71 shows IC50 curves of A-1155463 in senescent cells and in non-senescent cells. The IC50 curve is a plot of the percentage of cell survival following treatment.

### Example 33 Effect of Clearance of Senescent Cells in an Adult Subject on Lifespan

The INK ATTAC (ATTAC) mouse is a transgenic mouse in which a drug-inducible suicide transgene and green fluorescent protein (GFP) are produced behind the promoter for the INK4A gene, which is actively induced in cells that have undergone senescence. Upon delivery of the compound AP20187 (AP), the suicide transgene is activated and the senescent cell is cleared by programmed cell death.

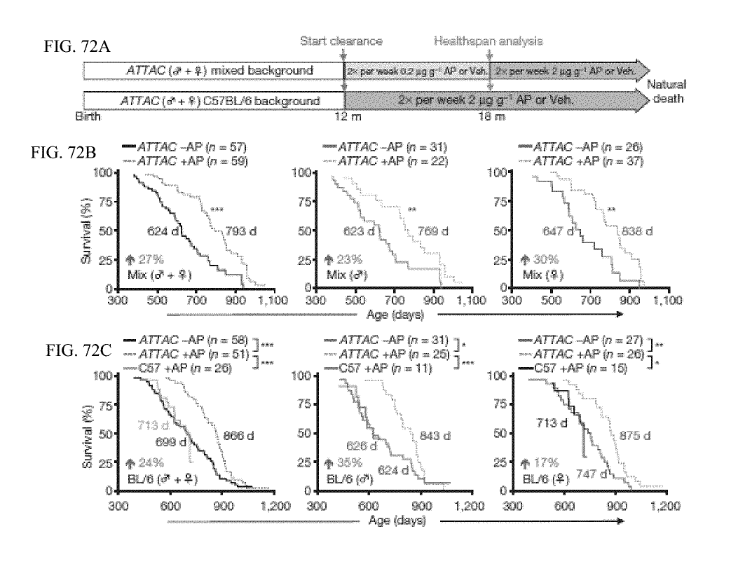

As described in FIG. 72A, cohorts of ATTAC mice of C57BL/6J×FVB mixed or C57BL/6J pure backgrounds of both sexes were aged for 12 months. At 12 months, the ATTAC mice began senescent cell clearance by twice weekly injections of vehicle (Veh) or AP at 0.2 micrograms (μg) per gram (g) mouse body weight for six months. Animals were analyzed for healthspan at 18 months and aged with continued twice weekly vehicle or AP dosing at 2 μg/g mouse body weight until the end of life.

FIG. 72B shows survival data for C57BL/6J×FVB mixed background (Mix) ATTAC mice in males and females (♂+♀), males (♂), or females (♀) treated with vehicle (−AP) or AP (+AP) as described in FIG. 72A. FIG. 72C shows survival data for C57BL/6J pure background (BL/6) ATTAC mice in the treatment groups of in males and females (♂+♀), males (♂), or females (♀) treated with vehicle (−AP) or AP (+AP) as described in FIG. 72A.

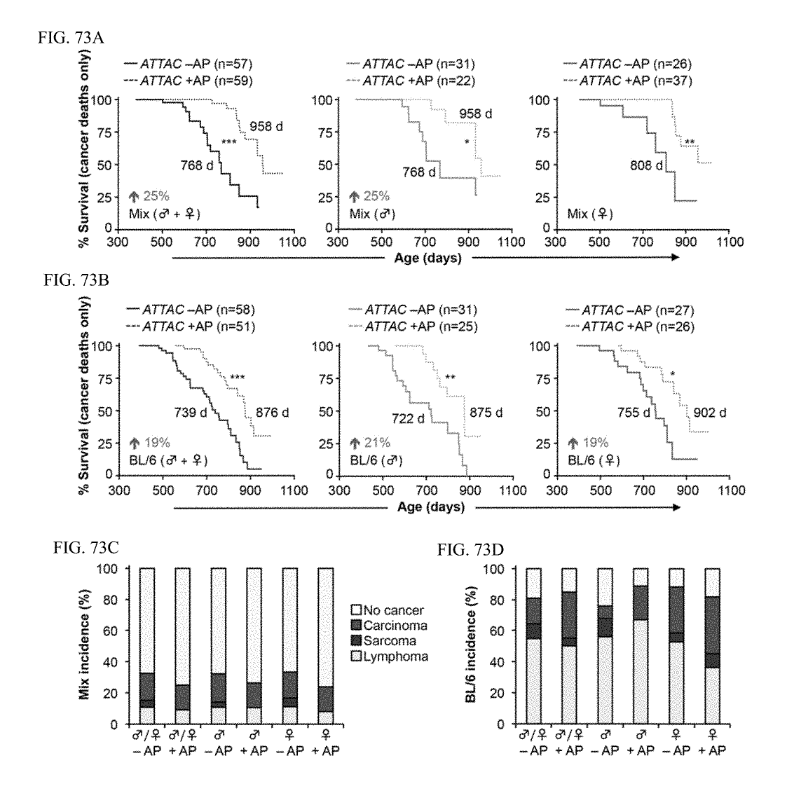

FIG. 73A shows cancer survival data for ATTAC mice in the treatment groups described in FIG. 72B. FIG. 73B shows cancer survival data for ATTAC mice in the treatment groups described in FIG. 72C. FIG. 73C shows the cancer type spectrum for ATTAC mice in the treatment groups described in FIG. 72B. FIG. 73D shows the cancer type spectrum for ATTAC mice in the treatment groups described in FIG. 72C.

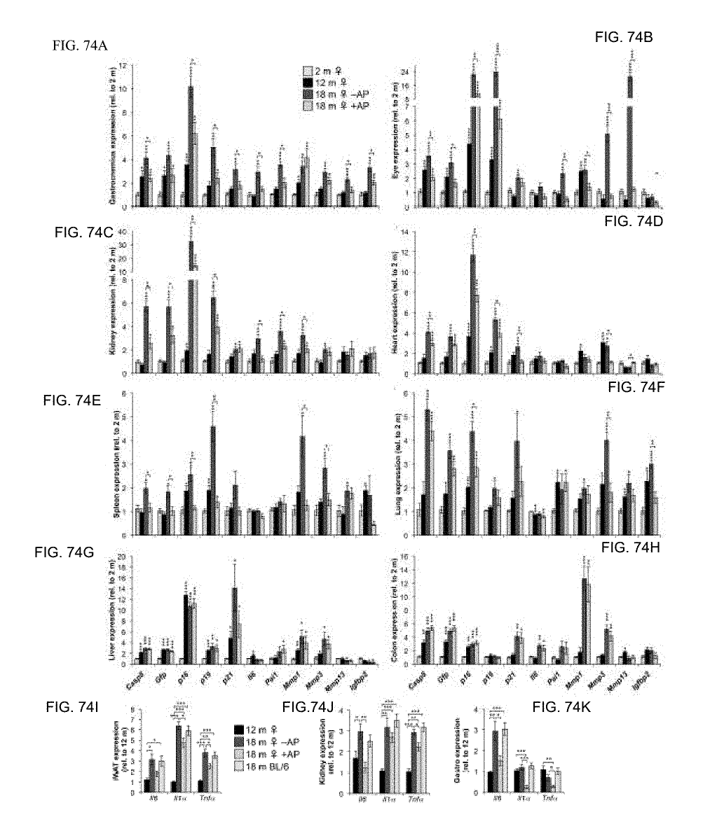

FIG. 74 shows the expression of transcripts determined by quantitative reverse-transcription PCR (qRT-PCR) in ATTAC female mice at 2 months of age (2 m), 12 months of age (12 m), or 18 months of age (18 m), with 18 m animals being treated with vehicle (−AP) or (+AP) as described in FIG. 72A. FIG. 74A shows transcript expression in gastrocnemius. FIG. 74B shows transcript expression in the eye. FIG. 74C shows transcript expression in the kidney. FIG. 74D shows transcript expression in the heart. FIG. 74E shows transcript expression in the spleen. FIG. 74F shows transcript expression in the lung. FIG. 74G shows transcript expression in the liver. FIG. 74H shows transcript expression in the colon. FIGS. 74I-K show expression of inflammation markers. FIG. 74I shows transcript expression in iWAT. FIG. 74J shows transcript expression in the kidney. FIG. 74K shows transcript expression in gastrocnemius.

### Example 34 Effect of Clearance of Senescent Cells in an Adult Subject on Exploratory Activity and Behavior

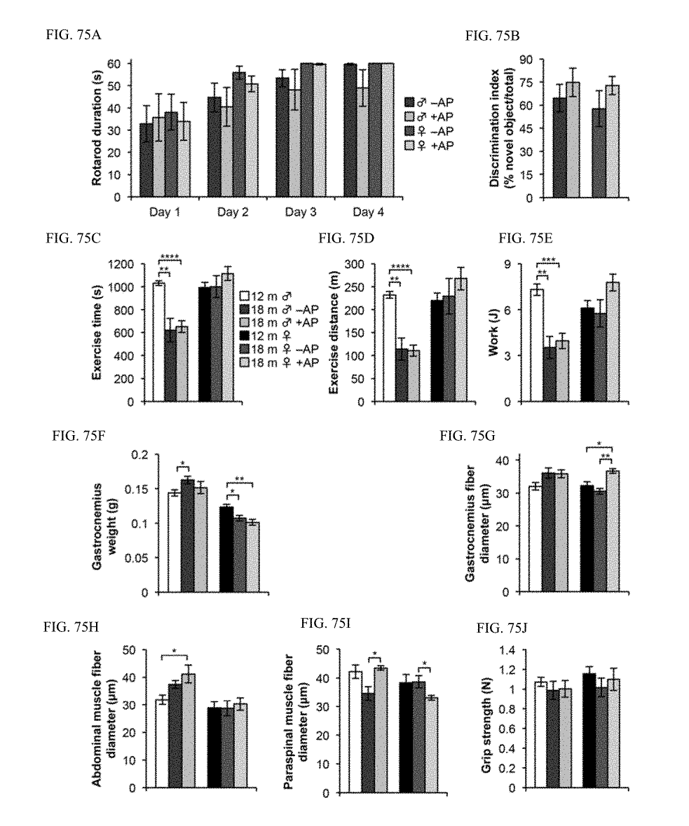

FIG. 75A shows duration of time spent balanced on a rotarod in s for 18 m ATTAC male (columns 1-2, 5-6, 9-10, and 13-14) and female (columns 3-4, 7-8, 11-12, and 15-16) mice −AP (columns 1, 3, 5, 7, 9, 11, 13, and 15) or +AP (columns 2, 4, 6, 8, 10, 12, 14, and 16). FIG. 75B shows the percentage of investigations of a novel object (% novel object/total) in 18 m ATTAC male (columns 1-2) and female (columns 3-4) mice −AP (columns 1 and 3) and +AP (columns 2 and 4). FIG. 75C shows the exercise time to exhaustion in seconds (s) for ATTAC male (columns 1-3) and female (columns 4-6) mice at 12 m (columns 1 and 4), 18 m −AP (columns 2 and 5), or 18 m+AP (columns 3 and 6). FIG. 75D shows the exercise distance to exhaustion in m for ATTAC mice in the treatment groups described in FIG. 75C. FIG. 75E shows the work in Joules (J) for ATTAC mice in the treatment groups described in FIG. 75C. FIG. 75F shows the gastrocnemius weight in g for ATTAC mice in the treatment groups described in FIG. 75C. FIG. 75G shows the gastrocnemius fiber diameter in microns (μm) for ATTAC mice in the treatment groups described in FIG. 75C. FIG. 75H shows the abdominal muscle fiber diameter in μm for ATTAC mice in the treatment groups described in FIG. 75C. FIG. 75I shows the paraspinal muscle fiber diameter in μm for ATTAC mice in the treatment groups described in FIG. 75C. FIG. 75J shows the grip strength in Newtons (N) for ATTAC mice in the treatment groups described in FIG. 75C.

### Example 35 Effect of Clearance of Senescent Cells in an Adult Subject on Adiposity

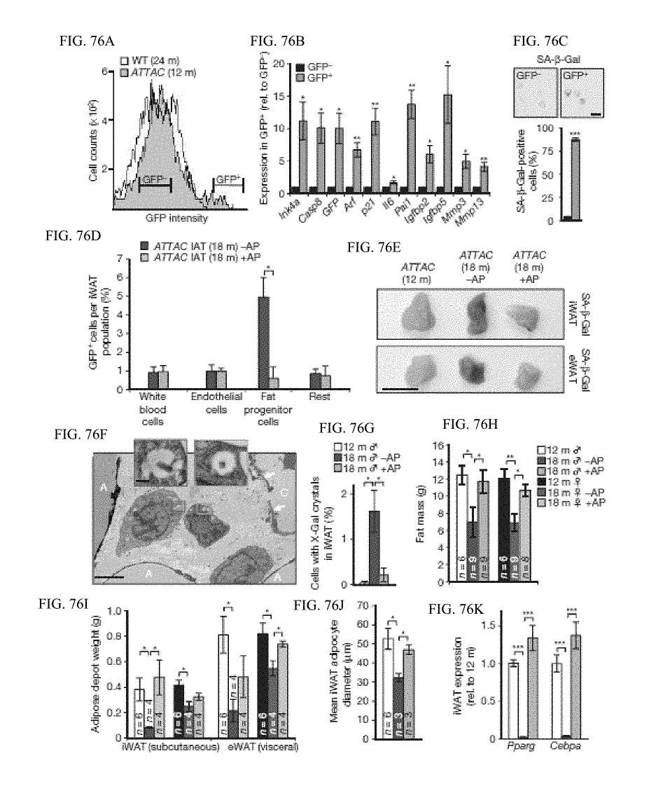

FIG. 76A shows GFP-non-expressing (GFP−) and GFP-expressing (GFP+) cells in inguinal adipose tissue (IAT) of wild type (WT) or ATTAC mice. As used herein, the terms IAT and iWAT are interchangeable. FIG. 76B shows the expression of transcripts determined by qRT-PCR in cells from GFP+ or GFP− IAT. FIG. 76C shows senescence-associated β-galactosidase (SA-(3-Gal) staining in cells from GFP+ or GFP− IAT and a graph of the percentage (%) of SA-β-Gal-positive cells from GFP− (column 1) or GFP+(column 2) IAT. The scale bar is 10 microns (μm). FIG. 76D shows the percentage of GFP+ cells in white blood cells, endothelial cells, fat progenitor cells, or remaining cells (“Rest”) from the IAT of ATTAC mice at 18 m −AP or +AP. FIG. 76E shows whole-mount SA-β-Gal staining of IAT and epididymal fat (EPI) from ATTAC mice at 12 m, 18 m −AP, or 18 m+AP. The scale bar is 0.5 cm. FIG. 76H shows fat mass measurements from ATTAC mice that are 12 m, 18 m −AP or 18 m+AP that are male (♂) or female (♀). FIG. 76F shows perivascular X-Gal-positive cells from an 18-month-old vehicle-treated C57BL6 ATTAC male. A, adipocyte; C, capillary. Arrows mark endothelial cells. FIG. 76G shows quantification of cells containing X-Gal crystals (n=4 mice per treatment). FIG. 76H shows fat mass measurements from ATTAC mice that are 12 m, 18 m −AP or 18 m+AP that are male (♂) or female (?). FIG. 76I shows IAT (subcutaneous) and perigonadal (visceral) adipose depot weight from ATTAC mice that are 12 m, 18 m −AP or 18 m+AP that are male (♂) or female (♀). FIG. 76J shows the mean adipocyte diameter in μm in ATTAC male mice at 12 m, 18 m −AP, or 18 m+AP. FIG. 76K shows expression of Pparg and Cebpa transcripts determined by qRT-PCR in ATTAC male mice at 12 m, 18 m −AP, or 18 m+AP.

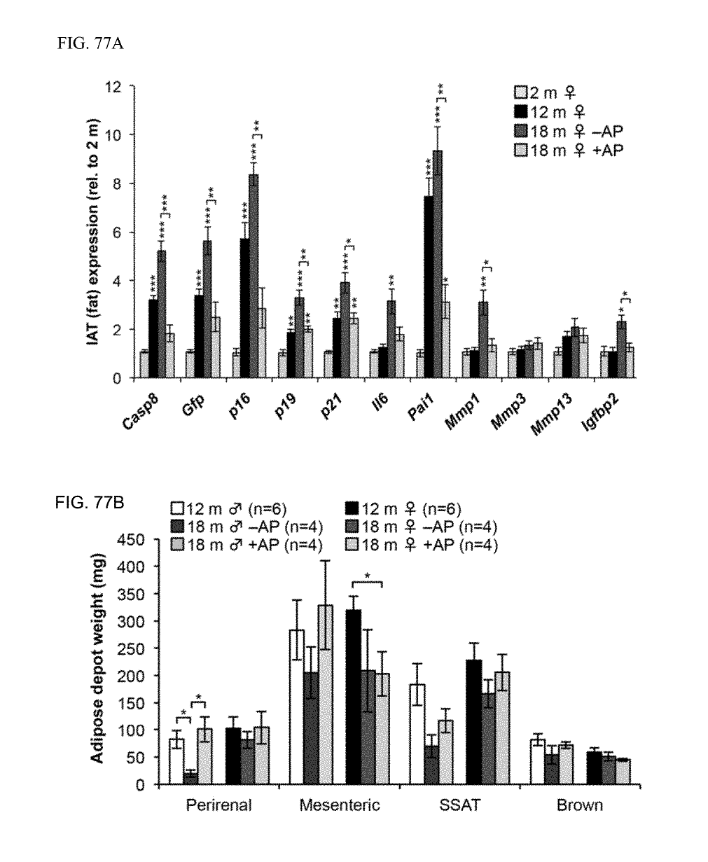

FIG. 77A shows the expression of transcripts determined by qRT-PCR in female ATTAC mice at 2 months of age (2 m), 12 m, 18 m −AP, or 18 m+AP. FIG. 77B shows perirenal, mesenteric, subscapular adipose tissue (SSAT), and brown adipose tissue weight in ATTAC male mice at 12 m, 18 m −AP, or 18 m+AP.

### Example 36 Effect of Clearance of Senescent Cells in an Adult Subject on Kidney Function

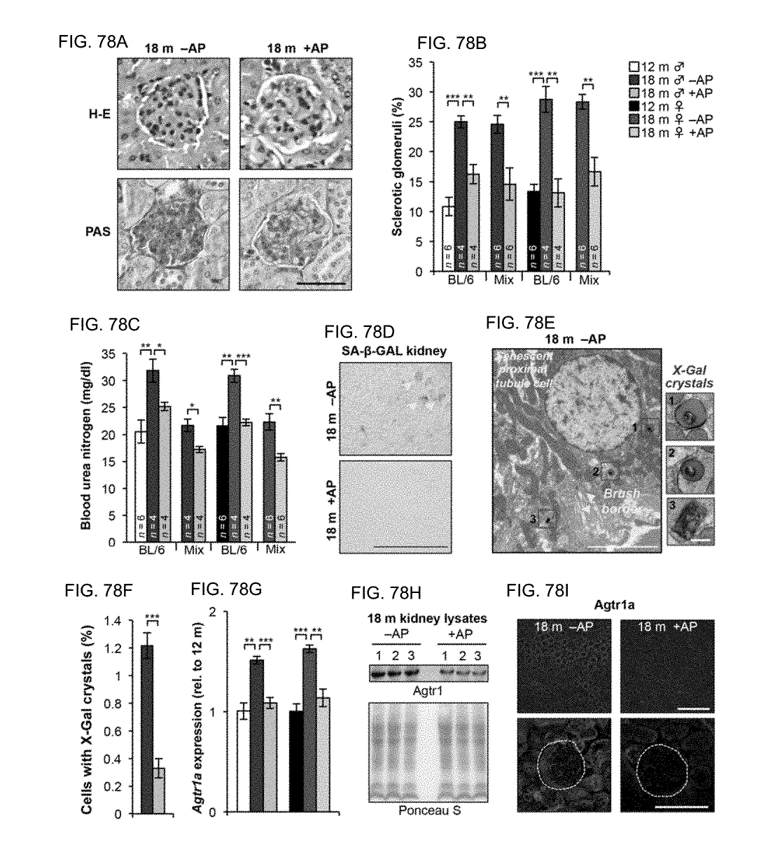

FIG. 78A shows hematoxylin and eosin (H-E) staining and Periodic acid-Schiff (PAS) staining of kidney tissue from ATTAC mice at 18 m −AP or 18 m+AP. The scale bar is 50 μm. FIG. 78B shows the percentage (%) of sclerotic glomeruli in ATTAC mice from the treatment groups described in FIG. 72F. FIG. 78C shows the concentration of blood urea nitrogen in milligrams (mg) per deciliter (dl) in ATTAC mice from the treatment groups described in FIG. 72F. FIG. 78D shows whole-mount SA-β-Gal staining of kidney tissue from ATTAC mice at 18 m −AP or 18 m+AP. The scale bar is 250 μm. FIG. 78E is an electron micrograph of kidney tissue from a BL/6 ATTAC male mouse 18 m −AP. Inset images 1-3 show X-Gal crystals in the kidney tissue. The scale bar is 5 μm for the main micrograph and 200 nanometers (nm) for the inset images. FIG. 78F shows the percentage (%) of cells with X-Gal crystals in BL/6 ATTAC males at 18 m −AP (column 1) or 18 m+AP (column 2). FIG. 78G shows expression of Atgr1a transcript determined by qRT-PCR in the kidneys from BL/6 ATTAC male (columns 1-3) and female (columns 4-6) mice at 12 m (columns 1 and 4), 18 m −AP (columns 2 and 5), or 18 m+AP (columns 3 and 6). FIG. 78H shows expression of Atgr1a protein determined by Western blotting in kidneys from ATTAC mice 18 m −AP (lanes 1-3) or 18 m +AP (lanes 4-6), with Ponceau S staining of the membrane as a loading control. FIG. 78I shows immunofluorescent staining for Atgr1a protein from kidney tissue of ATTAC mice 18 m −AP (left images) or 18 m+AP (right images). The scale bar is 100 μm for the top images and 50 μm for the bottom images.

### Example 37 Effect of Clearance of Senescent Cells in an Adult Subject on Cardiac Function

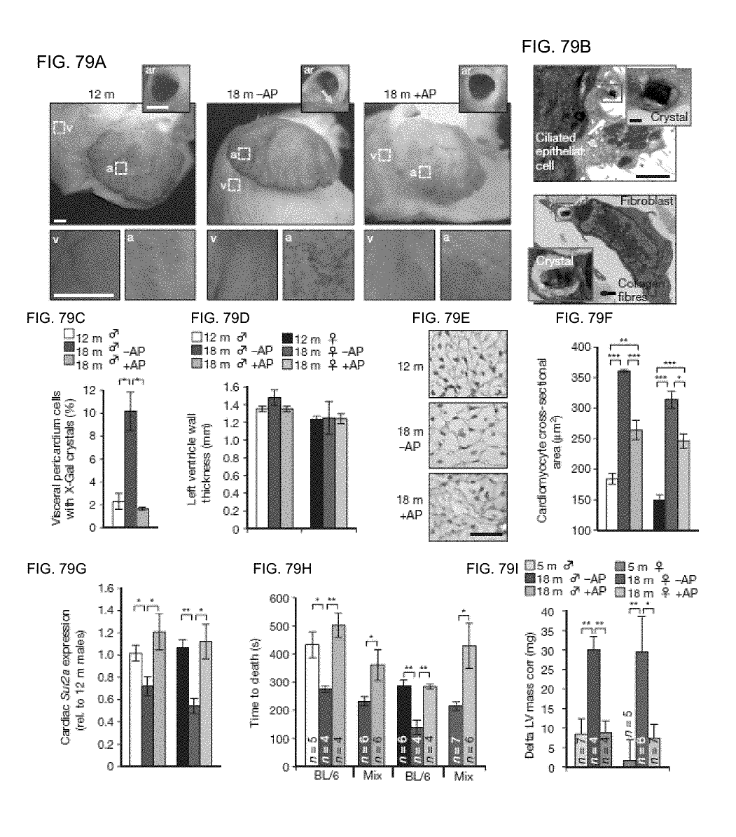

FIG. 79A shows whole-mount SA-β-Gal staining of hearts from BL/6 ATTAC mice at 12 m, 18 m −AP, or 18 m+AP, with the asterisk marking the position of the aortic root. Top inset images show aortic roots (ar) from a transverse plane, with the arrow marking the aortic root wall. Bottom inset images show ventricular (v) and arterial (a) boxed areas. The scale bar is 1 millimeter (mm) for all images. FIG. 79B is a set of electron micrographs of SA-β-Gal positive cells in the pericardium. Inset images show X-Gal images from the boxed areas. The asterisk marks cilia. The circular arrow marks collagen fibers. VSMC is a vascular smooth muscle cell. The scale bar is 2 μm in the main images and 200 nm in the inset images. FIG. 79C shows quantification of cells with X-Gal crystals in the visceral pericardium (n=4 mice per treatment). FIG. 79D shows the left ventricle free-wall thickness in μm of BL/6 ATTAC mice in the treatment groups described in FIG. 78G. FIG. 79E shows representative cardiomyocyte cross-sectional images (n=4 mice per group). FIG. 79F shows cardiomyocyte cross-sectional area in square microns (μm²) of BL/6 ATTAC mice in the treatment groups described in FIG. 78G. FIG. 79G shows expression of Sur2a transcript determined by qRT-PCR in the hearts of BL/6 ATTAC mice in the treatment groups described in FIG. 78G. FIG. 79H shows cardiac stress resistance as measured by the time to death in seconds (s) after injection with a lethal dose of isoproterenol in ATTAC mice from the treatment groups described in FIG. 72F. FIG. 79I shows change in left ventricular mass (LV) in response to sublethal doses of isoproterenol (10 mg/kg) after ten doses administered over five days.

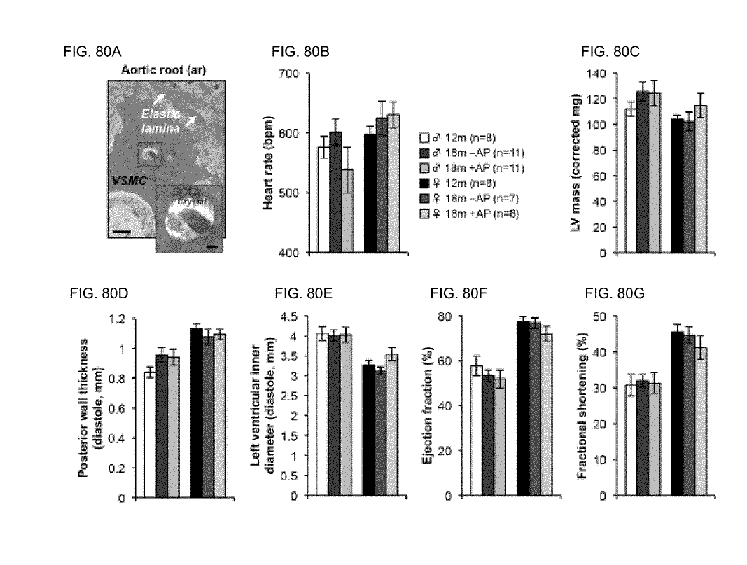

FIG. 80A shows electron micrographs of X-Gal crystal containing cells in the aortic root. VSMC, vascular smooth muscle cell. FIG. 80B shows echocardiograph measurements of heart rate in beats per minute (bpm) for ATTAC mice in the treatment groups described in FIG. 75B. FIG. 80C shows echocardiograph measurements of left ventricular (LV) mass in corrected milligrams (mg) for ATTAC mice in the treatment groups described in FIG. 75B. FIG. 80D shows echocardiograph measurements of posterior wall thickness at diastole in millimeters (mm) for ATTAC mice in the treatment groups described in FIG. 75B. FIG. 80E shows echocardiograph measurements of left ventricular inner diameter at diastole in millimeters (mm) for ATTAC mice in the treatment groups described in FIG. 75B. FIG. 80F shows echocardiograph measurements of percentage (%) ejection fraction of the heart for ATTAC mice in the treatment groups described in FIG. 75B. FIG. 80G shows echocardiograph measurements of percentage (%) fractional shortening of the heart for ATTAC mice in the treatment groups described in FIG. 75B.

### Example 38 Effect of Clearance of Senescent Cells in an Adult Subject on Cataract Incidence

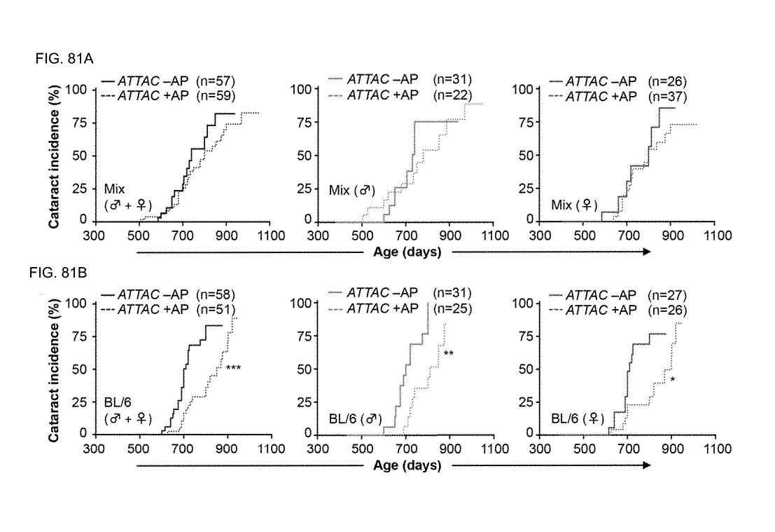

FIG. 81A shows the percentage (%) cataract incidence for ATTAC mice in the treatment groups described in FIG. 72B. FIG. 81B shows the percentage (%) cataract incidence for ATTAC mice in the treatment groups described in FIG. 72C.

### Example 39 Effect of Clearance of Senescent Cells in Young Subjects and Cells

FIG. 82A shows SA-β-Gal staining of IAT from ATTAC mice at 2 m treated with vehicle (−AP) or AP (+AP) beginning at weaning age. FIG. 82B shows the expression of p16 and actin protein determined by Western blotting in IAT from ATTAC mice treated as described in FIG. 82A. FIG. 82C shows the expression of transcripts determined by qRT-PCR in IAT from ATTAC mice treated as described in FIG. 82A. FIG. 82D shows the expression of p16 and actin protein determined by Western blotting in three mouse embryonic fibroblast (MEF) lines untreated (−) or treated with AP (+) at cell passage 3 (P3). FIG. 82E shows cell number over five days (day 0 to day 4) in P3 MEF lines untreated (−AP) or treated with AP (+AP). FIG. 82F shows the expression of transcripts determined by qRT-PCR in P3 MEF lines −AP or +AP. FIG. 82G shows the expression of p16 and actin protein determined by Western blotting in passage 4 (P4) primary ATTAC MEFs and SV40 Large-T antigen (LT) immortalized ATTAC MEFs. FIG. 82H shows the expression of p16 and actin protein determined by Western blotting in SV40 LT immortalized ATTAC MEFs treated with vehicle (−AP), 10 nanomolar AP (+AP (10 nM)), or 500 nM AP (+AP (500 nM)). FIG. 82I shows the expression of transcripts determined by qRT-PCR in P4 primary ATTAC MEFs or SV40 LT immortalized ATTAC MEFs treated as in FIG. 82H.

### Example 40 Effect of AP20187 Treatment in a Wild Type Subject

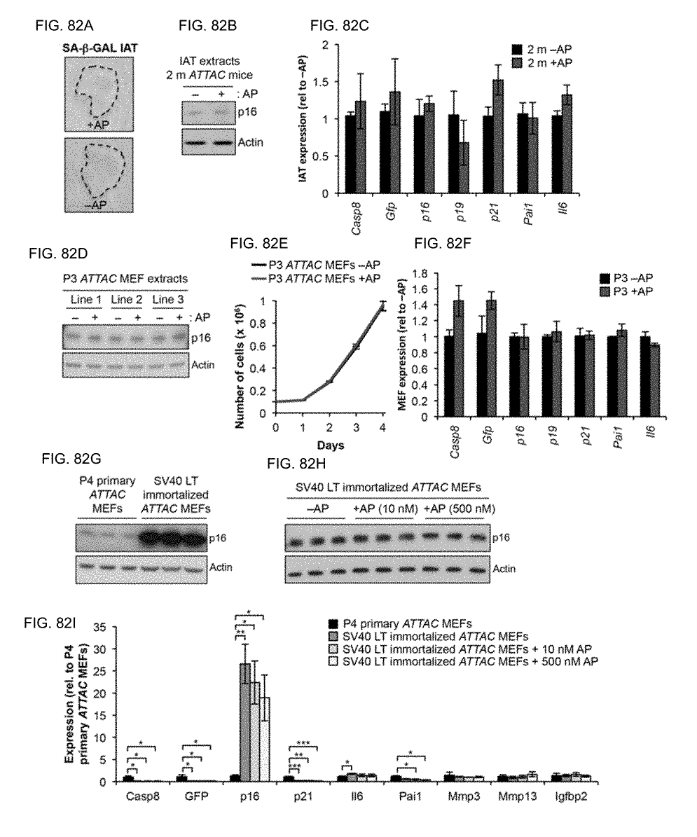

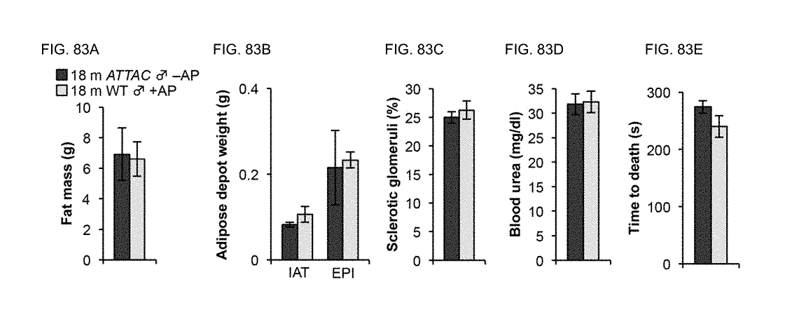

FIG. 83A shows the fat mass in g in ATTAC male mice 18 m −AP and wild type mice at 18 months of age treated with AP (18 m+AP) as described in FIG. 72A. FIG. 83B shows the adipose depot weight in g in IAT and EPI in ATTAC and wild type male mice as described in FIG. 83A. FIG. 83C shows the % of sclerotic glomeruli in ATTAC and wild type male mice as described in FIG. 83A. FIG. 83D shows the concentration of blood urea in mg/dl in ATTAC and wild type male mice as described in FIG. 83A. FIG. 83E shows the time to death in s after injection with a lethal dose of isoproterenol in ATTAC and wild type male mice as described in FIG. 83A.

### Example 41 Effect of Clearance of Senescent Cells in an Adult Subject on Wound Healing and Fibrosis

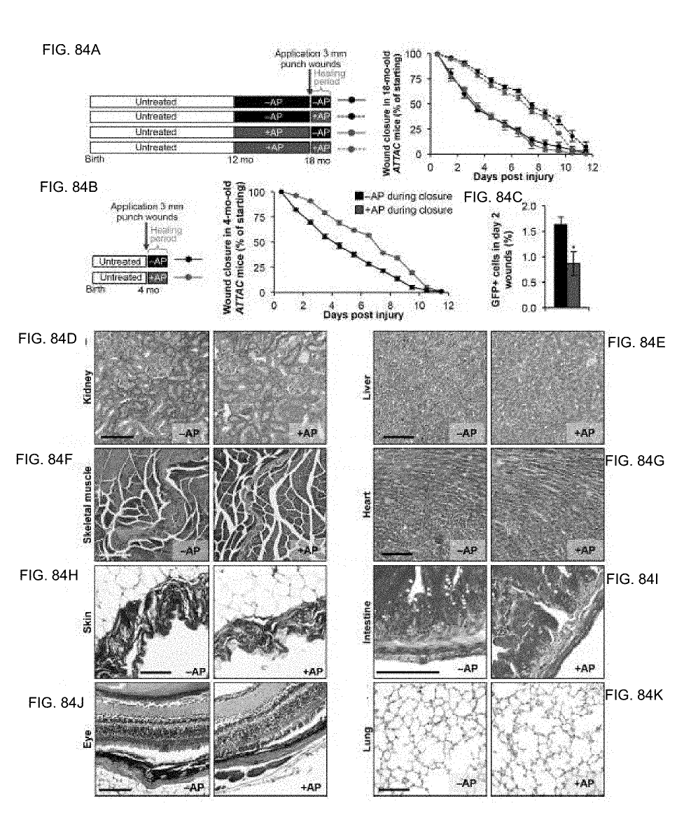

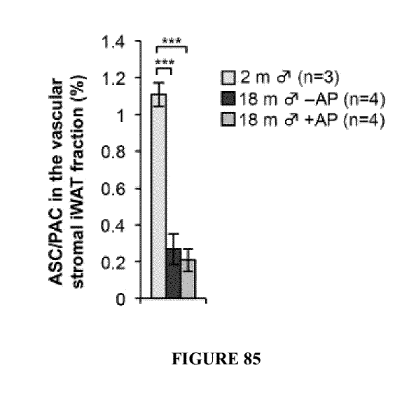

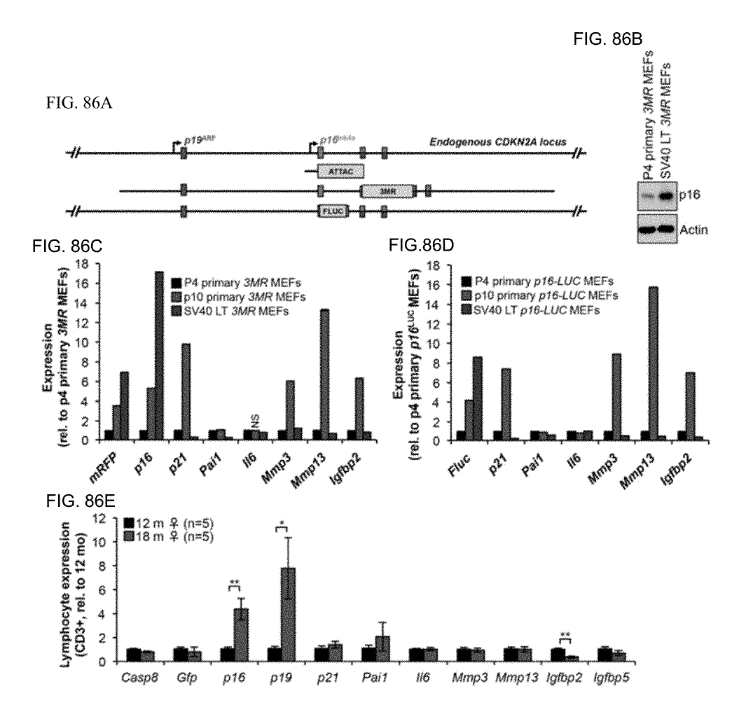

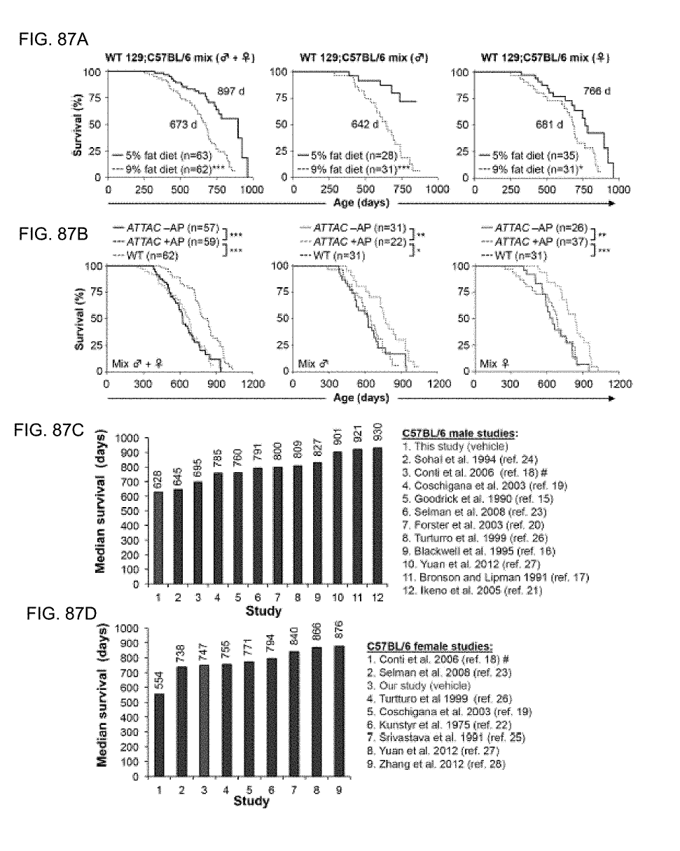

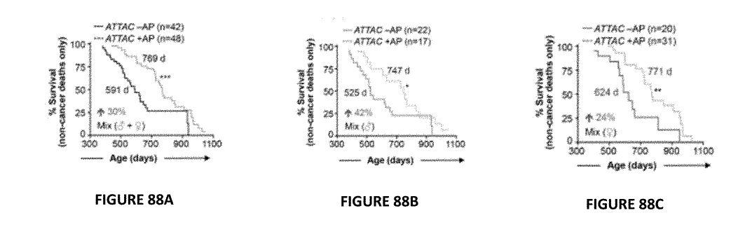

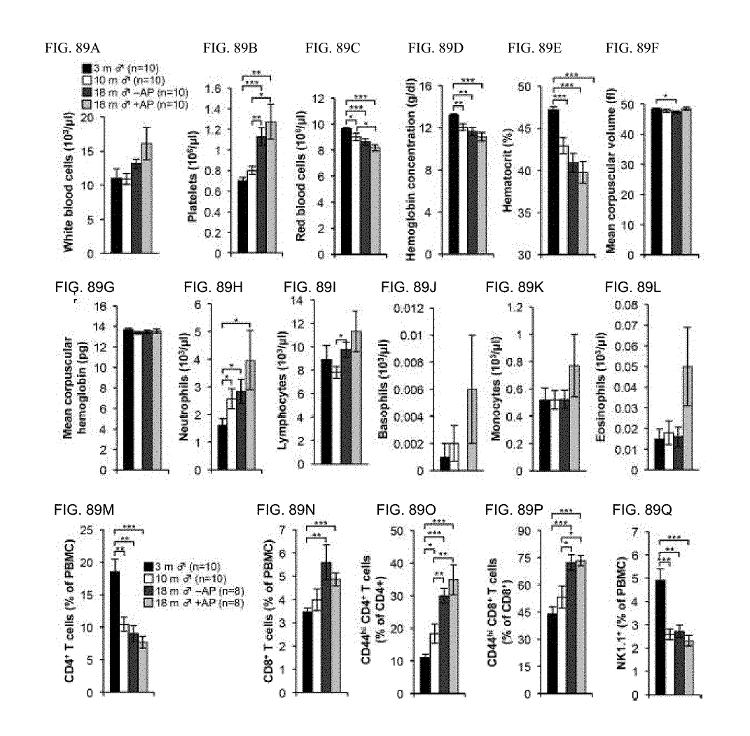

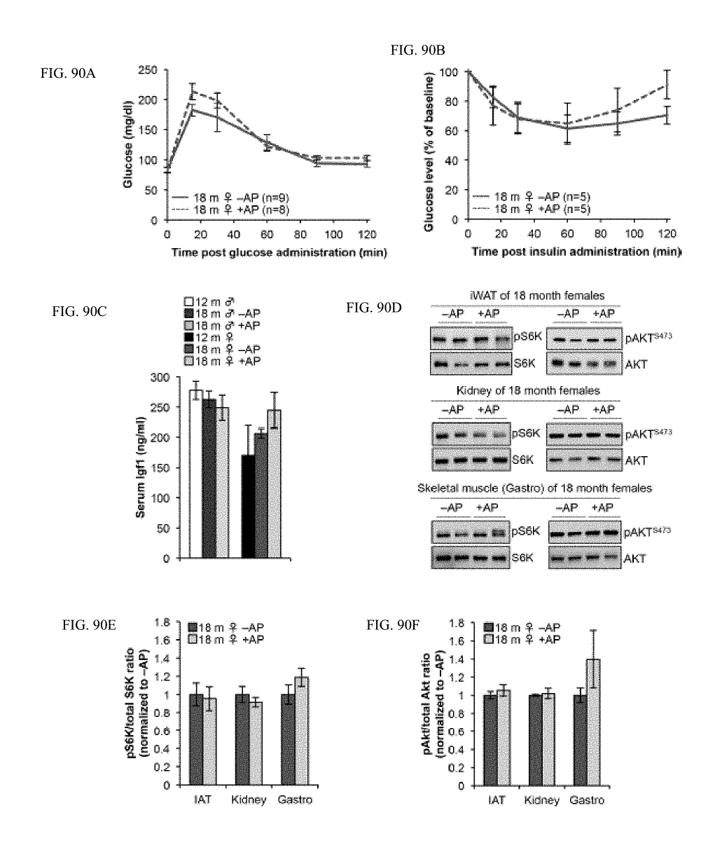

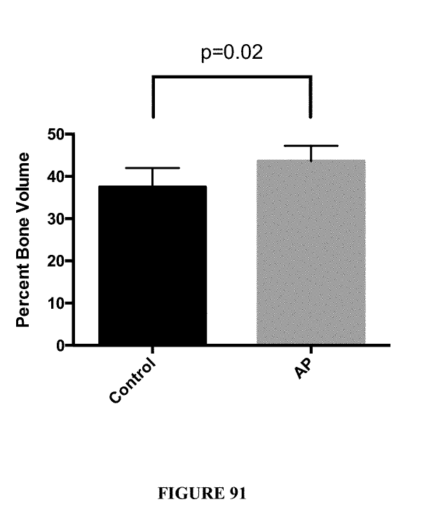

FIG. 84A shows wound closure of a 3-mm punch biopsy measured as the percentage (%) of starting wound size in ATTAC female mice 18 m −AP or 18 m+AP. Treatment was stopped two days prior to the biopsy and during wound closure. FIG. 84B shows wound closure of 3-mm punch biopsy wounds in 4-month-old female ATTAC mice after treatment with vehicle or AP following wounding. FIG. 84C shows quantification of total GFP⁺ cells isolated from 3-mm punch biopsy wounds of 4-month-old mice two days into the wound healing process treated with vehicle (black) or AP (grey). FIG. 84D-84K shows phosphotungstic acid haematoxylin (PTAH) staining of tissue sections from ATTAC mice at 18 m −AP or +AP. FIG. 84D shows PTAH staining in the kidney. FIG. 84E shows PTAH staining in the liver. FIG. 84F shows PTAH staining in skeletal muscle. FIG. 84G shows PTAH staining in the heart. FIG. 84H shows PTAH staining in the skin. FIG. 84I shows PTAH staining in the intestine. FIG. 84J shows PTAH staining in the eye. FIG. 84K shows PTAH staining in the lung. The scale bars are each 100 μm.

### Example 42: Effect of MDM2 Inhibitors on Survival of Senescent Cells

The table below shows EC50 doses for senescent and high density non senescent cells treated with MDM2 inhibitors. Senescent cells (SnC) and quiescent high density non senescent cells (HD NsC) were treated with a MDM2 inhibitors at a range of doses and cell survival was scored to determine that MDM2 inhibitor dose that resulted in 50% cell survival for each cell type. EC50 dose ratios were computed (HD NsC/SnC) and are shown in the table below. Boronate, RG-7112, JNJ 26854165 and Me123 showed particularly high selectivity for senescent cells over quiescent cells.

TABLE
EC50 doses for selected MDM2 inhibitors

| | | Cell survival data | | |
|---|---|---|---|---|
| Proposed mechanism | | EC50 uM | | HD NsC/ |
| of action | Compound | SnC | HD NsC | SnC |
| | AMG-232 | 3.4 | 4.1 | 1.2 |
| | NVP-CGM097 | 1.5 | 1.9 | 1.3 |
| | RG7388 | 32.8 | 31.5 | 1.0 |
| | MI-773 | 4.7 | 9.5 | 2.0 |
| | CAY10681 | 11.0 | 17.6 | 1.6 |
| | CAY10682 | 11.2 | 14.3 | 1.3 |
| | YH239-EE | 7.2 | 11.4 | 1.6 |
| | RG-7112 | 2.8 | 13.0 | 4.7 |
| | Boronate | 10.8 | 153.2 | 14.1 |
| Bind N-term | RO-5963 | 22.7 | 39.4 | 1.7 |
| MDM2/X | | | | |
| Bind to p53 | RITA | 254.2 | 223.2 | 0.9 |
| Block MDM2 E3 | HLI 373 | 10.9 | 9.1 | 0.8 |
| ligase activity | JNJ 26854165 | 1.0 | 6.6 | 6.5 |
| block MDM2/X | MEL23 | 4.0 | 20.9 | 5.2 |
| E3 act | | | | |

### Example 43 Micro Computerized Tomography Outcomes in Bones of Mice Treated with a Senolytic Agent

This example describes a method for assessing the effect of senotlytic agents or killing of senescent cells on bone structure. Two groups of INKattac mice are used for this method: mice treated from 12-28 months of age with vehicle (“old” controls) and mice treated from 12-28 months of age with AP20187. All mice used in this example were male. After sacrifice the cadavers were fixed in 10% neutral buffered formalin, and stored at 4° C.

### Preparation of Mice for Micro CT.

Mice were cleaned of excess tissue, and placed in a Bruker 1176 “Skyscanner” for micro computerized tomographic (mCT) scanning. A variety of settings were explored to visualize whole skeletons, the following settings were used in this example: 65 Kv, 385 μA, 17.58 μm, 0.5 mm Al filter, rotation step 0.5, frame averaging 6, smoothing 2, smoothing kernel 2, ring artifact correction 4, and beam hardening correction 30%. Typical acquisition consisted of a whole body scan, followed by additional scans if necessary for further data exploration.

Reconstruction of the raw data was carried out. Subvoluming of anatomical features of interest was then further performed on the whole skeleton, using DataViewer, or CtAn software (Bruker).

Once a feature had been digitally isolated (for example mid cortical shaft), it was then imported into CtAn (Bruker), or Mimics v18 (Materialise) for further analysis/image processing.

### Example 44 Mid Femoral Cortical Bone Volume is Retained with Age by Senolytic Treatment

The amount of bone synthesized declines with age. This results in a net loss of bone, and impaired structural integrity. A number of reports have shown that femurs from aging mice have a pronounced loss of bone, consistent with changes seen in human aging. Long bones (for example the femur) broadly consist of two types of bone, dense hard bone (cortical), and thin rods of bone located within the marrow space (trabecular bone). Both types of bone play a structural role, but in aged mice, there is little trabecular bone at the mid-diaphysis (shaft) of the femur.

The method of example 43 was used to analyze a 2 mm subvolume of the mid-diaphysis of the femur in treated mice (AP) versus non-treated mice (control) to determine if cortical age-related bone loss could be attenuated by chronic treatment with a senolytic agent.

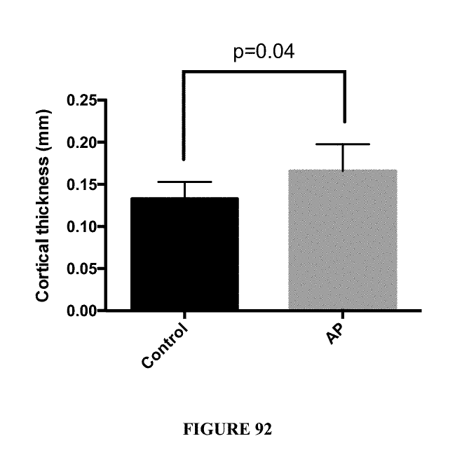

The percent bone volume (BV/TV—Bone Volume over Total Volume) is a common metric used in the assessment of aging bone. This represents the amount of bone (BV) contained within a specific volume (TV), hence higher numbers indicate a greater mass of bone in the volume measured. FIG. 92 shows that chronic treatment with AP20187 results in retention of 6.1% cortical bone volume with age.

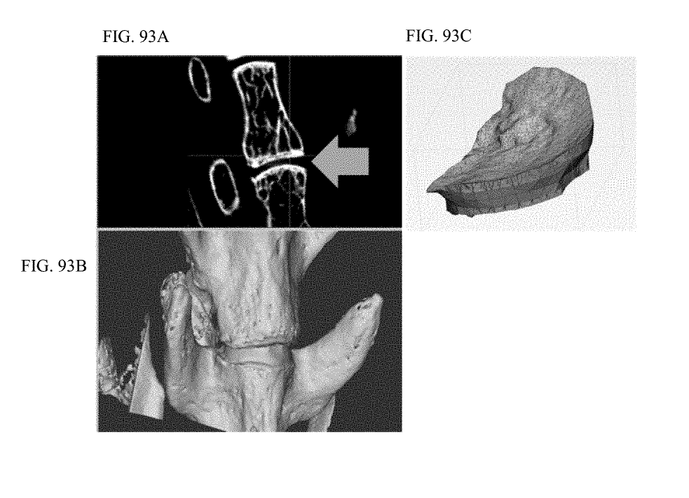

Consistent with the retention of bone volume with age, the thickness of femoral bone within the 2 mm sub-volume was also improved by senolytic treatment. The average thickness of each 2 mm subvolume was measured in treated versus non-treated animals. Similar to BV/TV, the t-test was permuted to determine whether there was a difference with cortical thickness and senolytic treatment. Cortical thickness was improved on average by 20% with senolysis (p=0.04, FIG. 93).

### Example 45 Intervertebral Disc Space is Retained with Senolytic Treatment

Intervertebral discs are type of cartilage filling the space between vertebras of the spine. They are known to decrease in function and volume with age. We estimated the intervertebral volumes between specific vertebra in spines of aged mice, with and without senolytic treatment, to determine if intervertebral disc space was maintained in aged animals through senolysis.

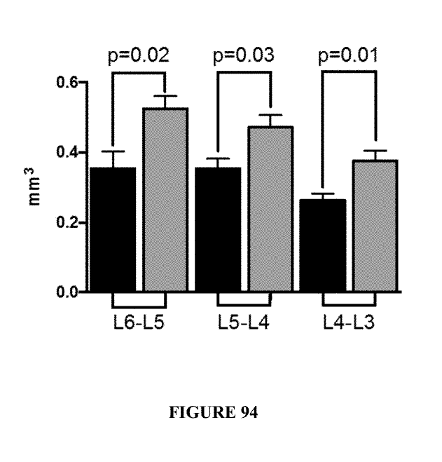

While IVDs cannot be directly visualized with the micro-CT method of example 43, an image segmentation protocol was developed to infer disc volume by determining the volume between lumbar vertebra L6 and L5, L5 and L4, and L4 and L3 to estimate the intervertebral disc space (IVS) (FIG. 94).

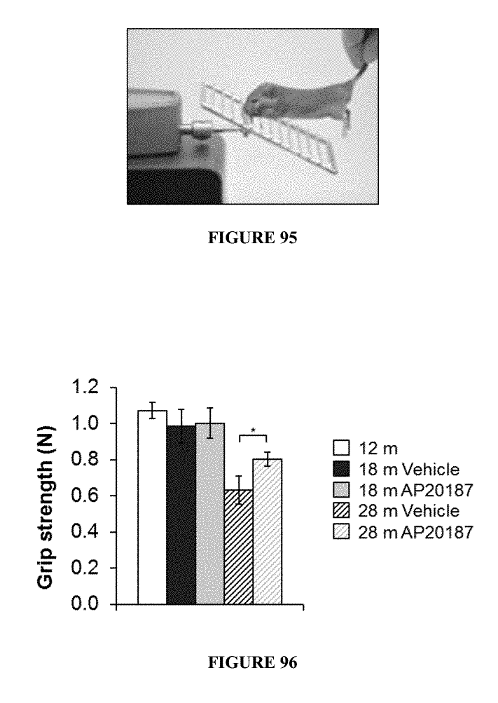

As shown in FIG. 95, IVS was determined at three distinct sites in the spine, and in each case, the IVS was statistically significantly greater in AP20187 treated animals than vehicle treated controls (25-33% improved).

Similarly to the femoral analysis, we carried out permutation of a t-test up to 10,000 times to ensure statistical robustness, and determined that each of the distinct IVS were statistically greater in AP20187 treated animals.

### Example 46 Senolytic Treatment Decreases Late Life Muscle Strength Decline

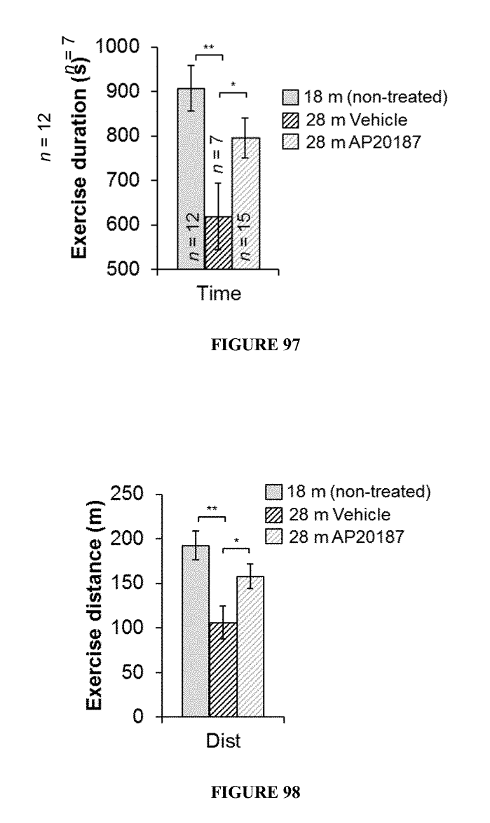

Grip strength was measured in Newtons using standard methods involving a grip strength meter as shown in FIG. 96. Grip strength was measured in untreated 12 month old mice, and in mice treated with either vehicle or AP20187 at 18 and 28 months of age. As seen in FIG. 97 mice receiving AP20187 treatment showed decreased loss of muscle strength compared to mice receiving vehicle at 28 months.

Muscle strength was also assessed by determining exercise duration and distance on a treadmill. As shown in FIGS. 97 and 98 mice at 28 months of age exercised for longer, and for greater distances, when treated with AP20187 compared to vehicle.

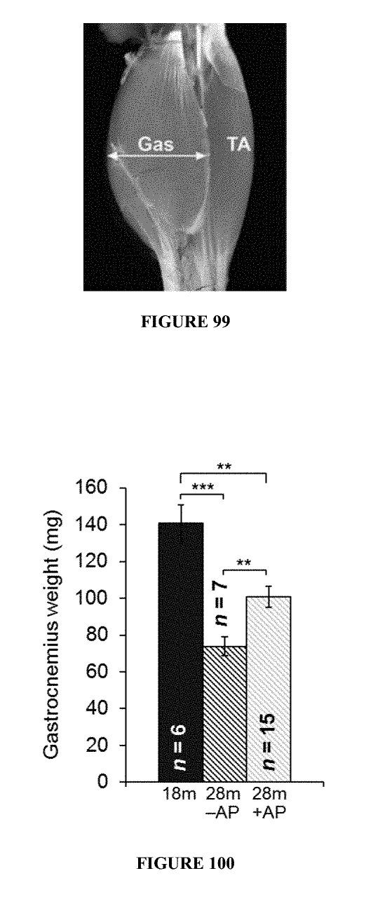

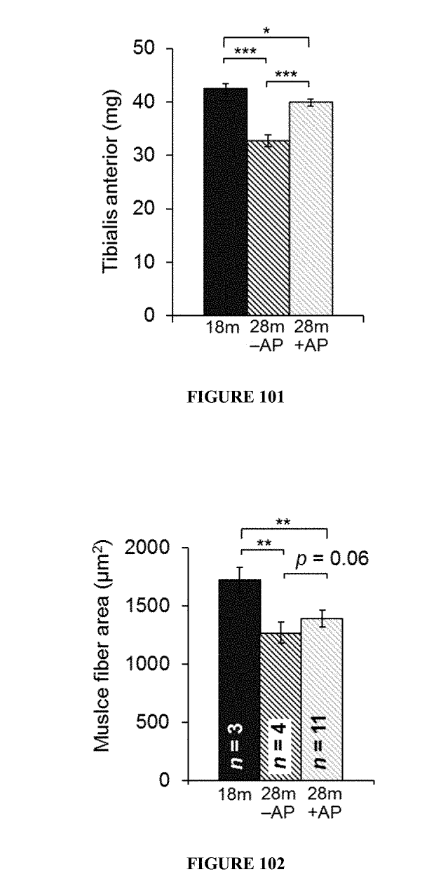

As an additional measure of muscle condition gastrocnemius and tibialis anterior muscles, shown in FIG. 99, where dissected from senolysis treated and untreated 28 month old mice and weighed. As shown in FIGS. 100 and 101 the gastrocnemius and tibialis anterior muscles are heavier in treated mice than in untreated mice, muscle weights from untreated 18 month old mice are included to show the age related decline. Muscle fiber area of the gastrocnemius and tibialis anterior muscles was also used to assess muscle condition. As shown in FIG. 100 mice treated as above had a larger muscle fiber area than vehicle treated controls.

### Example 47 Senolytic Treatment Improves Rotarod Performance

A group of 34 C57BL/6J mice were tested for motor coordination in a rotarod at 23 months of age. Mice were tested for 4 consecutive days. Each session consisted of 4 runs in which the speed of the rotating rod increased from 4 to 40 rpm over 5 minutes. Mice were allowed to run until they fell. The latency to fall was recorded for each run, and averaged to obtain a single value for each session. Half of the mice were then treated with 50 mg/kg nutlin-3a for 14 consecutive days, via IP injection, while the rest of them were sham injected. 3 weeks after the last day of treatment, motor coordination was assessed following the described procedure.

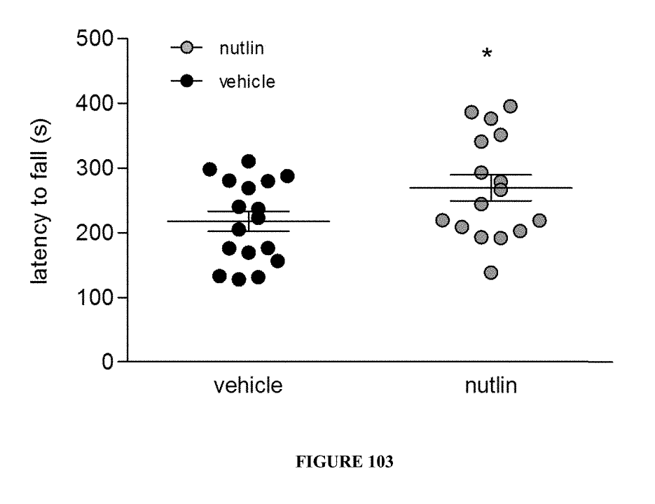

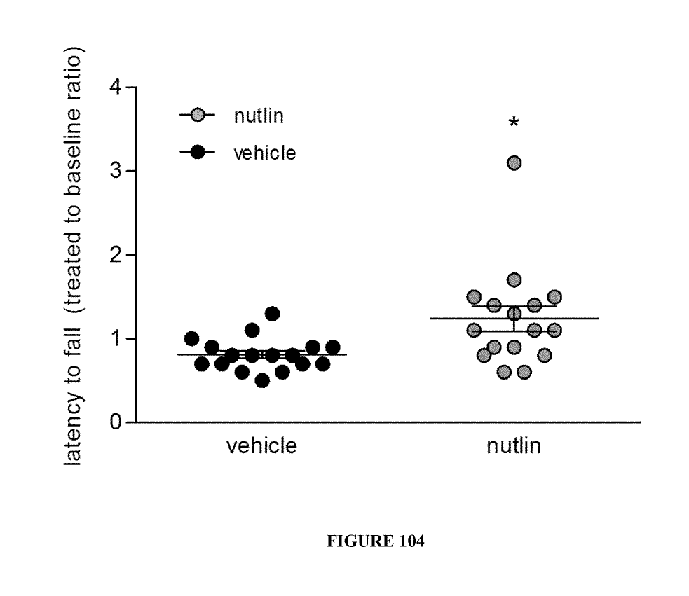

Mice treated with nutlin-3a showed an increased rotarod performance compared to controls after three days of training (p<0.05, t-test, n=17). The average of 4 runs for each animal and the average (±SEM) for each group are shown in FIG. 103. FIG. 104 shows the ratio between the latency to fall during the fourth session after treatment and the fourth session of baseline measurements is shown. Nutlin-3a treatment results in a higher average ratio (p<0.01, t-test, n=17). Values higher than 1 correspond to an increase in motor coordination after treatment. The average of 4 runs for each animal and the average (±SEM) for each group are shown.

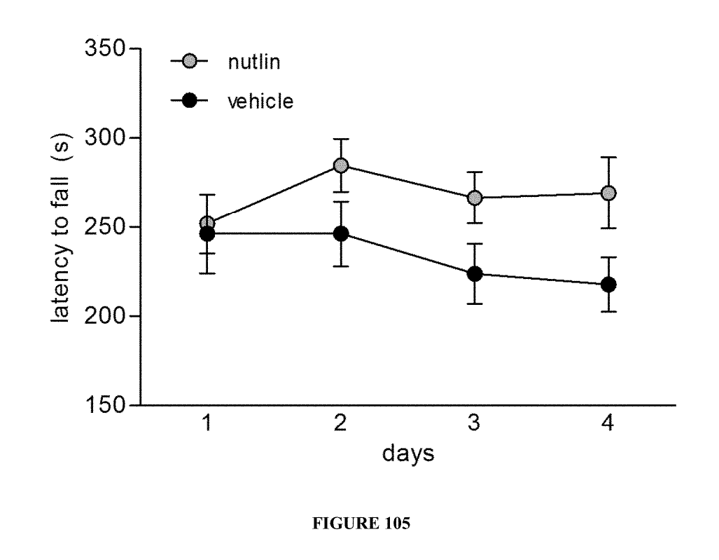

Nutlin-treated mice exhibit a better performance on the rotarod over the training period as shown in FIG. 105, (p<0.01, 2-way ANOVA, n=17). The average of 4 runs is shown for each day (±SEM).

What is claimed is:

1. A method of increasing life expectancy of a test subject,
wherein the test subject is a non-human mammal that has a transgene in its genome that contains a tissue-specific promoter sequence controlling expression of a polypeptide so as to cause said polypeptide to be expressed selectively in senescent cells in the test subject;
wherein contacting senescent cells in the subject that express the polypeptide with a particular small molecule compound results in selective elimination of at least some of the senescent cells;
wherein the method comprises administering said compound to the subject so as to selectively eliminate senescent cells in the subject, thereby increasing the life expectancy of the subject.

2. The method of claim 1, wherein contacting a senescent cell in the test subject with the compound causes the polypeptide expressed from the transgene to directly initiate apoptosis in the cell, thereby selectively eliminating the senescent cell.

3. The method of claim 2, wherein the polypeptide is a caspase.

4. The method of claim 2, wherein the polypeptide comprises an FKBP polypeptide sequence.

5. The method of claim 2, wherein the polypeptide is an FKBP-caspase 8 fusion polypeptide.

6. The method of claim 2, wherein the compound is AP20187.

7. The method of claim 1, wherein the compound is a prodrug, and contacting a senescent cell in the test subject with the prodrug causes the polypeptide expressed from the transgene to convert the prodrug to an active form of the prodrug that is lethal to the cell.

8. The method of claim 7, wherein the polypeptide is a thymidine kinase.

9. The method of claim 7, wherein the prodrug is ganciclovir.

10. The method of claim 1, wherein the tissue-specific promoter is a p16 promoter.

11. The method of claim 1, wherein the compound is administered to the test subject twice weekly.

12. The method of claim 1, wherein the increase in life expectancy is attributable at least in part to sustained cardiac performance resulting from elimination of senescent cells from the test subject.

13. The method of claim 1, wherein the increase in life expectancy is attributable at least in part to increased tumor latency resulting from elimination of senescent cells from the test subject.

14. The method of claim 1, wherein the increase in life expectancy is attributable at least in part to reduced glomerulosclerosis resulting from elimination of senescent cells from the test subject.

15. The method of claim 1, wherein the average increase in lifespan resulting from administration of the compound to test subjects having said transgene in their genome is at least 20%.


SEQUENCE LISTING (22 sequences)

SEQ ID NO: 1 - DNA, 58 bp, Artificial Sequence
  Description of Artificial Sequence Synthetic oligonucleotide
ccggccgggagatagtgatgaagtactcgagtacttcatcactatctcccggtttttg

SEQ ID NO: 2 - DNA, 21 bp, Artificial Sequence
  Description of Artificial Sequence Synthetic oligonucleotide
ccgggagatagtgatgaagta

SEQ ID NO: 3 - DNA, 58 bp, Artificial Sequence
  Description of Artificial Sequence Synthetic oligonucleotide
ccgggtgatgaagtacatccattatctcgagataatggatgtacttcatcactttttg

SEQ ID NO: 4 - DNA, 21 bp, Artificial Sequence
  Description of Artificial Sequence Synthetic oligonucleotide
gtgatgaagtacatccattat

SEQ ID NO: 5 - DNA, 58 bp, Artificial Sequence
  Description of Artificial Sequence Synthetic oligonucleotide
ccggagagtgacagtggattgcattctcgagaatgcaatccactgtcactcttttttg

SEQ ID NO: 6 - DNA, 21 bp, Artificial Sequence
  Description of Artificial Sequence Synthetic oligonucleotide
agagtgacagtggattgcatt

SEQ ID NO: 7 - DNA, 58 bp, Artificial Sequence
  Description of Artificial Sequence Synthetic oligonucleotide
ccgggctcactcttcagtcggaaatctcgagatttccgactgaagagtgagctttttg

SEQ ID NO: 8 - DNA, 19 bp, Artificial Sequence
  Description of Artificial Sequence Synthetic oligonucleotide
gctcactcttcagtcggaa

SEQ ID NO: 9 - DNA, 58 bp, Artificial Sequence
  Description of Artificial Sequence Synthetic oligonucleotide
ccgggtggaactctatgggaacaatctcgagattgttcccatagagttccactttttg

SEQ ID NO: 10 - DNA, 19 bp, Artificial Sequence
  Description of Artificial Sequence Synthetic oligonucleotide
gtggaactctatgggaaca

SEQ ID NO: 11 - DNA, 58 bp, Artificial Sequence
  Description of Artificial Sequence Synthetic oligonucleotide
ccgggtttagtgatgtggaagagaactcgagttctcttccacatcactaaactttttg

SEQ ID NO: 12 - DNA, 19 bp, Artificial Sequence
  Description of Artificial Sequence Synthetic oligonucleotide
gtttagtgatgtggaagag

SEQ ID NO: 13 - DNA, 58 bp, Artificial Sequence; identical to SEQ ID NO: 7
  Description of Artificial Sequence Synthetic oligonucleotide

SEQ ID NO: 14 - DNA, 21 bp, Artificial Sequence
  Description of Artificial Sequence Synthetic oligonucleotide
gctcactcttcagtcggaaat

SEQ ID NO: 15 - DNA, 58 bp, Artificial Sequence
  Description of Artificial Sequence Synthetic oligonucleotide
ccggtggcagactttgtaggttatactcgagtataacctacaaagtctgccatttttg

SEQ ID NO: 16 - DNA, 19 bp, Artificial Sequence
  Description of Artificial Sequence Synthetic oligonucleotide
tggcagactttgtaggtta

SEQ ID NO: 17 - DNA, 58 bp, Artificial Sequence
  Description of Artificial Sequence Synthetic oligonucleotide
ccgggtcaacaaggagatggaaccactcgagtggttccatctccttgttgactttttg

SEQ ID NO: 18 - DNA, 19 bp, Artificial Sequence
  Description of Artificial Sequence Synthetic oligonucleotide
gtcaacaaggagatggaac

SEQ ID NO: 19 - DNA, 58 bp, Artificial Sequence
  Description of Artificial Sequence Synthetic oligonucleotide
ccggcagaagggttatgtctgtggactcgagtccacagacataacccttctgtttttg

SEQ ID NO: 20 - DNA, 19 bp, Artificial Sequence
  Description of Artificial Sequence Synthetic oligonucleotide
cagaagggttatgtctgtg

SEQ ID NO: 21 - DNA, 59 bp, Artificial Sequence
  Description of Artificial Sequence Synthetic oligonucleotide
ccggccattagatgagtgggatttactcgagtaaatcccactcatctaatggttttttg

SEQ ID NO: 22 - DNA, 21 bp, Artificial Sequence
  Description of Artificial Sequence Synthetic oligonucleotide
ccattagatgagtgggattta